(19) United States
(12) Patent Application Publication
(10) Pub. No.: US 2008/0200501 A1
(43) Pub. Date: Aug. 21, 2008

(54) NOVEL CANNABINOID RECEPTOR LIGANDS, PHARMACEUTICAL COMPOSITIONS CONTAINING THEM, AND PROCESSES FOR THEIR PREPARATION

(75) Inventors: Meyyappan Muthuppalaniappan, Navi Mumbai (IN); Kumar Sukeerthi, Navi Mumbai (IN); Gopalan Balasubramanian, Secunderabad (IN); Srinivas Gullapalli, Navi Mumbai (IN); Neelima Khairatkar Joshi, Thane (west) (IN); Shridhar Narayanan, Than (west) (IN); Pallavi V. Kamik, Thane (IN)

Correspondence Address: DARBY & DARBY P.C., P.O. BOX 770, Church Street Station, New York, NY, 10008-0770, US

(73) Assignee: Glenmark Pharmaceuticals S.A., La Chaux-de-Fonds (CH)

(21) Appl. No.: 11/934,186

(22) Filed: Nov. 2, 2007

(30) Foreign Application Priority Data
Nov. 3, 2006 (IN) 1838/MUM/2006

(51) Int. Cl. A61K 31/416; A61P 3/00; C07D 413/02; C07D 231/54; A61K 31/4245; A61K 31/422; C07D 471/00; A61K 31/4709; A61K 31/4725

(52) U.S. Cl. 514/307; 514/374; 514/403; 514/364; 548/143; 548/215; 548/359.1; 546/146; 546/168; 514/314

(57) ABSTRACT

The present invention relates to novel cannabinoid receptor modulators, in particular cannabinoid 1 (CB1) or cannabinoid 2 (CB2) receptor modulators, and uses thereof for treating diseases, conditions and/or disorders modulated by a cannabinoid receptor (such as pain, neurodegenative disorders, eating disorders, weight loss or control, and obesity).

This application claims the benefit of Indian Patent Application No. 1838/MUM/2006, filed Nov. 3, 2006.

### FIELD OF THE INVENTION

The present invention relates to novel cannabinoid receptor modulators, in particular cannabinoid 1 (CB1) or cannabinoid 2 (CB2) receptor modulators, and uses thereof for treating diseases, conditions and/or disorders modulated by a cannabinoid receptor (such as pain, neurodegenative disorders, eating disorders, weight loss or control, and obesity).

### BACKGROUND OF THE INVENTION

The endogenous cannabinoid system comprises two main receptors, CB1 and CB2, and a number of ligands including anandamide and virodhamine which demonstrate the greatest activity at the cannabinoid receptor (Jonathan A W & Louis J A, Obes Man., 5-19, 2005). Anandamide, which is produced postsynaptically, is the main fatty acid involved in the system. It gains access to the extra cellular space and activates CB1 cannabinoid receptors located on presynaptic nerve terminals. This activation causes presynaptic inhibition of γ-aminobutyric acid or glutamate through inhibition of calcium channels, while simultaneously interfering with vesicle release and activating potassium channels.

However, anandamide is prone to rapid enzymatic hydrolysis. This represents a serious drawback in its use as a drug because, inter alia, substances which are susceptible to hydrolytic cleavage may undergo changes in the gastrointestinal tract.

CB1 receptors are predominantly located in the brain and other neurons, while CB2 receptors are predominantly located in immune cells. Stimulation of these receptors is known to affect the central and peripheral action on lipid and glucose metabolism in adipose tissue and most notably, helps to regulate food intake, energy balance and nicotine dependence as well as regulate fear and anxiety.

There is evidence suggesting that CB1 agonists or antagonists, respectively, increase or decrease the motivation to work for palatable ingesta (Gallate J E and McGregor I S, Psychopharmacology, 142, 302-308, 1999 and Gallate J E, Saharov T, Mallet P E and McGregor I S, 1999, Eur. J. Pharmacol., 370, 233-240, 1999). Cannabinoids appear to directly stimulate eating by actions on appetitive processes, making food stimuli more salient and rapidly inducing eating even in satiated animals (Williams C M and Kirkham T C, Physiol. Behav., 76, 241-250, 2002).

Current data reveals that cannabinoids mediate suppression of inflammation in vitro and in vivo through stimulation of CB2 receptors (Ehrhart J, et. al. J. Neuroinflammation, 2, 29, 2005). The inflammatory mediators such as nitric oxide, cytokines, and chemokines play an important role in microglial cell-associated neuron cell damage. Activated microglial cells have been implicated in a number of neurodegenerative disorders, including Alzheimer's disease, multiple sclerosis, HIV and dementia.

Known CB modulators include naphthalen-1yl-(4-pentyloxy-naphthalen-1-yl)methanone (believed to be SAB-378), 4-(2,4-dichlorophenylamino)-N-(terahydro-pyran-4ylmethyl)-2-trifluromethyl-benzamide (GW-842166X), N-(1-piperidinyl)-5-(4-chlorophenyl)-1-(2,4-dichlorophenyl)-4-methylpyrazole-3-carbox-amide (SR141716A), 3-(4-chlorophenyl-N′-(4-chlorophenyl)sulfonyl-N-methyl-4-phenyl-4,5-dihydro-1H-pyrazole-1-carboxamide (SLV-319), and (R)-(+)-[2,3-dihydro-5-methyl-3-[4-morpholinylmethyl]-pyrrolo-[1,2,3-de]-1,4-benzoxazin-6-yl](1-naphthyl)methanone (WIN 55212-2).

These modulators have reached advanced stages of clinical trials for the treatment of pain, neurodegenerative disorders, psychotic disorders, neurological syndromes, diseases or disorders, eating disorders, Alzheimer's disease, alcohol dependency, diabetes, obesity and/or smoking cessation.

U.S. Pat. Nos. 5,624,941, 6,028,084, and 6,509,367, PCT Publication Nos. WO 98/31227, WO 98/41519, WO 98/43636, WO 98/43635, and WO 06/129178, and European Publication No. EP 0 658 546 disclose certain substituted pyrazoles having activity against the cannabinoid receptors.

There exists an unmet need for treatment of alcohol abuse. Health risks associated with alcoholism include impaired motor control and decision making, cancer, liver disease, birth defects, heart disease, drug/drug interactions, pancreatitis and interpersonal problems. Studies have suggested that endogenous cannabinoid tone plays a critical role in the control of ethanol intake. The endogenous CB1 receptor antagonist SR-141716A has been shown to block voluntary ethanol intake in rats and mice. (See, Arnone, M., et al., “Selective Inhibition of Sucrose and Ethanol Intake by SR141716, an Antagonist of Central Cannabinoid (CB1) Receptors,” Psychopharmacol, 132,104-106 (1997)). For a review, see, Hungund, B. L and B. S. Basavarajappa, “Are Anadamide and Cannabinoid Receptors involved in Ethanol Tolerance? A Review of the Evidence,” Alcohol & Alcoholism, 35(2) 126-133, 2000.

Current treatments for alcohol abuse or dependence generally suffer from non-compliance or potential hepatotoxicity. There is an unmet need for more effective treatments of alcohol abuse/dependence. A drug candidate should have good pharmacokinetic properties enabling a suitable dosage regimen for the required pharmacodynamic action.

There still exists a need for safer and more effective therapeutic treatments for diseases, conditions and/or disorders modulated by cannabinoid receptors (such as pain and obesity), including those modulated by CB1 or CB2 receptors.

### SUMMARY OF THE INVENTION

The present invention is directed to bridged bicyclic compounds which are cannibinoid modulators. These compounds are particularly useful as CB2 agonists. These compounds are suitable for the treatment of diseases, conditions or disorders mediated by cannibinoid modulators (such as by CB2 agonists) (e.g., inflammatory disorders and pain).

The present invention relates to a compound of formula (I):

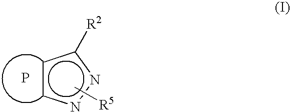

or a pharmaceutically acceptable salt thereof, a pharmaceutically acceptable solvate thereof, a regioisomer thereof, a stereoisomer thereof, a prodrug thereof, or a N-oxide thereof,
wherein

ring P is a bridged bicyclic ring system having 0-2 double bonds, which is optionally substituted with up to 10 R¹ groups;

each occurrence of R¹ is independently hydrogen, nitro, cyano, halogen, alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclyl, heterocyclylalkyl, NR³R⁴, C(═B)R⁴, C(O)OR⁴, C(O)NR³R⁴, S(O)ₘR⁴, S(O)ₘNR³R⁴, OR⁴, SR⁴ or a protecting group;

alternatively, two R¹ groups, together with the atoms to which they are attached, form an aryl or heteroaryl group;

each occurrence of R³ and R⁴ is independently hydrogen, nitro, halo, alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclyl, heterocyclylalkyl, NR^{a}R^{b}, C(═B)R^{b}, C(O)OR^{b}, C(O)NR^{a}R^{b}, S(O)ₘR^{b}, S(O)ₘNR^{a}R^{b}, OR^{b}, or SR^{b}, or R^{a} and R^{b}, when bound to a common atom, are joined to form a 3-7 membered cyclic ring containing one or more heteroatoms or groups selected from N, O, S, C(O) or SO₂, wherein the 3-7 membered cyclic ring is optionally substituted with one or more R^{c} groups;

each occurrence of R^{a} and R^{b} is independently hydrogen, alkyl, alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclyl, or heterocyclylalkyl;

each occurrence of R^{c} is independently hydrogen, alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclyl, or heterocyclylalkyl;

R² is
i) an optionally mono-, di-, or tri-substituted group selected from alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclyl, or heterocyclylalkyl, wherein the optional substituents are independently selected from —C(O)H, alkyl, aryl, and cycloalkyl (e.g., bridged cycloalkyl) which are unsubstituted or substituted with one or more of hydroxy, halogen, nitro, alkyl, alkoxy, COOR″ (wherein R″ is hydrogen or alkyl), or CONR^{3a}R^{4a}; or
ii) C(O)NHNHR^{3a}, C(O)NHNHC(O)R^{4a}, C(═S)NH₂, C(═NR^{3a})R^{4a}, (CH₂)ₚNR^{3a}R^{4a}, CH═CR^{3a}R^{4a}, CF₂R^{4a}, CHFR^{4a}, (CH₂)ₚOR^{3a}, C(═B)R^{3a}, C(O)OR^{3a}, NR^{3aa}CONR^{3a}R^{4a}, S(O)ₘR^{3a}, S(O)ₘNR^{3a}R^{4a}, NR^{3a}COR^{4a}, NR^{3a}CSR^{4a}, NR^{3a}SO₂R^{4a}, C(═NR^{3aa})NR^{3a}R^{4a}, C(═NOR^{3a})R^{4a}, C(═NNR^{3a})R^{4a}, (CH₂)ₚ—CONHR^{3a}, C≡C—R^{3a}, C(O)NH(CH₂)ₚC(O)R^{3a}, (CH₂)ₚ—CONR^{3a}R^{4a}, or C(OR^{3a})R^{4a},
   with the proviso that R² is not

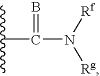

where B is O, S, or NR^{h}, and R^{f}, R^{g}, and R^{h} are independently any atom or group;

each occurrence of R^{3a}, R^{3aa}, and R^{4a} is independently i) hydrogen, nitro, or halogen, or ii) an optionally substituted group selected from alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclyl, heterocyclylalkyl, NR^{a}R^{b}, C(═B)R^{b}, C(O)OR^{b}, C(O)NR^{a}R^{b}, S(O)ₘR^{b}, S(O)ₘNR^{a}R^{b}, OR^{b}, or SR^{b}, or R^{3a} and R^{4a}, when bound to a common atom, are joined to form a 3-7 membered cyclic ring containing one or more heteroatoms or groups selected from N, O, S, C(O) or SO₂, wherein the 3-7 membered cyclic ring is optionally substituted with one or more R^{c} groups;

R⁵ is hydrogen, alkyl, aryl, heteroaryl, or heterocyclyl, and R⁵ is optionally mono-, di- or tri-substituted with substituents selected from nitro, cyano, acyl, halogen, alkyl, haloalkyl, alkenyl, alkynyl, cycloalkyl, cycloalkylalkyl, cycloalkenyl, cycloalkenylalkyl, aryl, arylalkyl, heteroaryl, heteroarylalkyl, heterocyclic, heterocyclylalkyl, NR³R⁴, C(═B)R⁴, C(O)OR⁴, C(O)NR³R⁴, S(O)ₘR⁴, S(O)ₘNR³R⁴, OR⁴, SR⁴ or a protecting group;

each occurrence of m and p is independently 0, 1 or 2; and

each occurrence of B is O, S or NR^{b}.

Preferably, ring P in the compound of formula (I) is selected from

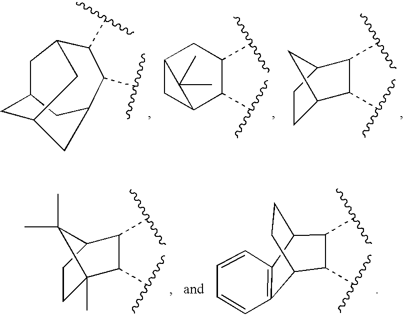

In a more preferred embodiment, ring P is

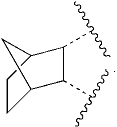

According to one embodiment, R⁵ is attached to the nitrogen ring atom in formula (I) adjacent to ring P.

According to another embodiment, R⁵ is attached to the nitrogen ring atom in formula (I) distal to ring P.

Preferably, R⁵ in the compound of formula (I) is optionally substituted aryl. According to one embodiment, R⁵ is attached to the nitrogen ring atom in formula (I) adjacent to ring P, and R⁵ is an optionally substituted aryl. According to another embodiment, R⁵ is attached to the nitrogen ring atom in formula (I) distal to ring P and R⁵ is an optionally substituted aryl.

According to one more preferred embodiment, R⁵ is a mono- or di-halogenated aryl, such as a R⁵ is a mono- or di-halogenated phenyl. Suitable R⁵ groups include, but are not limited to, 2,4-difluorophenyl, 2,4-dichlorophenyl, 4-fluorophenyl, 4-chlorophenyl, 2-fluoro-4-chlorophenyl, and 4-bromophenyl. A preferred R⁵ group is 2,4-difluorophenyl.

According to one preferred embodiment, R² in the compound of formula (I) is COR^{3a}, where R^{3a} is an optionally substituted group selected from alkyl or aryl.

According to another preferred embodiment, R² in the compound of formula (I) is CH═CR^{3a}R^{4a} or C≡C—R^{3a}.

According to another embodiment, the invention relates to compounds of the formula (Ia)

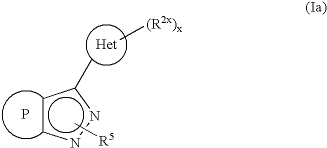

or a pharmaceutically acceptable salt thereof, a pharmaceutically acceptable solvate thereof, a regioisomer thereof, a stereoisomer thereof, or a N-oxide thereof,
wherein

‘Het’ is a 5-membered heteroaryl or heterocyclyl;

each occurrence of R^{2x} is indepedently an optionally substituted alkyl, cycloalkyl, or aryl, where the optional substituents are selected from hydroxy, halogen, nitro, alkyl, alkoxy, COOR″ (where R″ is hydrogen or alkyl), and CONH₂;

ring P and R⁵ is as defined with respect to formula (I); and

x is an integer selected from 0-3.

The various embodiments of ring P and R⁵ described for formula (I) equally apply to formula (Ia).

According to another embodiment, the invention relates to compounds of the formula (Ib)

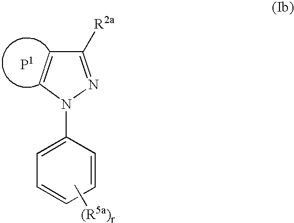

or a pharmaceutically acceptable salt thereof, a pharmaceutically acceptable solvate thereof, a regioisomer thereof, a stereoisomer thereof, a prodrug thereof, or a N-oxide thereof,
wherein

ring P¹ is

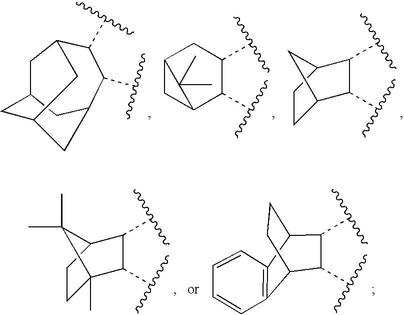

R^{2a} is selected from
i) an optionally substituted group selected form alkyl and aryl, wherein the optional substituents are hydroxy, CH₂OH, halogen, formaldehyde, and alkyl;
ii) COR^{3a1} wherein R^{3a1} is selected from an optionally substituted alkyl or aryl, wherein the optional substituents are selected from hydroxy and halogen;
iii) (CH₂)_{q1}—NR^{3a2}R^{3a3} wherein R^{3a2} and R^{3a3} are independently selected from hydrogen, substituted or unsubstituted alkyl, and substituted or unsubstituted arylalkyl, and q1 is 0 or 1;
iv) (CH₂)_{q2}—OR^{3a4} wherein R^{3a4} is arylalkyl and q2 is 1 or 2;
v) (CH₂)_{q3}—CONHR^{3a5} wherein R^{3a5} is alkyl and q3 is 1 or 2;
vi) NHCOR^{3a6} wherein R^{3a6} is alkyl;
vii) NHSO₂R^{3a7} wherein R^{3a7} is aryl;
viii) (CH═CH)—R^{3a8} wherein R^{3a8} is alkyl;
ix) COOR^{3a9} wherein R^{3a9} is alkyl;
x) C≡C—R^{3a10} wherein R^{3a10} is selected from alkyl and aryl;
xi) an optionally substituted 5-membered heteroaryl or an optionally substituted 5-membered heterocyclyl, wherein the optional substituents are selected from alkyl, aryl, cycloalkyl (e.g., bridged cycloalkyl), each of which is optionally substituted with hydroxy, halogen, COOH, or CONH₂;
xii) C(═NOR^{3a11})R^{3a12} wherein R^{3a11} and R^{3a12} are independently selected from hydrogen and optionally substituted alkyl;
xiii) CF₂R^{4a}; and
xiv) CHFR^{4a};

each occurrence of R^{5a} is selected from nitro, halogen, and alkoxy; and

r is an integer selected from 0-3.

According to one preferred embodiment, r is 1 or 2 and each occurrence of R^{5a} is independently halogen (and preferably chloro or fluoro).

According to another embodiment, the invention relates to compounds of the formula (II)

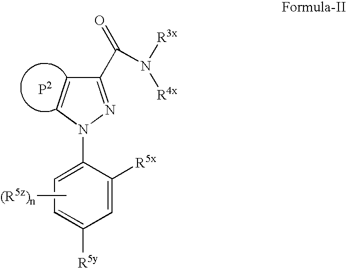

or a pharmaceutically acceptable salt thereof, a pharmaceutically acceptable solvate thereof, a regioisomer thereof, a stereoisomer thereof, a prodrug thereof, or a N-oxide thereof,
wherein ring P² is

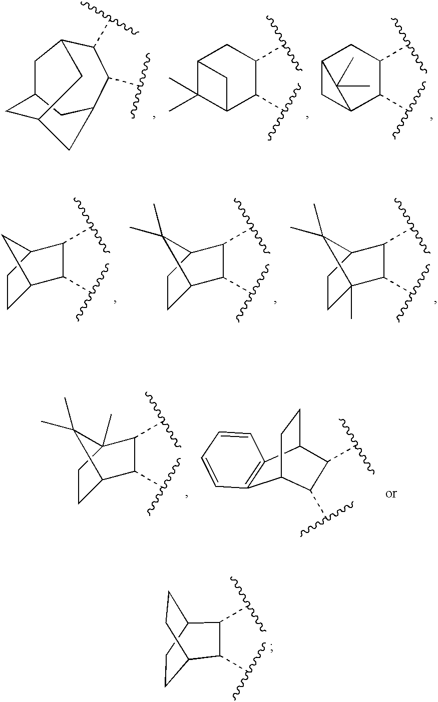

R^{3x} is hydrogen or substituted or unsubstituted alkyl and R^{4x} is substituted or unsubstituted alkyl or substituted or unsubstituted cycloalkyl, or R^{3x} and R^{4x} are joined together to form a substituted or unsubstituted heterocyclyl;

B is O or S;

R^{5x} and R^{5y} are independently hydrogen, halogen (F, Cl or Br), nitro, C₁-C₄ alkyl, C₁-C₄ alkoxy, trifluoromethyl, C(O)O(C₁-C₃)alkyl, N(H)C(O)O(C₁-C₃)alkyl or NHC(O)CH₃;

R^{5z} is halogen (e.g., F, Cl or Br); and

n is 0, 1 or 2.

According to another embodiment, the invention relates to compounds of the formula (IIa)

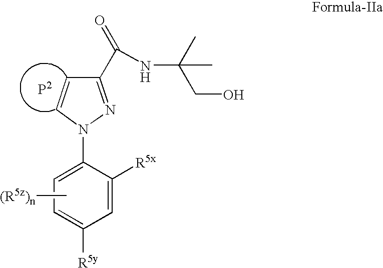

or a pharmaceutically acceptable salt thereof, a pharmaceutically acceptable solvate thereof, a regioisomer thereof, a stereoisomer thereof, a prodrug thereof, or a N-oxide thereof,
wherein ring P^{2 is}

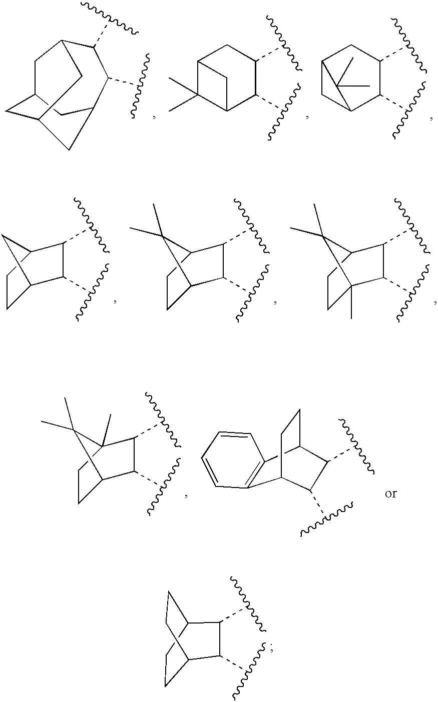

R^{5x} and R^{5y} are independently hydrogen, halogen (e.g., F, Cl or Br), nitro, C₁-C₄ alkyl, C₁-C₄ alkoxy, trifluoromethyl, C(O)O(C₁-C₃ alkyl), N(H)C(O)O(C₁-C₃ alkyl) or NHC(O)CH₃;

R^{5z} is halogen (e.g., F, Cl or Br); and

n is 0, 1 or 2.

Preferably, one or both of R^{5x} and R^{5y} are halogen (such as bromo, chloro, or fluoro). According to a preferred embodiment, R^{5x} and R^{5y} are independently halogen. For example, in one embodiment, R^{5x} and R^{5y} are both fluoro. In another embodiment, R^{5x} is fluoro and R^{5y} is chloro. In yet another embodiment, R^{5x} is hydrogen and R^{5y} is halogen (such as bromo, chloro, or fluoro).

According to one preferred embodiment, ring P² is

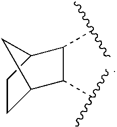

Preferably, n is 0 or 1. According to one preferred embodiment, n is 0.

According to another embodiment, the invention relates to compounds of the formula (IIb)

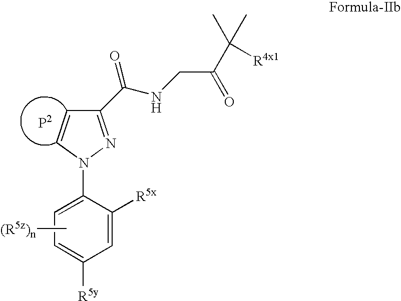

or a pharmaceutically acceptable salt thereof, a pharmaceutically acceptable solvate thereof, a regioisomer thereof, a stereoisomer thereof, a prodrug thereof, or a N-oxide thereof,
wherein ring P² is

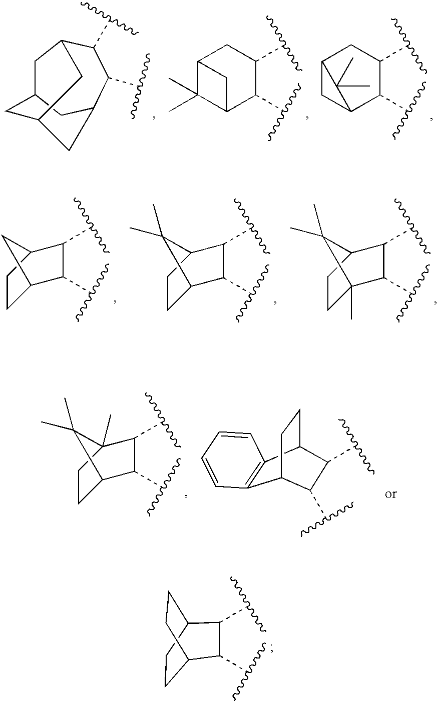

R^{4x1} is independently selected from H and COOR′″ wherein R′″ is H or alkyl;

R^{5x} and R^{5y} are independently hydrogen, halogen (e.g., F, Cl or Br), nitro, C₁-C₄ alkyl, C₁-C₄ alkoxy, trifluoromethyl, C(O)O(C₁-C₃ alkyl), N(H)C(O)O(C₁-C₃ alkyl) or NHC(O)CH₃;

R^{5z} is halogen (e.g., F, Cl or Br); and

n is 0, 1 or 2.

Preferably, one or both of R^{5x} and R^{5y} are halogen (such as bromo, chloro, or fluoro). According to a preferred embodiment, R^{5x} and R^{5y} are independently halogen. For example, in one embodiment, R^{5x} and R^{5y} are both fluoro. In another embodiment, R^{5x} is fluoro and R^{5y} is chloro. In yet another embodiment, R^{5x} is hydrogen and R^{5y} is halogen (such as bromo, chloro, or fluoro), nitro, or C₁-C₄ alkoxy.

According to one preferred embodiment, ring P² is

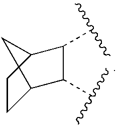

Preferably, n is 0 or 1. According to one preferred embodiment, n is 0.

According to another embodiment, the invention relates to compounds of the formula (IIc)

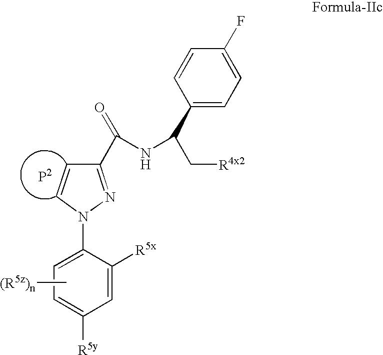

or a pharmaceutically acceptable salt thereof, a pharmaceutically acceptable solvate thereof, a regioisomer thereof, a stereoisomer thereof, a prodrug thereof, or a N-oxide thereof,
wherein ring P² is

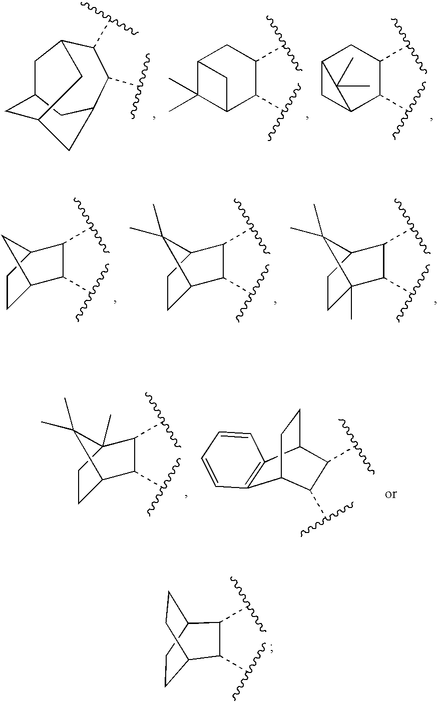

R^{4x2} is hydrogen, OR′″, or COOR′″ wherein R′″ is hydrogen or alkyl;

R^{5x} and R^{5y} are independently hydrogen, halogen (e.g., F, Cl or Br), nitro, C₁-C₄ alkyl, C₁-C₄ alkoxy, trifluoromethyl, C(O)O(C₁-C₃ alkyl), N(H)C(O)O(C₁-C₃ alkyl) or NHC(O)CH₃;

R^{5z} is halogen (e.g., F, Cl or Br); and

n is 0, 1 or 2.

According to one embodiment, R^{4x2} is selected from hydroxy, methoxy, and CO₂CH₃.

Preferably, one or both of R^{5x} and R^{5y} are halogen (such as bromo, chloro, or fluoro). According to a preferred embodiment, R^{5x} and R^{5y} are independently halogen. For example, in one embodiment, R^{5x} and R^{5y} are both fluoro.

According to one preferred embodiment, ring P² is

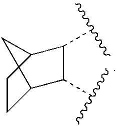

Preferably, n is 0 or 1. According to one preferred embodiment, n is 0.

According to another embodiment, the invention relates to compounds of the formula (IId)

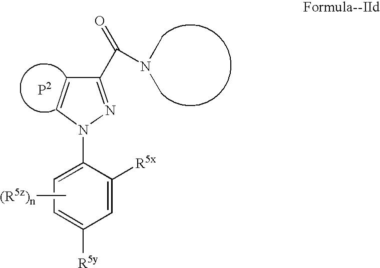

or a pharmaceutically acceptable salt thereof, a pharmaceutically acceptable solvate thereof, a regioisomer thereof, a stereoisomer thereof, a prodrug thereof, or a N-oxide thereof,
wherein, ring P² is

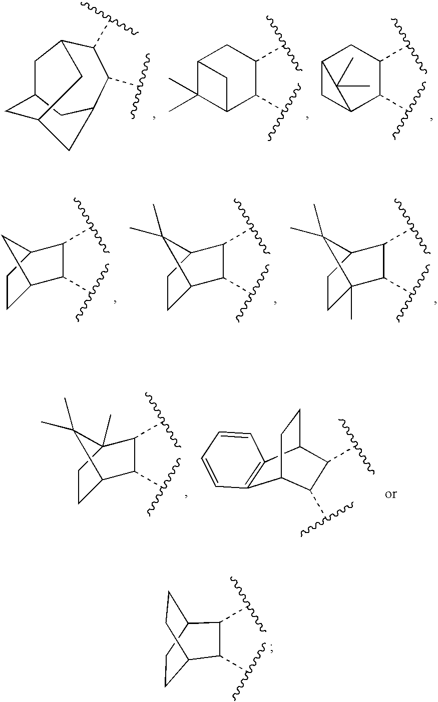

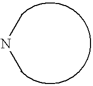

is independently selected from tetrahydroquinoline and tetrahydroisoquinoline;

R^{5x} and R^{5y} are independently hydrogen, halogen (e.g., F, Cl or Br), nitro, C₁-C₄ alkyl, C₁-C₄ alkoxy, trifluoromethyl, C(O)O(C₁-C₃ alkyl), N(H)C(O)O(C₁-C₃ alkyl) or NHC(O)CH₃;

R^{5z} is halogen (e.g., F, Cl or Br); and

n is 0, 1 or 2.

Preferably, one or both of R^{5x} and R^{5y} are halogen (such as bromo, chloro, or fluoro). According to a preferred embodiment, R^{5x} and R^{5y} are independently halogen. For example, in one embodiment, R^{5x} and R^{5y} are both fluoro.

According to one preferred embodiment, ring P² is

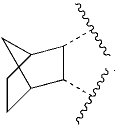

According to one embodiment,

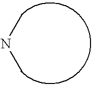

is tetrahydroquinoline. According to another embodiment,

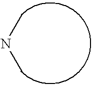

is tetrahydroisoquinoline.

Preferably, n is 0 or 1. According to one preferred embodiment, n is 0.

According to another embodiment, the invention relates to compounds of the formula (IIe)

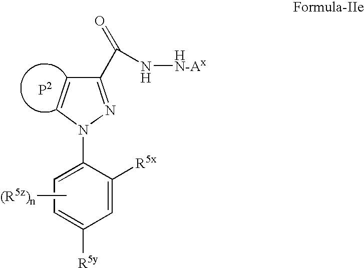

or a pharmaceutically acceptable salt thereof, a pharmaceutically acceptable solvate thereof, a regioisomer thereof, a stereoisomer thereof, a prodrug thereof, or a N-oxide thereof,
wherein ring P² is

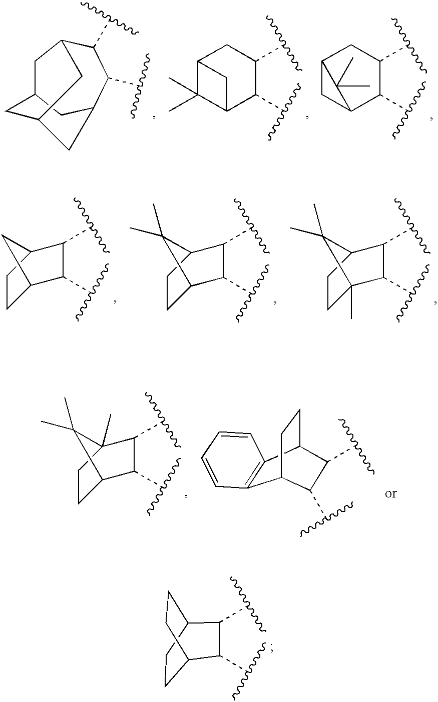

A^{x} is independently selected from alkyl and C(O)A^{y}, where A^{y} is alkyl or cycloalkyl;

R^{5x} and R^{5y} are independently hydrogen, halogen (e.g., F, Cl or Br), nitro, C₁-C₄ alkyl, C₁-C₄ alkoxy, trifluoromethyl, C(O)O(C₁-C₃ alkyl), N(H)C(O)O(C₁-C₃ alkyl) or NHC(O)CH₃;

R^{5z} is halogen (e.g., F, Cl or Br); and

n is 0, 1 or 2.

Preferably, one or both of R^{5x} and R^{5y} are halogen (such as bromo, chloro, or fluoro). According to a preferred embodiment, R^{5x} and R^{5y} are independently halogen. For example, in one embodiment, R^{5x} and R^{5y} are both fluoro.

According to one preferred embodiment, ring P² is

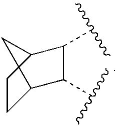

According to one embodiment, A^{x} is C₁-C₆ alkyl (e.g., tert-butyl). According to another embodiment, A^{x} is C(O)(C₁-C₆ alkyl) (e.g., C(O)C(CH₃)₃ or n-pentyl). According to yet another embodiment, A^{x} is C(O)A^{y} where A^{y} is cycloalkyl.

Preferably, n is 0 or 1. According to one preferred embodiment, n is 0.

Below are representative compounds, which are illustrative in nature only and do not limit to the scope of the invention.

### Compound Compound Name

### No

1. 5-(2,4-difluorophenyl)-4,5-diazatricyclo[5.2.1.0^{2,6}.]deca-2(6),3-dien-3-yl-phenylmethanone;
2. 1-[5-(2,4-diflurophenyl)-4,5-diazatricyclo[5.2.1.0^{2,6}]deca-2(6),3-dien-3-yl]-1-hexanone;
3. 5-(2,4-diflurophenyl)-4,5-diazatricyclo[5.2.1.0^{2,6}.]deca-2(6),3-dien-3-yl-1-naphthylmethanone;
4. 4-(2,4-Dichlorophenyl)-4,5-diazatricyclo[5.2.1.0^{2,6}]deca-2,5-dien-3-yl-phenylmethanone;
5. 1-[5-(2,4-diflurophenyl)-4,5-diazatricyclo[5.2.1.0^{2,6}]deca-2(6),3-dien-3-yl]-3,3,-dimethyl-1-butanol;
6. 1-{5-(2,4-diflurophenyl)-4,5-diazatricyclo[5.2.1.0^{2,6}.]deca-2(6),3-dien-3-yl]}-3,3,-dimethyl-1-butanone;
7. N2-[5-(2,4-diflurophenyl)-4,5-diazatricyclo[5.2.1.0^{2,6}]deca-2(6),3-dien-3-ylmethyl]-2-methyhyl-2-propanamine;
8. N2-[5-(2,4-diflurophenyl)-4,5-diazatricyclo[5.2.1.0^{2,6}]deca-2(6),3-dien-3-ylmethyl]-2-phenyl-2-propanamine hydrochloride;
9. N1-[5-(2,4-Difluorophenyl)-4,5-diazatricyclo[5.2.1.0^{2,6}]deca-2(6),3-dien-3-yl]-2,2-dimethylpropanamide;
10. 3-(2,4-Difluorophenyl)-5-sulfonamide-3,4-diazatricyclo[5.2.1.0^{2,6}]deca-2(6),4-diene;
11. N5,N5-Dibenzyl-3-(2,4-difluorophenyl)-3,4-diazatricyclo[5.2.1.0^{2,6}]deca-2(6),4-dien-5-amine;
12. (1S,8S)-N1-[5-(2,4-difluorophenyl)-9,9-dimethyl-4,5-diazatricyclo(6,1,1,0^{2,6})-deac-2(6), 3-dien-3-yl]-2,2-dimethyl propanamide;
13. 5-benzyloxymethyl-3-(2,4-diflurophenyl)-3,4-diazatricylo[5.2.1.0^{2,6}.]deca-2(6),4diene;
14. tert-Butyl-3-(2,4-difluorophenyl)-3,4-diazatricyclo[5.2.1.0^{2,6}]deca-2(6),4-diene-5-carboxylate;
15. 3-(2,4-Difluorophenyl)-5-(1-fluro-3,3-dimethylbutyl)-3,4-diazatricyclo[5.2.1.0^{2,6}]deca-2(6),4-diene;
16. N′-Pivaloyl-3-(2,4-Diflurophenyl)-3,4-diazatricyclo[5.2.1.0^{2,6}]deca-2(6),4-diene-5-carbohydrazide;
17. N′-1-Hexanoyl-3-(2,4-diflurophenyl)-3,4-diazatricyclo[5.2.1.0^{2,6}]deca-2(6),4-diene-5-carbohydrazide;
18. N′-1-(Adamantanecarbonyl)-3-(2,4-diflurophenyl)-3,4-diazatricyclo[5.2.1.0^{2,6}]deca-2(6),4-diene-5-carbohydrazide;
19. N′-(Cyclohexanecarbonyl)-3-(2,4-diflurophenyl)-3,4-diazatricyclo[5.2.1.0^{2,6}]deca-2(6),4-diene-5-carbohydrazide;
20. 2-(tert-Butyl)-5-[5-(2,4-difluorophenyl)-4,5-diazatricyclo[5.2.1.0^{2,6}]deca-2(6),3-dien-3yl]-1,3,4-oxadiazole;
21. 2-[5-(2,4-Difluorophenyl)-4,5-diazatricyclo[5.2.1.0^{2,6}]deca-2(6),3-dien-3yl]-5-Pentyl-1,3,4-oxadiazole;
22. 2-(1-Adamantly)-5-[5-(2,4-difluorophenyl)-4,5-diazatricyclo[5.2.1.0^{2,6}]deca-2(6),3-dien-3yl]-1,3,4-oxadiazole;
23. 2-(Cyclohexyl)-5-[5-(2,4-difluorophenyl)-4,5-diazatricyclo[5.2.1.0^{2,6}]deca-2(6),3-dien-3yl]-1,3,4-oxadiazole;
24. 2-(tert-Butyl)-5-[5-(2,4-difluorophenyl)-4,5-diazatricyclo[5.2.1.0^{2,6}]deca-2(6),3-dien-3yl]-1,3,4-thiadiazole;
25. 5-[5-(tert-Butyl)-1H-1,2,4-triazol-3-yl]-3-(2,4-difluorophenyl)-3,4-diazatricyclo[5.2.1.0^{2,6}]deca-2(6),4-diene;
26. 5-(tert-Butyl)-3-[5-(2,4-difluorophenyl)-4,5-diazatricyclo[5.2.1.0^{2,6}]deca-2(6),3-dien-3yl]-1,2,4-oxadiazole;
27. 5-(tert-Butyl)-3-[(1S,7R)-5-(2,4-difluorophenyl)-4,5-diazatricyclo[5.2.1.0^{2,6}]deca-2(6),3-dien-3yl]-1,2,4-oxadiazole;
28. 5-(tert-Butyl)-3-[(1R,7S)-5-(2,4-difluorophenyl)-4,5-diazatricyclo[5.2.1.0^{2,6}]deca-2(6),3-dien-3yl]-1,2,4-oxadiazole;
29. 3-[5-(2,4-Difluorophenyl)-4,5-diazatricyclo[5.2.1.0^{2,6}]deca-2(6),3-dien-3yl]-5-phenyl-1,2,4-oxadiazole;
30. 3-(2,4-diflurophenyl)-5[(E)-3,3-dimethyl-1-butenyl}-3,4-diazatricyclo[5.2.1.0^{2,6}]deca-2(6),4-diene;
31. 3-(2,4-Difluorophenyl)-5-(3,3-dimethylbutyl)-3,4-diazatricyclo[5.2.1.0^{2,6}]deca-2(6),4-diene;
32. 3-(2,4-Difluorophenyl)-5-(3,3-dimethyl-1-butynyl)-3,4-diazatricyclo[5.2.1.0^{2,6}]deca-2(6),4-diene;
33. (1S,7R)-3-(2,4-Difluorophenyl)-5-(3,3-dimethyl-1-butynyl)-3,4-diazatricyclo[5.2.1.0^{2,6}]deca-2(6),4-diene;
34. (1R,7S)-3-(2,4-Difluorophenyl)-5-(3,3-dimethyl-1-butynyl)-3,4-diazatricyclo[5.2.1.0^{2,6}]deca-2(6),4-diene;
35. 1-[5-(2,4-Difluorophenyl)-4,5-diazatricyclo[5.2.1.0^{2,6}]deca-2(6),3-dien-3-yl]-2-phenylacetylene;
36. N5-(3,3-Dimethyl-2-oxobutyl)-3-(2,4-difluorophenyl)-3,4-diazatricyclo[5.2.1.0^{2,6}]deca-2(6),4-dien-5-carboxamide;
37. N5-(3,3-Dimethyl-2-oxobutyl)-3-(1R,7S)-(2,4-Difluorophenyl)-3,4-diazatricyclo[5.2.1.0^{2,6}]deca-2(6),4-dien-5-carboxamide;
38. N5-(3,3-Dimethyl-2-oxobutyl)-3-(1S,7R)-(2,4-Difluorophenyl)-3,4-diazatricyclo[5.2.1.0^{2,6}]deca-2(6),4-dien-5-carboxamide;
39. N5-(3,3-Dimethyl-2-oxobutyl)-3-(4-chlorophenyl)-3,4-diazatricyclo[5.2.1.0^{2,6}]deca-2(6),4-dien-5-carboxamide;
40. N5-(3,3-Dimethyl-2-oxobutyl)-3-(4-chloro-2-fluorophenyl)-3,4-diazatricyclo[5.2.1.0^{2,6}]deca-2(6),4-dien-5-carboxamide;
41. N5-(3,3-Dimethyl-2-oxobutyl)-3-(2,4,6-trifluorophenyl)-3,4-diazatricyclo[5.2.1.0^{2,6}]deca-2(6),4-dien-5-carboxamide;
42. Ethyl 4-[5-(2,4-difluorophenyl)-4,5-diazatricyclo[5.2.1.0^{2,6}]deca-2(6),3-dien-3-yl-carboxamido]-2,2-dimethyl-3-oxobutanoate;
43. Ethyl 4-[(1S,7R)-5-(2,4-difluorophenyl)-4,5-diazatricyclo[5.2.1.0^{2,6}]deca-2(6),3-dien-3-yl-carboxamido]-2,2-dimethyl-3-oxobutanoate;
44. Ethyl 4-[(1R,7S) 5-(2,4-difluorophenyl)-4,5-diazatricyclo[5.2.1.0^{2,6}]deca-2(6),3-dien-3-yl-carboxamido]-2,2-dimethyl-3-oxobutanoate;
45. N5-(3,3-Dimethyl-2-oxobutyl)-3-(4-chloro-2-fluorophenyl)-3,4-diazatricyclo[5.2.1.0^{2,6}]deca-2(6),4-dien-5-carboxamide;
46a (1S,7R)- or (1R,7S)-N5-(3,3-Dimethyl-2-oxobutyl)-3-(4-chloro-2-fluorophenyl)-3,4-diazatricyclo[5.2.1.0^{2,6}]deca-2(6),4-dien-5-carboxamide;
46b (1R,7S) or (1S,7R)-N5-(3,3-Dimethyl-2-oxobutyl)-3-(4-chloro-2-fluorophenyl)-3,4-diazatricyclo[5.2.1.0^{2,6}]deca-2(6),4-dien-5-carboxamide;
47. N5-(3,3-Dimethyl-2-oxobutyl)-3-(4-methoxyphenyl)-3,4-diazatricyclo[5.2.1.0^{2,6}]deca-2(6),4-dien-5-carboxamide;
48. N5-(3,3-Dimethyl-2-oxobutyl)-3-(4-bromophenyl)-3,4-diazatricyclo[5.2.1.0^{2,6}]deca-2(6),4-dien-5-carboxamide;
49. N5-(3,3-Dimethyl-2-oxobutyl)-3-(4-nitrophenyl)-3,4-diazatricyclo[5.2.1.0^{2,6}]deca-2(6),4-dien-5-carboxamide;
50. N5-(3,3-Dimethyl-2-oxobutyl)-3-(4-fluorophenyl)-3,4-diazatricyclo[5.2.1.0^{2,6}]deca-2(6),4-dien-5-carboxamide;
51. N5-(3,3-Dimethyl-2-oxobutyl)-3-(2,4-difluorophenyl)-1,10,10-trimethyl-3,4-diazatricyclo[5.2.1.0^{2,6}]deca-2(6),4-dien-5-carboxamide;
52. N12-(3,3-Dimethyl-2-oxobutyl)-10-(2,4-difluorophenyl)-10,11-diazatetracyclo[6.5.2.1.0^{2,7}]pentadeca-2,4,6,9(13),11-pentaene-12-carboxamide;
53. N7-(3,3-Dimethyl-2-oxobutyl)-5-(4-chlorophenyl)-5,6-diazatetracyclo[7.3.1.1^{3,11}.0^{4,8}]tetradeca-4(8),6-dien-7-carboxamide;
54. 5-(tert-Butyl)-2-[5-(2,4-difluorophenyl)-4,5-diazatricyclo[5.2.1.0^{2,6}]deca-2(6),3-dien-3yl]-1,3-oxazole;
55. 5-(tert-Butyl)-2-[(1S,7R)-5-(2,4-Difluorophenyl)-4,5-diazatricyclo[5.2.1.0^{2,6}]deca-2(6),3-dien-3yl]-1,3-oxazole;
56. 5-(tert-Butyl)-2-[(1R,7S)-5-(2,4-Difluorophenyl)-4,5-diazatricyclo[5.2.1.0^{2,6}]deca-2(6),3-dien-3yl]-1,3-oxazole;
57. Ethyl 2-{2-[5-(2,4-difluorophenyl)-4,5-diazatricyclo[5.2.1.0^{2,6}]deca-2(6),3-dien-3-yl]-1,3-oxazol-5-yl}-2-methyl propanoate;
58. Ethyl 2-{2-[(1S,7R)-5-(2,4-difluorophenyl)-4,5-diazatricyclo[5.2.1.0^{2,6}]deca-2(6),3-dien-3-yl]-1,3-oxazol-5-yl}-2-methyl propanoate;
59. Ethyl 2-{2-[(1R,7S)-5-(2,4-difluorophenyl)-4,5-diazatricyclo[5.2.1.0^{2,6}]deca-2(6),3-dien-3-yl]-1,3-oxazol-5-yl}-2-methyl propanoate;
60. 2-{2-[5-(2,4-Difurophenyl)-4,5-diazatricycyclo[5.2.1.0^{2,6}]deca-2(6),3-dien-3-yl]-1,3-oxazol-5-yl}-2-methylpropanoic acid;
61. 2-{2-[(1R,7S)-5-(2,4-Difluorophenyl)-4,5-diazatricyclo[5.2.1.0^{2,6}]deca-2(6),3-dien-3-yl]-1,3-oxazol-5-yl}-2-methyl propanoic acid;
62. 2-{2-[(1S,7R)-5-(2,4-Difluorophenyl)-4,5-diazatricyclo[5.2.1.0^{2,6}]deca-2(6),3-dien-3-yl]-1,3-oxazol-5-yl}-2-methyl propanoic acid;
63. 2-{2-[5-(2,4-Difluorophenyl)-4,5-diazatricyclo[5.2.1.0^{2,6}]deca-2(6),3-dien-3yl]-1,3-oxazol-5-yl}-2-methyl-1-propanol;
64. (1S,7R)-2-{2-[5-(2,4-Difluorophenyl)-4,5-diazatricyclo[5.2.1.0^{2,6}]deca-2(6),3-dien-3yl]-1,3-oxazol-5-yl}-2-methyl-1-propanol;
65. (1R,7S)-2-{2-[5-(2,4-Difluorophenyl)-4,5-diazatricyclo[5.2.1.0^{2,6}]deca-2(6),3-dien-3yl]-1,3-oxazol-5-yl}-2-methyl-1-propanol;
66. 2-{2-[5-(2,4-Difurophenyl)-4,5-diazatricycyclo[5.2.1.0^{2,6}]deca-2(6),3-dien-3-yl]-1,3-oxazol-5-yl}-2-methylpropanamide;
67. 5-(tert-Butyl)-2-[5-(4-fluorophenyl)-4,5-diazatricyclo[5.2.1.0^{2,6}]deca-2(6),3-dien-3yl]-1,3-oxazole;
68. 5-(tert-Butyl)-2-[-5-(4-chlorophenyl)-4,5-diazatricyclo[5.2.1.0^{2,6}]deca-2(6),3-dien-3yl]-1,3-oxazole;
69. 5-(tert-Butyl)-2-[5-(4-chloro-2-fluorophenyl)-4,5-diazatricyclo[5.2.1.0^{2,6}]deca-2(6),3-dien-3yl]-1,3-oxazole;
70. 5-(tert-Butyl)-2-[(1R,7S)- or (1S,7R)-5-(4-chloro-2-fluorophenyl)-4,5-diazatricyclo[5.2.1.0^{2,6}]deca-2(6),3-dien-3yl]-1,3-oxazole;
71. 5-(tert-Butyl)-2-[(1S,7R)- or (1R,7S)-5-(4-chloro-2-fluorophenyl)-4,5-diazatricyclo[5.2.1.0^{2,6}]deca-2(6),3-dien-3yl]-1,3-oxazole;
72. 5-(tert-Butyl)-2-[5-(2,4,6-trifluorophenyl)-4,5-diazatricyclo[5.2.1.0^{2,6}]deca-2(6),3-dien-3yl]-1,3-oxazole;
73. 5-(tert-Butyl)-2-[5-(4-methoxyphenyl)-4,5-diazatricyclo[5.2.1.0^{2,6}]deca-2(6),3-dien-3yl]-1,3-oxazole;
74. 5-(tert-Butyl)-2-[5-(4-bromophenyl)-4,5-diazatricyclo[5.2.1.0^{2,6}]deca-2(6),3-dien-3yl]-1,3-oxazole;
75. 5-(tert-Butyl)-2-[5-(4-nitrophenyl)-4,5-diazatricyclo[5.2.1.0^{2,6}]deca-2(6),3-dien-3yl]-1,3-oxazole;
76. 5-(tert-Butyl)-2-[5-(2,4-difluorophenyl)-7,10,10-trimethyl-4,5-diazatricyclo[5.2.1.0^{2,6}]deca-2(6),3-dien-3yl]-1,3-oxazole;
77. 5-(tert-Butyl)-2-[12-(2,4-difluorophenyl)-11,12-diazatetracyclo[6.5.2.0^{2,7}.0^{9,13}]pentadeca-2(7),3,5,9(13),10-pentaen-10-yl]-1,3-oxazole;
78. 5-(tert-Butyl)-2-[5-(4-chlorophenyl)-5,6-diazatetracyclo[7.3.1.1^{3,11}.0^{4,8}]tetradeca-4(8),6-dien-3-yl]-1,3-oxazole;
79. 2-[5-(2,4-Difluorophenyl)-4,5-diazatricyclo[5.2.1.0^{2,6}]deca-2(6),3-dien-3yl]-4-phenyl-1,3-thiazole;
80. 4-(tert-Butyl)-2-[5-(2,4-difluorophenyl)-4,5-diazatricyclo[5.2.1.0^{2,6}]deca-2(6),3-dien-3yl]-1,3-thiazole;
81. 5-(tert-Butyl)-2-[5-(2,4-difluorophenyl)-4,5-diazatricyclo[5.2.1.0^{2,6}]deca-2(6),3-dien-3yl]-1,3-thiazole;
82. 5-(tert-Butyl)-2-[(1S,7R)-5-(2,4-difluorophenyl)-4,5-diazatricyclo[5.2.1.0^{2,6}]deca-2(6),3-dien-3yl]-1,3-thiazole;
83. 5-(tert-Butyl)-2-[(1R,7S)-5-(2,4-difluorophenyl)-4,5-diazatricyclo[5.2.1.0^{2,6}]deca-2(6),3-dien-3yl]-1,3-thiazole;
84. 5-[5-(tert-Butyl)-1H-2-imidozolyl]-3-(2,4-difluorophenyl)-3,4-diazatricyclo[5.2.1.0^{2,6}]deca-2(6),4-diene;
85. 5-[4-(tert-Butyl)-1-methyl-1H-2-imidozolyl]-3-(2,4-difluorophenyl)-3,4-diazatricyclo[5.2.1.0^{2,6}]deca-2(6),4-diene or 5-[5-(tert-Butyl)-1-methyl-1H-2-imidozolyl]-3-(2,4-difluorophenyl)-3,4-diazatricyclo[5.2.1.0^{2,6}]deca-2(6),4-diene;
86. E or Z-1-{5-(2,4-Diflurophenyl)-4,5-diazatricyclo[5.2.1.0^{2,6}.]deca-2(6),3-dien-3-yl]}-3,3,-dimethyl-1-butanone-O-methyl-oxime;
87. 5-[4-(tert-Butyl)phenyl]-3-(2,4-difluorophenyl)-3,4-diazatricyclo[5.2.1.0^{2,6}]deca-2(6),4-diene;
88. 3-[5-(2,4-Difluorophenyl)-4,5-diazatricyclo[5.2.1.0^{2,6}]deca-2(6),3-dien-3-yl]benzaldehyde;
89. 3-[5-(2,4-Difluorophenyl)-4,5-diazatricyclo[5.2.1.0^{2,6}]deca-2(6),3-dien-3-yl]phenylmethanol;
90. N1-(tert-Butyl)-3-[4-(2,4-Difluorophenyl)-4,5-diazatricyclo[5.2.1.0^{2,6}]deca-2(6),3-dien-3-yl]propanamide or N1-(tert-Butyl)-3-[5-(2,4-Difluorophenyl)-4,5-diazatricyclo[5.2.1.0^{2,6}]deca-2(6),3-dien-3-yl]propanamide;
91. N1-(tert-Butyl)-3-[5-(2,4-Difluorophenyl)-4,5-diazatricyclo[5.2.1.0^{2,6}]deca-2(6),3-dien-3-yl]propanamide or N1-(tert-Butyl)-3-[4-(2,4-Difluorophenyl)-4,5-diazatricyclo[5.2.1.0^{2,6}]deca-2(6),3-dien-3-yl]propanamide;
92a. Methyl (2S)-2-[5-(2,4-difluorophenyl)-4,5-diazatricyclo[5.2.1.0.^{2,6}]deca-2(6),3-dien-3-ylcarboxamido]-2-(4-fluorophenyl)ethanoate;
92b. N5-[(1S)-2-Hydroxy-1-(4-fluorophenyl)ethyl 3-(2,4-difluorophenyl)-3,4-diazatricyclo[5.2.1.0.^{2,6}]deca-2(6),4-diene-5-carboxamide;
93. N5-(2-hydroxy-1,1-dimethylethyl)-3-(2,4-diflurophenyl)-3,4-diazatricyclo[5.2.1.0^{2,6}]deca-2(6),4-diene-5-carboxamide;
94. (1R,7S)-N5-(2-hydroxy-1,1-dimethylethyl)-3-(2,4-difluorophenyl)-3,4-diazatricyclo[5.2.1.0^{2,6}]deca-2(6),4-diene-5-carboxamide;
95. (1S,7R)-N5-(2-Hydroxy-1,1-dimethylethyl)-3-(2,4-difluorophenyl)-3,4-diazatricyclo[5.2.1.0^{2,6}]deca-2(6),4-diene-5-carboxamide;
96. N5-(2-Hydroxy-1,1-dimethylethyl)-3-(4-chlorophenyl)-3,4-diazatricyclo[5.2.1.0^{2,6}]deca-2(6),4-diene-5-carboxamide;
97. (1R,7S)- or (1S,7R)-N5-(2-Hydroxy-1,1-dimethylethyl)-3-(4-chlorophenyl)-3,4-diazatricyclo[5.2.1.0^{2,6}]deca-2(6),4-diene-5-carboxamide;
98. (1S,7R)- or (1R,7S)-N5-(2-Hydroxy-1,1-dimethylethyl)-3-(4-chlorophenyl)-3,4-diazatricyclo[5.2.1.0^{2,6}]deca-2(6),4-diene-5-carboxamide;
99. N5-(2-Hydroxy-1,1-dimethylethyl)-3-(4-bromophenyl)-3,4-diazatricyclo[5.2.1.0^{2,6}]deca-2(6),4-diene-5-carboxamide;
100. N5-(2-Hydroxy-1,1-dimethylethyl)-3-(4-chloro-2-fluorophenyl)-3,4-diazatricyclo[5.2.1.0^{2,6}]deca-2(6),4-diene-5-carboxamide;
101. N5-(2-Hydroxy-1,1-dimethylethyl)-3-(2,4,6-trifluorophenyl)-3,4diazatricyclo[5.2.1.0^{2,6}]deca-2(6),4-diene-5-carboxamide;
102. (1R,7S)- or (1S,7R)-N5-(tert-Butyl)-3-(4-bromophenyl)-3,4-diazatricyclo[5.2.1.0^{2,6}]deca-2(6),4-diene-5-carboxamide;
103. (1S,7R)- or (1R,7S)-N5-(tert-Butyl)-3-(4-bromophenyl)-3,4-diazatricyclo[5.2.1.0^{2,6}]deca-2(6),4-diene-5-carboxamide;
104. (1R,7S)- or (1S,7R)-N5-(tert-Butyl)-3-(4-chlorophenyl)-3,4-diazatricyclo[5.2.1.0^{2,6}]deca-2(6),4-diene-5-carboxamide;
105. (1S,7R)- or (1R,7S)-N5-(tert-Butyl)-3-(4-chlorophenyl)-3,4-diazatricyclo[5.2.1.0^{2,6}]deca-2(6),4-diene-5-carboxamide;
106. N5-(tert-Butyl)-3-[2,4-difluorophenyl]-3,4-diazatricyclo[5.2.1.0^{2,6}]deca-2(6),4-diene-5-carboxamide;
107 N5-(tert-Butyl)-3-[4-Nitrophenyl]-3,4-diazatricyclo[5.2.1.0^{2,6}]deca-2(6),4-diene-5-carboxamide;
108. N5-(tert-Butyl)-3-[4-aminophenyl]-3,4-diazatricyclo[5.2.1.0^{2,6}]deca-2(6),4-diene-5-carboxamide;
109. N1-{4-[5-(tert-Butylcarbamoyl)-3,4-diazatricyclo[5.2.1.0^{2,6}]deca-2(6),4-dien-3-yl]phenyl}acetamide;
110. N1-{4-[5-(tert-Butylcarbamoyl)-3,4-diazatricyclo[5.2.1.0^{2,6}]deca-2(6),4-dien-3-yl]phenyl}carbamic acid ethyl ester;
111. N5,N5-Diisopropyl-3-(2,4-difluorophenyl)-3,4-diazatricyclo[5.2.1.0^{2,6}]deca-2(6),4-diene-5-carboxamide;
112. N5-(1,1-Dimethylhexyl)-3-(2,4-difluorophenyl)-3,4-diazatricyclo[5.2.1.0^{2,6}]deca-2(6),4-diene-5-carboxamide;
113. N5-(2,2,2-trifluoroethyl)-3-(2,4-diflurophenyl)-3,4-diazatricyclo[5.2.1.0^{2,6}]deca-2(6),4-diene-5-carboxamide;
114. N5-(tert-Butyl)-3-(4-methylphenyl)-3,4-diazatricyclo[5.2.1.0^{2,6}]deca-2(6),4-diene-5-carboxamide;
115. N5-(tert-Butyl)-3-(4-trifluoromethyphenyl)-3,4-diazatricyclo[5.2.1.0^{2,6}]deca-2(6),4-diene-5-carboxamide;
116. N5-(tert-Butyl)-3-[4-(tert-butyl)phenyl]-3,4-diazatricyclo[5.2.1.0^{2,6}]deca-2(6),4-diene-5-carboxamide;
117. N5-(tert-Butyl)-3-(phenyl)-3,4-diazatricyclo[5.2.1.0^{2,6}]deca-2(6),4-diene-5-carboxamide;
118. N5-(tert-Butyl)-3-[4-methoxyphenyl]-3,4-diazatricyclo[5.2.1.0^{2,6}]deca-2(6),4-diene-5-carboxamide;
119. N5-(tert-Butyl)-3-(4-chloro-2-fluorophenyl)-3,4-diazatricyclo[5.2.1.0^{2,6}]deca-2(6),4-diene-5-carboxamide;
120. (1R,7S)- or (1S,7R)-N5-(tert-Butyl)-3-(4-chloro-2-fluorophenyl)-3,4-diazatricyclo[5.2.1.0^{2,6}]deca-2(6),4-diene-5-carboxamide;
121. (1S,7R)- or (1R,7S)-N5-(tert-Butyl)-3-(4-chloro-2-fluorophenyl)-3,4-diazatricyclo[5.2.1.0^{2,6}]deca-2(6),4-diene-5-carboxamide;
122. N5-(tert-Butyl)-3-(2-chloro-4-fluorophenyl)-3,4-diazatricyclo[5.2.1.0^{2,6}]deca-2(6),4-diene-5-carboxamide;
123. N5-(tert-Butyl)-3-(2,4,6-trifluorophenyl)-3,4-diazatricyclo[5.2.1.0^{2,6}]deca-2(6),4-diene-5-carboxamide;
124. N5-(tert-Butyl)-3-(2,4,5-trifluorophenyl)-3,4-diazatricyclo[5.2.1.0^{2,6}]deca-2(6),4-diene-5-carboxamide;
125. N5-(tert-Butyl)-3-(3,5-difluorophenyl)-3,4-diazatricyclo[5.2.1.0^{2,6}]deca-2(6),4-diene-5-carboxamide;
126. N5-(tert-Butyl)-4-(2,4-difluorophenyl)-3,4-Diazatricyclo[5.2.1.0^{2,6}]deca-2,5- diene-5-carboxamide;
127. 5-(2,4-Difluorophenyl)-4,5-diazatricyclo[5.2.1.0^{2,6}]deca-2(6),3-dien-3-yl 1,2,3,4-tetrahydro-2-isoquinolinyl methanone;
128. 5-(2,4-Difluorophenyl)-4,5-diazatricyclo[5.2.1.0^{2,6}]deca-2(6),3-dien-3-yl 1,2,3,4-tetrahydro-1-quinolinyl methanone;
129. N5-[(1R)-Indan-1-yl]-3-(2,4-difluorophenyl)-3,4-diazatricyclo[5.2.1.0^{2,6}]deca-2(6),4-diene-5-carboxamide;
130. 3-(2,4-Difluorophenyl)-3,4-diazatricyclo[5.2.1.0^{2,6}]deca-2(6),4-dien-5-carboxylic N′-tert-butylhydrazide;
131. N5-[(1S)-1-(4-fluorophenyl)-2-methoxyethyl]-3-(2,4-difluorophenyl)-3,4-diazatricyclo[5.2.1.0^{2,6}]deca-2(6),4-diene-5-carboxmide;
132. N5-(2-Methoxy-1,1-dimethylethyl)-3-(2,4-difluorophenyl)-3,4-diazatricyclo[5.2.1.0^{2,6}]deca-2(6),4-dien-5-carboxamide;
133. tert-Butylamino 5-(2,4-difluorophenyl)-4,5-diazatricyclo[5.2.1.0^{2,6}]deca-2(6),3-diene-3-yl methanethione;
134. N5-(2-Hydroxy-1,1-dimethylethyl)-3-(2,4-difluorophenyl)-3,4-diazatricyclo[5.2.2.0^{2,6}]undeca-2(6),4-diene-5-carboxamide;
135. N5-(2-Hydroxy-1,1-dimethylethyl)-3-(4-fluorophenyl)-3,4-diazatricyclo[5.2.2.0^{2,6}]undeca-2(6),4-diene-5-carboxamide;
136. N5-(tert-Butyl)-3-(4-chlorophenyl)-3,4-diazatricyclo[5.2.2.0^{2,6}]undeca-2(6),4-diene-5-carboxamide;
137. N5-(tert-Butyl)-3-(4-trifluoromethylphenyl)-3,4-diazatricyclo[5.2.2.0^{2,6}]undeca-2(6),4-diene-5-carboxamide;
138. N5-(tert-Butyl)-3-(4-bromophenyl)-3,4-diazatricyclo[5.2.2.0^{2,6}]undeca-2(6),4-diene-5-carboxamide;
139. N5-(tert-Butyl)-3-(4-fluorophenyl)-3,4-diazatricyclo[5.2.2.0^{2,6}]undeca-2(6),4-diene-5-carboxamide;
140. (1S,7R)-N5-(tert-Butyl)-3-(2,4-difluorophenyl)-1,10,10-trimethyl-3,4-diazatricyclo[5.2.1.0^{2,6}]deca-2(6),4-diene-5-carboxamide
141. N5-(2-Hydroxy-1,1-dimethyl)-3-(2,4-difluorophenyl)-1,10,10-trimethyl-3,4-diazatricyclo[5.2.1.0^{2,6}]deca-2(6),4-diene-5-carboxmide;
142. N(7)-(tert-Butyl)-5-(2,4-difluorophenyl)-5,6-diazatetracyclo[7.3.1.1^{3,11}.0^{4,8}]tetradeca-4(8),6-diene-7-carboxamide;
143. N(7)-(tert-Butyl)-5-(4-fluorophenyl)-5,6-diazatetracyclo[7.3.1.1^{3,11}.0^{4,8}]tetradeca-4(8),6-diene-7-carboxamide;
144. (1R,8R)-N5-(tert-Butyl)-3-(2,4-diflurophenyl)-9,9-dimethyl-3,4-diazatricyclo[6.1.1.0^{2,6}]deca-2(6),4-diene-5-carboxamide;
145. (1S,8S)-N5-(tert-Butyl)-3-(2,4-diflurophenyl)-9,9-dimethyl-3,4-diazatricyclo[6.1.1.0^{2,6}]deca-2(6),4-diene-5-carboxamide;
146. 2-[5-(2,4-diflurophenyl)-4,5-diazatricyclo[5.2.1.0^{2,6}]deca-2(6),3-dien-3-yl]-4,4-dimethyl-4,5-dihydro-1,3-oxazole;
   and pharmaceutically acceptable salts thereof, pharmaceutically acceptable solvates thereof, regioisomers thereof, stereoisomers thereof, prodrugs thereof, and N-oxides thereof.

All of the aforementioned compounds are CB2 agonists and, therefore, useful for disorders treatable with such agonists. These compounds, in many instances, are selective CB2 agonists, i.e., they have minimal binding to CB1.

Another aspect of the present invention is a pharmaceutical composition comprising at least one compound of the present invention and a pharmaceutically acceptable excipient (such as a pharmaceutically acceptable carrier or diluent). Preferably, the pharmaceutical composition comprises a therapeutically effective amount of one or more compounds of the present invention.

Another aspect of the present invention is a method for preventing, ameliorating or treating a cannabinoid receptor mediated disease, disorder or syndrome (such as a disease, disorder or syndrome mediated by interaction with the CB1 or CB2 receptor) in a subject in need thereof by administering to the subject a therapeutically effective amount of one or more compounds of the present invention. Such conditions include, but are not limited to, appetite disorders, metabolism disorders, catabolism disorders, diabetes, obesity, ophthalmic diseases, social related disorders, mood disorders, seizures, substance abuse, learning disorders, cognition disorders, memory disorders, organ contraction, muscle spasm, respiratory disorders, disorders and diseases, locomotor activity disorders, movement disorders, immune disorders (such as autoimmune disorders), inflammation, cell growth, pain and neurodegenerative related syndromes.

Yet another aspect of the present invention is a method for preventing, ameliorating or treating an appetite disorder, social related disorder, autoimmune disorder, inflammation, pain, neurodegenerative related syndrome, disorder or disease, or substance abuse, in a subject in need thereof by administering to the subject a therapeutically effective amount of a compound of the present invention.

Yet another aspect of the present invention is a method for preventing, ameliorating or treating an appetite related disease, disorder or syndrome in a subject in need thereof by administering to the subject a therapeutically effective amount of a compound of the present invention. Non-limiting examples of appetite related disorders include obesity, an overweight condition, anorexia, bulimia, cachexia, dysregulated appetite, and an obesity related syndrome, disorder, disease or symptom (including, but not limited to, obesity as a result of genetics, diet, food intake volume, metabolic syndrome, disorder or disease, hypothalmic disorder or disease, age, abnormal adipose mass distribution, abnormal adipose compartment distribution, a compulsive eating disorder, or a motivational disorder which includes the desire to consume sugars, carbohydrates, alcohols or drugs or any ingredient with hedonic value, and/or reduced activity).

Yet another aspect of the present invention is a method for preventing, ameliorating or treating a social related disease, disorder or syndrome in a subject in need thereof by administering to the subject a therapeutically effective amount of one or more compounds of the present invention. Non-limiting examples of social related disorders include depression and its types (e.g., major depressive disorder), bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia, anxiety, psychosis, social affective disorders, and/or cognitive disorders.

Yet another aspect of the present invention is a method for preventing, ameliorating or treating an autoimmune or inflammation related disease, disorder or syndrome in a subject in need thereof by administering to the subject a therapeutically effective amount of a compound of the present invention. Non-limiting examples of such disorders include psoriasis, lupus erythematosus, diseases of the connective tissue, Sjögren's syndrome, ankylosing spondylarthritis, undifferentiated spondylarthritis, Behcet's disease, autoimmune hemolytic anaemias, multiple sclerosis, amyotrophic lateral sclerosis, amyloses, graft rejection or diseases affecting the plasma cell line, allergic diseases (such as delayed or immediate hypersensitivity, allergic rhinitis, contact dermatitis or allergic conjunctivitis infectious parasitic), viral or bacterial diseases (such as AIDS and meningitis), inflammatory diseases (such as diseases of the joints including, but not limited to, arthritis, rheumatoid arthritis, reactional arthritis, osteoarthritis, spondylitis, gout, vasculitis, Crohn's disease, inflammatory bowel disease (IBD) and irritable bowel syndrome (IBS)) and osteoporosis.

Yet another aspect of the present invention is a method for preventing, ameliorating or treating pain or a neurodegenerative related syndrome, disorder or disease in a subject in need thereof by administering to the subject a therapeutically effective amount of a compound of the present invention. Non-limiting examples of such disorders include central and peripheral pathway mediated pain, bone and joint pain, migraine headache associated pain, cancer pain, dental pain, menstrual cramps, labor pain, chronic pain of the inflammatory type, pain associated with allergies, rheumatoid arthritis, dermatitis or immunodeficiency, chronic neuropathic pain (including pain associated with diabetic neuropathy, sciatica, non specific lower back pain, fibromyalgia, and HIV-related neuropathy), post herpetic neuralgia, trigeminal neuralgia, pain resulting from physical trauma, amputation, cancer, toxins or chronic inflammatory conditions, Hodgkin's disease, myasthenia gravis, nephrotic syndrome, scleroderma and thyroiditis,

Still yet another aspect of the present invention is a method for preventing, ameliorating or treating a substance abuse related syndrome, disorder or disease in a subject in need thereof by administering to the subject a therapeutically effective amount of a compound of the present invention. Non-limiting examples of such disorders include drug abuse and drug withdrawal in which, for example, the substance of abuse or dependence is alcohol, amphetamines, amphetamine like substances, caffeine, cannabis, cocaine, hallucinogens, inhalants, opioids, nicotine (and/or tobacco products), heroin abuse, barbiturates, phencyclidine (or phencyclidine-like compounds), sedative-hypnotics, benzodiazepines, or combinations of substances of abuse.

Still yet another aspect of the present invention is a method for reducing tobacco craving in a subject in need thereof by administering to the subject a therapeutically effective amount of one or more compounds of the present invention.

Still yet another aspect of the present invention is a method for treating nicotine dependency, addiction, withdrawal or aiding in the cessation or lessening of tobacco use in a subject in need thereof by administering to the subject a therapeutically effective amount of one or more compounds of the present invention.

Still yet another aspect, provided herein are processes for preparing compounds described herein.

### DETAILED DESCRIPTION OF THE INVENTION

### Definitions

The term “aryl” refers to aromatic radicals having 6 to 14 carbon atoms such as phenyl, naphthyl, tetrahydronapthyl, indanyl, and biphenyl.

The term “arylalkyl” refers to an aryl group as defined above directly bonded to an alkyl group as defined above, e.g., —CH₂C₆H₅ and —C₂H₅C₆H₅. The arylalkyl radical may be attached to the main structure at any carbon atom from the alkyl group that results in the creation of a stable structure.

The term “heterocyclic ring” (or “heterocyclyl”) refers to a stable 3- to 15-membered ring radical which consists of carbon atoms and from one to five heteroatoms selected from nitrogen, phosphorus, oxygen and sulfur. For purposes of this invention, the heterocyclic ring radical may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heterocyclic or heteroaryl). Examples of such heterocyclic ring radicals include, but are not limited to, azetidinyl, acridinyl, benzodioxolyl, benzodioxanyl, benzofurnyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, quinoxalinyl, quinolinyl, isoquinolinyl, tetrazoyl, imidazolyl, tetrahydroisouinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, oxasolidinyl, triazolyl, indanyl, isoxazolyl, isoxasolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, furyl, tetrahydrofurtyl, tetrahydropyranyl, thienyl, benzothienyl, thiamorpholinyl, thiamorpholinyl sulfoxide, thiamorpholinyl sulfone, dioxaphospholanyl, oxadiazolyl, chromanyl, and isochromanyl. The heterocyclic ring radical may be attached to the main structure at any heteroatom or carbon atom that results in the creation of a stable structure.

The term “heterocyclylalkyl” refers to a heterocyclic ring radical directly bonded to an alkyl group. The heterocyclylalkyl radical may be attached to the main structure at any carbon atom in the alkyl group that results in the creation of a stable structure.

The term “heteroaryl” refers to aromatic radicals having 5 to 14 atoms in the aromatic heterocyclic ring radical. The heteroaryl ring radical may be attached to the main structure at any heteroatom or carbon atom that results in the creation of a stable structure.

The term “heteroarylalkyl” refers to a heteroaryl ring radical directly bonded to an alkyl group. The heteroarylalkyl radical may be attached to the main structure at any carbon atom from the alkyl group that results in the creation of a stable structure.

The term “bridged bicyclic ring system”, unless otherwise defined, refers to an optionally substituted cycloalkyl or heterocyclyl ring system which is bicyclic and contains up to 2 double bonds, and in which one of the rings is bridged. The cycloalkyl or heterocyclyl ring system may further be fused with an aryl or heteroaryl group. The optional substituents can be selected from nitro, cyano, oxo, thio, acyl, halogen, alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclic, heterocyclylalkyl, NR^{c}R^{d}, C(═B)R^{d}, C(O)OR^{d}, C(O)NR^{c}R^{d}, S(O)ₘR^{d}, S(O)ₘNR^{c}R^{d}, OR^{d}, SR^{d}, wherein R^{c} and R^{d} can be independently hydrogen, nitro, halo, acyl, alkyl, alkenyl, alkynyl, cycloalkyl, cycloalkylalkyl, cycloalkenyl, cycloalkenylalkyl, aryl, arylalkyl, heteroaryl, heteroarylalkyl, heterocycly, heterocyclylalkyl, NR^{e}R^{f}, C(═B)R^{f}, C(O)OR^{f}, C(O)NR^{e}R^{f}, S(O)ₘR^{f}, S(O)ₘNR^{e}R^{f}, OR^{f}, SR^{f} [(wherein R^{e} and R^{f}, when bound to a common atom, may join to form an optionally substituted 3-7 membered cyclic ring containing one or more heteroatom(s) selected from N, S or O, (wherein optional substituent(s) can be CO, SO₂ or R^{f})], wherein R^{e} and R^{f} can be independently hydrogen, alkyl, acyl, alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclyl, heterocyclylalkyl. Bridged bicyclo ring system can also be substituted with substituents as defined hereinbelow.

Non-limiting examples of ring P include, but are not limited to,

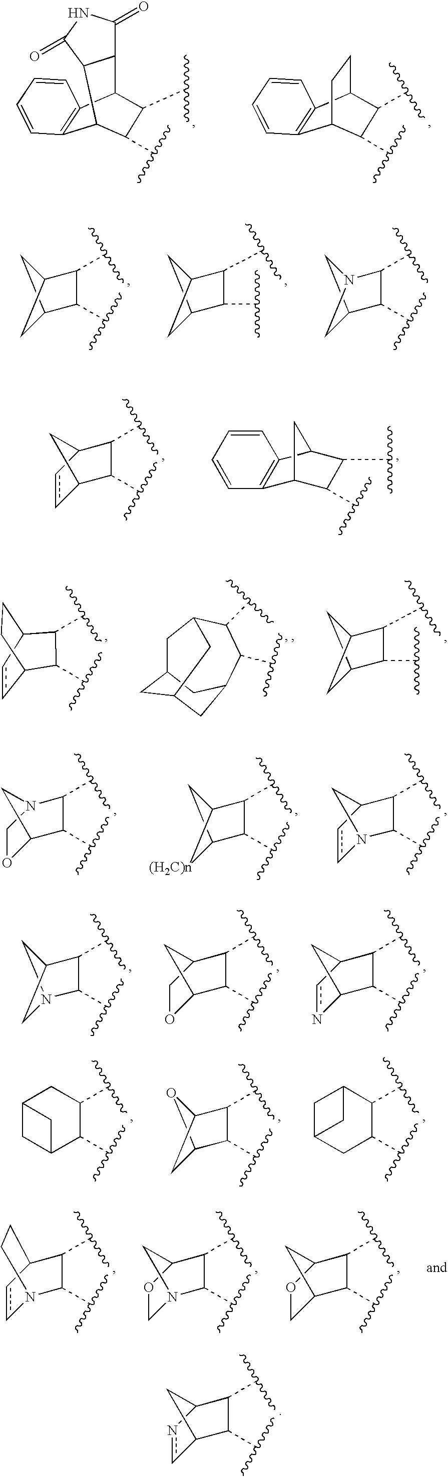

wherein ‘- - -’ represents a bond or no bond.

The term “alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl(isopropyl), n-butyl, n-pentyl, and 1,1-dimethylethyl (t-butyl).

The term “alkenyl” refers to a straight or branched aliphatic hydrocarbon group containing a carbon-carbon double bond and having 2 to about 10 carbon atoms, e.g., ethenyl, 1-propenyl, 2-propenyl (allyl), iso-propenyl, 2-methyl-1-propenyl, 1-butenyl, and 2-butenyl.

The term “alkynyl” refers to a straight or branched chain hydrocarbyl radical having at least one carbon-carbon triple bond, and having 2 to about 12 carbon atoms (with radicals having 2 to about 10 carbon atoms being preferred), e.g., ethynyl, propynyl, and butynyl.

The term “haloalkyl” refers to a group containing at least one halogen and an alkyl portion as define above, that is, a haloalkyl is a substituted alkyl group that is substituted with one or more halogens. Exemplary haloalkyl groups include fluoromethyl, chloromethyl, fluoroethyl, chloroethyl, trifluoromethyl, and the like.

The term “alkoxy” denotes an alkyl group attached via an oxygen linkage to the rest of the molecule. Representative examples of such groups are —OCH₃ and —OC₂H₅.

The term “cycloalkyl” denotes a non-aromatic mono or multicyclic ring system of 3 to about 12 carbon atoms, such as cyclopropyl, cyclobutyl, cyclopentyl, and cyclohexyl. Examples of multicyclic cycloalkyl groups include, but are not limited to, perhydronapththyl, adamantyl and norbornyl groups, bridged cyclic groups and sprirobicyclic groups, e.g., sprio (4,4) non-2-yl.

The term “cycloalkylalkyl” refers to a cyclic ring-containing radical, having 3 to about 8 carbon atoms, directly attached to an alkyl group. The cycloalkylalkyl group may be attached to the main structure at any carbon atom in the alkyl group that results in the creation of a stable structure. Non-limiting examples of such groups include cyclopropylmethyl, cyclobutylethyl, and cyclopentylethyl.

The term “cycloalkenyl” refers to a cyclic ring-containing radical having 3 to about 8 carbon atoms with at least one carbon-carbon double bond, such as cyclopropenyl, cyclobutenyl, and cyclopentenyl.

The term “cycloalkenylalkyl” refers to a cyclic ring-containing radical having 3 to about 8 carbon atoms, with at least one carbon-carbon double bond, directly attached to an alkyl group. The cycloalkenylalkyl group may be attached to the main structure at any carbon atom in the alkyl group that results in the creation of a stable structure. Non-limiting examples of such groups include such as cyclopropenylmethyl, cyclobutenylmethyl, and cyclopentenylethyl.

Unless otherwise specified, the term “substituted” as used herein refers to substitution with any one or any combination of the following substituents: hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted or unsubstituted heterocyclylalkyl ring, substituted or unsubstituted heteroarylalkyl, substituted or unsubstituted heterocyclic ring, substituted or unsubstituted guanidine, —COORₓ, —C(O)Rₓ, —C(S)Rₓ, —C(O)NRₓR_{y}, —C(O)ONRₓR_{y}, —NRₓCONR_{y}R_{z}, —N(Rₓ)SOR_{y}, —N(Rₓ)SO₂R_{y}, —(═N—N(Rₓ)R_{y}), —NRₓC(O)OR_{y}, —NRₓR_{y}, —NRₓC(O)R_{y}, —NRₓC(S)R_{y}, —NRₓC(S)NR_{y}R_{z}, —SONRₓR_{y}, —SO₂NRₓR_{y}, —ORₓ, —ORₓC(O)NR_{y}R_{z}, —ORₓC(O)OR_{y}, —OC(O)Rₓ, —OC(O)NRₓR_{y}, —RₓNR_{y}C(O)R_{z}, —RₓOR_{y}, —RₓC(O)OR_{y}, —RₓC(O)NR_{y}R_{z}, —RₓC(O)R_{y}, —RₓOC(O)R_{y}, —SRₓ, —SORₓ, —SO₂Rₓ, and —ONO₂, wherein Rₓ, R_{y} and R_{z} are independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted heterocyclylalkyl ring, substituted or unsubstituted heteroarylalkyl, or substituted or unsubstituted heterocyclic ring. The substituents in the aforementioned “substituted” groups cannot be further substituted. For example, when the substituent on “substituted alkyl” is “substituted aryl”, the substituent on “substituted aryl” cannot be “substituted alkenyl”.

The term “protecting group” or “PG” refers to a substituent that is employed to block or protect a particular functionality. Other functional groups on the compound may remain reactive. For example, an “amino-protecting group” is a substituent attached to an amino group that blocks or protects the amino functionality in the compound. Suitable amino-protecting groups include, but are not limited to, acetyl, trifluoroacetyl, t-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable hydroxy-protecting groups include, but are not limited to, acetyl and silyl. A “carboxy-protecting group” refers to a substituent of the carboxy group that blocks or protects the carboxy functionality. Suitable carboxy-protecting groups include, but are not limited to, —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, and nitroethyl. For a general description of protecting groups and their use, see T. W. Greene, Protective Groups in Organic Synthesis, John Wiley & Sons, New York, 1991.

The term “cannabinoid receptor” refers to any one of the known or heretofore unknown subtypes of the class of cannabinoid receptors, including CB1 and/or CB2 receptors, that may be bound by a cannabinoid modulator compound of the present invention.

The term “modulator” further refers to the use of a compound of the invention as a CB (e.g., CB1 and/or CB2) receptor agonist, partial agonist, antagonist or inverse-agonist.

The term “treating” or “treatment” of a state, disorder or condition includes:
(1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition;
(2) inhibiting the state, disorder or condition, i.e., arresting or reducing the development of the disease or at least one clinical or subclinical symptom thereof; or
(3) relieving the disease, i.e., causing regression of the state, disorder or condition or at least one of its clinical or subclinical symptoms.

The benefit to a subject to be treated is either statistically significant or at least perceptible to the subject or to the physician.

The term “subject” includes mammals (especially humans) and other animals, such as domestic animals (e.g., household pets including cats and dogs) and non-domestic animals (such as wildlife).

A “therapeutically effective amount” means the amount of a compound that, when administered to a subject for treating a state, disorder or condition, is sufficient to effect such treatment. The “therapeutically effective amount” will vary depending on the compound, the disease and its severity and the age, weight, physical condition and responsiveness of the subject to be treated.

Pharmaceutically acceptable salts forming part of this invention include salts derived from inorganic bases (such as Li, Na, K, Ca, Mg, Fe, Cu, Zn, and Mn), salts of organic bases (such as N,N′-diacetylethylenediamine, glucamine, triethylamine, choline, hydroxide, dicyclohexylamine, metformin, benzylamine, trialkylamine, and thiamine), salts of chiral bases (such as alkylphenylamine, glycinol, and phenyl glycinol), salts of natural amino acids (such as glycine, alanine, valine, leucine, isoleucine, norleucine, tyrosine, cystine, cysteine, methionine, proline, hydroxy proline, histidine, ornithine, lysine, arginine, and serine), salts of non-natural amino acids (such as D-isomers or substituted amino acids), salts of guanidine, salts of substituted guanidine (wherein the substituents are selected from nitro, amino, alkyl, alkenyl, or alkynyl), ammonium salts, substituted ammonium salts, and aluminum salts. Other pharmaceutically acceptable salts include acid addition salts (where appropriate) such as sulphates, nitrates, phosphates, perchlorates, borates, hydrohalides, acetates (such as trifluroacetate), tartrates, maleates, citrates, fumarates, succinates, palmoates, methanesulphonates, benzoates, salicylates, benzenesulfonates, ascorbates, glycerophosphates, and ketoglutarates. Yet other pharmaceutically acceptable salts include, but are not limited to, quaternary ammonium salts of the compounds of invention with alkyl halides or alkyl sulphates (such as MeI or (Me)₂SO₄).

Pharmaceutically acceptable solvates includes hydrates and other solvents of crystallization (such as alcohols). The compounds of the present invention may form solvates with standard low molecular weight solvents by methods known in the art.

Compounds described herein can comprise one or more asymmetric carbon atoms and thus can occur as racemic mixtures, enantiomers and diastereomers. These compounds can also exist as conformers/rotamers. All such isomeric forms of these compounds are expressly included in the present invention. Stereoisomers of the compounds described herein may be prepared by using starting materials in enantiomerically pure form in the process wherever possible or by performing reactions in the presence of optically pure catalysts or reagents or by resolving the mixture of stereoisomers by methods known to those skilled in the art. Preferred methods for resolution of racemic compounds include, but are not limited to: microbial resolution; resolving the diastereomeric salts formed with chiral acids such as mandelic acid, camphorsulfonic acid, tartaric acid, lactic acid, and the like wherever applicable; or resolving the diastereomeric salts formed with chiral bases such as optionally substituted α-methylbenzylamines, e.g., R— or S-α-methylbenzylamine, R— or S-1-(4-chlorophenyl)-ethylamine, brucine, cinchona alkaloids and their derivatives; and the like. Commonly used methods are compiled in Jaques, et al. in Enantiomers, Racemates and Resolution; Wiley-Interscience, 1981. Other examples of suitable resolution methods include, where appropriate, (i) treating the compounds of the present invention with chiral alcohols, chiral amines, amino acids, or aminoalcohols or amides derived from amino acids, or (ii) using conventional reaction conditions to convert an acid into an amide or an ester and by separation of diastereomeric ester or amides by fractional crystallization or by chromatography and then hydrolyzing the pure diastereomeric amide or ester.

### Pharmaceutical Compositions

The pharmaceutical composition of the present invention comprises at least one compound of the present invention and a pharmaceutically acceptable excipient (such as a pharmaceutically acceptable carrier or diluent). Preferably, the pharmaceutical composition comprises a therapeutically effective amount of the compound(s) of the present invention. The compound of the present invention may be associated with a pharmaceutically acceptable excipient (such as a carrier or a diluent) or be diluted by a carrier, or enclosed within a carrier which can be in the form of a capsule, sachet, paper or other container.

Examples of suitable carriers include, but are not limited to, water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethyl cellulose and polyvinylpyrrolidone.

The carrier or diluent may include a sustained release material, such as glyceryl monostearate or glyceryl distearate, alone or mixed with a wax.

The pharmaceutical composition may also include one or more pharmaceutically acceptable auxiliary agents, wetting agents, emulsifying agents, suspending agents, preserving agents, salts for influencing oxmetic pressure, buffers, sweetening agents, flavoring agents, colorants, or any combination of the foregoing. The pharmaceutical composition of the invention may be formulated so as to provide quick, sustained, or delayed release of the active ingredient after administration to the subject by employing methods known in the art.

The pharmaceutical compositions of the present invention may be prepared by conventional techniques, e.g., as described in Remington: The Science and Practice of Pharmacy, 20^{th} Ed., 2003 (Lippincott Williams & Wilkins). For example, the active compound can be mixed with a carrier, or diluted by a carrier, or enclosed within a carrier, which may be in the form of an ampoule, capsule, sachet, paper, or other container. When the carrier serves as a diluent, it may be a solid, semi-solid, or liquid material that acts as a vehicle, excipient, or medium for the active compound. The active compound can be adsorbed on a granular solid container, for example, in a sachet.

The pharmaceutical compositions may be in conventional forms, for example, capsules, tablets, aerosols, solutions, suspensions or products for topical application.

The route of administration may be any route which effectively transports the active compound of the invention to the appropriate or desired site of action. Suitable routes of administration include, but are not limited to, oral, nasal, pulmonary, buccal, subdermal, intradermal, transdermal, parenteral, rectal, depot, subcutaneous, intravenous, intraurethral, intramuscular, intranasal, ophthalmic (such as with an ophthalmic solution) or topical (such as with a topical ointment). The oral route is preferred.

Solid oral formulations include, but are not limited to, tablets, capsules (soft or hard gelatin), dragees (containing the active ingredient in powder or pellet form), troches and lozenges. Tablets, dragees, or capsules having talc and/or a carbohydrate carrier or binder or the like are particularly suitable for oral application. Preferable carriers for tablets, dragees, or capsules include lactose, cornstarch, and/or potato starch. A syrup or elixir can be used in cases where a sweetened vehicle can be employed.

A typical tablet that may be prepared by conventional tabletting techniques may contain: (1) Core: Active compound (as free compound or salt thereof), 250 mg colloidal silicon dioxide (Aerosil®), 1.5 mg microcrystalline cellulose (Avicel®), 70 mg modified cellulose gum (Ac-Di-Sol®), and 7.5 mg magnesium stearate; (2) Coating: HPMC, approx. 9 mg Mywacett 9-40 T and approx. 0.9 mg acylated monoglyceride

Liquid formulations include, but are not limited to, syrups, emulsions, soft gelatin and sterile injectable liquids, such as aqueous or non-aqueous liquid suspensions or solutions.

For parenteral application, particularly suitable are injectable solutions or suspensions, preferably aqueous solutions with the active compound dissolved in polyhydroxylated castor oil.

### Methods of Treatment

The present invention provides compounds and pharmaceutical formulations thereof that are useful in the treatment, amelioration, and/or prevention of diseases, conditions and/or disorders modulated by a cannabinoid (CB) receptor, especially those modulated by the CB1 or CB2 receptor including those discussed below.

The present invention further provides a method of treating a disease, condition and/or disorder modulated by a cannabinoid receptor (CB), and in particular the CB1 or CB2 receptor, in a subject in need thereof by administering to the subject a therapeutically effective amount of a compound or a pharmaceutical composition of the present invention.

Diseases, conditions, and/or disorders that are modulated by a CB receptor, include, but are not limited to, appetite disorders, metabolism disorders, catabolism disorders, diabetes, obesity, social related disorders, mood disorders, seizures, substance abuse, learning disorders, cognition disorders, memory disorders, organ contraction, muscle spasm, respiratory disorders, locomotor activity disorders, movement disorders, immune disorders (such as autoimmune disorders), inflammation, cell growth, pain (such as neuropathic pain) and neurodegenerative related syndromes, disorders and diseases.

Appetite related syndromes, disorders or diseases include, but are not limited to, obesity, overweight conditions, anorexia, bulimia, cachexia, dysregulated appetite and the like. Obesity related syndromes, disorders or diseases include, but are not limited to, obesity as a result of genetics, diet, food intake volume, metabolic syndrome, disorder or disease, hypothalmic disorder or disease, age, abnormal adipose mass distribution, abnormal adipose compartment distribution, compulsive eating disorders, motivational disorders which include the desire to consume sugars, carbohydrates, alcohols or drugs or any ingredient with hedonic value and the like. Symptoms associated with obesity related syndromes, disorders, and diseases include, but are not limited to, reduced activity.

Metabolism related syndromes, disorders or diseases include, but are not limited to, metabolic syndrome, dyslipidemia, elevated blood pressure, insulin sensitivity or resistance, hyperinsulinemia, hypercholesterolemia, hyperlipidemias, atherosclerosis, hypertriglyceridemias, arteriosclerosis, other cardiovascular diseases, osteoarthritis, dermatological diseases, sleep disorders (disturbances of circadian rhythm, dyssomnia, insomnia, sleep apnea and narcolepsy), cholelithiasis, hepatomegaly, steatosis, abnormal alanine aminotransferase levels, polycystic ovarian disease, inflammation, and the like.

Diabetes related syndromes, disorders or diseases include, but are not limited to, glucose dysregulation, insulin resistance, glucose intolerance, hyperinsulinemia, dyslipidemia, hypertension, obesity, hyperglycemia and the like.

Catabolism related syndromes, disorders or diseases include, but are not limited to, catabolism in connection with pulmonary dysfunction and/or ventilator dependency; cardiac dysfunction, e.g., associated with valvular disease, myocardial infarction, cardiac hypertrophy or congestive heart failure.

Ophthalmic diseases include, but are not limited to, glaucoma, glaucoma-associated intraocular pressure retinitis, retinopathies, uveitis, acute injury to the eye tissue (e.g. conjunctivitis).

Social or mood related syndromes, disorders or diseases include, but are not limited to, depression (including, but not limited to, bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia, anxiety, psychosis, social affective disorders, cognitive disorders and the like).

Substance abuse related syndromes, disorders or diseases include, but are not limited to, drug abuse and drug withdrawal. Abused substances include, but are not limited to, alcohol, amphetamines (or amphetamine like substances), caffeine, cannabis, cocaine, hallucinogens, inhalants, opioids, nicotine (and/or tobacco products), heroin abuse, barbiturates, phencyclidine (or phencyclidine-like compounds), sedative-hypnotics, benzodiazepines, or combinations of any of the foregoing. The compounds and pharmaceutical compositions can also be used to treat withdrawal symptoms and substance-induced anxiety or mood disorder.

The present invention further provides a method of treating nicotine dependency, addiction, withdrawal or aiding in the cessation or lessening of tobacco in a subject in need thereof by administering to the subject a therapeutically effective amount of a compound or a pharmaceutical composition of the present invention.

Learning, cognition or memory related syndromes, disorders or diseases which can be treated with the compounds of the present invention include, but are not limited to, memory loss or impairment as a result of age, disease, side effects of medications (adverse events) or the like. Memory impairment is a primary symptom of dementia and can also be a symptom associated with such diseases as Alzheimer's disease, schizophrenia, Parkinson's disease, Huntington's disease, Pick's disease, Creutzfeld-Jakob disease, HIV, cardiovascular disease, and head trauma as well as age-related cognitive decline. Generally, dementias are diseases that include memory loss and additional intellectual impairment separate from memory. The compounds and pharmaceutical compositions of the present invention are also useful in treating cognitive impairments related to attentional deficits, such as attention deficit disorder.

Muscle spasm syndromes, disorders or diseases include, but are not limited to, multiple sclerosis, cerebral palsy and the like.

Locomotor activity and movement syndromes, disorders or diseases include, but are not limited to, stroke, Parkinson's disease, multiple sclerosis, epilepsy and the like.

Respiratory related syndromes, disorders or diseases include, but are not limited to, diseases of the respiratory tract, chronic obstructive pulmonary disorder, emphysema, asthma, and bronchitis.

Kidney dysfunction nephritis which can be treated with the modulators of the present invention include, but is not limited to, mesangial proliferative glomerulonephritis, nephritic syndrome, liver dysfunction (hepatitis, cirrhosis).

Autoimmune or inflammation related syndromes, disorders or diseases include, but are not limited to, psoriasis, lupus erythematosus, diseases of the connective tissue, Sjögren's syndrome, ankylosing spondylarthritis, rheumatoid arthritis, reactional arthritis, undifferentiated spondylarthritis, Behcet's disease, autoimmune hemolytic anaemias, multiple sclerosis, amyotrophic lateral sclerosis, amyloses, graft rejection or diseases affecting the plasma cell line. They also include, but are not limited to, allergic diseases (such as delayed or immediate hypersensitivity, allergic rhinitis, contact dermatitis or allergic conjunctivitis infectious parasitic), viral or bacterial diseases (such as AIDS and meningitis), inflammatory diseases (such as diseases of the joints including, but not limited to, arthritis, rheumatoid arthritis, osteoarthritis, spondylitis, gout, vasculitis, Crohn's disease, inflammatory bowel disease (IBD) and irritable bowel syndrome (IBS)) and osteoporosis.

Cell growth related syndromes, disorders or diseases include, but are not limited to, dysregulated mammalian cell proliferation, breast cancer cell proliferation, and prostrate cancer cell proliferation.

Pain related syndromes, disorders or diseases include, but are not limited to, central and peripheral pathway mediated pain, bone and joint pain, migraine headache associated pain, cancer pain, dental pain, menstrual cramps, labor pain, chronic pain of the inflammatory type, allergies, rheumatoid arthritis, dermatitis, immunodeficiency, chronic neuropathic pain (e.g. pain associated with diabetic neuropathy, fibromyalgia, and HIV-related neuropathy, as well as sciatica, non specific lower back pain, post herpetic neuralgia, trigeminal neuralgia, and pain resulting from physical trauma, amputation, cancer, toxins or chronic inflammatory conditions), Hodgkin's disease, Myasthenia gravis, nephrotic syndrome, scleroderma, and thyroiditis.

Neurodegenerative related syndromes, disorders or diseases include, but are not limited to, Parkinson's disease, multiple sclerosis, epilepsy, ischemia or secondary biochemical injury collateral to traumatic head or brain injury, brain inflammation, eye injury or stroke and the like.

The compounds of this invention may also be used in conjunction with other pharmaceutical agents for the treatment of the diseases, conditions and/or disorders described herein. Therefore, methods of treatment that include administering compounds of the present invention in combination with other pharmaceutical agents are also provided. Suitable pharmaceutical agents that may be used in combination with the compounds of the present invention include, but are not limited to, anti-obesity agents such as apolipoprotein-B secretion/microsomal triglyceride transfer protein (apo-B/MTP) inhibitors, 11β-hydroxy steroid dehydrogenase-1 (11β-HSD type 1) inhibitors, peptide YY₃₋₃₆ or analogs thereof, MCR-4 agonists, cholecystokinin-A (CCK-A) agonists, monoamine reuptake inhibitors (such as sibutramine), sympathomimetic agents, β₃ adrenergic receptor agonists, dopamine receptor agonists (such as bromocriptine), melanocyte-stimulating hormone receptor analogs, 5HT_{2c} receptor agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), neuropeptide-Y receptor antagonists, thyromimetic agents, dehydroepiandrosterone or an analog thereof, glucocorticoid receptor agonists or antagonists, orexin receptor antagonists, glucagon-like peptide-1 (GLP-1) receptor agonists, Protein Tyrosine Phosphatase (PTP-1B) inhibitors, dipeptidyl peptidase IV (DPP-IV) inhibitors, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin receptor antagonists, histamine 3 receptor antagonists or inverse agonists, and neuromedin U receptor agonists. Other anti-obesity agents, including the preferred agents set forth herein below, are well known, or will be readily apparent in light of the instant disclosure, to one of ordinary skill in the art.

Especially preferred are anti-obesity agents such as orlistat, sibutramine, bromocriptine, ephedrine, leptin, peptide YY₃₋₃₆ or an analog thereof (including the complete peptide YY), and pseudoephedrine. Preferably, compounds of the present invention and combination therapies are administered in conjunction with exercise and a sensible diet.

Anti-obesity agents for use in the combinations, pharmaceutical compositions, and methods of the invention can be prepared using methods known to one of ordinary skill in the art, for example, sibutramine can be prepared as described in U.S. Pat. No. 4,929,629; bromocriptine can be prepared as described in U.S. Pat. Nos. 3,752,814 and 3,752,888; orlistat can be prepared as described in U.S. Pat. Nos. 5,274,143, 5,420,305, 5,540,917, and 5,643,874; and PYY₃₋₃₆ (including analogs) can be prepared as described in U.S. Patent Publication No. 2002/0141985 and International Publication No. WO 03/027637. All of the above recited references are incorporated herein by reference.

Other suitable pharmaceutical agents that may be administered in combination with the compounds of the present invention include agents designed to treat tobacco abuse (e.g., nicotine receptor partial agonists, bupropion hypochloride (also known under the tradename Zyban™) and nicotine replacement therapies), agents to treat erectile dysfunction (e.g., dopaminergic agents, such as apomorphine), ADD/ADHD agents (e.g., Ritalin™ (methylphenidate hydrochloride), Strattera™ (atomoxetine hydrochloride), Concerta™ (methylphenidate hydrochloride) and Adderall™ (amphetamine aspartate; amphetamine sulfate; dextroamphetamine saccharate; and dextroamphetamine sulfate)), and agents to treat alcoholism, such as opioid antagonists (e.g., naltrexone (also known under the tradename ReVia™) and nalmefene), disulfiram (also known under the tradename Antabuse™), and acamprosate (also known under the tradename Campral™)). In addition, agents for reducing alcohol withdrawal symptoms may also be co-administered, such as benzodiazepines, beta-blockers, clonidine, carbamazepine, pregabalin, and gabapentin (Neurontin™). Treatment for alcoholism is preferably administered in combination with behavioral therapy including such components as motivational enhancement therapy, cognitive behavioral therapy, and referral to self-help groups, including Alcohol Anonymous (AA).

Other pharmaceutical agents that may be useful include antihypertensive agents; antidepressants (e.g., fluoxetine hydrochloride (Prozac™)); cognitive improvement agents (e.g., donepezil hydrochloride (Aircept™.) and other acetylcholinesterase inhibitors); neuroprotective agents (e.g., memantine); antipsychotic medications (e.g., ziprasidone (Geodon™), risperidone (Risperdal™), and olanzapine (Zyprexa™)); insulin and insulin analogs (e.g., LysPro insulin); GLP-1 (7-37) (insulinotropin) and GLP-1 (7-36)-NH₂; sulfonylureas and analogs thereof: chlorpropamide, glibenclamide, tolbutamide, tolazamide, acetohexamide, Glypizide®, glimepiride, repaglinide, meglitinide; biguanides: metformin, phenformin, buformin; α2-antagonists and imidazolines: midaglizole, isaglidole, deriglidole, idazoxan, efaroxan, fluparoxan; other insulin secretagogues: linogliride, A-4166; glitazones: ciglitazone, Actos® (pioglitazone), englitazone, troglitazone, darglitazone, Avandia® (BRL49653); fatty acid oxidation inhibitors: clomoxir, etomoxir; α-glucosidase inhibitors: acarbose, miglitol, emiglitate, voglibose, MDL-25,637, camiglibose, MDL-73,945; β-agonists: BRL 35135, BRL 37344, RO 16-8714, ICI D7114, CL 316,243; phosphodiesterase inhibitors: L-386,398; lipid-lowering agents: benfluorex: fenfluramine; vanadate and vanadium complexes (e.g., Naglivan®) and peroxovanadium complexes; amylin antagonists; glucagon antagonists; gluconeogenesis inhibitors; somatostatin analogs; antilipolytic agents: nicotinic acid, acipimox, WAG 994, pramlintide (Symlin™), AC 2993, nateglinide, aldose reductase inhibitors (e.g., zopolrestat), glycogen phosphorylase inhibitors, sorbitol dehydrogenase inhibitors, sodium-hydrogen exchanger type 1 (NHE-1) inhibitors and/or cholesterol biosynthesis inhibitors or cholesterol absorption inhibitors, especially a HMG-CoA reductase inhibitor, or a HMG-CoA synthase inhibitor, or a HMG-COA reductase or synthase gene expression inhibitor, a CETP inhibitor, a bile acid sequesterant, a fibrate, an ACAT inhibitor, a squalene synthetase inhibitor, an anti-oxidant or niacin. The compounds of the present invention may also be administered in combination with a naturally occurring compound that acts to lower plasma cholesterol levels. Such naturally occurring compounds are commonly called nutraceuticals and include, for example, garlic extract, Hoodia plant extracts, and niacin.

The compounds of the present invention (including the pharmaceutical compositions and processes used therein) may be used alone or in combination with other pharmaceutical agents in the manufacture of a medicament for the therapeutic applications described herein.

### General Methods of Preparation

The compounds described herein may be prepared by techniques known in the art. In addition, the compounds described herein may be prepared by following the reaction sequences as depicted in Schemes 1-17. Further, in the following schemes, where specific bases, acids, reagents, solvents, oxidizing agents, reducing agents, coupling agents, etc., are mentioned, it is understood that other bases, acids, reagents, solvents, oxidizing agents, reducing agents, coupling agents etc., known in the art may be also be used and are therefore included within the present invention. Variations in reaction conditions, for example, temperature and/or duration of the reaction, which may be used as known in the art are also within the scope of the present invention. All the stereoisomers of the compounds in these schemes, unless otherwise specified, are also encompassed within the scope of this invention.

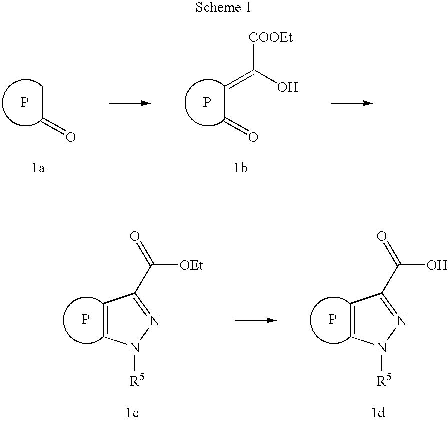

The compounds of Formula 1d can be prepared according to scheme 1. The compound of Formula 1a is converted to a compound of Formula 1b, for example, by reaction with a dialkyloxalate. The compound of Formula 1b is converted to a compound of Formula 1c, for example, by reaction with a hydrazine (e.g., of the formula R⁵NHNH₂, where R⁵ is as defined above).

Alternatively, the compound of Formula 1b upon reaction with an unsubstituted hydrazine can form two canonical structures of compounds of Formula 1c (R⁵═H), which can be reacted with a compound of Formula R⁵L (wherein R⁵ is as defined earlier and L is a leaving group, such as halogen, OTs, OMs, phenylsulfonyl halide or benzoyl halide) to form a compound with R⁵ being at 1 or 2 positions of the pyrazole ring of the compound of Formula 1c. These compounds can be separated by any techniques known in the art such as column chromatography (preferably, silica gel column chromatography).

The compound of Formula 1c is hydrolyzed to form a compound of Formula 1d. The compound of formula 1d, wherever it is a mixture of stereoisomers, can be resolved by known methods for resolution of carboxylic acids. Preferred methods of resolution include microbial resolution, resolution by preferential crystallization of diasteromeric salts formed by reacting the compounds of formula 1d with amines (e.g., of the formula R^{x}NR^{y}R^{z} wherein one, two or all of R^{x}, R^{y} and R^{z} contain at least one asymmetric centre). The compounds of formula 1d used in any of the schemes described in the present invention can be racemic or optically pure or enriched in one isomer wherever applicable. Scheme 1 can also be used to prepare compounds of Formula 1c and 1d with R⁵ being at the 2-position of the pyrazole ring.

Compounds of Formula 1a can be prepared by procedures known in the art, such as those described in Tabushi et. al., J. Amer. Chem. Soc., 6672 (1970); Black, R. M. and Gill, G. B., J. Chem. Soc. (C), 671 (1970); Kozmina, N and Pacquette, L. A. Synth. Commun. 26(10), 2027-2030 (1996); Yuasa, Y. et. al., Essent. Oil. Res., 10, 39-42 (1998); Diels, O. et al., Annalen, 478, 137-154 (1930); Hall, H. K., Journal of American Chemical Society, 82, 1209-1215 (1960); Carlsen, P. H. J., Synth. Commun., 17, 19-23 (1987); Muir, D. J., and Stotheres J. B., Can. J. Chem., 71(9), 1290-1296 (1993).

The compounds of Formula 1a can be reacted in one or more solvents, for example, protic polar solvents (e.g., methanol, ethanol or propanol), aromatic solvents (e.g., benzene, chlorobenzene, toluene or xylene), aliphatic solvents (e.g., hexane or heptane), ethers (e.g., diethyl ether, tetrahydrofuran, dioxane or dimethoxy ethane) or mixture thereof. The compound of Formula 1a can also be reacted in the presence of one or more bases, for example, metalalkoxides (e.g., sodium methoxide or sodium ethoxide), metal amides (e.g., potassium amide, sodium amide, lithium diisopropyl amide, or lithium bis(trimethylsilyl)amide), metal hydride (e.g., lithium hydride, sodium hydride or potassium hydride), or mixture thereof. During the course of the reaction all possible combinations of bases and solvents can be used and are therefore within the scope of the present invention.

The compounds of Formula 1b can be reacted in the presence of one or more acids, for example, acetic acid, hydrochloric, hydrobromic acid or mixture thereof, and/or in one or more protic polar solvents, for example, methanol, ethanol, propanol, isopropanol or mixture thereof. The compounds of Formula 1c (when R⁵═H) can be reacted in the presence of one or more inorganic bases, for example, lithium hydride, sodium hydride, potassium hydride or mixture thereof. The compounds of Formula 1c can be hydrolyzed in the presence of one or more inorganic bases, for example, sodium hydroxide, potassium hydroxide, lithium hydroxide or mixture thereof, and/or in one or more protic polar solvents, for example, water, methanol, ethanol, propanol, isopropanol or mixture thereof. The hydrolysis can also be carried out by using various other methods known in the art, for example, under acidic condition.

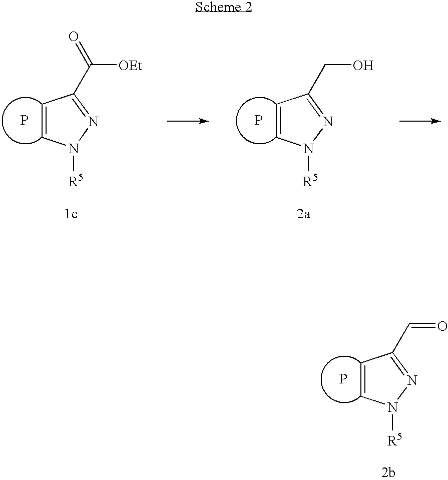

The compounds of Formula 2b can be prepared according to scheme 2. A compound of Formula 1c is reduced to form a compound of Formula 2a. The compound of Formula 2a is oxidized to form a compound of Formula 2b. The compounds of Formula 2a and 2b with R⁵ being at the 2-position of the pyrazole ring rather than the 1-position can also be produced using this reaction scheme.

The compound of Formula 1c can be reduced in the presence of one or more reducing agents such as, for example, lithium borohydride, lithium aluminium hydride or sodium borohydride. The compound of Formula 1c can be reduced in one or more solvents, for example, ethers (e.g., diethyl ether, tetrahydrofuran or dioxane), chlorinated solvents (e.g., dichloromethane or chloroform), alcohols (e.g., methanol or ethanol), or mixture thereof.

The compound of Formula 2a can be oxidized in the presence of one or more oxidizing agents such as, for example, Dess-Martin periodinane, N-chlorosuccinimide, pyridinium chlorochromate, pyridinium dichromate. Alternatively, the compound of Formula 2a may be oxidized by Swern or Pfitzner-Moffatt oxidation or an analogous procedure known in the art. The compounds of Formula 2a can be oxidized in one or more solvents, for example, chlorinated solvents (e.g., dichloromethane, chloroform, carbon tetrachloride or dichloroethane), aprotic polar solvents (e.g., dimethylformamide or dimethylsulfoxide), or mixture thereof.

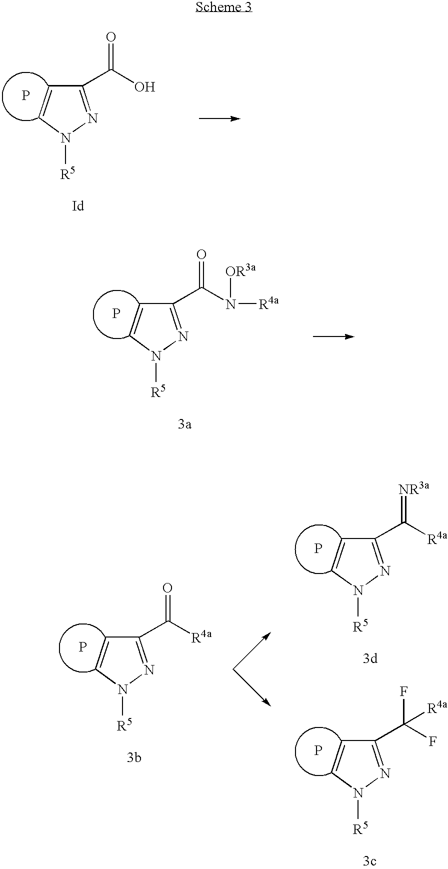

The compounds of Formula 3a, 3b, 3c, and 3d can be prepared according to scheme 3. The compound of Formula 1d is converted to a compound of Formula 3a, for example, by reaction with a compound of Formula R^{4a}NHOR^{3a}. The compound of Formula 3a can be converted to a compound of Formula 3b (Ketones, wherein R^{4a} can be alkyl, cycloalkyl, aryl or heteroaryl), for example, by reaction with a Grignard reagent of Formula R^{4a}MgX (wherein X is halogen and R^{4a} is alkyl, cycloalkyl,aryl or heteroaryl or organometallic compound of Formula R^{4a}M (wherein M can be lithium, sodium or potassium) followed by acid treatment. The compound of Formula 3b can be reacted with a fluorinating agent to form a compound of Formula 3c. Compounds of Formulae 3a, 3b and 3c with R⁵ being at the 2-position of the pyrazole ring can also be prepared by Scheme 3. The compound of Formula 3d can be prepared from the compound of Formula 3b, for example, by reacting the compound of Formula 3b with a compound of Formula NH₂OR^{3a} hydrochloride.

The compounds of Formula 3b can be reacted in one or more solvents, for example, polar aprotic solvents (e.g., dimethylformamide or dimethylsulfoxide), ethers (e.g., tetrahydrofuran or dioxane), chlorinated solvents (e.g., dichloromethane, chloroform or carbon tetrachloride), polar protic solvents (e.g., methanol or ethanol) or mixture thereof.

The compounds of Formula 1d can be reacted in the presence of one or more amide coupling reagents (e.g., benzotiazolyloxytris(dimethylamino)phosphonium hexafluorophosphate, or dicyclohexyl carbodiimide). Alternatively, the compounds of formula Id can be reacted with one or more chlorinating agents (e.g., oxalyl chloride, thionyl chloride, phosphorous pentachloride or phosphorous trichloride or mixture thereof). These reactions can also be carried out in the presence of one or more organic bases, for example, triethylamine, diethylamine, diisopropylethylamine, pyridine, tetramethyl guanidine or mixture thereof, and/or in one or more solvents, for example, chlorinated solvents (e.g., dichloromethane, dichloroethane, chloroform or carbon tetrachloride), aprotic polar solvents (e.g., dimethylformamide or dimethylsulfoxide) or mixture thereof.

The compounds of Formula 3a can be reacted in the presence of one or more ether solvents, for example, diethyl ether, tetrahydrofuran or dioxane under anhydrous conditions, which can be further treated with an acid such as hydrochloric acid, acetic acid or ammonium chloride.

The compounds of Formula 3b can be reacted in the presence of one or more fluorinating agents, for example, dimethylaminosulfur trifluoride, Selectfluor® (1-chloromethyl-4-fluoro-1,4-diazoniabicyclo[2.2.2]octane bis(tetrafluoroborate)) (available from Air Products and Chemicals, Inc. of Allentown, Pa.), N-fluorobenzene sulphonamide or mixture thereof. The fluorination reaction can be carried out in one or more solvents, for example, polar aprotic solvents (e.g., dimethylformamide or dimethylsulfoxide), ethers (e.g., tetrahydrofuran or dioxane), chlorinated solvents (e.g., dichloromethane, chloroform or carbon tetrachloride), or mixture thereof.

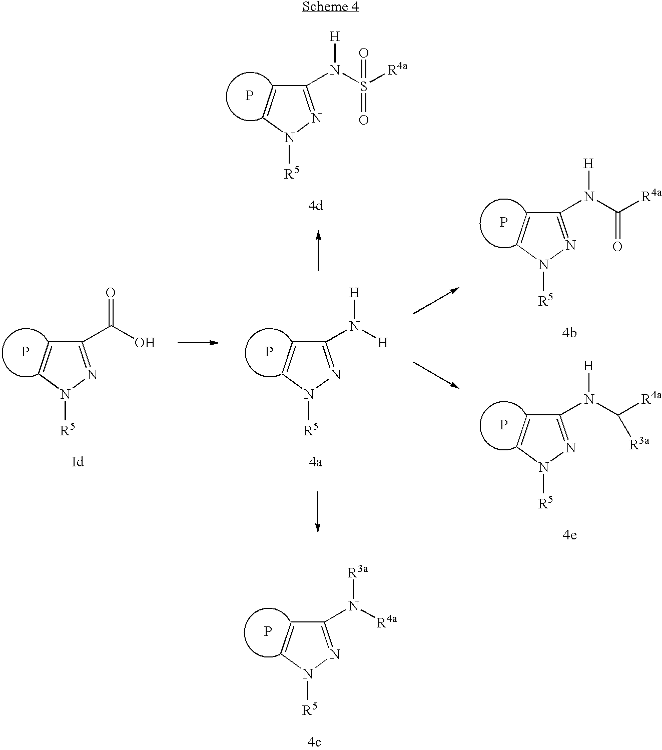

The compounds of Formulas 4b, 4c, 4d and 4e can be prepared according to scheme 4. The compound of Formula 1d is converted to an acyl azide, which is converted to a compound of Formula 4a (such as by treatment with aqueous sulphuric acid). The compound of Formula 4a is converted to a compound of Formula 4b, 4c, 4d, or 4e. For example, the compound of Formula 4a can be reacted with a compound of Formula R^{4a}COX (wherein X is a leaving group such as halogen, OTs or OMs) to form a compound of Formula 4b. The compound of Formula 4a can be reacted with a compound of Formula R^{4a}X followed by R^{3a}X to form a compound of Formula 4c. The compound of Formula 4a can be reacted with a compound of Formula R^{4a}SO₂X to form a compound of Formula 4d. The compound of Formula 4a can be reacted, preferably in the presence of a reducing agent, with a compound of Formula R^{4a}C(O)R^{3a} to form a compound of Formula 4e.

The compounds of Formulae 4a, 4b, 4c, 4d and 4e with R⁵ being at the 2-position of the pyrazole ring rather than the 1-position can also be produced using this reaction scheme.

The compounds of Formula 1d can be reacted with coupling agents, for example, benzotriazolyloxytris(dimethylamino)phosphonium hexafluorophosphate and metal azides (e.g., sodium azide or potassium azide). These reactions can be carried out in the presence of one or more tetraalkylammonium salts, for example, tetrabutyl ammonium bromide, benzyl triethylammonium chloride, cetyltriethylammonium bromide or mixture thereof, and/or in the presence of one or more organic bases, for example, triethylamine, diethylamine, diisopropylethylamine, pyridine, tetramethyl guanidine or mixture thereof. These reactions can also be carried out in one or more solvents, for example, chlorinated solvents (e.g., dichloromethane, chloroform or tetrachloromethane), aprotic polar solvents (e.g., dimethylformamide or dimethylsulfoxide), aromatic solvents (e.g., benzene, toluene or xylene), ethers (e.g., diethyl ether, tetrahydrofuran or dioxane) or mixture thereof. The reaction of acyl azides with sulfuric acid can also be carried out in one or more solvents, for example, chlorinated solvents (e.g., dichloromethane, chloroform or tetrachloromethane), aprotic polar solvents (e.g., dimethylformamide or dimethylsulfoxide), aromatic solvents (e.g., benzene, toluene or xylene), ethers (e.g., tetrahydrofuran or dioxane) or mixture thereof.

Compounds of Formula 4a can be reacted with R^{4a}SO₂X or R^{4a}° COX in the presence of one or more organic bases, for example, triethylamine, diethylamine, diisopropylethylamine, pyridine, tetramethyl guanidine or mixture thereof, and/or in one or more solvents, for example, chlorinated solvents (e.g., dichloromethane, chloroform or tetrachloromethane), aprotic polar solvents (e.g., dimethylformamide or dimethylsulfoxide), aromatic solvents (e.g., benzene, toluene or xylene), ethers (e.g., diethyl ether, tetrahydrofuran or dioxane) or mixture thereof.

Compounds of Formula 4a can be reacted with R^{4a}X in the presence of one or more inorganic bases, for example, potassium carbonate, sodium carbonate, cesium carbonate, sodium bisulphate, sodium acetate or mixture thereof, and/or in one or more solvents, for example, chlorinated solvents (e.g., dichloromethane, chloroform or tetrachloromethane), aprotic polar solvents (e.g., acetone, dimethylformamide or dimethylsulfoxide), aromatic solvents (e.g., benzene, toluene or xylene), ethers (e.g., diethyl ether, tetrahydrofuran or dioxane) or mixture thereof.

Formula 4a can be reacted with compounds of formula R^{4a}C(O)R^{3a} in the presence of a metal borohydride of Formula MB(R⁰)₃H, wherein M is lithium, sodium or potassium and each occurrence of R⁰ is the same or different and independently selected from hydrogen, alkyl, alkoxy, CN and acyloxy, and B is boron. These reactions can be carried out in one or more solvents, for example, polar aprotic solvents (e.g., dimethylformamide or dimethylsulfoxide), ethers (e.g., tetrahydrofuran or dioxane), chlorinated solvents (e.g., dichloromethane, 1,2-dichloroethane chloroform or carbon tetrachloride), nitrites (e.g., acetonitrile or propionitrile), acetates (e.g., ethyl acetate or propyl acetate) or mixture thereof. These reactions can also be carried out in the presence of one or more organic acids, for example, acetic, benzoic, trifluoroacetic acid or mixture thereof.

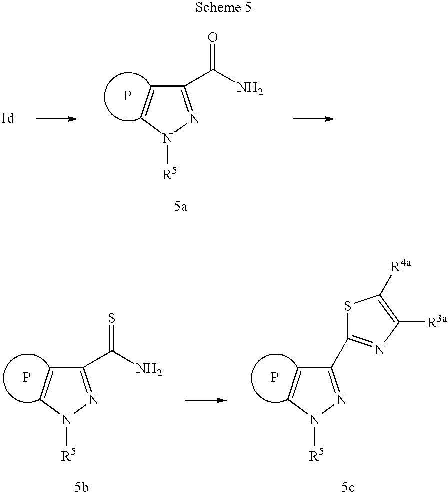

The compounds of Formula 5c can be prepared according to scheme 5. The compound of Formula 1d is converted to a compound of Formula 5a, for example, by reaction with ammonia. The compound of Formula 5a is converted to a compound of Formula 5b, for example, by reaction with a thionating agent such as phosphorous pentasulfide or Lawesson's reagent. The compound of Formula 5b is then converted to a compound of Formula 5c, for example, by reaction with a compound of Formula R^{4a}COCHR^{3a}X (wherein X is leaving group such as halogen, OTs or OMs) to form a compound of Formula 6c. The compounds of Formulae 5a-5c with R⁵ being at the 2-position can also be prepared by this reaction scheme using the appropriate reagents.

Compounds of Formula 1d can be reacted in the presence of one or more chlorinating agents, for example, oxalyl chloride, thionyl chloride, phosphorous pentachloride, phosphorous oxychloride or mixture thereof, and/or in one or more solvents, for example, chlorinated solvents (e.g., dichloromethane, chloroform or tetrachloromethane), aprotic polar solvents (e.g., dimethylformamide or dimethylsulfoxide), aromatic solvents (e.g., benzene, toluene or xylene), ethers (e.g., diethyl ether, tetrahydrofuran or dioxane) or mixture thereof.

Compounds of Formula 5a can be reacted in solvents such as toluene and pyridine.

Compounds of Formula 5b can be reacted in one or more solvents, for example, chlorinated solvents (e.g., dichloromethane, chloroform or tetrachloromethane), aprotic polar solvents (e.g., dimethylformamide or dimethylsulfoxide), aromatic solvents (e.g., benzene, toluene or xylene), ethers (e.g., diethyl ether, tetrahydrofuran or dioxane) or mixture thereof.

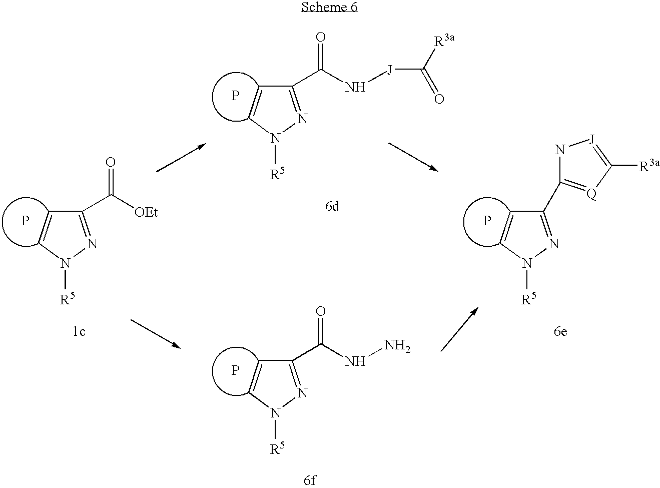

Compounds of Formula 6e can be prepared according to scheme 6. The compound of Formula 1c is converted to a compound of formula 6d, for example, by coupling a compound of formula R^{3a}COJHNH₂ (wherein J is CH or N) to it. The compound of Formula 6d can be cyclised to form a compound of Formula 6e (wherein Q is O, and J is CH or N). The compound of formula 6d can also be reacted with a thionating agent to form a compound of formula 6e (wherein Q is S, and J is CH or N).

The compound of Formula 1c can also be converted into a compound of Formula 6f, for example, by reaction with a hydrazine hydrate. The compound of Formula 6f can be converted into a compound of Formula 6e by reacting it with an acylating agent of the Formula R^{3a}COCl to form a N,N′-diacylhydrazine, which can be cyclized (e.g., in situ) to form the compound of formula 6e (wherein Q is O).

Compounds of formula 1c can be reacted with R^{3a}COJHNH₂ in presence of one or more coupling reagents, for example, phosphonium salts, for example, benzotriazol-1-yloxytris(dimethylamino)-phophonium hexafluorophosphate, benzotriazol-1-yloxytris(pyrrolidino)-phophonium hexafluorophosphate or bromotri(pyrrolidino) phosphonium hexafluorophosphate, carbodiimides, for example, dicylohexylcarbodiimide, N,N′-diisopropylcarbodiimide or 1-(3-dimethylaminopropyl)-3-ethylcarbodiimide. The coupling can also be carried out in the presence or one or more bases, for example, triethylamine, diethylamine, diisopropylethylamine, pyridine, tetramethyl quanidine or mixture thereof, and/or in one or more solvents, for example, aprotic polar solvents (e.g., dimethylformamide or dimethylsulfoxide), protic polar solvents (e.g., methanol, ethanol or propanol), ethers (e.g., diethyl ether, tetrahydrofuran, dioxane or dimethoxyethane), aromatic solvents (e.g., benzene, chlorobenzene, toluene or xylene) or mixture thereof.

The compounds of formula 6d can be cyclized in the presence of a dehydrating agent, for example, phosphorous pentoxide or a chlorinating agent (for example, phosphorous oxychloride or phosphorouspentachloride) and/or in one or more solvents, for example, aprotic polar solvents (e.g., dimethylformamide or dimethylsulfoxide), aromatic solvents (e.g., benzene, chlorobenzene, toluene or xylene) or mixture thereof.

Suitable thionating agents for converting the compound of Formula 6d to a compound of Formula 6e include, but are not limited to, phosphorus pentasulfide or lawesson reagent. The thionating reaction can be performed in one or more solvents, for example, aprotic polar solvents (e.g., dimethylformamide or dimethylsulfoxide), aromatic solvents (e.g., benzene, chlorobenzene, toluene or xylene) or mixture thereof.

Compounds of Formula 6f can be reacted with compounds of Formula R^{4a}COCl in the presence of one or more organic bases, for example, triethylamine, diethylamine, diisopropylethylamine, pyridine, tetramethyl quanidine or mixture thereof, and/or in one or more solvents, for example, chlorinated solvents (e.g., dichloromethane, chloroform or tetrachloromethane), aprotic polar solvents (e.g., dimethylformamide or dimethylsulfoxide), aromatic solvents (e.g., benzene, toluene or xylene), ethers (e.g., diethyl ether, tetrahydrofuran or dioxane) or mixture thereof.

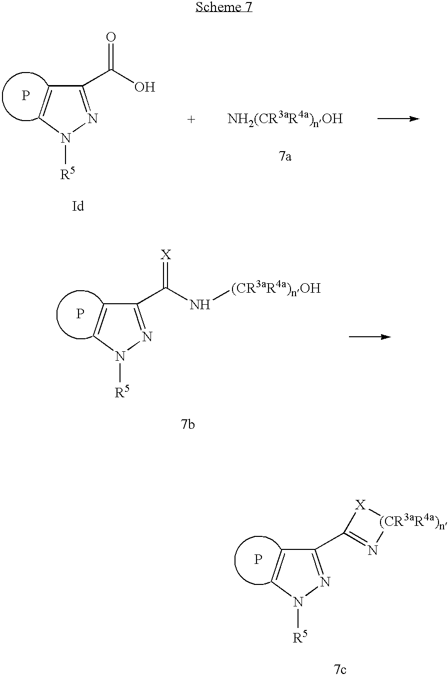

Compounds of Formula 7c can be prepared according to scheme 7. The compound of Formula 1d is converted to a compound of Formula 7b, such as by reaction with a compound of the Formula NH₂(CR^{3a}R^{4a})ₙOH (wherein X is O), which on reaction with a thionating agent yields the compound of Formula 7b, wherein X is S. The compound of Formula 7b can be cyclized to form a compound of Formula 7c. Compounds of Formulae 7b and 7c with R⁵ being at the 2-position of the pyrazole ring can also be produced by this reaction scheme using the appropriate starting materials.

Compounds of Formula 1d can be reacted in the presence of peptide coupling agents, for example, benzotriazolyloxytris(dimethylamino)phosphonium hexafluorophosphate, dicyclohexyl carbodiimide and/or one or more organic bases, for example, triethylamine, dimethylamine, diisopropylethylamine, pyridine or mixture thereof. These reactions can also be carried out in one or more solvents, for example, aprotic polar solvents (e.g., dimethylformamide or dimethylsufoxide), chlorinated solvents (e.g., dichloromethane, chloroform or tetrachloromethane), aromatic solvents (e.g., benzene, toluene or xylene), ethers (e.g., diethyl ether, tetrahydrofuran or dioxane), nitriles (e.g., acetonitrile or propionitrile) or mixture thereof.

Compounds of Formula 7b can be reacted with thionating agents, for example, phosphorous pentasulfide or Lawesson's reagent, in one or more solvents, for example, aprotic polar solvents (e.g., dimethylformamide or dimethylsufoxide), chlorinated solvents (e.g., dichloromethane, chloroform or tetrachloromethane), aromatic solvents (e.g., benzene, toluene, pyridine or xylene), ethers (e.g., diethyl ether, tetrahydrofuran or dioxane), nitriles (e.g., acetonitrile or propionitrile) or mixture thereof.

Compounds of Formula 7b can be cyclized in the presence of one or more haloginating agents, for example, thionyl chloride, phosphoryl chloride, phosphorous pentachloride or mixture thereof. These reactions can also be carried out in one or more solvents, for example, aprotic polar solvents (e.g., dimethylformamide or dimethylsufoxide), chlorinated solvents (e.g., dichloromethane, chloroform or tetrachloromethane), aromatic solvents (e.g., benzene, toluene or xylene), ethers (e.g., diethyl ether, tetrahydrofuran or dioxane), nitriles (e.g., acetonitrile or propionitrile), acetates (e.g., ethyl acetate or propyl acetate) or mixture thereof. Compounds of Formula 7b can also be cyclized by heating with phosphorous pentaoxide, at a temperature of about 140-180° C.

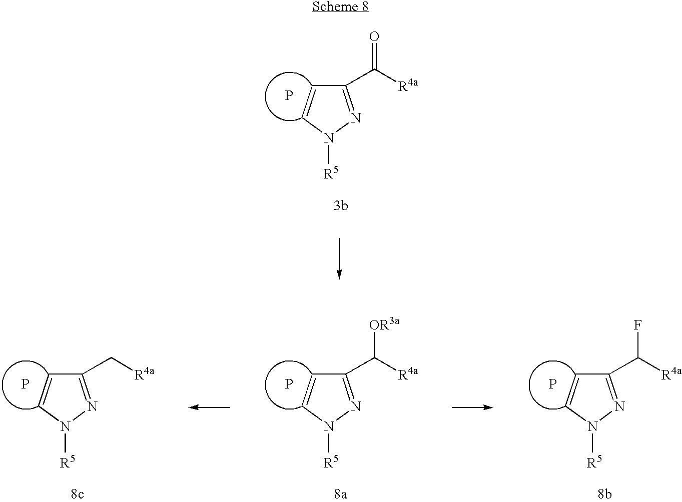

Compounds of Formulas 8a and 8b and 8c can be prepared according to scheme 8. The compound of formula 8a (wherein R^{3a} is H) can be prepared by reduction of the compound of formula 3b. The compound of Formula 8a can be (a) fluorinated to form a compound of Formula 8b, or (b) converted to a compound of Formula 8c, such as by reaction with R^{3a}SiH (wherein R^{3a} can be alkyl). Compounds of Formula 8a (when R^{3a} is hydrogen) can also be reacted with compounds of Formula R^{3a}X or R^{3a}COX to form compounds of Formula 8a (wherein R^{3a} can be alkyl or aryl). Compounds of Formulae 8a, 8b and 8c with R⁵ being at the 2-position of the pyrazole ring can also be prepared by this reaction scheme using the appropriate starting materials.

Compounds of Formula 3b can be reacted in the presence of reducing agents, such as sodium borohydrate, lithium borohydrate and lithium aluminium hydrate, in one or more solvents such as alcohols (e.g., methanol or ethanol), ethers (e.g., diethylether or tetrahydrofuran), acetates (e.g., ethyl acetate or propyl acetate), or mixture thereof.

Compounds of Formula 8a can be reacted in the presence of one or more fluorinating agents, for example, dimethylaminosulfur trifluoride, Selectfluor®, N-fluorobenzene sulphonamide or mixture thereof. These reactions can also be carried out in one or more solvents, for example, polar aprotic solvents (e.g., dimethylformamide or dimethylsulfoxide), ethers (e.g., tetrahydrofuran or dioxane), chlorinated solvents (e.g., dichloromethane, chloroform or carbon tetrachloride), nitriles (e.g., acetonitrile or propionitrile), acetates (e.g., ethyl acetate or propyl acetate) or mixture thereof. These reactions can also be carried out in the presence of one or more bases, for example, potassium carbonate, sodium hydride, sodium acetate, sodium thiosulpahte, potassium-t-butoxide, sodium-t-butoxide, lithium diisopropylamide, sodium methoxide, sodium ethoxide, potassium methoxide, potassium ethoxide, lithium carbonate or mixture thereof.

Compounds of Formula 8a can be reacted with R^{3a}SiH in the presence of one or more acids, for example, organic acids (e.g., acetic, benzoic or trifluoroacetic acid), Lewis acid (e.g., boron trifluoride) or mixture thereof.

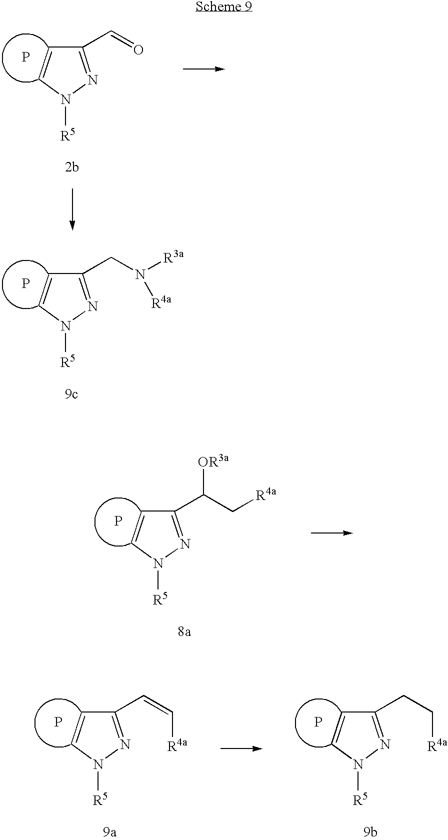

Compounds of Formula 8a, 9a, 9b and 9c can be prepared according to scheme 9. The compound of Formula 2b can be converted to a compound of Formula 8a, such as by reaction with R^{4a}CH₂Li or R^{4a}CH₂MgX (wherein R^{3a} in Formula 8a is hydrogen). The compound of Formula 2b can alternatively be reacted with an amine of Formula R^{3a}R^{4a}NH to form a compound of Formula 9c. The compound of Formula 8a can be subjected to an elimination reaction to form a compound of Formula 9a. The compound of Formula 9a can be hydrogenated to form a compound of Formula 9b. Compounds of Formulae 8a, 9a, 9b and 9c with R⁵ being at the 2-position of the pyrazole ring can also be prepared by this reaction scheme using appropriate starting materials.

Compounds of Formula 2b can be reacted with Grignard reagents of the formula R^{4a}CH₂MgX or R^{4a}CH₂Li, in one or more ethers, for example, diethyl ether, tetrahydrofuran, dioxane or mixture thereof. Compounds of Formula 2b can be reacted with amines of Formula R^{3a}R^{4a}NH in the presence of a metal borohydride of Formula MB(R⁰)₃H, wherein M is lithium, sodium or potassium, each occurrence of R⁰ is the same or different and is independently selected from hydrogen, alkyl, alkoxy, CN or acyloxy, and B is boron. These reactions can also be carried out in one or more solvents, for example, polar aprotic solvents (e.g., dimethylformamide or dimethylsulfoxide), ethers (e.g., tetrahydrofuran or dioxane), chlorinated solvents (e.g., dichloromethane, 1,2-dichloroethane chloroform or carbon tetrachloride), nitriles (e.g., acetonitrile or propionitrile), acetates (e.g., ethyl acetate or propyl acetate) or mixture thereof. These reactions can also be carried out in the presence of one or more organic acids, for example, acetic, benzoic, trifluoroacetic acid or mixture thereof.

Compounds of Formula 8a can be reacted in the presence of one or more acids, for example, sulfuric, orthophosphoric, hydrochloric, hydrobromic or mixture thereof. These reactions can also be carried out in one or more solvents, for example, polar aprotic solvents (e.g., dimethylformamide or dimethylsulfoxide), ethers (e.g., tetrahydrofuran or dioxane), chlorinated solvents (e.g., dichloromethane, chloroform or carbon tetrachloride), nitriles (e.g., acetonitrile or propionitrile), acetates (e.g., ethyl acetate or propyl acetate) or mixture thereof. Compounds of Formula 9a can be hydrogenated by procedures known in the art.

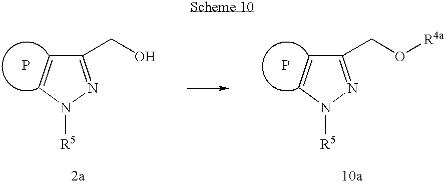

Compounds of Formula 10a can be prepared according to scheme 10. The compound of Formula 2a is converted to a compound of Formula 10a, for example, by reaction with a compound of Formula R^{4a}X (wherein X is a halogen). Compounds of Formula 10a with R⁵ being at the 2-position of the pyrazole ring can be prepared by this reaction scheme using appropriate starting materials.

Compounds of Formula 2a can be reacted in the presence of one or more inorganic bases, for example, sodium hydride, potassium hydride, potassium carbonate, sodium carbonate, sodium thiosulphate, sodium methoxide, sodium ethoxide, potassium methoxide, potassium ethoxide, lithium carbonate or mixture thereof. These reactions can also be carried out in one or more solvents, for example, polar aprotic solvents (e.g., dimethylformamide or dimethylsulfoxide), ethers (e.g., tetrahydrofuran or dioxane), chlorinated solvents (e.g., dichloromethane, chloroform or carbon tetrachloride), nitriles (e.g., acetonitrile or propionitrile), acetates (e.g., ethyl acetate or propyl acetate) or mixture thereof.

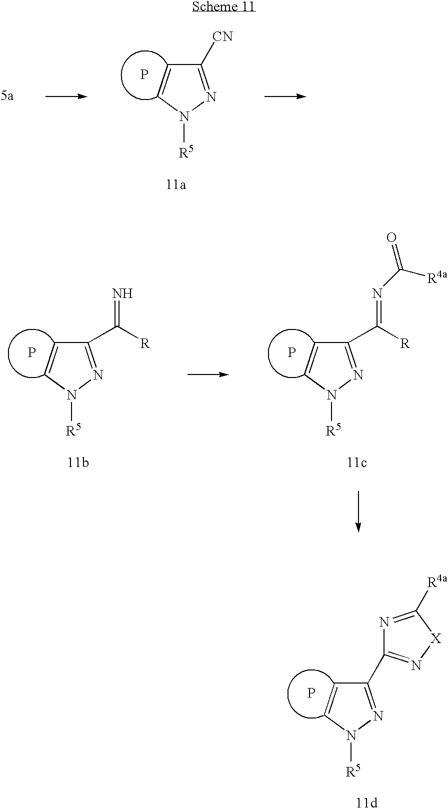

Compounds of Formula 11d can be prepared according to scheme 11. The compound of Formula 5a is converted to a nitrile of Formula 11A. Such methods are known in the art. The compound of Formula 11a IS converted to a compound of Formula 11b, for example, by reaction with an alcohol (wherein R is alkoxy). The compound of Formula 11b is converted to a compound of Formula 11c, such as by reaction with R^{4a}COX″ (wherein X″ is halogen). The compound of Formula 11c is converted to a compound of Formula 11d, such as by reaction with a compound of Formula R′NHNH₂ (wherein R′ is hydrogen, alkyl, aryl or heteroaryl). Compounds of Formula 11c can be reacted with hydroxylamine to form compounds of Formula 11d (wherein X is O). Compounds of Formulae 11a-11d with R⁵ being at the 2-position of the pyrazole ring can be prepared by this reaction scheme using appropriate starting materials.

Compounds of Formula 5a can be reacted in the presence of chlorinating agents, for example, thionyl chloride, oxalyl chloride, phosphorous pentachloride or mixture thereof. These reactions can also be carried out in the presence of one or more dehydrating agents, for example, phosphorous trichloride, phosphorous pentaoxide or mixture thereof. These reactions can also be carried out in one or more solvents, for example, polar aprotic solvents (e.g., dimethylformamide or dimethylsulfoxide), ethers (e.g., tetrahydrofuran or dioxane), chlorinated solvents (e.g., dichloromethane, chloroform or carbon tetrachloride), nitrites (e.g., acetonitrile or propionitrile), acetates (e.g., ethyl acetate or propyl acetate) or mixture thereof.

Compounds of Formula 11a can be reacted in the presence of hydrochloric acid, and/or in a solvent such as ethanol, methanol, propanol or mixture thereof (preferably under dry conditions). The products obtained as their salts can be treated with bases such as potassium hydroxide, sodium hydroxide, lithium hydroxide or mixtures thereof to obtain compounds of formula 11b.

Compounds of Formula 11b can be reacted in the presence of one or more bases, for example, triethylamine, trimethylamine, diethylamine, disopropylethylamine, pyridine or mixture thereof. These reactions can also be carried out in one or more solvents, for example, polar aprotic solvents (e.g., dimethylformamide or dimethylsulfoxide), ethers (e.g., tetrahydrofuran or dioxane), chlorinated solvents (e.g., dichloromethane, chloroform or carbon tetrachloride), nitriles (e.g., acetonitrile or propionitrile), acetates (e.g., ethyl acetate or propyl acetate) or mixture thereof.

Compounds of Formula 11c can be reacted with R′NHNH₂ in the presence of one or more bases, for example, triethylamine, trimethylamine, diethylamine, diisopropylethylamine, pyridine or mixture thereof. These reactions can also be carried out in one or more solvents, for example, polar aprotic solvents (e.g., dimethylformamide or dimethylsulfoxide), ethers (e.g., tetrahydrofuran or dioxane), chlorinated solvents (e.g., dichloromethane, chloroform or carbon tetrachloride), nitriles (e.g., acetonitrile or propionitrile), acetates (e.g., ethyl acetate or propyl acetate) or mixture thereof.

Compounds of Formula 11c can be reacted with hydroxylamine in the presence of one or more bases, for example, triethylamine, trimethylamine, diethylamine, isopropylethylamine, pyridine, sodium methoxide, sodium ethoxide or mixture thereof. These reactions can also be carried out in one or more solvents, for example, polar aprotic solvents (e.g., dimethylformamide or dimethylsulfoxide), ethers (e.g., tetrahydrofuran or dioxane), chlorinated solvents (e.g., dichloromethane, chloroform or carbon tetrachloride), nitriles (e.g., acetonitrile or propionitrile), acetates (e.g., ethyl acetate or propyl acetate) or mixture thereof.

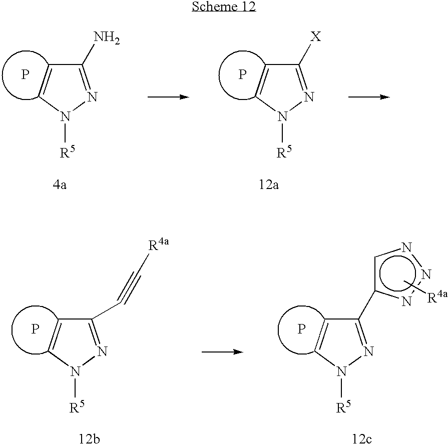

Compounds of Formula 12c can be prepared according to scheme 12. The compound of Formula 12a is prepared from the compound of Formula 4a, for example, by diazotization with nitrous acid followed by treatment with a metal halide such as sodium chloride, sodium bromide, or potassium iodide. This reaction is performed either in the presence or absence of copper (I) halide, such as cuprous chloride (as described by Sandmeyer, T. in Chem. Ber. 1884, 17, 1633 or any known modification of the procedure, e.g., Condret, C. et. al. Synth. Commun. 1996, 26, 3143).

The compound of Formula 12a is converted to a compound of Formula 12b, for example, by reaction with a compound of Formula R^{4a}—C≡CH. The compound of Formula 12b can be reacted with an azide to form a compound of Formula 12c.

Compounds of Formula 12a can be reacted by following the procedures known in the art, for example, Takashashi et al., Synthesis, 1980, 627-314. These reactions can also be carried out in the presence of one or more palladium catalysts (e.g., bis[triphenylphosphine]palladium dichloride, tetrakis(triphenylphosphine)palladium(0)) and/or copper catalysts such as a copper halide, e.g., copper(I) iodide, in the presence of one or more bases, for example, organic bases, (e.g., triethylamine, pyridine, N,N-diisopropylethylamine or trimethylamine), inorganic bases (e.g., potassium carbonate, sodium carbonate or lithium carbonate) or mixture thereof and/or in a solvent such as dimethylformamide or acetonitrile.

Compounds of Formula 12b can be reacted with one or more azides, for example, metal azide (e.g., sodium azide or potassium azide), organic azide (e.g., alkyl azide or aryl azide) or mixture thereof. These reactions can also be carried out in one or more solvents, for example, protic polar solvents (e.g., methanol, ethanol or propanol), aromatic solvents (e.g., benzene, chlorobenzene, toluene or xylene), aliphatic solvents (e.g., hexane or heptane), ethers (e.g., diethyl ether, tetrahydrofuran, dioxane or dimethoxy ethane) or mixture thereof.

Compounds of Formulae 12a-12c with R⁵ being at the 2-position of the pyrazole ring can be prepared by this reaction scheme using appropriate starting materials.

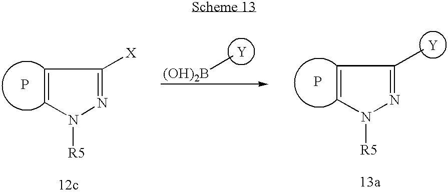

Compounds of Formula 13a can be prepared according to scheme 13. The compound of Formula 12c is converted to a compound of Formula 13a, for example, by reaction with a boronic acid of the formula (OH)₂B(Y) (wherein Y can be alkyl, aryl heteroaryl or heterocyclyl). Compounds of Formula 13a with R⁵ being at the 2-position of the pyrazole ring can be prepared by this reaction scheme using appropriate starting materials.

Compounds of Formula 12c can be reacted in the presence of one or more palladium catalysts, for example, palladium(II) acetate (Pd(OAc)₂), palladium(II) chloride (PdCl₂), bis(triphenylphosphine)palladium(II) chloride ([(C₆H₅)₃P]₂PdCl₂), or mixture thereof, and/or in one or more solvents, for example, protic polar solvents (e.g., methanol, ethanol or propanol), aromatic solvents (e.g., benzene, chlorobenzene, toluene or xylene), acetonitrile, aliphatic solvents (e.g., hexane or heptane), ethers (e.g., diethyl ether, tetrahydrofuran, dioxane or dimethoxy ethane), or mixture thereof.

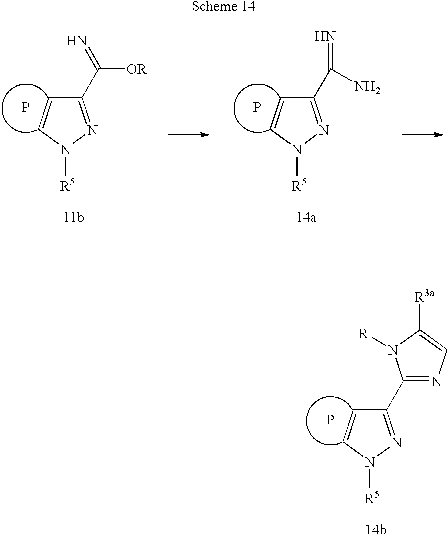

Compounds of Formula 14b can be prepared according to scheme 14. The compound of Formula 11b (wherein R is alkoxy) is converted to an amidine of Formula 14a, for example, by methods known in the art. The amidine of Formula 14a is converted to the compound of Formula 14b, for example, by reaction with a compound of Formula R^{3a}COCH₂X (where X is a leaving group). Compounds of formula 14b wherein R is hydrogen, can be alkylated, arylated or heteroarylated under standard conditions to give regioisomeric 14b wherein R is alkyl, aryl or heteroaryl, respectively. Compounds of Formulae 14a and 14b with R⁵ being at the 2-position of the pyrazole ring can be prepared by this reaction scheme using appropriate starting materials.

Compounds of Formula 11b can be reacted in one or more solvents, for example, protic polar solvents (e.g., methanol, ethanol or propanol), aromatic solvents (e.g., benzene, chlorobenzene, toluene or xylene), aliphatic solvents (e.g., hexane or heptane), ethers (e.g., diethyl ether, tetrahydrofuran, dioxane or dimethoxy ethane), or mixture thereof.

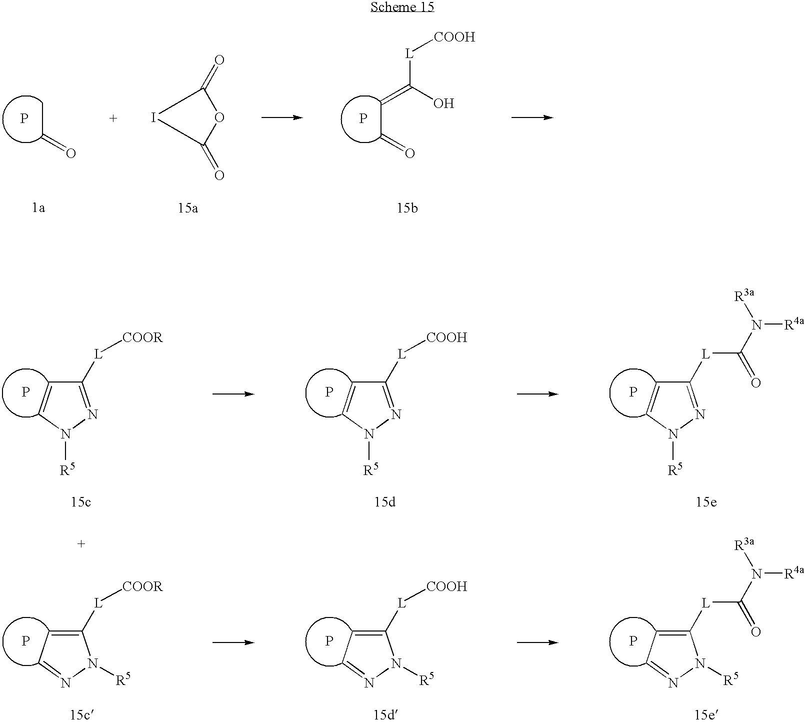

Compounds of Formula 15e and 15e′ can be prepared according to scheme 15. The compound of formula 1a is reacted with a cyclic anhydride of formula 15a (wherein L is (CH₂)ₙ where n is 2-5 to form a diketoacid of Formula 15b. The diketoacid of Formula 15b is then condensed with a hydrazine (such as a hydrazine of the formula R⁵NHNH₂), preferably in the presence of an acid, to furnish isomeric pyrazole carboxylic acid esters of Formulas 15c and 15c′ (wherein R is alkyl). Compounds of Formula 15c and 15c′ are hydrolyzed to form compounds of Formula 15d and 15d′, respectively. Compounds of formula 15d and 15d′ are converted to compounds of Formulas 15e and 15e′, respectively, for example, by reaction with a tertiary amine, preferably in the presence of a base or coupling agent.

Compounds of Formula 1a can be reacted in one or more solvents, for example, protic polar solvents (e.g., methanol, ethanol or propanol), aromatic solvents (e.g., benzene, chlorobenzene, toluene or xylene), aliphatic solvents (e.g., hexane or heptane), ethers (e.g., diethyl ether, tetrahydrofuran, dioxane or dimethoxy ethane) or mixture thereof. Compounds of Formula 1a can also be reacted in the presence of one or more bases, for example, metal alkoxides (e.g., sodium methoxide or sodium ethoxide), metal amides (e.g., lithium diisopropyl amide or lithium bis(trimethylsilyl)amide), metal hydride (e.g., lithium hydride, sodium hydride or potassium hydride), or mixture thereof. During the course of the reaction all possible combinations of bases and solvents can be used and are therefore within the scope of the present invention.

Compounds of Formula 15b can be reacted in the presence of one or more acids, for example, hydrochloric, hydrobromic acid or mixture thereof, and/or in one or more protic polar solvents, for example, methanol, ethanol, propanol, isopropanol or mixture thereof. Compounds of Formula 1c (when R⁵ is hydrogen) can be reacted in the presence of one or more inorganic bases, for example, lithium hydride, sodium hydride, potassium hydride or mixture thereof. Compounds of Formula 15c and 15c′ can be hydrolyzed in the presence of one or more inorganic bases, for example, sodium hydroxide, potassium hydroxide, lithium hydroxide or mixture thereof, and/or in one or more protic polar solvents, for example, water, methanol, ethanol, propanol, isopropanol or mixture thereof. The hydrolysis can also be carried out by using various other methods known in the art, for example under acidic conditions.

Compounds of Formula 15d and 15d′ can be reacted in the presence of peptide coupling agents, for example, benzotriazolyloxytris(dimethylamino)phosphonium hexafluorophosphate, and/or one or more organic bases, for example, triethylamine, dimethylamine, diisopropylethylamine, pyridine or mixture thereof. These reactions can also be carried out in on or more solvents, for example, aprotic polar solvents (e.g., dimethylformamide or dimethylsufoxide), chlorinated solvents (e.g., dichloromethane, chloroform or tetrachloromethane), aromatic solvents (e.g., benzene, toluene or xylene), ethers (e.g., diethyl ether, tetrahydrofuran or dioxane), nitriles (e.g., acetonitrile or propionitrile) or mixture thereof.

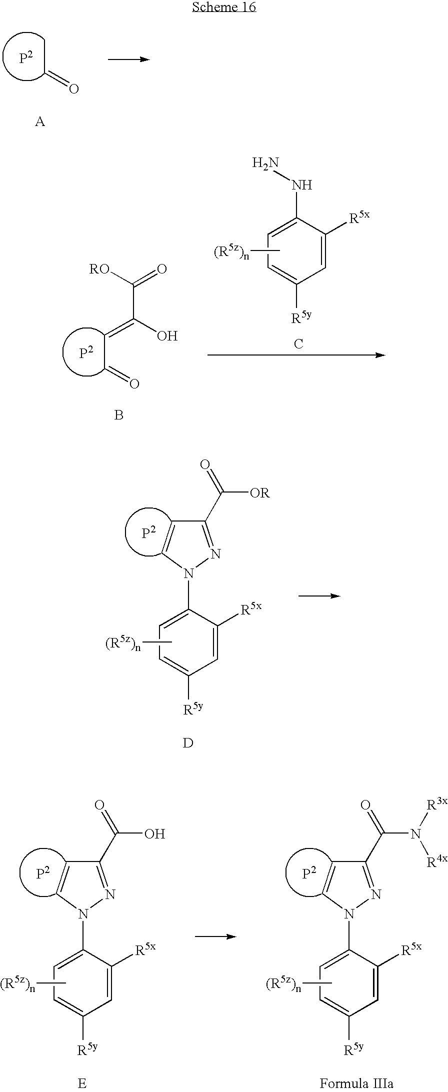

A compound of Formula IIIa (wherein P², n, R^{3x}, R^{4x}, R^{5x}, R^{5y} and R^{5z} are the same as defined earlier) can be prepared using the reaction sequence as shown in scheme 16 above. The compound of Formula A is converted to a compound of Formula B, for example, by reaction with a dialkyl oxalate (such as diethyl oxalate) to form an appropriate ethyl acetate compound of Formula B. The compound of Formula B is reacted with a compound of Formula C to form an appropriate pyrazole compound of formula D. The compound of Formula D is hydrolyzed to form a compound of Formula E. The compound of formula E is coupled with an amine of the formula NHR^{3x}R^{4x} to form an amide compound of formula IIIa.

The compound of Formula A can be reacted in the presence of one or more bases such as metal alkoxides (e.g., potassium tert-butoxide, sodium methoxide or sodium ethoxide) or organometallic bases (e.g., lithium hexamethyl disilazide, n-butyllithiun, sec-butyllithium or tert-butyllithium) and/or in one or more solvents such as alcoholic solvents (e.g., ethanol, methanol or isopropanol), ethers (e.g., diethylether or tetrahydrofuran), aromatic solvents (e.g., benzene, toluene or xylene), aprotic polar solvents (e.g., dimethylformamide or dimethylsulfoxide) or a mixture thereof.

The reaction between a compound of formula B and a compound of formula C can be carried out in the presence of one or more acids (e.g., hydrochloric acid, hydrobromic acid or aceticacid) and/or in one or more solvents such as alcoholic solvents (e.g., ethanol, methanol or isopropanol), aromatic solvents (e.g., benzene, toluene or xylene), aprotic polar solvents (e.g., dimethylformamide or dimethylsulfoxide), ethers (e.g., diethylether or tetrahydrofuran), halogenated solvents (e.g., dichloromethane, dibromomethane, chloroform or carbon tetrachloride), or a mixture thereof.

The compound of formula E can be hydrolyzed in the presence of one or more bases such as inorganic bases (e.g., potassium hydroxide or sodium hydroxide) and/or in one or more solvents such as polar protic solvents (e.g., water, ethanol, methanol or isopropanol), aprotic polar solvents (e.g., dimethylformamide or dimethylsulfoxide), ethers (e.g., diethylether, dioxane or tetrahydrofuran), halogenated solvents (e.g., dichloromethane, dibromomethane, chloroform or carbon tetrachloride), or a mixture thereof.

The compound of formula E can be reacted with one or more coupling agents (e.g., benzotriazol-1-yloxytris(dimethylamino)-phosphonium hexafluorophosphate, N,N′-diclyclohexylcarbodiimide or 1-(3-dimethylaminopropyl)-3-ethyl carbodiimide hydrochloride) optionally in the presence of one or more bases (e.g., N,N-diisopropyl ethylamine, triethylamine, diethylamine, pyridine, or 4-dimethylamino pyridine) and/or in one or more solvents such as ethers (e.g., diethylether or tetrahydrofuran), aprotic polar solvents (e.g., dimethylformamide or dimethylsulfoxide), aromatic solvents (e.g., benzene, toluene or xylene), halogenated solvents (e.g., dichloromethane, dibromomethane, chloroform or carbon tetrachloride), or a mixture thereof.

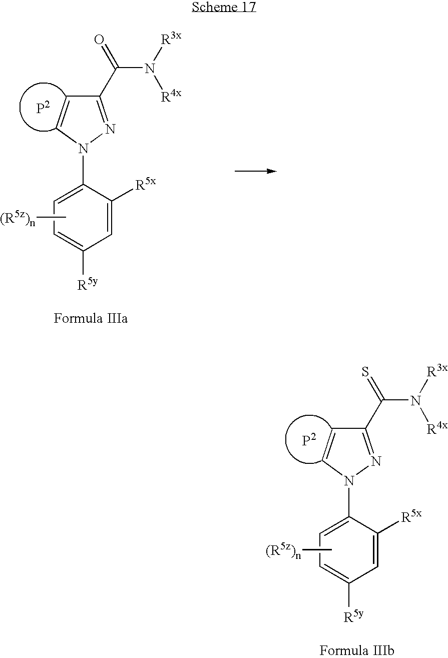

A compound of Formula IIIb (wherein P², n, R^{3x}, R^{4x}, R^{5x}, R^{5y} and R^{5z} are the same as defined earlier) can be prepared using the reaction sequence as shown in scheme 17 above. The compound of formula IIIa (wherein P², n, R^{3x}, R^{4x}, R^{5x}, R^{5y} and R^{5z} are the same as defined earlier) is converted to a compound of formula IIIb. The reaction can be carried out in the presence of one or more sulfurizing agents, such as phosphorus pentasulfide or the Lawesson's reagent, and/or in one or more solvents such as basic solvents (e.g., pyridine or tolidine), ethers (e.g., diethylether or tetrahydrofuran), aprotic polar solvents (e.g., dimethylformamide or dimethylsulfoxide), aromatic solvents (e.g., benzene, toluene or xylene), halogenated solvents (e.g., dichloromethane, chlorobenzene, dibromomethane, chloroform or carbon tetrachloride), or a mixture thereof.

### Experimental

The following acronyms, abbreviations, terms and definitions have been used throughout this disclosure. The following acronyms, abbreviations, terms and definitions have been used throughout the experimental section. Acronyms and abbreviations: THF (tetrahydrofuran), n-BuLi (n-butyllithium), AcOEt (ethyl acetate), CDCl₃ (deuterated chloroform), DMSO (dimethylsulfoxide), DMF (dimethylformamide), DMSO-d₆ (Hexadeuterodimethyl sulfoxide), M.P. (melting point), rt or RT (room temperature), aq (aqueous), min (minute), equiv, (equivalents) h or hr (hour), atm (atmosphere), TLC (Thin Layer Chromatography), MS or mass spec (mass spectroscopy/spectrometry), NMR (nuclear magnetic resonance), IR (infrared spectroscopy).

NMR abbreviations: br (broad), apt (apparent), s (singlet), d (doublet), t (triplet), q (quartet), dq (doublet of quartets), dd (doublet of doublets), dt (doublet of triplets), m (multiplet), J (Coupling constant in units of Hz).

Room temperature is defined as an ambient temperature range, typically from about 20° C. to about 35° C. Temperature at reflux is defined as +15° C. of the boiling point of the primary reaction solvent. Overnight is defined as a time range of from about 8 to about 16 hours.

Unless otherwise stated, work-up implies distribution of the reaction mixture between the organic and aqueous phase indicated within parenthesis, separation of layers and drying the organic layer over sodium sulphate, filteration and evaporation of the solvent. Purification, unless otherwise mentioned, implies purification by silica gel chromatographic techniques, generally using ethyl acetate/petroleum ether mixture of a suitable polarity as the mobile phase. Use of a different eluent system is indicated within parenthesis.

The present invention is further illustrated by the following examples, which are not to be construed in any way as imposing limitations upon the scope of this disclosure, but rather are intended to be illustrative only. On the contrary, it is to be clearly understood that resort may be had to various other embodiments, modifications, and equivalents thereof which, after reading the description herein, may suggest themselves to one of ordinary skill in the art without departing from the spirit of the present invention. Thus, the skilled artisan will appreciate how the experiments and Examples may be further implemented as disclosed by variously altering the following examples, substituents, reagents, or conditions.

### Intermediates

### Intermediate 1: Preparation of Ethyl 2-oxo-2(3-oxobicyclo[2.2.1]hept-2-yl)acetate

Method 1: A 1.6M soln. of n-BuLi in hexane was added to a solution of hexamethyldisilazane (4.2 ml, 20 mmol) in diethyl ether (91.0 ml) at −78° C. and stirred at that temperature for 15 min. To this mixture was added a solution of norcamphor (2.0 g, 18.2 mmol) in diethyl ether and stirring at −78° C. was continued for further 45 min. Diethyl oxalate (0.98 ml, 6.5 mmol) was added and the mixture was allowed to slowly warm up to 25° C. After stirring overnight, water (200 ml) was added to the solution and the layers separated. The aqueous layer was washed twice with diethyl ether (150 ml), acidified with 1N HCl and extracted into diethyl ether (3×20 ml), the organic layer was dried over Na₂SO₄, filtered and evaporated. Flash chromatography (petroleum ether/ethyl acetate 97:3) gave the title compound as yellow oil (2.0 g, 56%). ¹H-NMR (δ ppm, CDCl_{3,} 300 MHz): 11.41 (br. s, 1H); 4.35 (q, J=7.2, 2H); 3.81 (br. s, 1H); 2.81 (br. s, 1H); 2.05-1.85 (m, 3H); 1.80 (br. d, J=10.5, 1H); 1.59 (br. t, J=7.2, 2 H); 1.41 (t, J=7.2, 3H).

Method 2: A solution of norcamphor (85 g, 0.77 mol) in toluene (800 ml) was added to a slurry of sodium hydride (60% dispersion, 40 g, 1.0 mol) and diethyl oxalate (135 g, 0.92 mol) in toluene (400 ml) at 60° C. and the mixture stirred at the same temperature for 1 hour. The reaction mixture was quenched into ice, acidified with 1N HCl, extracted with ethyl acetate and the organic layers washed with brine and dried over Na₂SO₄ and the solvent was removed under vacuum. Flash chromatography (petroleum ether/ethyl acetate 95:5 gave the title compound as a yellow oil (128 g, 79%)) which was used without purification for the next step.

### Intermediate 2: Ethyl 3-(2,4-difluorophenyl)-3,4-diazatricyclo[5.2.1.0^{2,6}]deca-2(6),4-dien-5-carboxylate

A solution of intermediate 1 (260 g, 1.23 mol), and 2,4-difluorophenylhydrazine hydrochloride (246 g, 1.36 mol) in ethanol (2000 ml) was refluxed for overnight. The solvent was evaporated to 200 ml and cooled to RT and precipitated solid was filtered and washed with ethanol (200 ml), 20% ethanol in hexane (200 ml) and dried to furnish the title compound (334 g, 84%). ¹H-NMR (δ ppm, CDCl₃, 300 MHz): 7.80-7.68 (m, 1H); 7.05-6.95 (m, 2H); 4.42 (q, J=7.2, 2H); 3.67 (br. s, 1H); 3.47 (br. s, 1H); 2.12-2.08 (br. d, J=8.7, 1H); 2.03-1.90 (m, 2H); 1.72-1.65 (br. d, J=8.7, 1H); 1.41 (t, J=7.2, 3H); 1.30-1.17 (m, 2H).

### Intermediate 3: 3-(2,4-Difluorophenyl)-3,4-diazatricyclo[5.2.1.0^{2,6}]deca-2(6),4-dien-5-carboxylic acid

A solution of Intermediate 2 (280 g, 0.88 mol) in isopropyl alcohol (2000 ml) was treated with 1.25 M KOH (69 g, 1.23 mol) and stirred at RT overnight. After removal of isopropyl alcohol, the residue was dissolved in H₂O and acidified to pH 4.0 with aqueous 1N HCl. The precipitate was filtered and dried to give pure intermediate 3 (240 g, 94%). ¹H-NMR (δ ppm, CDCl₃, 300 MHz): 7.80-7.70 (m, 1H); 7.10-6.97 (m, 2H); 3.70 (br. s, 1H); 3.50 (br. s, 1H); 2.12 (d, J=7.2, 1H); 2.08-1.86 (m, 2H); 1.72 (d, J=8.7, 1H); 1.35-1.17 (m, 2H).

### Intermediates 4 and 5: Optical Resolution of 3-(2,4-Difluorophenyl)-3,4-diazatricyclo[5.2.1.0^{2,6}]deca-2(6),4-dien-5-carboxylic acid

### Intermediate 4: Preparation of (1S,7R)-3-(2,4-difluorophenyl)-3,4-diazatricyclo[5.2.1.0^{2,6}]deca-2(6),4-diene-5-carboxylic acid

A slurry of intermediate 3 (racemic, 15.0 g, 51.72 mmol) in acetonitrile (LR grade) (150 ml) was treated with (S)-(−)-α-methylbenylamine (3.66 ml, 28.44 mmol), stirred at RT for 5-10 min and the mixture was heated at reflux for 15 min. Methanol (24 ml) was added slowly till a clear solution resulted and heating was continued for further 30 min. after which the mixture was allowed to cool slowly to RT. The separated crystals were collected by filteration and washed with acetonitrile/MeOH 9:1 (˜15 ml). The acid was recovered from the diastereomeric salt by dissolving in CH₂Cl₂ and extraction with aq. 1N HCl. Reiteration of the same procedure several times gave a mixture (100 mg) enriched in the late eluting enantiomer [Intermediate 4, Rₜ=38.20 min. on a CHIRALCEL AS-H column (dimensions: 250×4.6 mm, particle size: 5μ) using a 90:10:0.1 mixture of n-hexane:isopropanol:trifluoroacetic acid as the eluent at 1 ml/min. flow rate]. M.P.: 114-115° C.; e.e=92%.

### Intermediate 5: Preparation of (1R,7S)-3-(2,4-difluorophenyl)-3,4-diazatricyclo[5.2.1.0^{2,6}]deca-2(6),4-diene-5-carboxylic acid

The mother liquor obtained in the first step of the process described above was evaporated, distributed between CH₂Cl₂ and aq. 1N HCl and the layers were separated. Drying (Na₂SO₄) and evaporation of the organic layer gave a mixture of the two enantiomeric acids (9 g) enriched in the fast eluting enantiomer (Rₜ=34.65 min. under the same conditions described above; e.e=34%). The mixture was enriched in this enantiomer to an e.e of 92% (Intermediate 5, yield=72 mg) by replacing (S)-(−)-α-methylbenylamine with (R)-(+)-α-methylbenylamine in the process described above for the late eluting enantiomer. M.P.: 110-112° C.

### Intermediate 6: Preparation of N5-Methoxy-N5-methyl-(2,4-Difluorophenyl)-3,4-diazatricyclo[5.2.1.0^{2,6}]deca-2(6),4-dien-5-carboxamide

Oxalyl chloride (1.78 ml, 20.65 mmol) was added at 0° C. to a solution of Intermediate 3 (5.0 g, 17.21 mmol) in dichloromethane (30 ml) followed by the addition catalytic amount of DMF and stirred at RT for 2 h. The solvent was evaporated under reduced pressure and the residue was dissolved in dichloromethane (20 ml) and added slowly to a solution of N,O-dimethylhydroxylamine hydrochloride (1.84 g, 19.55 mmol) and Et₃N (5.71 ml, 41.30 mmol) in dichloromethane (20 ml) at 0° C. and the mixture was stirred at RT for 2 h. The reaction mixture was distributed between water and dichloromethane and the organic extracts were washed with brine and dried over Na₂SO₄. Evaporation and purification by column chromatography furnished the title compound (5.20 g, 90%). ¹H-NMR (δ ppm, CDCl₃, 300 MHz): 7.71 (q, J=8.4, 1H); 6.99 (t, J=8.4, 2H); 3.78 (s, 3H); 3.61 (br. s, 1H); 3.47 (br. s, 4H); 2.08 (d, J=7.8, 1H); 1.96 (d, J=7.8, 2H); 1.69-1.59 (m, 1H); 1.27 (d, J=7.2, 2H).

### Intermediate 7: Preparation of 5-(2,4-Difluorophenyl)-4,5-diazatricyclo[5.2.1.0^{2,6}]deca-2(6),3-dien-3-ylmethanol

Intermediate 2 (5.0 g, 15.72 mmol) in dry THF (50 ml) was refluxed for 2 h with LiBH₄ (855 mg, 40.71 mmol). Aq. 1N HCl was added and the mixture was extracted with ethyl acetate and the combined organic layers were washed with brine and dried over Na₂SO₄. Purification of crude product by column chromatography furnished the title compound as a yellow solid (3.9 g, 90%). ¹H-NMR (δ ppm, CDCl₃, 300 MHz): 7.63 (q, J=8.4, 1H); 6.97 (t, J=8.7, 2H); 4.70 (br. s, 2H); 3.44 (br. s, 2H); 2.02 (d, J=7.8, 1H); 1.93 (d, J=6.9, 2H); 1.68-1.60 (m, 1H); 1.30-1.20 (m, 2H).

### Intermediate 8: Preparation of 3-(2,4-Difluorophenyl)-3,4-diazatricyclo[5.2.1.0^{2,6]}deca-2(6),4-dien-5-carbaldehyde

Pyridinium dichromate (406 mg, 1.08 mmol) was added to Intermediate 7 (300 mg, 1.08 mmol) in dichloromethane (2 ml) stirred at RT for 3 h. Ethyl acetate was added and stirred for 15 min and filtered through a pad of celite and the solvent was evaporated. Purification of crude product by SiO₂ column chromatography furnished the title compound as white solid (114 mg, 39%). ¹H-NMR (δ ppm, CDCl₃, 300 MHz): 10.00 (br. s, 1H); 7.73-7.68 (m, 1H); 7.08-7.00 (m, 2H); 3.73 (br. s, 1H); 3.48 (br. s, 1H); 2.09 (d, J=7.5, 1H); 1.98 (d, J=8.1, 2H); 1.71 (d, J=8.7, 1H); 1.23 (d, J=11.7, 2H).

### Intermediate 9: Preparation of 3-(2,4-difluorophenyl)-3,4diazatricyclo[5.2.1.0^{2,6} ]deca-2(6),4-dien-5-amine

To a solution of intermediate 3 (500 mg, 1.72 mmol) in 1,4-dioxane (12 mL), Et₃N (45 μl, 0.33 mmol) and BOP reagent (838 mg, 1.89 mmol) were added and stirred at RT for 20 min. Sodium azide (224 mg, 3.44 mmol) and tetrabutyl ammonium bromide (1.10 g, 3.44 mmol) were added and stirring continued for an additional hour. The reaction was then diluted with 1,4-dioxane (12 mL) and an aqueous 2 M H₂SO₄ (4 ml) was added and heated at 100° C. for 2 h. The solvent was evaporated and the residue diluted water and extracted with ethyl acetate. Organic layers were washed with brine, dried over Na₂SO₄ and the solvent was evaporated. Purification by column chromatography furnished Intermediate 6 as a thick brownish mass (230 mg, 51%). ¹H-NMR (δ ppm, CDCl₃, 300 MHz): 7.62-7.54 (m, 1H), 6.96-6.89 (m, 2H); 3.39 (br. s, 1H); 3.28 (br. s, 1H); 3.30-2.50 (br. s, 2H); 1.99 (d, J=9.0, 1H); 1.88 (d, J=9.0, 2H); 1.58 (d, J=6.0, 1H); 1.24 (d, J=6.0, 2H). ¹H-NMR (δ ppm, DMSO-d₆, 300 MHz): 7.62-7.50 (m, 1H), 7.48-7.38 (m, 1H); 7.18-7.10 (m, 1H); 4.95 (br. s, 2H); 3.36 (br. s, 1H); 3.25 (br. s, 1H); 1.83 (d, J=9.0, 3H); 1.52 (d, J=8.4, 1H); 1.06 (d, J=7.5, 2H).

### Intermediate 10: Preparation of (1S,7R)-3-(2,4-difluorophenyl)-3,4diazatricyclo[5.2.1.0^{2,6}]deca-2(6),4-dien-5-amine hydrochloride

To a cooled solution of Intermediate 4 (3.0 g, 10.34 mmol) in dichloromethane (30 mL), oxalyl chloride (1.07 ml, 12.41 mmol) was added dropwise followed by catalytic amount of dry DMF and the reaction was stirred at RT for 3 h. The solvent was evaporated and dried under vacuum and dissolved in 1,2-dichloroethane (30 mL) and treated with sodium azide (1.34 g, 20.68 mmol) and stirred at RT for 2 h. The reaction mixture was then diluted with dichloromethane and washed with water, brine and dried over Na₂SO₄. The solvent was evaporated and diluted with 1,4-dioxane (25 mL) and an aqueous 2 M H₂SO₄ (25 ml) was added and heated at 100° C. for overnight. The solvent was evaporated and the residue diluted water and extracted with dichloromethane. Organic layers were washed with brine, dried over Na₂SO₄ and the solvent was evaporated and dissolved in dry ether (10 mL) and treated with saturated ether-HCl (15 mL) and stirred at RT for 1 h. The reaction mixture was concentrated to obtain the product as light brown solid (2.58 g, 86%). ¹H-NMR (δ ppm, CDCl₃, 300 MHz): 7.72-7.54 (m, 2H), 7.30-7.22 (m, 1H); 3.47 (br. s, 1H); 3.45 (br. s, 1H); 1.95-1.90 (m, 2H); 1.64 (d, J=8.7, 1H); 1.18 (d, J=9.0, 2H).

### Intermediate 11: Preparation of (1R,7S)-3-(2,4-difluorophenyl)-3,4diazatricyclo[5.2.1.0^{2,6}]deca-2(6),4-dien-5-amine hydrochloride

The title compound was synthesized by a procedure similar to that described for Intermediate 10. Intermediate 5 (2.5 g, 8.62 mmol), dichloromethane (30 mL), oxalyl chloride (886 μl, 10.34 mmol), a catalytic amount of dry DMF, dichloroethane (30 mL), sodium azide (1.12 g, 17.24 mmol), 1,4-dioxane (25 mL) and an aqueous 2 M H₂SO₄ (25 ml), dry ether (10 mL) and saturated ether-HCl (15 mL) furnished as light brown solid (2.16 g, 84%). ¹H-NMR (δ ppm, CDCl₃, 300 MHz): 7.72-7.54 (m, 2H), 7.30-7.23 (m, 1H); 3.47 (br. s, 1H); 3.45 (br. s, 1H); 2.00-1.89 (m, 2H); 1.65 (d, J=8.7, 1H); 1.17 (d, J=9.0, 2H).

### Intermediate 12: Preparation of 5-Iodo-3-(2,4-difluorophenyl)-3,4-diazatricyclo[5.2.1.0^{2,6}]deca-2(6),4-diene

Intermediate 9 (850 mg, 3.25 mmol) was dissolved in dry diethyl ether (3 ml), cooled in an ice bath and treated with saturated solution of HCl in diethyl ether and the precipitated salt was filtered and washed with diethyl ether. The salt (845 mg, 2.84 mmol) was dissolved in aqueous 6N HCl (2.48 ml) and cooled in an ice bath and treated with a solution of sodium nitrite (360 mg, 5.22 mmol) maintaining the temperature below 10° C. After stirring for 15 min, a solution of potassium iodide (810 mg, 4.88 mmol) in water (0.9 ml) was added and stirred at RT for 2 h. The mixture was heated at 80-90° C. for 1 h. The mixture was poured into water and extracted with diethyl ether and combined organic layers were washed with saturated solution of potassium thiosulphate and brine. The crude product was purified by column chromatography to yield the title compound as an oil (497 mg, 45%). ¹H-NMR (δ ppm, CDCl₃, 300 MHz): 7.73-7.60 (m, 1H); 7.04-6.92 (m, 2H); 3.53 (br. s, 1H); 3.27 (br. s, 1H); 2.07 (d, J=9.6, 1H); 1.94 (d, J=9.3, 2H); 1.65 (d, J=7.5, 1H); 1.25 (d, 8.1, 2H). MS (m/z): 373.12 ([M+H]⁺).

### Intermediate 13: Preparation of (1S,7R)-5-Iodo-3-(2,4-difluorophenyl)-3,4-diazatricyclo[5.2.1.0^{2,6}]deca-2(6),4-diene

Intermediate 10 (2.5 g, 8.40 mmol) was dissolved in concentrated HCl (5 mL) and cooled in an ice bath and a solution of sodium nitrite (1.06 g, 15.46 mmol) in water (5 mL) was added maintaining the temperature below 10° C. After stirring for 15 min, a solution of potassium iodide (2.39 g, 14.45 mmol) in water (5 mL) was added and slowly heated at 80° C. for 2 h. The reaction mass was decomposed into water and extracted with ethyl acetate and the combined organic layers were washed with saturated solution of potassium thiosulphate and brine. The crude product was purified by column chromatography to yield the title compound as yellow solid (1.42 g, 47%). ¹H-NMP (δ ppm, CDCl₃, 300 MHz): 7.74-7.58 (m, 1H); 7.05-6.90 (m, 2H); 3.52 (br. s, 1H); 3.25 (br. s, 1H); 2.06 (d, J=8.7, 1H); 1.93 (d, J=9.0, 2H); 1.64 (d, J=8.1, 1H); 1.32-1.22 (m, 2H).

### Intermediate 14: Preparation of (1R,7S)-5-Iodo-3-(2,4-difluorophenyl)-3,4-diazatricyclo[5.2.1.0^{2,6}]deca-2(6),4-diene

The title compound was synthesized by a procedure similar to that described for Intermediate 13. Intermediate 11 (2.15 g, 7.23 mmol), concentrated HCl (5 mL) and sodium nitrite (917 mg, 13.29 mmol), water (12 mL) and potassium iodide (2.05 g, 12.43 mmol) furnished the title compound as yellow solid (871 mg, 32%). ¹H-NMR (δ ppm, CDCl₃, 300 MHz): 7.74-7.58 (m, 1H); 7.04-6.89 (m, 2H); 3.52 (br. s, 1H); 3.26 br. s, 1H); 2.06 (d, J=8.7, 1H); 1.93 (d, J=9.0, 2H); 1.64 (d, J=8.7, 1H); 1.33-1.23 (m, 2H).

### Intermediate 15: Preparation of 3-(2,4-Diflurophenyl)-3,4-diazatricyclo[5.2.1.0^{2,6}]deca-2(6),4-diene-5-carbohydrazide

Hydrazine hydrate (736 μl, 15.89 mmol) was added to intermediate 2 (1.0 g, mmol) in ethanol (15 ml) and the reaction was refluxed for 8 h. Solvent was evaporated and diluted with water, extracted into dichloromethane and the combined organic layers were washed with brine and dried over Na₂SO₄ and removal of solvent furnished the title compound (850 mg, 89%) as an off-white solid. ¹H-NMR (δ ppm, CDCl₃, 300 MHz): 7.92 (br. s, 1H); 7.64 (q, J=8.4, 1H); 7.01 (t, J=8.4, 2H); 4.11-3.93 (m, 2H); 3.73 (br. s, 1H); 3.46 (br. s, 1H); 2.09 (d, J=7.5, 1H); 1.97 (d, J=7.5, 2H); 1.70 (d, J=9.0, 1H); 1.25 (d, J=5.4, 2H).

### Intermediate 16: Preparation of 3-(2,4-Diflurophenyl)-3,4-diazatricyclo[5.2.1.0^{2,6}]deca-2(6),4-diene-5-carboxamide

A solution of intermediate 3 (3.0 g, 10.34 mmol) was in dichloromethane (25 mL) was cooled to 0° C., oxalyl chloride (1.0 mL, 12.41 mmol) was added dropwise followed by catalytic amount of DMF. Cooling bath was removed and the mixture stirred at RT for 3 h. The solvent evaporated and the residue was dissolved in dry acetone and the solution added dropwise to a cooled solution of aqueous ammonia and the mixture stirred at RT for an additional 1 h. The reaction mixture was poured into water and solid obtained was filtered and dried under vacuum. Yield: 2.85 g (96%, white solid). ¹H-NMR (δ ppm, CDCl₃, 300 MHz): 7.66 (q, J=8.7, 1H); 7.01 (t, J=8.1, 2H); 6.73 (br. s, 1H); 5.51 (br. s, 1H); 3.74 (br. s, 1H); 3.46 (br. s, 1H); 2.09 (d, J=8.1, 1H); 2.02-1.92 (m, 2H); 1.74-1.68 (m, 1H); 1.32-1.22 (m, 2H).

### Intermediate 17: Preparation of (1R,7S)-3-(2,4-Diflurophenyl)-3,4-diazatricyclo[5.2.1.0^{2,6}]deca-2(6),4-diene-5-carboxamide

The title compound was synthesized by a procedure similar to that described for Intermediate 16. The Intermediate 5 (600 mg, 2.06 mmol), dichloromethane (10 mL), oxalyl chloride (214 μl, 2.48 mmol), catalytic amount of DMF, dry acetone (10 mL) and aqueous ammonia (20 mL) afforded the title compound as white solid (430 mg, 72%,). ¹H-NMR (δ ppm, CDCl₃, 300 MHz): 7.63 (q, J=8.7, 1H); 6.99 (t, J=8.4, 2H); 6.70 (br. s, 1H); 5.48 (br. s, 1H); 3.73 (br. s, 1H); 3.45 (br. s, 1H); 2.08 (d, J=9.3, 1H); 1.96 (d, J=7.8, 2H); 1.72-1.68 (m, 1H); 1.26 (d, J=6.6, 2H).

### Intermediate 18: Preparation of (1S,7R)-3-(2,4-Diflurophenyl)-3,4-diazatricyclo[5.2.1.0^{2,6}]deca-2(6),4-diene-5-carboxamide

The title compound was synthesized by a procedure similar to that described for intermediate 9. The Intermediate 4 (1.0 g, 3.44 mmol), dichloromethane (20 mL), oxalyl chloride (358 μl, 4.13 mmol), catalytic amount of DMF, dry acetone (10 mL) and aqueous ammonia (20 mL) afforded the title compound as white solid (790 mg, 80%,). ¹H-NMR (δ ppm, CDCl₃, 300 MHz): 7.64 (q, J=8.7, 1H); 6.99 (t, J=9.0, 2H); 6.70 (br. s, 1H); 5.45 (br. s, 1H); 3.73 (br. s, 1H); 3.45 (br. s, 1H); 2.09 (d, J=6.9, 1H); 2.03-1.91 (m, 2H); 1.69 (d, J=8.7, 1H); 1.26 (d, J=6.0, 2H).

### Intermediate 19: Preparation of Amino-5-(2,4-Diflurophenyl)-4,5-diazatricyclo[5.2.1.0^{2,6}]deca-2(6),3-diene-3yl-methanethione

A solution of intermediate 16 (500 mg, 1.73 mmol) and phosphorous pentasufide (461 mg, 2.07 mmol) in pyridine (5 mL) was refluxed for 3 h. The reaction mixture was diluted with water and extracted with ethyl acetate and the combined organic extracts were washed with brine and dried over Na₂SO₄. Solvent was removed and the residue purified chromatographically to furnished intermediate 19 (260 mg, 49%) as a yellow solid. ¹H-NMR (δ ppm, CDCl₃, 300 MHz): 8.23 (br. s, 1H); 7.66 (q, J=8.1, 1H); 7.02 (t, J=8.1, 2H); 3.94 (br. s, 1H); 3.57-3.49 (m, 1H); 3.45 (br. s, 1H); 2.12 (d, J=6.0, 1H); 2.09-1.92 (m, 2H); 1.40-1.22 (m, 3H).

### Intermediate 20: Preparation of 3-(2,4-Difluorophenyl)-3,4-diazatricyclo[5.2.1.0^{2,6}]deca-2(6),4-diene-5-carbonitrile

A solution of Intermediate 16 (3.0 g, 10.41 mmol) in 1,4-dioxane (25 ml) was cooled in an ice bath and thionyl chloride (1.5 ml, 20.82 mmol) was added dropwise and stirred at that temperature for 10 min. after which the mixture was heated at reflux for 5 h. The solvent was evaporated and the residue obtained was treated with saturated solution of sodium bicarbonate and extracted with ethyl acetate. The organic extracts were washed successively with water and brine, dried over Na₂SO₄ and the solvent was removed. Purification of the residue by SiO₂ column chromatography furnished the title compound as a yellow solid (2.66 g, 95%). ¹H-NMR (δ ppm, CDCl₃, 300 MHz): 7.72-7.61 (m, 1H); 7.06-7.01 (m, 2H); 3.56 (br. s, 1H); 3.52 (br. s, 1H); 2.12 (d, J=7.8, 1H); 2.00 (d, J=8.4, 2H); 1.72 (d, J=8.7, 1H); 1.27 (d, 10.4, 2H). IR (cm⁻¹, KBr): 3409 (w), 3077 (m), 2984 (m), 2956 (m), 2876 (m), 2237 (s), 1613 (s), 1524 (s), 1445 (m), 1432 (m), 1350 (m), 1322 (w), 1292 (m), 1269 (m), 1232 (m), 1163 (m), 1143 (m), 1095 (m), 965 (m), 950 (m), 865 (m), 848 (m), 831(m), 808 (m).

### Intermediate 21: Preparation of (1R,7S)-3-(2,4-Difluorophenyl)-3,4-diazatricyclo[5.2.1.0^{2,6}]deca-2(6),4-diene-5-carbonitrile

The title compound was synthesized by a procedure similar to that described for intermediate 20. The intermediate 17 (900 g, 3.12 mmol), 1,4-dioxane (20 ml) and thionyl chloride (452 μl, 6.25 mmol) furnished the title compound as a yellow oil (790 mg, 88%). ¹H-NMR (δ ppm, CDCl₃, 300 MHz): 7.73-7.60 (m, 1H); 7.04-6.96 (m, 2H); 3.55 (br. s, 1H); 3.51 (br. s, 1H); 2.11 (d, J=7.8, 1H); 2.00 (d, J=9.0, 2H); 1.72 (d, J=9.0, 1H); 1.26 (d, J=9.0, 2H).

### Intermediate 22: Preparation of 3-(1S,7R)-(2,4-Difluorophenyl)-3,4-diazatricyclo[5.2.1.0^{2,6}]deca-2(6),4-diene-5-carbonitrile

The title compound was synthesized by a procedure similar to that described for intermediate 20. Intermediate 18 (790 mg, 2.74 mmol), 1,4-dioxane (15 ml) and thionyl chloride (397 μl, 5.48 mmol) furnished the title compound as a yellow oil (725 mg, 98%). ¹H-NMR (δ ppm, CDCl₃, 300 MHz): 7.64 (q, J=7.8, 1H); 7.04-6.98 (m, 2H); 3.55 (br. s, 1H); 3.50 (br. s, 1H); 2.11 (d, J=8.1, 1H); 1.99 (d, J=9.0, 2H); 1.72 (d, J=8.4, 1H); 1.26 (d, 9.0, 2H).

### Intermediate 23: Preparation of 5-(2,4-Difluorophenyl)-4,5-diazatricyclo[5.2.1.0^{2,6}]deca-2(6),3-diene-3-yl-methoxymethaneimine

A solution of Intermediate 20 (2.5 g, 9.25 mmol) and dry methanol (487 μl, 12.03 mmol) in dry diethyl ether (25 ml) was cooled 0-5° C. and dry hydrogen chloride gas was bubbled during 1 h. The mixture was kept at 0-5° C. for 24 h. The precipitate was collected by filtration and washed with diethyl ether and distributed between aqueous 10% KOH (25 ml) and dichloromethane (50 ml). The organic layers were separated, washed with brine, dried with Na₂SO₄. The solvent was evaporated to yield the title compound in pure form as a white solid (1.84 g, 88%). ¹H-NMR (δ ppm, CDCl₃, 300 MHz): 8.07 (br. s, 1H); 7.69 (q, J=8.4, 1H); 7.05-6.93 (m, 2H); 3.95 (s, 3H); 3.56 (br. s, 1H); 3.45 (br. s, 1H); 2.08 (d, J=8.4, 1H); 1.96 (d, J=8.4, 2H); 1.68 (d, J=8.4, 1H); 1.24 (d, 9.6, 2H).

### Intermediate 24: Preparation of (1R,7S)-5-(2,4-Difluorophenyl)-4,5-diazatricyclo[5.2.1.0^{2,6}]deca-2(6),3-diene-3-yl-methoxymethaneimine

The title compound was synthesized by a procedure similar to that described for intermediate 23. Intermediate 21 (750 mg, 2.77 mmol), dry methanol (146 μl, 3.61 mmol), dry diethyl ether (20 ml), dry hydrogen chloride gas, 10% KOH (25 ml) and dichloromethane (50 ml) furnished the title compound in pure form as a white foam (840 mg, 99%). ¹H-NMR (δ ppm, CDCl₃, 300 MHz): 8.04 (br. s, 1H); 7.67 (q, J=8.1, 1H); 7.04-6.94 (m, 2H); 3.94 (s, 3H); 3.56 (br. s, 1H); 3.45 (br. s, 1H); 2.08 (d, J=7.8, 1H); 1.95 (d, J=8.4, 2H); 1.67 (d, J=8.7, 1H); 1.23 (d, 9.9, 2H).

### Intermediate 25: Preparation of 5-(1S,7R)-(2,4-Difluorophenyl)-4,5-diazatricyclo[5.2.1.0^{2,6}]deca-2(6),3-diene-3-yl-methoxymethaneimine

The title compound was synthesized by a procedure similar to that described for intermediate 23. Intermediate 22 (657 mg, 2.43 mmol) and dry methanol (128 μl, 3.16 mmol), dry diethyl ether (20 ml), 10% KOH (25 ml) and dichloromethane (50 ml) to yield the title compound in pure form as a white solid (720 mg, 98%). ¹H-NMR (δ ppm, CDCl₃, 300 MHz): 8.06 (br. s, 1H); 7.67 (q, J=7.8, 1H); 7.04-6.94 (m, 2H); 3.94 (s, 3H); 3.55 (br. s, 1H); 3.45 (br. s, 1H); 2.07 (d, J=8.4, 1H); 1.95 (d, J=8.4, 2H); 1.67 (d, J=8.4, 1H); 1.23 (d, 9.0, 2H).

### Intermediate 26: Preparation of N1-{1-[5-(2,4-Difluorophenyl)-4,5-diazatricyclo[5.2.1.0^{2,6}]deca-2(6),3-dien-3-yl]-1-methoxymethylidene}-2,2-dimethyl propanamide

Pivaloyl chloride (265 μl, 1.92 mmol) was added dropwise to a solution of intermediate 23 (390 mg, 1.28 mmol) and triethyl amine (389 μl, 2.81 mmol) in dry toluene (5 ml) at 0-5° C. and the mixture was stirred at RT for 48 h. The precipitated triethylammonium chloride was filtered off and washed with toluene. The combined filtrate and washings were evaporated and the residue was dissolved in dichloromethane, washed successively with water, brine and dried (Na₂SO₄). The solvent removal furnished the title compound as white solid (411 mg, 82%) which was used without purification. ¹H-NMR (δ ppm, CDCl₃, 300 MHz): 7.63 (q, J=8.1, 1H); 6.96 (t, J=8.4, 2H); 3.88 (s, 3H); 3.52 (br. s, 1H); 3.46 (br. s, 1H); 2.06 (d, J=8.7, 1H); 1.93 (d, J=7.2, 2H); 1.69-1.62 (m, 1H); 1.25 (s, 11H). MS (m/z): 388.55([M+H]⁺).

### Intermediate 27: Preparation of N1-{1-[(1R,7S)-5-(2,4-Difluorophenyl)-4,5-diazatricyclo[5.2.1.0^{2,6}]deca-2(6),3-dien-3-yl]-1-methoxymethylidene}-2,2-dimethyl propanamide

The title compound was synthesized by a procedure similar to that described for intermediate 26. Intermediate 24 (820 mg, 2.70 mmol), pivaloyl chloride (497 μl, 4.05 mmol), Et₃N (823 μl, 5.95 mmol) and dry toluene (10 ml) furnished the title compound as a white solid (790 mg, 74%). ¹H-NMR (δ ppm, CDCl₃, 300 MHz): 7.60 (q, J=8.7, 1H); 6.94 (t, J=8.4, 2H); 3.87 (s, 3H); 3.51 (br. s, 1H); 3.45 (br. s, 1H); 2.05 (d, J=8.7, 1H); 1.93 (d, J=7.5, 2H); 1.64 (d, d=8.7, 1H); 1.24 (s, 11H).

### Intermediate 28: Preparation of N1-{1-[5-(1S,7R)-(2,4-Difluorophenyl)-4,5-diazatricyclo[5.2.1.0^{2,6}]deca-2(6),3-dien-3-yl]-1-methoxymethylidene}-2,2-dimethyl propanamide

The title compound was synthesized by a procedure similar to that described for intermediate 26. Intermediate 25 (660 mg, 2.17 mmol), pivaloyl chloride (400 μl, 3.26 mmol), Et₃N (663 μl, 4.79 mmol) and dry toluene (10 ml) after purification by SiO₂ column chromatography furnished the title compound as white solid (618 mg, 73%). ¹H-NMR (δ ppm, CDCl₃, 300 MHz): 7.60 (q, J=9.0, 1H); 6.94 (t, J=9.0, 2H); 3.87 (s, 3H); 3.51 (br. s, 1H); 3.45 (br. s, 1H); 2.05 (d, J=8.1, 1H); 1.93 (d, J=6.3, 2H); 1.64 (d, J=8.7, 1H); 1.24 (s, 11H).

### Intermediate 29: Preparation of 3-(2,4-Difluorophenyl)-5-[methoxy(phenylcarbonylimino)methyl]-3,4-diazatricyclo[5.2.1.0^{2,6}]deca-2(6),4-diene

The title compound was synthesized by a procedure similar to that described for Intermediate 26. Intermediate 23 (350 mg, 1.15 mmol), benzoyl chloride (200 μl, 1.73 mmol), triethylamine (191 μl, 1.38 mmol) and dry toluene (5 ml) furnished the title compound as yellow solid (265 mg, 56%). ¹H-NMR (δ ppm, CDCl₃, 300 MHz): 8.00 (d, J=6.9, 2 H); 7.54-7.36 (m, 3H); 7.25-7.15 (m, 1H); 6.94-6.79 (m, 2H); 4.06 (s, 3H); 3.42 (br. s, 2H); 1.97 (d, J=8.7, 1H); 1.92-1.80 (m, 2H); 1.62-1.58 (m, 1H); 1.22-1.08 (m, 2H). MS (m/z): 408.33 ([M+H]⁺).

### Intermediate 30: Preparation of 5-(2,4-Difluorophenyl)-4,5-diazatricyclo[5.2.1.0^{2,6}]deca-2(6),3-dien-3-yl(imino)methylamine

Intermediate 23 (350 mg, 1.15 mmol) and ammonium chloride (74 mg, 1.38 mmol) were mixed in dry ethanol (5 mL) and heated to reflux for 4 h. The solvent was completely removed under reduced pressure and the residue obtained was treated with saturated solution of Na₂CO₃ and extracted with ethyl acetate. The combined organic extracts were washed with brine and dried over Na₂SO₄. The solvent was evaporated and crude was dissolved in dry ether and cooled in ice bath, saturated ether-HCl was added and stirred at RT for 1 h. The solid obtained was filtered and washed with dry ether and treated with saturated solution of Na₂CO₃ and extracted with ethyl acetate and washed with brine and dried over Na₂SO₄. The solvent removal gave the title compound in pure form as a white solid (234 mg, 69%). ¹H-NMR (δ ppm, CDCl₃, 300 MHz): 8.07 (br. s, 1H); 7.66 (q, J=7.8, 1H); 7.01 (t, J=9.0, 2H); 3.67 (br. s, 3H); 3.49 (br. s, 2H); 2.13 (br. d., J=8.9, 1H); 2.00 (br. d, J=8.9, 2H); 1.73 (d, J=9.0, 1H); 1.27 (d, 7.5, 2H). MS (m/z): 289.53 ([M+H]⁺).

### Intermediate 31: Preparation of Ethyl 3-(4-fluorophenyl)-3,4-diazatricyclo[5.2.1.0^{2,6}]deca-2(6),4-dien-5-carboxylate

The intermediate 31 was prepared by a procedure similar to that described for intermediate 2. A solution of Intermediate 1 (1.0 g, 4.8 mmol), 4-fluorophenylhydrazine hydrochloride (0.85 g, 5.2 mmol) and ethanol (16.0 ml) gave pure Intermediate 22 (1.2 g, 86%). ¹H-NMR (δ ppm, CDCl₃, 300 MHz): 7.69 (dd, J=9.0, 4.8, 2H); 7.15 (t, J=9.0, 2H); 4.42 (q, J=7.2, 2H); 3.67 (br. s, 2H); 3.40 (br. s, 1H); 2.16 (br. d, J=8.7, 1H); 2.03-1.85 (m, 2H); 1.72 (br. d, J=9.0, 1H); 1.43 (t, J=7.2, 3H); 1.32-1.17 (m, 2H).

### Intermediate 32: Preparation of 3-(4-fluorophenyl)-3,4-diazatricyclo[5.2.1.0^{2,6}deca-2(6),4-dien-5-carboxylic acid

Intermediate 32 was prepared by a procedure similar to that described for intermediate 3. Intermediate 31 (1.6 g, 5.3 mmol), KOH (596 mg, 10.62 mmol), ethanol and H₂O (0.5 ml) to give Intermediate 32 (870 mg, 60%). ¹H-NMR (δ ppm, CDCl₃, 300 MHz): 7.70 (dd, J=8.7, 4.8, 2H); 7.18 (t, J=8.7, 2H); 3.70 (s, 2H); 2.17 (br. d, J=8.7, 1H); 2.10-1.90 (m, 2H); 1.74 (d, J=8.7, 1H); 1.35-1.18 (m, 2H).

### Intermediate 33: Preparation of Ethyl 3-(4-chlorophenyl)-3,4-diazatricyclo[5.2.1.0^{2,6}]deca-2(6),4-dien-5-carboxylate

The intermediate 33 was prepared by a procedure similar to that described for intermediate 2. A solution of intermediate 1 (3.0 g, 1.42 mmol), 4-chlorophenylhydrazine hydrochloride (3.06 g, 1.71 mol) and ethanol (50 ml) yielded Intermediate (3.2 g, 71%). ¹H-NMR (δ ppm, CDCl₃, 300 MHz): 7.68 (d, J=9.0, 1H); 7.42 (d, J=9.0, 2H); 4.41 (q, J=7.2, 2H); 3.69 (br. s, 1H); 3.66 (br. s, 1H); 2.13 (d, J=8.7, 1H); 2.00 (d, J=8.1, 2H); 1.72 (d, J=9.0, 1H); 1.41 (t, J=7.5, 3H); 1.30-1.17 (m, 2H).

### Intermediate 34: Preparation of 3-(4-Chlorophenyl)-3,4-diazatricyclo[5.2.1.0^{2,6}]deca-2(6),4-dien-5-carboxylic acid

Intermediate 34 was prepared by a procedure similar to that described for intermediate 3. Intermediate 33 (3.2 g, 10.10 mol), ethyl alcohol (20 ml), KOH (1.1 g, 20.20 mol) and water (1.5 ml) yielded intermediate 32 (2.4 g, 82%). ¹H-NMR (δ ppm, CDCl₃, 300 MHz): 7.67 (d, J=9.0, 1H); 7.43 (d, J=9.0, 2H); 3.70 (br. s, 2H); 2.15 (d, J=8.1, 1H); 2.01 (d, J=8.1, 2H); 1.74 (d, J=8.7, 1H); 1.23 (d, J=7.8, 2H).

### Intermediates 35 and 36: Optical Resolution of 3-(4-chlorophenyl)-3,4-diazatricyclo[5.2.1.0^{2,6}]deca-2(6),4-dien-5-carboxylic acid

### Intermediate 35: Preparation of (1R,7S)- or (1S,7R)-3-(4-chlorophenyl)-3,4-diazatricyclo[5.2.1.0^{2,6}]deca-2(6),4-dien-5-carboxylic acid

A slurry of Intermediate 34 (racemic, 4.0 g, 13.91 mmol) in acetonitrile (LR grade) (40 ml) was treated with (R)-(−)-1-(4-chlorophenyl)ethylamine (1.46 ml, 10.43 mmol), stirred at an ambient temperature for about 5-10 minutes and the mixture was heated at reflux for about 15 minutes. Methanol (15-20 mL) was added slowly till a clear solution resulted and heating was continued for further about 30 minutes. The reaction mixture was allowed to cool slowly to an ambient temperature. The separated crystals were collected by filteration and washed with acetonitrile/Methanol 9:1 (10-20 mL). The acid was recovered from the diastereomeric salt by dissolving in dichloromethane and extraction with aqueous 1N hydrogenchloride. Reiteration of the same procedure several times gave a mixture enriched in the late eluting enantiomer [Intermediate 35, Rₜ=9.22 min. on a CHIRAL PAK OD-H column (dimensions: 250×4.6 mm, particle size: 5μ) using a 60:40:0.1 mixture of n-hexane:isopropanol:trifluoroacetic acid as the eluent at 0.5 ml/min. flow rate]. e.e=93%.

### Intermediate 36: Preparation of (1S,7R)- or (1R,7S)-3-(4-chlorophenyl)-3,4-diazatricyclo[5.2.1.0^{2,6}]deca-2(6),4-dien-5-carboxylic acid

The mother liquor obtained in the first step of the process described above was evaporated, distributed between dichloromethane and aqueous 1N hydrogenchloride and the layers were separated. Dried over sodiumsulphate and evaporated of the organic layer gave a mixture of the two enantiomeric acids (1.8 g) enriched in the fast eluting enantiomer (Rₜ=8.02 min. under the same conditions described above; e.e=41%). The mixture was enriched in this enantiomer to an e.e of 94% (Intermediate 36, Rₜ=7.94 min. under the same conditions described for the late eluting enantiomer; yield=90 mg) by replacing (S)-(−)-1-(4-chlorophenyl)ethylamine with (R)-(−)-1-(4-chlorophenyl)ethylamine in the process described above for the late eluting enantiomer. M.P.: 110-112° C.

### Intermediate 37: Preparation of Ethyl 3-(4-chloro-2-fluorophenyl)-3,4-diazatricyclo[5.2.1.0^{2,6}]deca-2(6),4-dien-5-carboxylate

The intermediate 37 was prepared by a procedure similar to that described for intermediate 2. A solution of Intermediate 1 (4.25 g, 20.23 mmol), 4-chloro-2-fluorophenyl hydrazine hydrochloride (prepared from 4-chloro-2-fluoro-aniline by diazotization followed by reduction with SnCl₂) (3.2 g, 16.24 mmol) and ethanol (20 ml) yielded pyrazole ester (4.16 g, 62%). ¹H-NMR (δ ppm, CDCl₃, 300 MHz): 7.24 (t, J=8.1, 1H); 7.28-7.23 (m, 2H); 4.41 (q, J=6.9, 2H); 3.65 (br. s, 1H); 3.48 (br. s, 1H); 2.09 (d, J=9.0, 1H); 1.96 (d, J=9.0, 2H); 1.68 (d, J=9.0, 1H); 1.40 (t, J=6.9, 3H); 1.23 (d, J=9.0, 2H).

### Intermediate 38: Preparation of 3-(4-Chloro-2-fluorophenyl)-3,4-diazatricyclo[5.2.1.0^{2,6}]deca-2(6),4-dien-5-carboxylic acid

Intermediate 38 was prepared by a procedure similar to that described for intermediate 3. Intermediate 37 (4.1 g, 12.27 mol), isopropyl alcohol (40 ml), KOH (960 mg, 17.14 mol) and water (13.6 ml) yielded intermediate 37 (3.3 g, 88%). ¹H-NMR (δ ppm, CDCl₃, 300 MHz): 7.73 (d, J=8.1, 1H); 7.32-7.24 (m, 2H); 3.69 (br. s, 1H); 3.51 (br. s, 1H); 2.11 (d, J=9.0, 1H); 1.99 (d, J=9.0, 2H); 1.71 (d, J=9.0, 1H); 1.26 (d, J=9.0, 2H).

### Intermediates 39 and 40: Optical Resolution of 3-(4-chloro-2-fluorophenyl)-3,4-diazatricyclo[5.2.1.0^{2,6}]deca-2(6),4-dien-5-carboxylic acid

### Intermediate 39: Preparation of (1S,7R)- or (1R,7S)-3-(4-chloro-2-fluorophenyl)-3,4-diazatricyclo[5.2.1.0^{2,6}]deca-2(6),4-dien-5-carboxylic acid

A slurry of Intermediate 38 (racemic, 2.75 g, 8.98 mmol) in acetonitrile (LR grade) (25-30 mL) was treated with (R)-(+)-1-(4-chlorophenyl)ethylamine (1.26 mL, 8.98 mmol) and stirred at an ambient temperature for about 5-10 minutes and the mixture was heated at reflux for about 15 minutes. Methanol (12.5-15.0 mL) was added slowly till a clear solution resulted and heating was continued for further about 30 minutes. The reaction mixture was allowed to cool slowly to an ambient temperature. The separated crystals of the diastereomeric salt were collected by filteration and washed with acetonitrile/methanol 9:1 (5-10 mL). Recrystalisation of this salt several times from boiling acetonitrile/methanol gave a mixture enriched in the fast eluting enantiomer [Intermediate 39, HPLC: Rₜ=18.03 min. on a CHIRAL PAK AS-H column (dimensions: 250×4.6 mm, particle size: 5μ) using a 80:20:0.1 mixture of n-hexane:isopropanol:trifluoroacetic acid as the eluent at 0.9 ml/min. flow rate]. Enantiomeric Excess=93%.The acid was recovered from the diastereomeric salt by dissolving in dichloromethane and washing with aqueous 1N Hydrogenchloride.

### Intermediate 40: Preparation of (1R,7S)- or (1S,7R)-3-(4-chloro-2-fluorophenyl)-3,4-diazatricyclo[5.2.1.0^{2,6}]deca-2(6),4-dien-5-carboxylic acid

The mother liquor obtained in the first step of the process described above was evaporated, distributed between dichloromethane and aqueous 1N hydrogen chloride and the layers were separated and dried over sodium sulphate gave a non-racemic mixture of the two enantiomeric acids. From this mixture, by replacing (R)-(+)-1-(4-chlorophenyl)ethylamine with (S)-(−)-1-(4-chlorophenyl)ethylamine in the process described above, the late eluting enantiomer (HPLC: Rₜ=22.86 min. under the same conditions described for the fast eluting enantiomer) with an e.e. of 91% (Intermediate 40, yield=176 mg) was obtained.

### Intermediate 41: Preparation of Ethyl 3-(2,4,6-Trifluorophenyl)-3,4-diazatricyclo[5.2.1.0^{2,6}]deca-2(6),4-dien-5-carboxylate

The intermediate 41 was prepared by a procedure similar to that described for intermediate 2. A solution of Intermediate 1 (1.5 g, 7.14 mmol), 2,4,6-trifluorophenyl hydrazine hydrochloride (prepared from 2,4,6-trifluoro-aniline by diazotization followed by reduction with SnCl₂) (1.55 g, 7.87 mmol) and ethanol (25 ml) yielded pyrazole ester (1.2 g, 50%). ¹H-NMR (δ ppm, CDCl₃, 300 MHz): 6.84 (t, J=7.8, 2H); 4.40 (q, J=9.0, 2H); 3.69 (br. s, 1H); 3.30 (br. s, 1H); 2.11 (d, J=9.0, 1H); 1.98-1.89 (m, 2H); 1.69 (d, J=9.0, 1H); 1.40 (t, J=9.0, 3H); 1.28-1.18 (m, 2H).

### Intermediate 42: Preparation of 3-(2,4,6-Trifluorophenyl)-3,4-diazatricyclo[5.2.1.0^{2,6}]deca-2(6),4-dien-5-carboxylic acid

Intermediate 42 was prepared by a procedure similar to that described for intermediate 3. Intermediate 41 (1.2 g, 3.57 mmol), isopropyl alcohol (12 ml), KOH (279 mg, 4.99 mol) and water (4.0 ml) yielded intermediate 37 (800 mg, 72%). ¹H-NMR (δ ppm, CDCl₃, 300 MHz): 6.87 (t, J=9.0, 1H); 3.72 (br. s, 2H); 3.33 (br. s, 1H); 2.14 (d, J=9.0, 1H); 2.00-1.96 (m, 2H); 1.71 (d, J=9.0, 1H); 1.24-1.22 (m, 2H).

### Intermediate 43: Preparation of Ethyl 3-(4-Methoxyphenyl)-3,4-diazatricyclo[5.2.1.0^{2,6}]deca-2(6),4-dien-5-carboxylate

The title compound was synthesized by a procedure similar to that described for Intermediate 2. Intermediate 1 (500 mg, 2.38 mmol), 4-methoxyphenyl hydrazine hydrochloride (457 mg, 2.61 mmol) and ethanol (5 ml) furnished the title compound (250 mg, 33%). ¹H-NMR (δ ppm, CDCl₃, 300 MHz): 7.62 (d, J=9.0, 2H); 6.96 (d, J=9.0, 2H); 4.41 (q, J=7.2, 2H); 3.85 (s, 3H); 3.65 (br. s, 2H); 2.12 (d, J=8.7, 1H); 1.97 (d, J=8.7, 2H); 1.71 (d, J=8.7, 1H); 1.41 (t, J=6.9, 3H); 1.22 (d, J=7.8, 2H).

### Intermediate 44: Preparation of 3-(4-Methoxyphenyl)-3,4-diazatricyclo[5.2.1.0^{2,6}]deca-2(6),4-dien-5-carboxylic acid

The title compound was synthesized by a procedure similar to that described for Intermediate 3. Intermediate 43 (250 mg, 0.80 mmol), isopropyl alcohol (3 ml), KOH (58 mg, 1.04 mmol) and water (0.8 ml) gave Intermediate 44 (200 g, 88%). ¹H-NMR (δ ppm, CDCl₃, 300 MHz): 7.62 (d, J=9.0, 2H); 6.98 (d, J=8.7, 2H); 3.85 (s, 3H); 3.70 (br. s, 1H); 3.67 (br. s, 1H); 2.14 (d, J=8.1, 1H); 2.00 (d, J=9.0, 2H); 1.72 (d, J=8.4, 1H); 1.24 (d, J=8.1, 2H).

### Intermediate 45: Preparation of Ethyl 3-(4-bromophenyl)-3,4-diazatricyclo[5.2.1.0^{2,6}]deca-2(6),4-dien-5-carboxylate

The intermediate 45 was prepared by a procedure similar to that described for intermediate 2. Intermediate 1 (1.0 g, 4.8 mmol), 4-bromophenylhydrazine hydrochloride (1.28 g, 5.6 mmol) and ethanol (16.0 ml) gave intermediate 45 (1.4 g, 81%). ¹H-NMR (δ ppm, CDCl₃, 300 MHz): 7.65-7.50 (m, 4H); 4.42 (q, J=7.2, 2H); 3.69 (s, 2H); 3.67 (s, 2H); 2.13 (br. d, J=8.7, 1H); 2.06-1.95 (m, 2H); 1.74 (d, J=8.7, 1H); 1.42 (t, J=7.2, 3H); 1.28-1.15 (m, 2H).

### Intermediate 46: Preparation of 3-(4-Bromophenyl)-3,4-diazatricyclo[5.2.1.0^{2,6}]deca-2(6),4-dien-5-carboxylic acid

Intermediate 46 was prepared by a procedure similar to that described for intermediate 3. Intermediate 45 (1.4 g, 3.9 mmol), KOH (435 mg, 7.8 mmol), ethanol (8.0 ml) and H₂O (0.5 ml) gave intermediate 46 (1.1 g, 83%). ¹H-NMR (δ ppm, CDCl₃, 300 MHz): 7.62 (s, 4H); 3.71 (s, 2H); 2.17 (br. d, J=9.0, 1H); 2.06-2.01 (m, 2H); 1.75 (d, J=9.0, 1H); 1.30-1.17 (m, 2H).

### Intermediates 47 and 48: Optical Resolution of 3-(4-bromophenyl)-3,4-diazatricyclo[5.2.1.0^{2,6}]deca-2(6),4-dien-5-carboxylic acid

### Intermediate 47: Preparation of (1S,7R)- or (1R,7S)-3-(4-Bromophenyl)-3,4-diazatricyclo[5.2.1.0^{2,6}]deca-2(6),4-dien-5-carboxylic acid

A slurry of intermediate 46 (3.40 g, 10.24 mmol) in acetonitrile (LR grade) (30-40 mL) was treated with (R)-(+)-1-(4-chlorophenyl)ethylamine (1.07 mL, 7.68 mmol), stirred at an ambient temperature for about 5-10 minutes and the mixture was heated at reflux for about 15 minutes. Methanol (30-40 mL) was added slowly till a clear solution resulted and heating was continued for further about 30 minutes. The reaction mixture was allowed to cool slowly to an ambient temperature. The separated crystals were collected by filteration and washed with acetonitrile/MeOH 9:1 (5-10 mL). The acid was recovered from the diastereomeric salt by dissolving in dichloro methane and extraction with aqueous 1N hydrogen chloride. Reiteration of the same procedure several times gave a mixture (78 mg) enriched in the late eluting enantiomer [Intermediate 47, Rₜ=9.39 min. on a CHIRAL PAK OD-H column (dimensions: 250×4.6 mm, particle size: 5μ) using a 60:40:0.1 mixture of n-hexane:isopropanol:trifluoroacetic acid as the eluent at 0.5 ml/min. flow rate]. Enantiomeric Excess=93%.

### Intermediate 48: Preparation of (1R,7S)- or (1S,7R)-3-(4-Bromophenyl)-3,4-diazatricyclo[5.2.1.0^{2,6}]deca-2(6),4-dien-5-carboxylic acid

The mother liquor obtained in the first step of the process described above was evaporated, distributed between dichloromethane and aqueous 1N hydrogen chloride and the layers were separated and dried over sodium sulphate gave a non-racemic mixture of the two enantiomeric acids. From this mixture, by replacing (R)-(+)-1-(4-chlorophenyl)ethylamine with (S)-(−)-1-(4-chlorophenyl)ethylamine in the process described above, the fast eluting enantiomer (HPLC: Rₜ=8.15 min. under the same conditions described for the late eluting enantiomer) with an e.e of 91% (Intermediate 48, yield=80 mg) was obtained.

### Intermediate 49: Preparation of Ethyl 3-(4-nitrophenyl)-3,4-diazatricyclo[5.2.1.0^{2,6}]deca-2(6),4-dien-5-carboxylate

The title compound was synthesized by a procedure similar to that described for Intermediate 2. Intermediate 1 (1.0 g, 4.76 mmol), 4-nitrophenyl hydrazine hydrochloride (989 mg, 5.23 mmol), ethanol (15 ml) and acetic acid (10 ml) furnished the title compound (895 mg, 78%). ¹H-NMR (δ ppm, CDCl₃, 300 MHz): 8.34 (d, J=9.0, 2H); 7.94 (d, J=9.0, 2H); 4.43 (q, J=7.2, 2H); 3.80 (br. s, 1H); 3.68 (br. s, 1H); 2.18 (d, J=7.2, 1H); 2.04 (d, J=6.9, 2H); 1.77 (d, J=8.4, 1H); 1.43 (t, J=7.2, 3H); 1.23 (d, J=9.0, 2H).

### Intermediate 50: Preparation of 3-(4-Nitrophenyl)-3,4-diazatricyclo[5.2.1.0^{2,6}]deca-2(6),4-dien-5-carboxylic acid

The title compound was synthesized by a procedure similar to that described for Intermediate 3. Intermediate 49 (750 mg, 2.29 mmol), ethanol (11 ml), KOH (166 mg, 2.98 mmol) and water (3.5 ml) gave intermediate 50 (580 g, 85%). ¹H-NMR (δ ppm, CDCl₃, 300 MHz): 8.37 (d, J=9.0, 2H); 7.96 (d, J=9.0, 2H); 3.83 (br. s, 1H); 3.73 (br. s, 1H); 2.21 (d, J=9.0, 1H); 2.07 (d, J=6.6, 2H); 1.79 (d, J=8.7, 1H); 1.26 (d, J=6.9, 1H).

### Intermediate 51: Preparation of Ethyl 3-(4-methylphenyl)-3,4-diazatricyclo[5.2.1.0^{2,6}]deca-2(6),4-dien-5-carboxylate

The title compound was synthesized by a procedure similar to that described for intermediate 2. Intermediate 1 (1.0 g, 4.76 mmol), 4-methylphenylhydrazine hydrochloride (830 mg, 5.23 mmol) and ethanol (10 ml) furnished the title compound (1.04 g, 81%). ¹H-NMR (δ ppm, CDCl₃, 300 MHz): 7.59 (d, J=8.4, 2H); 7.24 (d, J=7.5, 2H); 4.41 (q, J=6.9, 2H); 3.68 (br. s, 1H); 3.66 (br. s, 1H); 2.39 (s, 3H); 2.11 (d, J=9.0, 1H); 1.97 (d, J=9.0, 2H); 1.70 (d, J=9.0, 1H); 1.41 (t, J=9.0, 3H); 1.25-1.20 (m, 2H).

### Intermediate 52: Preparation of 3-(4-Methylphenyl)-3,4-diazatricyclo[5.2.1.0^{2,6}]deca-2(6),4-dien-5-carboxylic acid

Intermediate 52 was prepared from Intermediate 51 (1.03 g, 3.84 mmol), alcohol (13-15 mL), potassium hydroxide (258 mg, 4.61 mmol) and water (3-5 mL) according to the procedure described in intermediate 3. ¹H-NMR (δ ppm, CDCl₃, 300 MHz): 7.59 (d, J=9.0, 2H); 7.27 (d, J=9.0, 2H); 3.70 (br. s, 2H); 2.40 (s, 3H); 2.12 (d, J=8.4, 1H); 2.00 (d, J=7.8, 2H); 1.72 (d, J=8.4, 1H); 1.23 (d, J=9.0, 2H).

### Intermediate 53: Preparation of Ethyl 3-(4-trifuromethylphenyl)-3,4-diazatricyclo[5.2.1.0^{2,6}]deca-2(6),4-dien-5-carboxylate

Intermediate 1 (1.0 g, 4.76 mmol) and 4-trifuromethylphenyl hydrazine (922 mg, 5.23 mmol) in ethanol (10-20 mL) were refluxed between 2-6 hrs. After completion of the reaction, the solvent was removed and the residue dissolved in acetic acid (8-10 mL) and heated to 80-110° C. overnight. The mixture was poured into water, extracted into ethyl acetate and the organic layers washed with aq. satd. Sodium bicarbonate, dried over Na₂SO₄. Removal of solvent and purification of the residue afforded the title compound as as a yellow solid. ¹H-NMR (δ ppm, CDCl₃, 300 MHz): 7.88 (d, J=8.4, 2H); 7.72 (d, J=8.4, 2H); 4.42 (q, J=6.9, 2H); 3.75 (br. s, 1H); 3.68 (br. s, 1H); 2.16 (d, J=8.7, 1H); 2.02 (d, J=7.5, 2H); 1.74 (d, J=8.7, 2H); 1.42 (t, J=6.9, 3H); 1.22 (d, J=6.6, 2H).

### Intermediate 54: Preparation of 3-(4-Trifluromethylphenyl)-3,4-diazatricyclo[5.2.1.0^{2,6}]deca-2(6),4-dien-5-carboxylic acid

The intermediate 54 was prepared from intermediate 53 (500 mg, 0.41 mmol), ethanol (5-10 mL), water (1-5 mL) and potassium hydroxide (103 mg, 1.89 mmol) according to a procedure described in intermediate 3. ¹H-NMR (δ ppm, CDCl₃, 300 MHz): 7.89 (d, J=8.4, 2H); 7.74 (d, J=8.1, 2H); 3.78 (br. s, 1H); 3.72 (br. s, 1H); 2.18 (d, J=8.1, 1H); 2.04 (d, J=7.5, 2H); 1.77 (d, J=8.7, 2H); 1.25 (d, J=6.3, 2H).

### Intermediate 55: Preparation of Ethyl 3-(4-tert-butylphenyl)-3,4-diazatricyclo[5.2.1.0^{2,6}]deca-2(6),4-dien-5-carboxylate

The intermediate 55 was prepared from intermediate 1 (600 mg, 2.85 mmol), 4-tert-butylphenylhydrazine hydrochloride (628 mg, 3.14 mmol) and ethanol (10-20 mL) according to a procedure described for intermediate 2. ¹H-NMR (δ ppm, CDCl₃, 300 MHz): 7.62 (d, J=8.7, 2H); 7.45 (d, J=8.1, 2H); 4.41 (q, J=6.9, 2H); 3.69 (br. s, 1H); 3.65 (br. s, 1H); 2.14-2.06 (m, 1H); 1.98 (d, J=9.0, 2H); 1.70 (d, J=9.0, 1H); 1.41 (t, J=9.0, 3H); 1.34 (s, 9H); 1.28-1.20 (m, 2H).

### Intermediate 56: Preparation of 3-(4-tert-Butylphenyl)-3,4-diazatricyclo[5.2.1.0^{2,6}]deca-2(6),4-dien-5-carboxylic acid

The intermediate 56 was prepared from intermediate 55 (450 mg, 1.33 mmol), alcohol (5-10 mL), potassium hydroxide (96 mg, 1.73 mmol) and water (1-5 mL) according to a procedure described in intermediate 3. ¹H-NMR (δ ppm, CDCl₃, 300 MHz): 7.63 (d, J=9.0, 2H); 7.48 (d, J=9.0, 2H); 3.71 (br. s, 2H); 2.13 (d, J=9.0, 1H); 2.01 (d, J=8.7, 2H); 1.72 (d, J=9.0, 1H); 1.35 (s, 9H); 1.25 (d, J=7.8, 2H).

### Intermediate 57: Preparation of Ethyl 3-(2-chloro-4-fluorophenyl)-3,4-diazatricyclo[5.2.1.0^{2,6}]deca-2(6),4-dien-5-carboxylate

The intermediate 58 was prepared from a solution of intermediate 1 (2.00 g, 9.52 mmol), 2-chloro-4-fluorophenyl hydrazine hydrochloride (prepared according to the literature procedure starting from 2-chloro-4-fluoroaniline) (1.5 g, 10.40 mmol) and ethanol (10-20 mL) according to a procedure described for intermediate 2. ¹H-NMR (δ ppm, CDCl₃, 300 MHz): 7.55-7.48 (m, 1H); 7.28-7.23 (m, 1H); 7.12-7.05 (m, 1H); 4.41 (q, J=7.2, 2H); 3.68 (br. s, 1H); 3.65 (br. s, 1H); 2.14 (d, J=8.7, 1H); 2.04-1.86 (m, 2H); 1.70 (d, J=8.7, 1H); 1.40 (t, J=7.2, 3H); 1.28-1.16 (m, 2H).

### Intermediate 58: Preparation of 3-(2-Chloro-4-fluorophenyl)-3,4-diazatricyclo[5.2.1.0^{2,6}]deca-2(6),4-dien-5-carboxylic acid

Intermediate 58 was prepared by a procedure similar to that described for intermediate 3. Intermediate 57 (1.5 g, 4.40 mol), isopropyl alcohol (15 ml), KOH (352 mg, 6.2 mmol) and water (5.0 ml) yielded intermediate 47 (1.2 g, 87%). ¹H-NMR (δ ppm, CDCl₃, 300 MHz): 7.56-7.48 (m, 1H); 7.32-7.24 (m, 1H); 7.15-7.06 (m, 1H); 3.71 (br. s, 1H); 3.38 (br. s, 1H); 2.16 (d, J=8.4, 1H); 2.02-1.86 (m, 2H); 1.72 (d, J=8.7, 1H); 1.28-1.19 (m, 2H).

### Intermediate 59: Preparation of Ethyl 3-(2,4,5-trifluorophenyl)-3,4-diazatricyclo[5.2.1.0^{2,6}]deca-2(6),4-dien-5-carboxylate

The intermediate 59 was prepared from a solution of intermediate 1 (1.90 g, 9.04 mmol), 2,4,5-trifluorophenyl hydrazine hydrochloride (1.97 g, 9.95 mmol) [prepared from 2,4,5-trifluoroanilne by diazotization followed by reduction with tinchloride] and ethanol (10-30 mL) according to a procedure described for intermediate 2. ¹H-NMR (δ ppm, CDCl₃, 300 MHz): 7.69 (q, J=7.8, 1H); 7.12 (q, J=9.9, 1H); 4.41 (q, J=6.9, 2H); 3.66 (br. s, 1H); 3.49 (br. s, 1H); 2.10 (d, J=8.4, 1H); 1.98 (d, J=7.5, 2H); 1.70 (d, J=9.0, 1H); 1.41 (t, J=6.9, 3H); 1.24 (d, J=7.2, 2H).

### Intermediate 60: Preparation of 3-(2,4,5-trifluorophenyl)-3,4-diazatricyclo[5.2.1.0^{2,6}]deca-2(6),4-dien-5-carboxylic acid

The intermediate 60 was prepared from intermediate 59 (1.37 g, 4.07 mmol), isopropyl alcohol (10-20 mL), potassium hydroxide (319 mg, 5.69 mmol) and water (3-10 mL) according to a procedure described for intermediate 3. ¹H-NMR (δ ppm, CDCl₃, 300 MHz): 7.69 (q, J=7.8, 1H); 7.14 (q, J=9.9, 1H); 3.69 (br. s, 1H); 3.52 (br. s, 1H); 2.12 (d, J=8.1, 1H); 2.00 (d, J=7.2, 2H); 1.72 (d, J=9.0, 1H); 1.26 (d, J=9.0, 2H).

### Intermediate 61: Ethyl 3-(3,5-difluorophenyl)-3,4-diazatricyclo[5.2.1.0^{2,6}]deca-2(6),4-dien-5-carboxylate

The intermediate 61 was prepared from a solution of intermediate 1 (3.50 g, 16.90 mmol), 3,5-difluorophenyl hydrazine hydrochloride (3.31 g, 18.30 mmol) [prepared from 3,5-difluoroanilne by diazotization followed by reduction with tinchloride] and ethanol (30-50 mL) according to a procedure described for intermediate 2. ¹H-NMR (δ ppm, CDCl₃, 300 MHz): 7.36-7.30 (m, 2H); 6.80-6.72 (m, 1H); 4.42 (q, J=7.2, 2H); 3.74 (br. s, 1H); 3.66 (br. s, 1H); 2.14 (d, J=8.7, 1H); 2.01 (d, J=7.2, 2H); 1.73 (d, J=8.7, 1H); 1.39 (t, J=7.2, 3H); 1.32-1.18 (m, 2H).

### Intermediate 62: Preparation of 3-(3,5-Difluorophenyl)-3,4-diazatricyclo[5.2.1.0^{2,6}]deca-2(6),4-dien-5-carboxylic acid

The intermediate 62 was prepared from intermediate 61 (1.70 g, 5.34 mmol), isopropyl alcohol (15-20 mL), potassium hydroxide (415 mg, 7.40 mmol) and water (5-10 mL) according to a procedure described in intermediate 3. ¹H-NMR (δ ppm, CDCl₃, 300 MHz): 7.72 (q, J=9.0, 1H); 7.02-6.94 (m, 2H); 3.68 (br. s, 1H); 3.48 (br. s, 1H); 2.11 (d, J=8.1, 1H); 1.98 (d, J=7.8, 2H); 1.70 (d, J=8.7, 1H); 1.26 (d, J=9.0, 2H).

### Intermediate 63: Preparation of Ethyl-2-(3-chlorobicyclo[2.2.1]hept-2-en-2-yl)-2-oxoacetate

To a solution of intermediate 1 in 1,2-dichloroethane (90 ml), thionyl chloride (8.0 mL, 108.51 mmol) was added and the mixture was heated at reflux overnight. After cooling to ambient temperature, the mixture was diluted with ethyl acetate, organic washed twice with water and then with brine and dried over Na₂SO₄ and evaporated. Less polar of the two products formed was isolated by flash chromatography (petroleum ether/ethyl acetate 90:10) to furnish the title compound in pure form as a yellow oil (3.2 g, 36%). ¹H-NMR (δ ppm, CDCl_{3,} 300 MHz): 4.40-4.29 (m, 2H); 3.49 (br. s, 1H); 3.09 (br. s, 1H); 1.98-1.79 (m, 2H); 1.73 (d, J=8.7, 1H); 1.43-1.33 (m, 4H); 1.30 (d, J=9.0, 2H).

### Intermediate 64: Preparation of Ethyl 3-N′-[2,4-dichlorophenylhyrazino]bicyclo[2.2.1]hept-2-ene-2-carboxylate

A solution of 2,4-dichlorophenylhydrazine (890 mg, 4.16 mol) in dichloromethane (6 ml) was treated with Et₃N (1.5 ml, 10.41 mmol) for 15 min at RT. A solution of intermediate 62 (950 mg, 4.16 mol) in dichloromethane (4 ml) was added to this mixture and heated to reflux for 4 h. The solvent was evaporated and the residue was dissolved in ethyl acetate, washed successively with water and brine and dried over Na₂SO₄. Evaporation of solvent furnished the title compound (906 g, 59%) which was used in the next step without purification. ¹H-NMR (δ ppm, CDCl₃, 300 MHz): 10.54 (br. s, 1H); 8.93 (br. s, 1H); 7.55 (d, J=2.1, 1H); 7.36 (dd, J=8.7, 2.1, 1H); 7.12 (d, J=8.7, 1H); 4.22 (q, J=7.2, 2H); 3.75 (br. s, 1H); 3.65 (br. s, 1H); 1.85-1.72 (m, 2H); 1.59-1.32 (m, 4H); 1.27 (t, J=7.2, 3H).

### Intermediate 65: Preparation of Ethyl 4-(2,4-dichlorophenyl)-3,4-diazatricyclo[5.2.1.0^{2,6}]deca-2,5-diene-5-carboxylate

Acetyl chloride (176 μl, 2.46 mmol) was added at 0° C. to ethanol (10 ml), cooling bath was removed and stirred at RT for 15 min. Intermediate 64 (906 mg, 2.46 mol) was added and refluxed for 2 h. After removal of solvent, the residue was dissolved in ethyl acetate, washed with water and then with brine and dried over Na₂SO₄. Purification of the crude product by SiO₂ column chromatography furnished the title compound as a yellow oil (580 g, 67%). ¹H-NMR (δ ppm, CDCl₃, 300 MHz): 7.52-7.30 (m, 3H); 4.21 (q, J=6.9, 2H); 3.62 (br. s, 1H); 3.46 (br. s, 1H); 2.08 (d, J=7.5, 1H); 2.01 (d, J=8.1, 2H); 1.76 (d, J=8.7, 1H); 1.35 (d, J=11.4, 2H); 1.25 (t, J=7.2, 3H).

### Intermediate 66: Preparation of 4-(2,4-Dichlorophenyl)-3,4-diazatricyclo[5.2.1.0^{2,6}]deca-2,5-diene-5-carboxylic acid

The title compound was synthesized by a procedure similar to that described for Intermediate 3. Intermediate 65 (550 mg, 1.57 mol), ethyl alcohol (6 ml), 1.25 M KOH (115 mg, 2.04 mmol) in water (1.6 ml) furnished the title compound (460 mg, 91%). ¹H-NMR (δ ppm, CDCl₃, 300 MHz): 7.53-7.30 (m, 3H); 3.65 (br. s, 1H); 3.47 (br. s, 1H); 2.12-1.94 (m, 3H); 1.76 (d, J=9.0, 1H); 1.34 (d, J=10.2, 2H).

### Intermediate 67: Preparation of N5-Methoxy-N5-methyl-4-(2,4-Dichlorohenyl)-3,4-diazatricyclo[5.2.1.0^{2,6}]deca-2,5-diene-5-carboxamide

Intermediate 66 (450 mg, 1.39 mmol) in dry DMF (5 ml) was treated with BOP reagent (650 mg, 1.46 mmol) and Et₃N (434 μl, 3.07 mmol) at RT for about 15 min. and then N,O-dimethylhydroxylamine hydrochloride (204 mg, 2.09 mmol) was added. After stirring overnight, the mixture was poured into water, the precipitate formed was filtered and purified by column chromatography to give the title compound (300 mg, 58%). ¹H-NMR (δ ppm, CDCl₃, 300 MHz): 7.47-7.40 (m, 2H); 7.33 (dd, J=8.7, 1H); 3.68 (s, 3H), 3.53 (br. s, 1H); 3.46 (br. s, 1H); 3.29 (s, 3H); 2.07-1.97 (m, 3H); 1.72 (d, J=9.0, 1H); 1.44-1.30 (m, 2H).

### Intermediate 68: Preparation of Ethyl 2-[N′-(2,4-difluorophenyl)hyrazino]bicyclo[2.2.1]hept-2-en-2-yl 2-oxoacetate

Triethylamine (1.06 ml, 7.65 mmol) was added to a solution of 2,4-diflurophenylhydrazine hydrochloride (552 mg, 3.06 mol) in dichloromethane (10 mL) followed by intermediate 63 (700 mg, 3.06 mol) and refluxed for about 2 hours. The solvent was evaporated and diluted with water, extracted with ethyl acetate and organic layers were washed with water, brine and dried over sodium sulphate. Evaporation of solvent furnished ethyl 2-[N′-(2,4-difluorophenyl)hyrazino]bicyclo[2.2.1]hept-2-en-2-yl-2-oxoacetate which was used in the next step without purification. ¹H-NMR (δ ppm, CDCl₃, 300 MHz): 10.53 (br. s, 1H); 7.20-7.05 (m, 1H); 6.90-6.70 (m, 2H); 4.34 (q, J=6.9, 2H); 3.95 (br. s, 1H); 3.32 (br. s, 1H); 2.05-1.82 (m, 2H); 1.79-1.40 (m, 4H); 1.39 (t, J=6.9, 3H).

### Intermediate 69 Preparation of Ethyl 4-(2,4-Difluophenyl)-3,4-diazatricyclo[5.2.1.0^{2,6}]deca-2,5-diene-5-carboxylate

Acetyl chloride (201 μL, 2.82 mmol) was added to ethanol (9-10 mL) at about 0° C. then cooling bath was removed and stirred at an ambient temperature for about 15 minutes. Ethyl 2-[N′-(2,4-difluorophenyl)hyrazino]bicyclo[2.2.1]hept-2-en-2-yl 2-oxoacetate (950 mg, 2.82 mmol) was added and refluxed for about 2 hours. After removal of the solvent, the residue was dissolved in ethyl acetate, washed with water, brine and dried over sodium sulphate. Purification of the crude product by silicon dioxide column chromatography furnished intermediate 69 as an yellow oil. ¹H-NMR (δ ppm, CDCl₃, 300 MHz): 7.45 (q, J=8.4, 1H); 6.98-6.88 (m, 2H); 4.24 (q, J=6.9, 2H); 3.61 (br. s, 1H); 3.45 (br. s, 1H); 2.12-1.95 (m, 3H); 1.75 (d, J=9.0, 1H); 1.36 (d, J=9.0, 2H); 1.27 (t, J=6.9, 3H).

### Intermediate 70: Preparation of 4-(2,4-Difurophenyl)-3,4-diazatricyclo[5.2.1.0^{2,6}]deca-2,5-diene-5-carboxylic acid

The title compound was synthesized by a procedure similar to that described for Intermediate 3. Intermediate 69 (300 mg, 0.94 mol), ethyl alcohol (3 ml), water (0.9 ml) and KOH (63 mg, 1.13 mmol) furnished the title compound (228 mg, 83%). ¹H-NMR (δ ppm, CDCl₃, 300 MHz): 7.52-7.38 (m, 1H); 7.00-6.6.85 (m, 2H); 3.64 (br. s, 1H); 3.46 (br. s, 1H); 2.14-1.92 (m, 3H); 1.75 (d, J=9.0, 1H); 1.35 (d, J=9.0, 2H).

### Intermediate 71: Preparation of Ethyl 2-(3-hydroxy-4,7,7-trimethyl bicyclo[2.2.1]hept-2-en-2-yl-2-oxoacetate

A solution of DL-camphor (5 g, 33 mmol) in toluene (25 ml) was added to a slurry of sodium hydride (60% dispersion, 1.34 g, 56 mmol) and diethyl oxalate (6.69 g, 49 mmol) in toluene (30 ml) at 60° C. and the mixture stirred at the same temperature for 1 hour. The reaction mixture was quenched into ice, acidified with 1N HCl, extracted with ethyl acetate and the organic layers dried over Na₂SO₄ and the solvent was removed under vacuum to give Intermediate 71 (7.3 g, 88%) which was used without further purification for the next step. ¹H-NMR (δ ppm, CDCl₃, 300 MHz): 11.39 (br. s, 1H); 4.35 (q, J=7.2, 2H); 3.29 (d, J=4.2, 1H); 2.30-2.04 (m, 1H); 1.70-1.40 (m, 1H); 1.46 (br. d, J=8.7, 2H); 1.38 (t, J=7.2, 3H); 1.01, 0.97, 0.83 (3s, 9H).

### Intermediate 72: Preparation of Ethyl 3-(2,4-difluorophenyl)-1,10,10-trimethyl-3,4-diazatricyclo[5.2.1.0^{2,6}]deca-2(6),4-dien-5-carboxylate

The intermediate 72 was prepared by a procedure similar to that described for intermediate 2. Intermediate 71 (1.0 g, 3.97 mmol), 2,4-difluorophenylhydrazine hydrochloride (990 mg, 4.63 mmol) and ethanol (10 ml) yielded Intermediate 72 (660 mg, 42%). ¹H-NMR (δ ppm, CDCl₃, 300 MHz): 7.58-7.48 (m, 1H); 7.03-6.80 (m, 2H); 4.40 (q, J=7.2, 2H); 3.15 (d, J=4.2, 1H); 2.20-2.08 (m, 1H); 1.88-1.76 (m, 1H); 1.40 (t, J=7.2, 3H); 1.40-1.08 (m, 2H); 0.99, 0.92, 0.79 (3s, 9H).

### Intermediate 73: Preparation of 3-(2,4-Difluorophenyl)-1,10,10-trimethyl-3,4-diazatricyclo[5.2.1.0^{2,6}]deca-2(6),4-dien-5-carboxylic acid

Intermediate 73 was prepared by a procedure similar to that described for intermediate 3. Intermediate 72 (660 mg, 1.68 mmol), KOH (141 mg, 2.52 mmol), ethanol (4.0 ml) and H₂O (1.0 ml) yielded Intermediate 73 (570 mg, 72%). ¹H-NMR (δ ppm, DMSO-d₆, 300 MHz): 12.80 (br. s, 1H); 7.80-7.57 (m, 2H); 7.29 (br. t, J=8.4, 1H); 3.01 (d, J=3.6, 1H); 2.08-2.02 (m, 1H); 1.79 ( br. t, J=9.6, 1H); 1.32 (br. t, J=9.0, 1H); 1.06 (br. t, J=9.0 1H); 0.91, 0.88, 0.73 (3s, 9H).

### Intermediate 74: Preparation of Ethyl 2-((1S,4R)-3-hydroxy-4,7,7-trimethyl bicyclo[2.2.1]hept-2-en-2-yl)-2-oxoacetate

A solution of (R)-(+)-camphor (5 g, 33 mmol) in toluene (25 ml) was added to a slurry of sodium hydride (60% dispersion, 1.10 g, 45 mmol) and diethyl oxalate (5.75 g, 39 mmol) in toluene (35 ml) at 60° C. and the mixture stirred at the same temperature for 1 hour. The reaction mixture was quenched into ice, acidified with 1N HCl, extracted with ethyl acetate and the organic layers dried over Na₂SO₄ and the solvent was removed under vacuum to give Intermediate 74 (4.0 g, 48%) which was used without purification for the next step. ¹H-NMR (δ ppm, CDCl₃, 300 MHz): 11.38 (br. s, 1H); 4.34 (q, J=7.2, 2H); 3.28 (d, J=3.9, 1H); 2.18-2.04 (m, 1H); 1.71-1.42 (m, 1H); 1.46 (br. d, J=8.7, 2H); 1.38 (t, J=7.2, 3H); 1.01, 0.97, 0.83 (3s, 9H).

### Intermediate 75: Ethyl (1R,7S)-3-(2,4-difluorophenyl)-1,10,10-trimethyl-3,4-diazatricyclo[5.2.1.0^{2,6}]deca-2(6),4-diene-5-carboxylate

The intermediate 75 was prepared by a procedure similar to that described for intermediate 2. Intermediate 74 (800 mg, 3.17 mmol), 2,4-difluorophenylhydrazine hydrochloride (650 mg, 3.61 mmol) and ethanol (10 ml) yielded Intermediate 75 (750 mg, 66%). ¹H-NMR (δ ppm, CDCl₃, 300 MHz): 7.58-7.48 (m, 1H); 7.04-6.85 (m, 2H); 4.40 (q, J=7.2, 2H); 3.14 (d, J=3.3, 1H); 2.21-2.07 (m, 1H); 1.88-1.77 (m, 1H); 1.40 (t, J=7.2, 3H); 1.40-1.08 (m, 2H); 0.99, 0.92, 0.79 (3s, 9H).

### Intermediate 76: Preparation of (1R,7S )-3-(2,4-Difluorophenyl)-1,10,10-trimethyl-3,4-diazatricyclo[5.2.1.0^{2,6}]deca-2(6),4-diene-5-carboxylic acid

Intermediate 76 was prepared by a procedure similar to that described for intermediate 3. Intermediate 75 (750 mg, 2.08 mmol), KOH (233 mg, 4.16 mmol), ethanol (10 ml) and H₂O (0.5 ml) yielded Intermediate 76 (640 mg, 92%). ¹H-NMR (δ ppm, CDCl₃, 300 MHz): 7.60-7.48 (m, 1H); 7.04-6.94 (m, 2H); 3.16 (d, J=3.3, 1H); 2.22-2.10 (m, 1H); 1.84 (t, J=9.3, 1H); 1.36 (br. t, J=8.7, 1H); 1.25 (br. t, J=8.7, 1H); 1.01, 0.93, 0.79 (3s, 9H).

### Intermediate 77: Preparation of Ethyl (2E)-hydroxy(4,7,7-trimethyl-3-oxobicyclo[2.2.1]hept-2-ylidene)acetate

This compound was prepared from (1S)-(−)-Camphor (3 g, 19.7 mmol), dry toluene (60-75 mL), sodium hydride (60% dispersion, 662 mg, 27.5 mmol) and diethyl oxalate (3.45 g, 23.6 mmol) according to the procedure described for intermediate 74. The product obtained as a yellow oil was used without purification for the next step. ¹H-NMR (δ ppm, CDCl₃, 300 MHz): 11.39 (br. s, 1H); 4.34 (q, J=7.2, 2H); 3.28 (d, J=3.9, 1H); 2.18-2.04 (m, 1H); 1.71-1.42 (m, 1H); 1.46 (br. d, J=8.7, 2H); 1.38 (t, J=7.2, 3H); 1.01, 0.97, 0.83 (3s, 9H).

### Intermediate 78: Ethyl (1S,7R)-3-(2,4-difluorophenyl)-1,10,10-trimethyl-3,4-diazatricyclo[5.2.1.0^{2,6}]deca-2(6),4-diene-5-carboxylate

This compound was prepared from intermediate 77 (1.0 g, 3.96 mmol), 2,4-difluorophenylhydrazine hydrochloride (787 mg, 4.36 mmol) and ethanol (10-20 mL) according to the procedure described for intermediate 2. ¹H-NMR (δ ppm, CDCl₃, 300 MHz): 7.59-7.49 (m, 1H); 7.05-6.85 (m, 2H); 4.39 (q, J=7.2, 2H); 3.13 (d, J=3.0, 1H); 2.20-2.03 (m, 1H); 1.89-1.76 (m, 1H); 1.40 (t, J=7.2, 3H); 1.40-1.08 (m, 2H); 0.99, 0.92, 0.79 (3s, 9H).

### Intermediate 79: Preparation of (1S,7R)-3-(2,4-Difluorophenyl)-1,10,10-trimethyl-3,4-diazatricyclo[5.2.1.0^{2,6}]deca-2(6),4-diene-5-carboxylic acid

This intermediate 89 was prepared from Intermediate 88 (690 mg, 1.91 mmol), KOH (128 mg, 2.29 mmol), ethanol (12 ml) and H₂O (2.0 ml) according to the procedure described for intermediate 3. ¹H-NMR (δ ppm, CDCl₃, 300 MHz): 7.59-7.46 (m, 1H); 7.05-6.92 (m, 2H); 3.17 (br. s, 1H); 2.20-2.10 (m, 1H); 1.95-1.75 (m, 1H); 1.36 (br. t, J=8.7, 1H); 1.25 (br. t, J=8.7, 1H); 1.01, 0.93, 0.79 (3s, 9H).

### Intermediate 80: Preparation of 4-Hydroxy-4-(3-oxobicyclo[2.2.1]hept-2-ylidene)butanoic acid

A 1.6M soln. of n-BuLi in hexane was added to a solution of hexamethyldisilazane (2.44 g, 38.12 mmol) in dry THF (100 ml) at −40° C. and stirred at that temperature for 15 min. To this mixture was added a solution of norcamphor (3.0 g, 27.23 mmol) in dry THF (50 ml) and stirring at −40° C. was continued for further 45 min. Succinic anhydride (3.26 gm, 32.67 mmol) was added and the mixture was allowed to warm slowly up to 25° C. After stirring overnight, water (200 ml) was added to the solution and the layers separated. The aqueous layer was washed twice with diethyl ether (150 ml), acidified with 1N HCl and extracted into ethyl acetate (3×50 ml), the organic layer was dried over Na₂SO₄, filtered and evaporated. Flash chromatography (petroleum ether/ethyl acetate 92:8) gave the title compound as a thick paste (1.9 g, 33%). ¹H-NMR (δ ppm, CDCl_{3,} 300 MHz): 3.04 (br. s, 1H); 2.99 (br. s, 1H); 2.88-2.55 (m, 4H); 1.98-1.77 (m, 3H); 1.68-1.55 (m, 2H); 1.46-1.38 (m, 1 H).

### Intermediates 81a and 81b: Ethyl 3-[5-(2,4-difluorophenyl)-4,5-diazatetracyclo[5.2.1.0^{2,6}]deca-2(6),3-diene-3-yl]propanoate and Ethyl-3-[4-(2,4-difluorophenyl)-4,5-diazatetracyclo[5.2.1.0^{2,6}]deca-2(6),5-diene-3-yl]propanoate

Intermediate 80 (1.0 g, 4.76 mmol) and 2,4-diflurophenyl hydrazine hydrochloride (945 mg, 5.23 mmol) and ethanol (15 ml) were refluxed for 3 h. Evaporation of the solvent and purification of the residue by SiO₂ column chromatography furnished two isomeric ethyl pyrazole carboxylates. Intermediate 81a (fast eluting): Yield: 559 mg (34%). ¹H-NMR (δ ppm, CDCl₃, 300 MHz): 7.68-7.60 (m, 1H); 6.95 (t, J=8.1, 2H); 4.16 (q, J=7.2, 2H); 3.42 (br. s, 1H); 3.35 (br. s, 1H); 2.98 (t, J=7.5, 2H); 2.71 (t, J=7.5, 2H); 2.00 (d, J=7.2, 1H); 1.90 (d, J=7.2, 2H); 1.65-1.58 (m, 1H); 1.26 (t, J=7.2, 3H), 1.15 (d, J=8.1, 2H).

Intermediate 81b (late eluting): Yield: 392 mg (24%). ¹H-NMR (δ ppm, CDCl₃, 300 MHz): 7.46-7.38 (m, 1H); 7.00-6.92 (m, 2H); 4.12 (q, J=7.1, 2H); 3.36 (br. s, 2H); 2.84-2.52 (m, 4H); 2.05-1.85 (m, 3H); 1.68 (d, J=8.4, 1H); 1.36-1.25 (m, 2H); 1.24 (t, J=7.1, 3H).

### Intermediate 82: preparation of 3-[4-(2,4-Difluorophenyl)-4,5-diazatetracyclo[5.2.1.0^{2,6}]deca-2(6),5-dien-3-yl]propanoic acid or 3-[5-(2,4-Difluorophenyl)-4,5-diazatetracyclo[5.2.1.0^{2,6}]deca-2(6),3-dien-3-yl]propanoic acid

Intermediate 82 was prepared by a procedure similar to that described for intermediate 3. Intermediate 81a (150 mg, 0.43 mmol), KOH (31 mg, 0.56 mmol), ethanol (1.36 ml) and H₂O (0.44 ml) yielded Intermediate 82 (95 mg, 69%). ¹H-NMR (δ ppm, CDCl₃, 300 MHz): 7.61 (t, J=8.4, 1H); 6.98 (t, J=9.0, 2H); 3.44 (br. s, 1H); 3.36 (br. s, 1H); 3.00 (t, J=6.6, 2H); 2.80 (t, J=6.6, 2H); 2.03 (d, J=7.8, 1H); 1.93 (d, J=6.0, 2H); 1.65 (t, J=8.7, 1H); 1.18 (d, J=7.8, 2H).

### Intermediate 83: Preparation of 3-[5-(2,4-Difluorophenyl)-4,5-diazatetracyclo[5.2.1.0^{2,6}]deca-2(6),3-dien-3-yl]propanoic acid or 3-[4-(2,4-Difluorophenyl)-4,5-diazatetracyclo[5.2.1.0^{2,6}]deca-2(6),5-dien-3-yl]propanoic acid

Intermediate 83 was prepared by a procedure similar to that described for intermediate 3. Intermediate 81b (150 mg, 0.43 mmol), KOH (31 mg, 0.56 mmol), ethanol (1.36 ml) and H₂O (0.44 ml) yielded Intermediate 83 (290 mg, 64%). ¹H-NMR (δ ppm, CDCl₃, 300 MHz): 7.45-7.38 (m, 1H); 7.00-6.94 (m, 2H); 3.37 (br. s, 2H); 2.82-2.68 (m, 4H); 2.08-1.85 (m, 3H); 1.68 (t, J=9.0, 1H); 1.39-1.22 (m, 2H).

### Intermediate 84: Preparation of Ethyl 2-oxo-2-(10-oxotricyclo[6.2.2.0^{2,7}]dodeca-2,4,6-trien-9-yl)acetate

This intermediate was prepared according to a procedure similar to method 1 described for intermediate 1. Tricyclo[6.2.2.0^{2,7}]dodeca-2,4,6-trien-9-one [prepared by one of the methods available in the art of organic synthesis, e.g., as described in Hales et. al. Tetrahedron, 1995, 51, 7777-7790] (2.5 g, 14.53 mmol), hexamethyldisilazane (4.9 ml, 23.2 mmol), 2.34M n-BuLi (10 ml, 23.4 mmol) and diethyl oxalate (3.18 ml, 21.18 mmol) furnished yellow oil which was directly used for the next step (2.3 g, 63%). ¹H-NMR (δ ppm, CDCl_{3,} 400 MHz): 12.9 (s, 1H); 7.20-7.15 (m, 4H); 4.91 (s, 1H); 4.31 (q, J=7.2, 2H); 3.79 (s, 1H); 2.00-1.90 (m, 2H); 1.73-1.60 (m, 2H); 1.34 (t, J=7.2, 3H).

### Intermediate 85: Ethyl 10-(2,4-difluorophenyl)-10,11-diazatetracyclo[6.5.2.0^{2,7}.0^{9,13}]pentadeca-2,4,6,9(13),11-pentaene-12- carboxylate

This compound was prepared by a procedure similar to that described for intermediate 2. Intermediate 84 (810 mg, 2.97 mmol), 2,4-difluorophenylhydrazine hydrochloride (589 mg, 3.26 mmol) and ethanol (10 ml) yielded pyrazole ester (800 mg, 71%). ¹H-NMR (δ ppm, CDCl₃, 300 MHz): 7.67-7.57 (m, 1H); 7.34 (d, J=6.9, 1H); 7.20-7.01 (m, 5H); 4.90 (s, 1H); 4.44 (q, J=6.9, 2H); 4.39 (br. s, 1H); 1.79 (s, 4H); 1.45 (t, J=6.9, 3H).

### Intermediate 86: 10-(2,4-Difluorophenyl)-10,11-diazatetracyclo[6.5.2.0^{2,7}.0^{9,13}]pentadeca-2,4,6,9(13),11-pentaene-12-carboxylic acid

This was prepared by a procedure similar to that described for intermediate 3. Intermediate 85 (800 mg, 2.3 mmol), KOH (194 mg, 3.4 mmol), ethanol (10.0 ml) and H₂O (0.5 ml) afforded intermediate 86 (700 mg, 86%). ¹H-NMR (δ ppm, CDCl₃, 300 MHz): 7.68-7.58 (m, 1H); 7.35 (d, J=7.2, 1H); 7.21-7.04 (m, 5H); 4.94 (s, 1H); 4.41 (br. s, 1H); 1.81 (br. s, 4H).

### Intermediate 87: Preparation of Ethyl 2-oxo-(5-oxotricyclo[4.3.1.1.^{3,8}]undec-4-yl)acetate

A 1.6M soln. of n-BuLi in hexane was added to a solution of hexamethyldisilazane (1.27 ml, 5.4 mmol) in diethyl ether (10.0 ml) at −78° C. and stirred at that temperature for 15 min. To this mixture was added a solution of homoadamantanone [900 mg, 5.4 mmol, prepared according to: Black, R. M. and Gill, G. B., J. Chem. Soc. (C), 1970, 671] in diethyl ether (27.0 ml) and stirring at −78° C. was continued for further 45 min. Diethyl oxalate (0.98 ml, 6.5 mmol) was added and the mixture was allowed to slowly warm up to 25° C. After stirring overnight, water (25 ml) was added to the solution and the layers separated. The aqueous layer was washed twice with diethyl ether (20 ml), acidified with 1N HCl and extracted into diethyl ether (3×20 ml), the organic layer was dried over Na₂SO₄, filtered and evaporated. Flash chromatography (petroleum ether/ethyl acetate 97:3) gave the title compound as a yellow oil (589 mg, 36%). ¹H-NMR (δ ppm, CDCl₃, 300 MHz): 15.75 (s, 1H); 4.33 (q, J=7.2, 2H); 2.80 (br. t, J=6, 1H); 2.75-2.70 (m, 1H); 2.13-85 (m, 8H); 1.81-1.69 (m, 4 H); 1.36 (m, t, J=7.2, 3H). IR (cm⁻¹ neat): 3423 (br.), 2982 (w), 2919 (s), 2851 (m), 1741 (s), 1599 (s, br.).

### Intermediate 88: Preparation of Ethyl 5-(4-chlorophenyl)-5,6-diazatetracyclo[7,3,1.1^{3,11},0^{4,8}]tetradeca-4(8),6-diene-7-carboxylate

This compound was prepared by a procedure similar to that described for intermediate 2. From intermediate 87 (1 g, 3.8 mmol), 4-Chlorophenylhydrazine hydrochloride (711 mg, 4.0 mmol) and ethanol (10 ml), pure pyrazole ester (760 mg, 54%) was obtained. ¹H-NMR (δ ppm, CDCl₃, 300 MHz): 7.43, 7.30 (AB, J=10, 4H); 4.40 (q, J=7.5, 2H); 3.79 (t, J=5.1, 1H); 3.0 (t, J=5.4, 1H); 2.21 (br. s, 2H); 2.06-1.77 (m, 10H); 1.40 (t, J=7.5, 3H).

### Intermediate 89: Preparation of 5-(4-Chlorophenyl)-5,6-diazatetracyclo[7,3,1.1^{3,11},0^{4,8}]tetradeca-4(8),6-diene-7-carboxylic acid

This was prepared by a procedure similar to that described for intermediate 2. Intermediate 88 (760 mg, 2.1 mmol), KOH (229 mg, 4.1 mmol) and ethanol (4 ml) furnished Intermediate 89 (630 mg (89%). ¹H-NMR (δ ppm, DMSO-d₆): 7.59 (d, J=8.7, 2H); 7.39 (d, J=8.7, 2H); 3.76 (br. s, 1H); 2.97 (br. s, 1H); 2.14 (br. s, 2H); 1.67-1.98 (m, 10H).

### Intermediate 90: Preparation of Ethyl 5-(2,4-Difluorophenyl)-5,6-diazatetracyclo[7.3.1.1^{3,11}.0^{4,8}]tetradeca-4(8),6-diene-7-carboxylate

The intermediate 90 was prepared by a procedure similar to that described for intermediate 2. From intermediate 87 (1.2 g, 4.5 mmol), 2,4-difluorophenyl hydrazine hydrochloride (820 mg, 4.5 mmol) and ethanol (10 ml), the yield of intermediate 90 was 1.5 g (96%). ¹H-NMR (δ ppm, CDCl₃, 300 MHz): 7.50-7.40 (m, 1H); 7.02-6.90 (m, 2H); 4.38 (q, J=7.2, 2H); 3.76 (br. s, 1H); 2.66 (br. s, 1H); 2.17 (br. s, 2H); 2.05-1.70 (m, 10H), 1.37 (t, J=7.2, 3H).

### Intermediate 91: Preparation of 5-(2,4-Difluorophenyl)-5,6-diazatetracyclo[7.3.1.1^{3,11}.0^{4,8}]tetradeca-4(8) 6-diene-7-carboxylic acid

Intermediate 91 was prepared by a procedure similar to that described for intermediate 3. From intermediate 90 (1.5 g, 4.36 mmol), KOH (554 mg, 9.87 mmol), ethanol (10.0 ml) and water (1.0 ml) intermediate 91 (1.30 g, 95%) was obtained. ¹H-NMR (δ ppm, CDCl₃, 300 MHz): 12.80 (br. s, 1H); 7.70-7.55 (m, 2H); 7.30 (br. t, J=7.5, 1H); 3.66 (br. s, 1H); 2.63 (br. s, 1H); 2.13 (s, 2H); 2.00-1.71 (m, 10H).

### Intermediate 92: Preparation of Ethyl 5-(4-Fluorophenyl)-5,6-diazatetracyclo[7.3.1.1^{3,11}.0^{4,8}]tetradeca-4(8),6-diene-7-carboxylate

The intermediate 92 was prepared by a procedure similar to that described for intermediate 2. Intermediate 87 (1.165 g, 4.40 mmol), 4-fluorophenylhydrazine hydrochloride (860 mg, 5.29 mmol) and ethanol (20 ml) yielded intermediate 92 (1.20 g, 55%). ¹H-NMR (δ ppm, CDCl₃, 300 MHz): 7.38-7.31 (m, 2H); 7.18-7.11 (m, 2H); 4.39 (q, J=7.5, 2H); 3.78 (br. s, 1H); 2.95 (br. s, 1H); 2.20 (br. s, 2H); 2.06-1.76 (m, 10H); 1.39 (t, J=7.2, 3H).

### Intermediate 93: Preparation of 5-(4-Fluorophenyl)-5,6-diazatetracyclo[7.3.1.1^{3,11}.0^{4,8}]tetradeca-4(8),6-diene-7-carboxylic acid

Intermediate 57 was prepared by a procedure similar to that described for intermediate 3. Intermediate 92 (1.20 g, 3.4 mmol), KOH (380 mg, 6.8 mmol), ethanol (20.0 ml) and H₂O (0.5 ml) afforded intermediate 93 (900 mg, 81%). ¹H-NMR (δ ppm, DMSO-d₆, 300 MHz): 12.60 (br. s, 1H); 7.47-7.35 (m, 4H); 3.67 (br. s, 1H); 2.91 (br. s, 1H); 2.14 (br. s, 2H); 1.98-1.71 (m, 10H).

### Intermediate 94: Preparation of Ethyl 2-(3-oxo-bicyclo[2.2.2]oct-2-yliden]-2-hydroxyacetate

This intermediate was prepared from Bicyclo[2.2.2]octan-2-one (2.4 g, 19.35 mmol), toluene (30-50 mL), sodium hydride (60% dispersion, 603 mg, 25.16 mmol) and diethyl oxalate (3.15 ml, 23.2 mmol) according to a procedure described in method 2 for intermediate 1 and was obtained as a yellow oil, which was used in the next step without purification. ¹H-NMR (δ ppm, CDCl₃, 300 MHz): 13.58 (br. s, 1H); 4.36 (q, J=7.2, 2H); 3.58 (br. s, 1H); 2.52 (br. s, 1H); 1.86-1.52 (m, 8H); 1.38 (t, J=7.2, 3H).

### Intermediate 95: Preparation of Ethyl 3-(4-chlorophenyl)-3,4-diazatricyclo[5.2.2.0^{2,6}]undeca-2(6),4-dien-5-carboxylate

This compound was prepared as a yellow solid from intermediate 94 (400 mg, 1.26 mmol), ethanol (5-10 mL), 4-chlorophenylhydrazine hydrochloride (249 mg, 1.39 mmol) according to a procedure described for intermediate 2. ¹H-NMR (δ ppm, CDCl₃, 300 MHz): 7.52 (d, J=9.0, 1H); 7.45 (d, J=9.0, 2H); 4.43 (q, J=7.2, 2H); 3.70 (br. s, 1H); 3.44 (br. s, 1H); 1.80 (d, J=8.4, 4H); 1.45-1.35 (m, 6H).

### Intermediate 96: Preparation of 3-(4-Chlorophenyl)-3,4-diazatricyclo[5.2.2.0^{2,6}]undeca-2(6),4-dien-5-carboxylic acid

The intermediate 96 was prepared from intermediate 95 (340 mg, 1.02 mmol), ethanol (3-5 mL), water (1-5 mL) and potassium hydroxide (74 mg, 1.33 mmol) according to a procedure described in intermediate 3. ¹H-NMR (δ ppm, CDCl₃, 300 MHz): 7.52 (d, J=9.0, 1H); 7.47 (d, J=9.0, 2H); 3.74 (br. s, 1H); 3.47 (br. s, 1H); 1.82 (d, J=7.5, 4H); 1.41 (d, J=7.5, 4H).

### Intermediate 97: Preparation of Ethyl 3-(4-trifluoromethylphenyl)-3,4-diazatricyclo[5.2.2.0^{2,6}]undeca-2(6),4-dien-5-carboxylate

The intermediate 97 was prepared from intermediate 94 (400 mg, 1.26 mmol), ethanol (5-10 mL), 4-trifluoromethylphenylhydrazine hydrochloride (245 mg, 1.39 mmol) according to a procedure described for intermediate 2 and was obtained as a yellow solid. ¹H-NMR (δ ppm, CDCl₃, 300 MHz): 7.74 (br. s, 4H); 4.44 (q, J=6.9, 2H); 3.72 (br. s, 1H); 3.51 (br. s, 1H); 1.82 (d, J=9.0, 4H); 1.48-1.35 (m, 7H).

### Intermediate 98: Preparation of 3-(4trifluoromethylphenyl)-3,4-diazatricyclo[5.2.2.0^{2,6}]undeca-2(6),4-dien-5-carboxylic acid

The intermediate 98 was prepared from intermediate 97 (150 mg, 0.41 mmol), ethanol (1-5 mL), water (1-5 mL) and potassium hydroxide (27 mg, 0.49 mmol) according to a procedure described in intermediate 3. ¹H-NMR (δ ppm, CDCl₃, 300 MHz): 7.80-7.68 (m, 4H); 3.75 (br. s, 1H); 3.53 (br. s, 1H); 1.84 (d, J=6.3, 4H); 1.43 (d, J=6.6, 4H).

### Intermediate 99: Preparation of Ethyl 3-(4-bromophenyl)-3,4-diazatricyclo[5.2.2.0^{2,6}]undeca-2(6),4-dien-5-carboxylate

The intermediate 99 was prepared from Intermediate 94 (400 mg, 1.26 mmol), ethanol (5-10 mL) and 4-bromophenylhydrazine hydrochloride (311 mg, 1.39 mmol) according to a procedure described for intermediate 2. Yellow solid. ¹H-NMR (δ ppm, CDCl₃, 300 MHz): 7.60 (d, J=8.7, 2H); 7.45 (d, J=9.0, 2H); 4.43 (q, J=7.2, 2H); 3.70 (br. s, 1H); 3.44 (br. s, 1H); 1.80 (d, J=8.4, 4H); 1.45-1.32 (m, 7H).

### Intermediate 100: Preparation of 4-Bomophenyl)-3,4-diazatricyclo[5.2.2.0^{2,6}]undeca-2(6),4-dien-5-carboxylic acid

The intermediate 100 was prepared from Intermediate 99 (330 mg, 0.87 mmol), ethanol (2-5 mL), water (1-5 mL) and potassium hydroxide (63 mg, 1.30 mmol) according to a procedure described for intermediate 3. ¹H-NMR (δ ppm, CDCl₃, 300 MHz): 7.63 (d, J=8.4, 2H); 7.47 (d, J=8.1, 2H); 3.74 (br. s, 1H); 3.47 (br. s, 1H); 1.82 (d, J=6.6, 4H); 1.41 (d, J=6.9, 4H).

### Intermediate 101: Preparation of Ethyl 3-(4-Fluorophenyl)-3,4-diazatricyclo[5.2.2.0^{2,6}]undeca-2(6),4-dien-5-carboxylate

The intermediate 101 was prepared from Intermediate 94 (400 mg, 1.26 mmol), ethanol (5-10 mL) and 4-flurophenylhydrazine hydrochloride (226 mg, 1.39 mmol) according to a procedure described in intermediate 2 as a yellow solid. ¹H-NMR (δ ppm, CDCl₃, 300 MHz): 7.58-7.48 (m, 2H); 7.17 (t, J=8.4, 2H); 4.43 (q, J=6.9, 2H); 3.70 (br. s, 1H); 3.40 (br. s, 1H); 1.80 (d, J=7.5, 4H); 1.46-1.33 (m, 7H).

### Intermediate 102: Preparation of 3-(4-Fluorophenyl)-3,4-diazatricyclo[5.2.2.0^{2,6}]undeca-2(6),4-dien-5-carboxylic acid

The intermediate 102 was prepared from intermediate 101 (221 mg, 0.70 mmol), ethanol (3-5 mL), water (1-5 mL) and potassium hydroxide (51 mg, 1.30 mmol) according to a procedure described in intermediate 3. ¹H-NMR (δ ppm, CDCl₃, 300 MHz): 7.59-7.48 (m, 2H); 7.20 (t, J=9.0, 2H); 3.70 (br. s, 1H); 3.44 (br. s, 1H); 1.82 (d, J=7.8, 4H); 1.41 (d, J=6.9, 4H).

### Intermediate 103: Preparation of ethyl-3-(2,4-difluorophenyl)-3,4-diazatricyclo[5.2.2.0^{2,6}]undeca-2(6),4-diene-5-carboxylate

The intermediate 103 was prepared by a procedure similar to that described for intermediate 2. Intermediate 94 (1.2 g, 5.35 mmol), 2,4-difluorophenylhydrazine hydrochloride (1.06 mg, 5.89 mmol), ethanol (20 ml) yielded Intermediate 103 (910 mg, 51%). ¹H-NMR (δ ppm, CDCl₃, 300 MHz): 7.70-7.60 (m, 1H); 7.06-6.94 (m, 2H); 4.43 (q, J=7.2, 2H); 3.70 (br. s, 1H); 3.15 (br. s, 1H); 1.78 (d, 7.8, 4H); 1.45-1.36 (m, 7H).

### Intermediate 104: Preparation of 3-(2,4-difluorophenyl)-3,4-diazatricyclo[5.2.2.0^{2,6}]undeca-2(6),4-diene-5-carboxylic acid

Intermediate 104 was prepared by a procedure similar to that described for intermediate 3. Intermediate 103 (850 mg, 2.55 mmol), ethanol (8 ml), H₂O (0.5 ml) and KOH (186 mg, 3.32 mmol) yielded Intermediate 104 (702 mg, 90%). ¹H-NMR (δ ppm, CDCl₃, 300 MHz): ¹H-NMR (δ ppm, CDCl₃, 300 MHz): 7.72-7.60 (m, 1H); 7.09-6.98 (m, 2H); 3.73 (br. s, 1H); 3.18 (br. s, 1H); 1.80 (d, J=6.6, 4H); 1.40 (d, J=7.8, 4H).

### Intermediate 105: Preparation of Ethyl 2-[(1S,8S)-7,7-dimethyl-3-oxo-bicyclo[2.2.1]hept-2-yliden]-2-hydroxyacetate

A solution of (1S)-(+)-nopinone (7.5 g, 53 mmol) in toluene (40 ml) was added to slurry of sodium hydride (60% dispersion, 1.94 g, 80.00 mmol) and diethyl oxalate (8.64 ml, 63.6 mmol) in toluene (40 ml) at 60° C. and the mixture stirred at the same temperature for 1 h. The reaction mixture was quenched into ice, acidified with 1N HCl, extracted with ethyl acetate and the organic layers dried over Na₂SO₄ and the solvent was removed under vacuum to give the diketoester (7.0 g, 54%) as a yellow oil, which was used without purification for the next step. ¹H-NMR (δ ppm, CDCl₃, 300 MHz): 14.37 (br. s, 1H); 4.36 (q, J=7.2, 2H); 2.94-2.88 (m, 2H); 2.66-2.54 (m, 2H); 2.31 (br. s, 1H); 1.44-1.35 (m, 7H); 0.91 (s, 3H).

### Intermediate 106: Preparation of Ethyl (1S,8S)-3-(2,4-difluorophenyl)-10,10-dimethyl-3,4-diazatricyclo[5.2.1.0^{2,6}]deca-2(6),4-dien-5-carboxylate

Intermediate 105 (500 mg, 2.10 mmol) was dissolved in ethanol (10 ml), treated with 2,4-difluorophenylhydrazine hydrochloride (832 mg, 4.60 mmol) and heated at reflux for 2 h. Solvent was evaporated, the residue diluted with water and extracted with ethyl acetate. Organic extracts were washed with brine and dried over Na₂SO₄. Chromatographic purification of crude product gave the title compound (700 mg, 96%) as a white solid. ¹H-NMR (δ ppm, CDCl₃, 300 MHz): 7.62-7.50 (m, 1H); 7.06-6.89 (m, 2H); 4.42 (q, J=6.9, 2H); 3.02-2.92 (m, 2H); 2.75-2.64 (m, 2H); 2.35 (br. s, 1H); 1.44-1.32 (m, 7H); 0.75 (br. s, 3H).

### Intermediate 107: Preparation of (1S,8S)-3-(2,4-difluorophenyl)-10,10-dimethyl-3,4-diazatricyclo[5.2.1.0^{2,6}]deca-2(6),4-dien-5-carboxylic acid

A solution of intermediate 106 (300 mg, 0.86 mmol) in ethanol (5 ml) and H₂O (0.5 ml) was treated with KOH (58 mg, 1.03 mmol) and stirred at RT for 4 h. The solvent was evaporated and the residue diluted with water and acidified with 1N HCl and the white precipitates of the title compound were collected by filteration (260 mg, 94%). ¹H-NMR (δ ppm, CDCl₃, 300 MHz): 7.62-7.50 (m, 1H); 7.08-6.95(m, 2H); 3.03-2.96 (m, 2H); 2.77-2.66 (m, 2H); 2.36 (br. s, 1H); 1.48-1.39 (m, 7H); 0.76 (br. s, 3H).

### Intermediate 108: Preparation of (1S,8S)-3-(2,4-difluorophenyl)-10,10-dimethyl-3,4-diazatricyclo[5.2.1.0^{2,6}]deca-2(6),4-dien-5-amine

The title compound was synthesized by a procedure similar to that described for Intermediate 9. Intermediate 107 (1.0 g, 3.14 mmol), 1,4-dioxane (8 mL), Et₃N (0.53 ml, 3.77 mmol), BOP reagent (1.52 g, 3.45 mmol), sodium azide (400 mg, 6.28 mmol) and tetrabutyl ammonium bromide (2.02 g, 6.28 mmol) furnished Intermediate 108 as a reddish oil (650 mg, 71%). ¹H-NMR (δ ppm, CDCl₃, 300 MHz): 7.43 (q, J=8.4, 1H), 6.91 (t, J=8.4, 2H); 2.68-2.55 (m, 4H); 2.31 (m, 1H); 1.38 (s, 4H); 0.80 (s, 3H).

### Intermediate 109: Preparation of Ethyl 2-[(1R,5R)-6,6-dimethyl-2-oxobicyclo[3.1.1]hept-2-yliden]-2-hydroxyacetate

A solution of (1R)-(+)-nopinone (3.5 g, 25.31 mmol) in toluene (20-50 mL) was added to slurry of sodium hydride (60% dispersion, 789 mg, 32.90 mmol) and diethyl oxalate (4.12 ml, 30.37 mmol) in toluene (20-50 mL) at about 60° C. and the reaction mixture stirred at the same temperature for 1 hour. The reaction mixture was quenched into ice, acidified with 1N Hydrogenchloride, extracted with ethyl acetate and the organic layers dried over sodium sulphate and the solvent was removed under vacuum to gave Ethyl 2-[(1R,5R)-6,6-dimethyl-2-oxobicyclo[3.1.1]hept-2-yliden]-2-hydroxyacetate as a yellow oil, which was used without purification. ¹H-NMR (δ ppm, CDCl₃, 300 MHz): 14.37 (br. s, 1H); 4.36 (q, J=7.2, 2H); 2.94-2.89 (m, 2H); 2.65-2.57 (m, 2H); 2.31 (br. s, 1H); 1.44-1.35 (m, 7H); 0.91 (s, 3H).

### Intermediate 110: Preparation of Ethyl (1R,8R)-3-(2,4-difluorophenyl)-9,9-dimethyl-3,4-diazatricyclo[6.1.1.0^{2,6}]deca-2(6),4-dien-5-carboxylate

To a solution of Ethyl 2-[(1R,5R)-6,6-dimethyl-2-oxobicyclo[3.1.1]hept-2-yliden]-2-hydroxyacetate (1.00 g, 4.20 mmol) in ethanol (10-20 mL) was added 2,4-difluorophenylhydrazine hydrochloride (832 mg, 4.60 mmol) and refluxed for about 2 hours. The solvent was evaporated and diluted with water and extracted with ethyl acetate and organic extracts was washed with brine and dried over sodiumsulphate. Purification of crude product by silical gel column chromatography gave Ethyl (1R,8R)-3-(2,4-difluorophenyl)-9,9-dimethyl-3,4-diazatricyclo[6.1.1.0^{2,6}]deca-2(6),4-dien-5-carboxylate as an off-white solid. ¹H-NMR (δ ppm, CDCl₃, 300 MHz): 7.62-7.50 (m, 1H); 7.04-6.90 (m, 2H); 4.42 (q, J=6.9, 2H); 3.03-2.90 (m, 2H); 2.76-2.65 (m, 2H); 2.35 (br. s, 1H); 1.44-1.1.34 (m, 7H); 0.75 (s, 3H).

### Intermediate 111: Preparation of (1R,8R)-3-(2,4-Difluorophenyl)-9,9-dimethyl-3,4-diazatricyclo[6.1.1.0^{2,6}]deca-2(6),4-dien-5-carboxylic acid

Ethyl (1R,8R)-3-(2,4-difluorophenyl)-9,9-dimethyl-3,4-diazatricyclo[6.1.1.0^{2,6}]deca-2(6),4-dien-5-carboxylate (1.0 g, 2.88 mmol) was dissolved in ethanol (10-20 mL) and treated with water (0.5-1.0 mL) and potassium hydroxide (322 mg, 5.76 mmol) at an ambient temperature for about 4 hours. After evaporation of the solvent, the residue was diluted with water and acidified with 1N hydrogenchloride to gave (1R,8R)-3-(2,4-Difluorophenyl)-9,9-dimethyl-3,4-diazatricyclo[6.1.1.0^{2,6}]deca-2(6),4-dien-5-carboxylic acid as a white solid. ¹H-NMR (δ ppm, CDCl₃, 300 MHz): 7.62-7.50 (m, 1H); 7.08-6.95(m, 2H); 3.04-2.94 (m, 2H); 2.78-2.66 (m, 2H); 2.36 (br. s, 1H); 1.46-1.38 (m, 7H); 0.75 (s, 3H).

### Intermediate 112: Preparation of 1-Azido-3,3-dimethyl-2-butanone

To a solution of sodium azide (399 mg, 6.14 mmol) in dry DMF (5 ml), 1-bromo pinacolone (751 μl, 5.58 mmol) was added dropwise at 0° C. during 5 min. After warming to room temperature over 5 h, the reaction was diluted with water and extracted with ethyl acetate/hexane (1:1). The layers were separated, the organic layers were washed with water, brine and dried over Na₂SO₄. The solvent was removed under reduced pressure to give yellow oil (689 mg, 88%). ¹H-NMR (δ ppm, CDCl_{3,} 300 MHz): 4.08 (s, 2H); 1.19 (s, 9H). IR (cm⁻¹, KBr): 3413 (w), 2971 (m), 2909 (m), 2875 (m), 2104 (s), 1718 (m), 1477 (w), 1282 (m), 1066 (m), 1014 (w), 910 (m).

### Intermediate 113: Preparation of 1-Amino-3,3-dimethyl-2-butanone hydrochloride

Acetyl chloride (131 μl, 1.84 mmol) was added to cooled solution of ethanol (5 ml) and intermediate 112 (200 mg, 1.41 mmol) was dissolved in it and evacuated and flushed with nitrogen. To the above solution was added 20 mg of palladium on carbon (10%). The resulting slurry was again flushed with nitrogen and stirred under 1 atm of hydrogen for 3 h. The reaction mixture was filtered through a pad of celite and solvent was removed under reduced pressure and dried under vacuum to furnish white solid (194 mg, 90%). ¹H-NMR (δ ppm, CDCl_{3,} 300 MHz): 8.39 (br. s, 3H); 4.33 (br. s, 2H); 1.20 (s, 9H). IR (cm⁻¹, KBr): 3431 (s), 2979 (s), 2619 (m), 1719 (s), 1638 (m), 1605 (w), 1480 (s), 1385 (m), 1141 (m), 987 (m), 893(m).

### Intermediate 114: Preparation of ethyl 2,2-dimethyl-3-oxobutanoate

Sodium metal (159 mg, 6.94 mmol) was dissolved completely in dry ethanol (20 ml), ethyl-2-methyl acetoacetate (981 μl, 6.94 mmol) was added to the solution and stirred at 0° C. for 15 min. Methyl iodide (472 μl, 7.63 mmol) was added dropwise and after stirring at room temperature overnight, the reaction mixture was heated at 50° C. for 4 h. After cooling to ambient temperature, the solvent was removed completely under reduced pressure, the residue diluted with water and extracted thrice into diethyl ether. The combined organic layers were washed successively with water and brine and dried over Na₂SO₄. The solvent removal gave the product as an yellow oil (920 mg, 83%). ¹H-NMR (δ ppm, CDCl₃, 300 MHz): 4.19 (d, J=7.5, 2H); 2.16 (s, 2H); 1.36 (s, 6H); 1.26 (t, J=7.5, 3H).

### Intermediate 115: Preparation of ethyl 4-bromo-2,2-dimethyl-3-oxobutanoate

Method 1: To a solution of intermediate 114 (250 mg, 1.58 mmol) in ethyl acetate-chloroform (9 ml, 3:1), cupric bromide (1.06 gm, 4.74 mmol) was added and stirred at reflux temperature for 24 h. The reaction mixture was filtered through celite and celite was washed with ethyl acetate. The filtrate and washings were combined and washed successively with water and brine and dried over Na₂SO₄. The solvent was evaporated to give a yellow oil (370 mg, 98%).

Method 2: To a solution of ethyl 2,2-dimethyl-3-oxobutanoate (500 mg, 3.16 mmol) in dry DMF (6 mL), N-bromosuccinimide (670 mg, 3.79 mmol) was added and heated at 80° C. for 4 h. The reaction mixture was diluted with water and extracted with ethyl acetate and the combined organic extracts were washed with water and then with brine and dried over Na₂SO₄. The solvent was evaporated to give a yellow oil (725 mg, 96%). ¹H-NMR (δ ppm, CDCl₃, 300 MHz): 4.25-4.14 (m, 2H); 4.12 (s, 2H); 1.53 (s, 3H); 1.45 (s, 3H); 1.31-1.22 (m, 3H).

### Intermediate 116: Preparation of Ethyl 4-azido-2,2-dimethyl-3-oxobutanoate

The title compound was synthesized by a procedure similar to that described for Intermediate 112. Intermediate 115 (373 mg, 1.57 mmol), sodium azide (122 mg, 1.88 mmol) and dry DMF (5 ml) to give yellow oil (100 mg, 32%). ¹H-NMR (δ ppm, CDCl_{3,} 300 MHz): 4.21 (q, J=7.2, 2H); 4.06 (s, 2H); 1.41 (s, 6H); 1.27 (t, J=7.2, 3H). IR (cm⁻¹, KBr): 3380 (w), 2986 (m), 2939 (m), 2877 (m), 2106 (s), 1720 (s), 1469 (m), 1415 (w), 1387 (w), 1274 (m), 1150 (m), 1052 (m), 1022 (m), 910 (w), 795 (m).

### Intermediate 117: Preparation of Ethyl 4-amino-2,2-dimethyl-3-oxobutanoate hydrochloride

The title compound was synthesized by a procedure similar to that described for Intermediate 113. Intermediate 112 (600 mg, 3.01 mmol), acetyl chloride (278 μl, 3.91 mmol), ethanol (20 ml) and 30 mg of palladium on carbon (10%) furnished the title compound as a yellow solid (507 mg, 80%). ¹H-NMR (δ ppm, CDCl_{3,} 300 MHz): 8.40 (br. s, 3H); 4.35 (br. s, 2H); 4.19 (br. s, 2H); 1.47 (s, 6H); 1.29-1.22 (m, 3H).

### Intermediate 118: Preparation of Ethyl 3-phenyl-3,4-diazatricyclo[5.2.1.0^{2,6}]deca-2(6),4-dien-5-carboxylate

The intermediate 118 was prepared by a procedure similar to that described for intermediate 2. From intermediate 1 (1.0 g, 4.76 mmol), ethanol (16.0 ml), and phenylhydrazine (0.52 ml, 5.23 mmol) and a pre-mixed solution of acetyl chloride (0.85 ml) in ethanol (4.0 ml), pure intermediate 118 (700 mg, 52%). ¹H-NMR (δ ppm, CDCl₃, 300 MHz): 7.73 (d, J=8.7, 2H); 7.47 (t, J=7.2, 2H); 7.33 (t, J=7.2, 1H); 4.42 (q, J=7.2, 2H); 3.72 (br. s, 1H); 3.68 (br. s, 1H); 2.13 (br. d, J=8.7, 1H); 2.05-1.95 (m, 2H); 1.72 (d, J=8.7, 1H); 1.42 (t, J=7.2, 3H); 1.30-1.18 (m, 2H).

### Intermediate 119: Preparation of 3-phenyl-3,4-diazatricyclo[5.2.1.0^{2,6}]deca-2(6),4-dien-5-carboxylic acid

Intermediate 119 was prepared by a procedure similar to that described for intermediate 3. Intermediate 118 (700 mg, 2.5 mmol), ethanol (4.0 ml), KOH (280 mg, 5.0 mmol) and H₂O (0.5 ml) afforded intermediate 119 (500 mg, 79%). ¹H-NMR (δ ppm, CDCl₃, 300 MHz): 7.73 (d, J=7.8, 2H); 7.50 (t, J=7.8, 2H); 7.36 (t, J=7.5, 1H); 3.75 (s, 1H); 3.72 (s, 1H); 2.17 (br. d, J=9.0, 1H); 2.10-1.93 (m, 2H); 1.74 (d, J=8.7, 1H); 1.38-1.08 (m, 2H).

### EXAMPLES

### Example 1

### Preparation of 5-(2,4-difluorophenyl)-4,5-diazatricyclo[5.2.1.0^{2,6}.]deca-2(6),3-dien-3-yl-phenylmethanone

Grignard reagent was generated from bromobenzene (187 μl, 1.80 mmol) and magnesium turnings (50 mg, 2.10 mgatom) in diethyl ether (15 ml) and treated drop-wise with Intermediate 6 (500 mg, 1.50 mmol) in diethyl ether (10 ml) was added and the mixture was stirred at RT for 2 h. An aqeous saturated NH₄Cl solution was added and extracted with ethyl acetate. The organic layers were washed with brine, Na₂SO₄ and the solvent evaporated. Purification by chromatography followed by preparative HPLC afforded the title compound as a waxy solid (269 mg, 51%). ¹H-NMR (δ ppm, CDCl₃, 300 MHz): 8.30 (d, J=7.2, 2H); 7.79-7.68 (m, 1H); 7.62-7.44 (m, 3H); 7.02 (t, J=9.0, 2H); 3.70 (br. s, 1H); 3.51 (br. s, 1H); 2.14 (d, J=8.4, 1H); 1.99 (d, J=7.5, 2H); 1.72 (d, J=8.4, 1H), 1.28 (d, J=9.3, 2H). MS (m/z): 351.41 ([M+H]⁺).

### Example 2

### Preparation of 1-[5-(2,4-diflurophenyl)-4,5-diazatricyclo[5.2.1.0^{2,6}]deca-2(6),3-dien-3-yl]-1-hexanone

The title compound was synthesized by a procedure similar to that described for Example 1. Intermediate 6 (500 mg, 1.50 mmol), Mg turnings (50 mg, 2.10 mmol), diethyl ether (15 ml), 1-bromo-n-pentane (223 ρl, 1.80 mmol) furnished the title compound as a pale yellow oil (125 mg, 24%). ¹H-NMR (δ ppm, CDCl₃, 300 MHz): 7.74-7.68 (m, 1H), 7.07-7.00 (m, 2H); 3.71 (br. s, 1H); 3.46 (br. s, 1H); 3.05-2.95 (m, 2H); 2.07 (d, J=9.0, 1H); 1.96 (d, J=9.0, 2H); 1.80-1.60 (m, 3H); 1.42-1.30 (m, 4H); 1.22 (d, J=10.2, 2H); 0.95-0.86 (m, 3H). IR (cm⁻¹, KBr): 3091 (m), 2956 (s), 2932 (s), 2872 (s), 1683 (s), 1610 (m), 1521 (s), 1428 (m), 1404 (m), 1367 (m), 1325 (w), 1270 (m), 1189 (w), 1145 (m), 1092 (m), 965 (m). MS (m/z): 345.38 ([M+H]⁺).

### Example 3

### Preparation of 5-(2,4-diflurophenyl)-4,5-diazatricyclo[5.2.1.0^{2,6}.]deca-2(6),3-dien-3-yl-1-naphthylmethanone

The title compound was synthesized by a procedure similar to that described for Example 1. Intermediate 6 (500 mg, 1.50 mmol), Mg turnings (50 mg, 2.10 mmol), diethyl ether (15 ml), 1-bromonaphthalene (250 μl, 1.80 mmol) furnished the title compound as an off-white solid after preparative HPLC purification (66 mg, 11%). M.P.: 81-84° C. ¹H-NMR (δ ppm, CDCl₃, 300 MHz): 8.37 (d, J=5.7, 1H); 8.02-7.98 (m, 2H); 7.90 (d, J=5.7, 1H); 7.74-7.66 (m, 1H); 7.60-7.50 (m, 3H); 7.09-6.90 (m, 2H); 3.50 (br. s, 1H); 3.39 (br. s, 1H); 2.10 (d, J=9.0, 1H); 2.02-1.89 (m, 2H); 1.66 (d, J=9.0, 1H); 1.30-1.19 (m, 2H). MS (m/z): 401.38([M+H]⁺).

### Example 4

### Preparation of 4-(2,4-Dichlorophenyl)-4,5-diazatricyclo[5.2.1.0^{2,6}]deca-2,5-dien-3-yl-phenylmethanone

The title compound was synthesized by a procedure similar to that described for Example 1. Intermediate 67 (265 mg, 0.72 mmol), bromobenzene (92 μl, 0.87 mmol), magnesium turnings (25 mg, 1.01 mmol) and dry diethyl ether (10 ml) furnished the title compound as an off-white solid (130 mg 47%). M.P.: 128-130° C. ¹H-NMR (δ ppm, CDCl₃, 300 MHz): 7.91 (d, J=7.8, 2H); 7.66-7.58 (m, 1H); 7.55-7.47 (m, 3H); 7.44-7.33 (m, 2H); 3.51 (br. s, 1H); 3.10 (br. s, 1H); 2.12 (d, J=6.8, 1H); 2.04-1.84 (m, 2H); 1.72 (d, J=9.3, 1H); 1.48-1.22 (m, 2H). IR (cm⁻¹, KBr): 3422 (m), 3063 (w), 2989 (w), 2967 (m), 2939 (m), 2969 (m), 1656 (m), 1597 (s), 1576 (w), 1498 (m), 1473 (m), 1445 (m), 1413 (w), 1396 (m), 1361 (m), 1327 (m), 1284 (m), 1208 (m), 1175 (m), 1112 (m), 1063 (m), 1037 (m), 909 (m), 883 (m), 822 (m), 810 (m). MS (m/z): 383.42 ([M+H]⁺).

### Example 5

### Preparation of 1-[5-(2,4-diflurophenyl)-4,5-diazatricyclo[5.2.1.0^{2,6}]deca-2(6),3-dien-3-yl]-3,3,-dimethyl-1-butanol

The title compound was synthesized by a procedure similar to that described for Example 1. Intermediate 8 (250 mg, 0.91 mmol), Mg turnings (30 mg, 1.27 mmol), dry THF (5 ml), 1-bromo-2,2-dimethylpropane (574 μl, 4.56 mmol) furnished the title compound as a yellow oil (55 mg, 17%). ¹H-NMR (δ ppm, CDCl₃, 300 MHz): 7.64 (q, J=8.7, 1H); 6.96 (t, J=8.7, 2H); 4.98-4.90 (m, 1H); 3.43 (br. s, 2H); 2.03 (d, J=7.2, 1H); 1.96-1.50 (m, 6H); 1.28-1.17 (m, 2H), 1.02 (s, 9H). MS (m/z): 347.93 ([M+H]⁺).

### Example 6

### Preparation of 1-{5-(2,4-diflurophenyl)-4,5-diazatricyclo[5.2.1.0^{2,6}]deca-2(6),3-dien-3-yl]}-3,3,-dimethyl-1-butanone

To a solution of oxalyl chloride (56 μl, 0.65 mmol) in CH₂Cl₂ (2 ml) at −50° C. was added a solution of DMSO (46 μl, 0.65 mmol) in CH₂Cl₂ (2 ml). The resulting reaction mixture further stirred at that temperature for another 15 min and a solution of Example 5 (200 mg, 0.57 mmol) in CH₂Cl₂ (2 ml) was added drop-wise. Stirring was continued for an additional 15 min and Et₃N (239 μl, 1.73 mmol) was added. The mixture was allowed to warm to RT overnight. After addition of water, the mixture was extracted with dichloromethane and the organic extract was washed with brine and dried over Na₂SO₄. Purification by chromatography furnished the title compound (107 mg, 54%) as a white solid. M.P.: 112-115° C. ¹H-NMR (δ ppm, CDCl₃, 300 MHz): 7.75-7.64 (m, 1H); 7.06-6.99 (m, 2H); 3.71 (br. s, 1H); 3.45 (br. s, 1H); 2.91 (d, J=5.4, 2H); 2.07 (d, J=7.5, 1H); 1.99-1.90 (m, 2H); 1.67 (d, J=8.1, 1H); 1.20 (d, J=10.2, 2H); 1.08 (s, 9H). MS (m/z): 345.34 ([M+H]⁺).

### Example 7

### Preparation of N2-[5-(2,4-diflurophenyl)-4,5-diazatricyclo[5.2.1.0^{2,6}]deca-2(6),3-dien-3-ylmethyl]-2-methyhyl-2-propanamine

The Intermediate 8 (200 mg, 0.72 mmol) and tert-butylamine (230 μl, 2.18 mmol) were mixed in 1,2-dichloroethane (2 ml), stirred at RT for 20 min and acetic acid (161 μl, 2.29 mmol) was added. To the mixture was added sodium triacetoxyborhydride (231 mg, 1.09 mmol) and the reaction mixture was then stirred at RT overnight. After addition of water the mixture was extracted with chloroform and organic layers were washed with brine, dried over Na₂SO₄ and the solvent evaporated. The crude product was then treated with ether saturated with hydrochloric acid, salt formed was hygroscopic so which was dissolved in water, basified with NaOH pellets and extracted with diethyl ether, organic layers were separated and dried over Na₂SO₄ and evaporated to furnish the title compound as a pale yellow solid (125 mg, 51%). M.P.: 39-41° C. ¹H-NMR (δ ppm, CDCl₃, 300 MHz): 7.66 (q, J=8.4, 1H); 6.95 (t, J=8.4, 2H); 3.78 (s, 2H); 3.41 (br. s, 1H); 3.38 (br. s, 1H); 2.04 (d, J=9.0, 1H); 1.90 (d, J=7.8, 2H); 1.63 (d, J=8.4, 1H); 1.19 (s, 9H); 1.24-1.20 (m, 2H). MS (m/z): 332.21 ([M+H]⁺).

### Example 8

### Preparation of N2-[5-(2,4-diflurophenyl)-4,5-diazatricyclo[5.2.1.0^{2,6]}deca-2(6),3-dien-3-ylmethyl]-2-phenyl-2-propanamine hydrochloride

A procedure similar to that described for Example 7 was employed. The Intermediate 8 (200 mg, 0.72 mmol), α,α-dimethylbenzylamine (295 mg, 2.18 mmol), 1,2-dichloroethane (2 ml), acetic acid (161 μl, 2.29 mmol) and sodium triacetoxyborhydride (231 mg, 1.09 mmol). After the work-up, crude product was treated with ether saturated with hydrochloric acid to furnish the pure salt as white crystals (125 mg, 40%). M.P.: 159-161° C. ¹H-NMR (δ ppm, CDCl₃, 300 MHz): 10.03 (br. s, 2H); 7.74 (d, J=7.5, 3H); 7.42-7.25 (m, 3H); 7.02-6.87 (m, 2H); 3.87 (br. s, 2H); 3.44 (br. s, 1H); 3.31 (br. s, 1H); 2.00-1.85 (m, 3H); 1.86 (s, 6H); 1.55 (d, J=8.4, 1H); 1.15 (d, J=6.6, 2H).

### Example 9

### Preparation of N1-[5-(2,4-Difluorophenyl)-4,5-diazatricyclo[5.2.1.0^{2,6}]deca-2(6),3-dien-3-yl]-2,2-dimethylpropanamide

Pivaloyl chloride (116 μl, 0.94 mmol) was added drop-wise at 0° C. to a suspension of intermediate 9 (190 mg, 0.72 mmol) and Et₃N (151 μl, 1.09 mmol) in dichloromethane (2 ml), and the reaction was stirred at RT overnight. The mixture was diluted with dichloromethane and washed with brine and dried over Na₂SO₄. Purification by column chromatography furnished the title compound as an off-white solid (70 mg, 28%). M.P.: 136-138° C. ¹H-NMR (δ ppm, CDCl₃, 300 MHz): 7.89 (br. s, 1H); 7.55-7.49 (m, 1H), 7.05-6.90 (m, 2H); 3.80 (br. s, 1H); 3.39 (br. s, 1H); 2.05 (d, J=9.0, 1H); 2.01-1.90 (m, 2H); 1.65-1.58 (m, 1H); 1.48-1.22 (m, 2H); 1.31 (s, 9H). IR (cm⁻, KBr): 3253 (m), 3066 (w), 2959 (m), 2933 (m), 1662 (s), 1606 (m), 1567 (s), 1510 (s), 1399 (w), 1366 (m), 1324 (w), 1306 (w), 1266 (m), 1230 (w), 1203 (m), 1142 (m), 1086 (w), 950 (w), 956 (w), 934 (w), 847 (m). MS (m/z): 346.44 (M+H⁺).

### Example 10

### Preparation of 3-(2,4-Difluorophenyl)-5-sulfonamide-3,4-diazatricyclo[5.2.1.0^{2,6}]deca-2(6),4-diene

The title compound was synthesized by a procedure similar to that described for Example 9. Intermediate 9 (190 mg, 0.72 mmol), benzene sulfonyl chloride (121 μl, 0.94 mmol), triethylamine (151 μl, 1.09 mmol) and CH₂Cl₂ (2 ml) furnished the title compound as a pale yellow solid (70 mg, 24%). M.P.: 130° C. ¹H-NMR (δ ppm, CDCl₃, 300 MHz): 7.85-7.75 (m, 3H); 7.58-7.41 (m, 4H); 6.95-6.87 (m, 2H); 3.50 (br. s, 1H); 3.34 (br. s, 1H); 1.98 (d, J=9.0, 1H); 1.95-1.82 (m, 2H); 1.60-1.56 (m, 1H), 1.24-1.16 (m, 2H). MS (m/z): 402.37 ([M+H]⁺).

### Example 11

### Preparation of N5,N5-Dibenzyl-3-(2,4-difluorophenyl)-3,4-diazatricyclo[5.2.1.0^{2,6}]deca-2(6),4-dien-5-amine

To a solution of Intermediate 9 (105 mg, 0.40 mmol) in dry DMF (1 mL), anhydrous K₂CO₃ (66 mg, 0.48 mmol) was added followed by benzyl bromide (52 μl, 0.44 mmol) and the reaction was stirred at RT overnight. The reaction mixture was poured into water and extracted with ethyl acetate and the combined organic extracts were washed with water, brine and dried over Na₂SO₄. Purification of the crude product by column chromatography furnished the title compound as viscous oil (58 mg, 49%). ¹H-NMR (δ ppm, CDCl₃, 300 MHz): 7.65 (q, J=8.4, 1H); 7.38-7.21 (m, 10H); 6.90 (t, J=8.4, 2H); 4.59, 4.47 (AB, J=15.6, 4H); 3.39 (br. s, 1H); 3.14 (br. s, 1H); 1.97 (d, J=8.4, 1H); 1.89-1.80 (m, 1H); 1.75-1.65 (m, 1H); 1.5 (d, J=8.4, 1H); 1.31-0.96 (m, 2H). MS (m/z): 442.39 ([M+H]⁺).

### Example 12

### Preparation of (1S,8S)-N1-[5-(2,4-difluorophenyl)-9,9-dimethyl-4,5-diazatricyclo (6,1,1,0^{2,6})-deac-2(6), 3-dien-3-yl]-2,2-dimethyl propanamide

The title compound was synthesized by a procedure similar to that described for Example 9. Intermediate 108 (120 mg, 0.40 mmol), Pivaloyl chloride (66 μl, 0.53 mmol), Et₃N (88 μl, 0.62 mmol) and dichloromethane (2 ml) furnished the title compound as a pale yellow solid (80 mg, 51%). M.P.: 54-56° C. ¹H-NMR (δ ppm, CDCl₃, 300 MHz): 7.91 (br. s, 1H); 7.46-7.34 (m, 1H), 7.02-6.89 (m, 2H); 3.00-2.80 (m, 2H); 2.70-2.58 (m, 2H); 2.30 (br. s, 1H); 1.45 (d, J=9.0, 1H); 1.32 (s, 9H); 1.22 (s, 3H); 0.81 (s, 9H). MS (m/z): 374.42 (M+H⁺).

### Example 13

### Preparation of 5-benzyloxymethyl-3-(2,4-diflurophenyl)-3,4-diazatricylo[5.2.1.0^{2,6}.]deca-2(6),4diene

Sodium hydride (64 mg, 1.64 mmol) was added at 0° C. to a solution of intermediate 7 (350 mg, 1.26 mmol) in dry DMF (4 mL) and the mixture was stirred for 15 min. Benzyl bromide (165 μl, 1.39 mmol) was added and stirring was continued at the same temperature for an additional 1 h. The reaction mixture was diluted with water and extracted with ethyl acetate and the combined organic extracts were washed with brine and dried over Na₂SO₄. Purification of the crude product by preparative HPLC furnished the title compound as a pale yellow solid (54 mg, 11%). ¹H-NMR (δ ppm, CDCl₃, 300 MHz): 7.69-7.63 (m, 1H); 7.38-7.25 (m, 5H); 6.96 (t, J=8.1, 2H); 4.68-4.53 (m, 4H); 3.44 (br. s, 2H); 2.06-2.03 (m, 1H); 1.92 (d, J=7.2, 2H); 1.65 (d, J=8.7, 1H), 1.26-1.19 (m, 2H). MS (m/z): 367.30 ([M+H]⁺).

### Example 14

### Preparation of tert-Butyl-3-(2,4-difluorophenyl)-3,4-diazatricyclo[5.2.1.0^{2,6}]deca-2(6),4-diene-5-carboxylate

To a solution of Intermediate 3 (500 mg, 1.72 mol) and N1-[tert-butoxy(cyclohexylamino)methyl]-1-cyclohexane (924 mg, 3.44 mmol) in dry toluene (10 mL), catalytic amount of BF₃ etherate was added and reaction was refluxed overnight. The reaction mixture was filtered through celite and concentrated. The purification of the residue by column chromatography afforded the title compound as an off-white solid (250 mg, 42%). M.P.: 97-99° C. ¹H-NMR (δ ppm, CDCl₃, 300 MHz): 7.80-7.70 (m, 1H); 7.05-6.90 (m, 2H), 3.60 (br. s, 1H); 3.46 (br. s, 1H); 2.09 (d, J=8.1, 1H); 1.95 (d, J=8.4, 2H); 1.67 (d, J=8.4, 1H); 1.61 (s, 9H), 1.24 (d, J=8.4, 2H). IR (cm⁻¹, KBr): 3093 (w), 3072 (w), 2994 (m), 2971 (s), 2957 (s), 1735 (s), 1607 (m), 1510 (s), 1521 (s), 1477 (w), 1454 (m), 1424 (m), 1376 (s), 1366 (s), 1301 (w), 1232 (s), 1181 (s), 1088 (s), 965 (m), 858 (m). MS (m/z): 347.04 (M+H⁺).

### Example 15

### Preparation of 3-(2,4-Difluorophenyl)-5-(1-fluro-3,3-dimethylbutyl)-3,4-diazatricyclo[5.2.1.0^{2,6}]deca-2(6),4-diene

A solution of Example 5 (100 mg, 0.28 mmol) in dry dichloromethane (1 mL) was added to a solution of diethylaminosulfur trifluoride (52 μl, 0.43 mmol) in dry dichloromethane (1 mL) at −60° C. The reaction was warmed to RT and stirred for 1 h. The reaction mixture was quenched by addition of saturated solution of sodium bicarbonate and extracted with dichloromethane and the organic layers washed with brine and dried over Na₂SO₄. Chromatographic purification of the crude product furnished the title compound as a pale yellow oil (40 mg, 40%). ¹H-NMR (δ ppm, CDCl₃, 300 MHz): 7.65 (q, J=8.4, 1H); 6.97 (t, J=8.4, 2H); 5.77, 5.61 (dt, J=48, 9.6, 1H); 3.49 (br. s, 1H); 3.44 (br. s, 1H); 2.14-2.02 (m, 2H); 2.00-1.85 (m, 2H); 1.66 (d, J=9.0, 1H), 1.32-1.14 (m, 3H); 1.04 (s, 9H). MS (m/z): 349.36 ([M+H]⁺).

### Example 16

### Preparation of N′-Pivaloyl-3-(2,4-Diflurophenyl)-3,4-diazatricyclo[5.2.1.0^{2,6}]deca-2(6),4-diene-5-carbohydrazide

To a mixture of Intermediate 15 (250 mg, 0.82 mmol) and triethyl amine (250 μl, 1.08 mmol) in dichloromethane cooled externally in an ice-bath, pivaloyl chloride (110 μl, 0.90 mmol) was added and the mixture stirred at RT for 3 h. The reaction mixture was diluted with dichloromethane and washed with saturated solution of bicarbonate, water and brine and dried over Na₂SO₄. The solvent was removed to furnish the title compound as a yellow solid (313 mg, 98%). ¹H-NMR (δ ppm, CDCl₃, 300 MHz): 9.15 (br. s, 1H); 8.41 (br. s, 1H); 7.68 (q, 8.4, 1H); 7.00 (t, J=8.4, 2H); 3.70 (br. s, 1H); 3.47 (br. s, 1H); 2.08 (d, J=7.5, 1H); 1.96 (d, J=6.9, 2H); 1.69 (d, J=8.7, 1H); 1.29 (s, 9H); 1.27-1.20 (m, 2H).

### Example 17

### Preparation of N′-1-Hexanoyl-3-(2,4-diflurophenyl)-3,4-diazatricyclo[5.2.1.0^{2,6}]deca-2(6),4-diene-5-carbohydrazide

Hexanoic acid (82 μl, 0.65 mmol) in dry DMF (2.0 ml) was treated with BOP reagent (319 mg, 0.72 mmol) and Et₃N (109 μl, 0.78 mmol) at RT for about 30 minutes and then with Intermediate 15 (200 mg, 0.65 mmol) overnight. The mixture was poured into water, extracted into AcOEt, washed with brine, organic layers dried over sodium sulphate and the solvents evaporated. The residue was purified by column chromatography to furnish the title compound as an off-white solid (169 mg, 64%). ¹H-NMR (δ ppm, CDCl₃, 300 MHz): 9.13 (br. s, 1H); 8.71 (br. s, 1H); 7.66 (q, J=9.0, 1H); 6.98 (t, J=7.8, 2H); 3.68 (br. s, 1H); 3.47 (br. s, 1H); 2.35-2.24 (m, 2H), 2.08 (d, J=7.8, 1H); 1.95 (d, J=8.7, 2H); 1.68 (d, J=7.5, 1H); 1.34-1.24 (m, 8H); 090-0.85 (m, 3H).

### Example 18

### Preparation of N′-1-(Adamantanecarbonyl)-3-(2,4-diflurophenyl)-3,4-diazatricyclo[5.2.1.0^{2,6}]deca-2(6),4-diene-5-carbohydrazide

The title compound was synthesized by a procedure similar to that described for example 17. 1-Adamantane carboxylic acid (148 mg, 0.82 mmol), BOP reagent (434 mg, 0.98 mmol), Et₃N (135 μl, 0.98 mmol), dry DMF (2.5 ml) and intermediate 15 (250 mg, 0.82 mmol) furnished the title compound as an off-white solid (310 mg, 80%). ¹H-NMR (δ ppm, CDCl₃, 300 MHz): 9.15 (d, J=6.3, 1H); 8.28 (d, J=5.7, 1H); 7.66 (q, J=8.4, 1H); 6.98 (t, J=7.8, 2H); 3.70 (br. s, 1H); 3.46 (br. s, 1H); 2.08-2.04 (m, 4H), 2.00-1.92 (m, 6H); 1.78-1.60 (m, 8H); 1.29-1.20 (m, 3H).

### Example 19

### Preparation of N′-(Cyclohexanecarbonyl)-3-(2,4-diflurophenyl)-3,4-diazatricyclo[5.2.1.0^{2,6}]deca-2(6),4-diene-5-carbohydrazide

The title compound was synthesized by a procedure similar to that described for example 17. Cycohexane carboxylic acid (81 μl, 0.65 mmol), BOP reagent (319 mg, 0.72 mmol), Et₃N (109 μl, 0.78 mmol), dry DMF (2.0 ml) and intermediate 15 (200 mg, 0.65 mmol) furnished the title compound as an off-white solid (174 mg, 64%). ¹H-NMR (δ ppm, CDCl₃, 300 MHz): 9.04 (d, J=5.7, 1H); 8.10 (br. s, 1H); 7.66 (q, J=8.7, 1H); 6.96 (t, J=7.8, 2H); 3.70 (br. s, 1H); 3.46 (br. s, 1H); 2.32-2.20 (m, 1H), 2.08 (d, J=8.4, 1H); 2.00-1.90 (m, 3H);1.85-1.76 (m, 2H); 1.68 (d, J=8.4, 2H); 1.62-1.52 (m, 2H); 1.32-1.24 (m, 6H).

### Example 20

### Preparation of 2-(tert-Butyl)-5-[5-(2,4-difluorophenyl)-4,5-diazatricyclo[5.2.1.0^{2,6}]deca-2(6),3-dien-3yl]-1,3,4-oxadiazole

Intermediate 15 (250 mg, 0.82 mmol) was dissolved in dichloromethane (3 ml), cooled in an ice-bath, triethylamine (250 μl, 1.80 mmol) and pivaloyl chloride (110 μl, 0.90 mmol) were added. After stirring at RT for 3 h, thionyl chloride (71 μl, 0.98 mmol) was added and refluxed overnight. The reaction mixture was quenched by addition of water and extracted with dichloromethane, washed with saturated solution of NaHCO₃, brine and dried over Na₂SO₄. Removal of solvent and purification by SiO₂ column chromatography furnished the title compound (197 mg, 65%) as a yellow solid. M.P.: 108-110° C. ¹H-NMR (δ ppm, CDCl₃, 300 MHz): 7.82-7.70 (m, 1H); 7.08-6.98 (m, 2H); 3.78 (br. s, 1H); 3.51 (br. s, 1H); 2.16 (d, J=7.8, 1H); 2.00 (d, J=7.5, 2H); 1.74 (d, J=7.8, 1H); 1.49 (s, 9H); 1.34-1.22 (m, 2H). IR (cm⁻¹, KBr): 3051 (m), 2975 (s), 2974 (m), 1607 (s), 1558 (s), 1519 (s), 1497 (m), 1462 (m), 1362 (m), 1295 (w), 1271 (s), 1235 (w), 1147 (s), 1124 (s), 1078 (s), 961 (m). MS (m/z): 371.33 ([M+H]⁺).

### Example 21

### Preparation of 2-[5-(2,4-Difluorophenyl)-4,5-diazatricyclo[5.2.1.0^{2,6}]deca-2(6),3-dien-3yl]-5-Pentyl-1,3,4-oxadiazole

Example 17 (150 mg, 0.37 mmol) and POCl₃ (102 μl, 1.11 mmol) in dry toluene (3.0 ml) was refluxed overnight. Dilution of the reaction mixture with AcOEt and washed with an aq. Satd. Sodium bicarbonate solution. The organic layers dried over sodium sulphate, solvent removed and the residue purified by column chromatography to furnish the title compound as a yellow oil (136 mg 95%). ¹H-NMR (δ ppm, CDCl₃, 300 MHz): 7.71 (q, J=8.1, 1H); 7.00 (t, J=8.1, 2H); 3.79 (br. s, 1H); 3.51 (br. s, 1H); 2.91 (t, J=7.8, 2H); 2.15 (d, J=7.8, 1H); 2.01 (d, J=8.4, 2H); 1.89-1.80 (m, 2H); 1.74 (d, J=8.1, 1H); 1.42-1.22 (m, 6H); 0.90 (t, J=6.9, 3H). IR (cm⁻¹, KBr): 3435 (m), 3080 (w), 2956 (s), 2931 (s), 2872 (m), 1607 (m), 1569 (m), 1519 (s), 1497 (m), 1455 (m), 1366 (w), 1322 (m), 1271 (s), 1233 (w), 1218 (w), 1159 (m), 1145 (m), 1124 (m), 1080 (m), 1046 (w), 1012 (w), 962 (m), 850 (m). MS (m/z): 385.42 ([M+H]⁺).

### Example 22

### Preparation of 2-(1-Adamantly)-5-[5-(2,4-difluorophenyl)-4,5-diazatricyclo[5.2.1.0^{2,6}]deca-2(6),3-dien-3yl]-1,3,4-oxadiazole

The title compound was synthesized by a procedure similar to that described for Example 21. Example 18 (280 mg, 0.60 mmol), dry toluene (3.0 ml) and POCl₃ (165 μl, 1.80 mmol) furnished the title compound as an off-white foam (170 mg 63%). M.P.: 76-78° C. ¹H-NMR (δ ppm, CDCl₃, 300 MHz): 7.72 (q, J=8.4, 1H); 6.98 (t, J=8.1, 2H); 3.77 (br. s, 1H); 3.50 (br. s, 1H); 2.10-2.05 (m, 9H), 1.99 (d, J=7.2, 2H); 1.82-1.72 (m, 6H); 1.73 (d, J=8.4, 1H); 1.30-1.23 (m, 3H). IR (cm⁻¹, KBr): 3443 (m), 2908 (s), 2853 (m), 1608 (m), 1556 (m), 1520 (s), 1497 (m), 1454 (m), 1364 (w), 1271 (m), 1223 (m), 1219 (w), 1159 (w), 1145 (m), 1085 (m), 1061 (m), 1038 (m), 962 (m), 849 (m). MS (m/z): 449.36 ([M+H]⁺).

### Example 23

### Preparation of 2-(Cyclohexyl)-5-[5-(2,4-difluorophenyl)-4,5-diazatricyclo[5.2.1.0^{2,6}]deca-2(6),3-dien-3yl]-1,3,4-oxadiazole

The title compound was synthesized by a procedure similar to that described for Example 21. Example 19 (160 mg, 0.38 mmol), dry toluene (2.0 ml) and POCl₃ (104 μl, 1.14 mmol) furnished the title compound as a pale yellow oil (99 mg, 65%). ¹H-NMR (δ ppm, CDCl₃, 300 MHz): 7.71 (q, J=8.4, 1H); 6.99 (t, J=8.1, 2H); 3.78 (br. s, 1H); 3.50 (br. s, 1H); 3.03-2.93 (m, 1H); 2.20-1.91 (m, 4H); 1.89-1.81 (m, 2H); 1.73 (d, J=8.7, 2H); 1.43-1.24 (m, 8H). IR (cm⁻¹, KBr): 3444 (s), 2933 (s), 2857 (m), 1608 (m), 1561 (m), 1520 (s), 1497 (m), 1451 (m), 1362 (w), 1271 (m), 1233 (w), 1159 (w), 1145 (w), 1081 (m), 1045 (w), 1022 (w), 962 (m), 849 (w). MS (m/z): 397.40 ([M+H]⁺).

### Example 24

### Preparation of 2-(tert-Butyl)-5-[5-(2,4-difluorophenyl)-4,5-diazatricyclo[5.2.1.0^{2,6}]deca-2(6),3-dien-3yl]-1,3,4-thiadiazole

Example 16 (200 mg, 0.51 mmol) and phosphorous pentasulphide (171 mg, 0.77 mmol) were heated at 150-160° C. for 2 h. The reaction was then quenched with 10% NaOH and extracted with ethyl acetate and the combined organic layers were washed with brine and dried over Na₂SO₄. Removal of solvent and purification of the crude mixture by column chromatography furnished the title compound as a yellow solid (103 mg, 52%). M.P.: 109-111° C. ¹H-NMR (δ ppm, CDCl₃, 300 MHz): 7.71 (q, J=8.1, 1H); 7.01 (t, J=8.1, 2H); 3.86 (br. s, 1H); 3.50 (br. s, 1H); 2.14 (d, J=8.1, 1H); 2.04-1.94 (m, 2H); 1.72 (d, J=8.4, 1H); 1.52 (s, 9H); 1.38-1.22 (m, 2H). IR (cm⁻¹, KBr): 3037 (m), 2961 (s), 2868 (s), 1609 (m), 1523 (s), 1482 (m), 1438 (m), 1365 (w), 1344 (w), 1267 (s), 1218 (w), 1143 (m), 1092 (m), 962 (m). MS (m/z): 387.43 ([M+H]⁺).

### Example 25

### Preparation of 5-[5-(tert-Butyl)-1H-1,2,4-triazol-3-yl]-3-(2,4-difluorophenyl)-3,4-diazatricyclo[5.2.1.0^{2,6}]deca-2(6),4-diene

A solution of Intermediate 26 (50 mg, 0.12 mmol) and hydrazine hydrate (9.65 μl, 0.19 mmol) in dry methanol (2 ml) was stirred at RT for 2 h. The solvent was removed and diluted with water and extracted with dichloromethane and organic layers were washed with brine and dried over Na₂SO₄ and the solvent was evaporated. Purification of the residue by SiO₂ column chromatography furnished the title compound as a white solid (41 mg, 87%). M.P.: 268-269° C. ¹H-NMR (δ ppm, DMSO-d₆, 300 MHz): 13.65 (br. s, 1H); 7.81-7.71 (m, 1H); 7.44-7.65 (m, 1H); 7.40-7.22 (m, 1H); 3.59 (br. s, 1H); 3.49 (br. s, 1H); 2.08-1.80 (m, 3H); 2.05-1.89 (m, 1H); 1.35 (s, 9H); 1.20-1.12 (m, 2H). IR (cm⁻¹, KBr): 3443 (m), 3133 (w), 2969 (m), 2869 (m), 1606 (w), 1523 (s), 1488 (w), 1446 (w), 1370 (m), 1272 (m), 1143 (m), 1090 (m), 859 (m). MS (m/z): 370.40 ([M+H]⁺).

### Example 26

### Preparation of 5-(tert-Butyl)-3-[5-(2,4-difluorophenyl)-4,5-diazatricyclo[5.2.1.0^{2,6}]deca-2(6),3-dien-3yl]-1,2,4-oxadiazole

A solution of sodium methoxide (30 mg, 0.56 mmol) and hydroxylamine hydrochloride (39 mg, 0.56 mmol) in dry methanol (5 ml) was stirred at RT for 15 min. Intermediate 26 (200 mg, 0.51 mmol) was added and stirring was continued for 12 h. The solvent was evaporated and the residue was dissolved in dichloromethane and washed with water and brine, dried and the solvent was removed. Purification of the crude product by SiO₂ column chromatography furnished the title compound as a white solid (163 mg, 85%). M.P.: 119-121° C. ¹H-NMR (δ ppm, CDCl₃, 300 MHz): 7.79 (q, J=8.4, 1H); 6.99 (t, J=8.1, 2H); 3.73 (br. s, 1H); 3.49 (br. s, 1H); 2.15 (d, 8.4, 1H); 1.98 (d, J=8.4, 2H); 1.72 (d, J=7.8, 1H); 1.50 (s, 9H); 1.27 (d, J=7.8, 2H). IR (cm⁻¹, KBr): 3445 (w), 3061 (m), 3011 (m), 2933 (m), 2977 (s), 2947 (m), 2872 (m), 1604 (m), 1576 (m), 1562 (m), 1521 (s), 1488 (w), 1364 (s), 1272 (m), 1169 (m), 1146 (m), 1099 (m), 963 (m). MS (m/z): 371.34 ([M+H]⁺).

### Example 27

### Preparation of 5-(tert-Butyl)-3-[(1S,7R)-5-(2,4-difluorophenyl)-4,5-diazatricyclo[5.2.1.0^{2,6}]deca-2(6),3-dien-3yl]-1,2,4-oxadiazole

The title compound was synthesized by a procedure similar to that described for Example 26. Intermediate 27 (720 mg, 1.86 mmol), sodium methoxide (110 mg, 2.04 mmol), hydroxylamine hydrochloride (142 mg, 2.04 mmol) and dry methanol (10 ml) furnished the title compound as a white solid (579 mg, 84%). Enantiomeric Excess: 72.35%. M.P.: 94-96° C. ¹H-NMR (δ ppm, CDCl₃, 300 MHz): 7.82-7.70 (m, 1H); 7.02-6.92 (m, 2H); 3.71 (br. s, 1H); 3.49 (br. s, 1H); 2.14 (d, 8.7, 1H); 1.97 (d, J=7.8, 2H); 1.71 (d, J=8.7, 1H); 1.50 (s, 9H); 1.27 (d, J=7.2, 2H). IR (cm⁻¹, KBr): 3434 (m), 3071 (w), 2978 (s), 2949 (m), 2872 (m), 1606 (m), 1574 (m), 1559 (m), 1529 (s), 1491 (w), 1359 (m), 1272 (m), 1254 (m), 1236 (m), 1168 (m), 1144 (m), 1090 (m), 961 (m), 874 (m). MS (m/z): 371.20 ([M+H]⁺).

### Example 28

### Preparation of 5-(tert-Butyl)-3-[(1R,7S)-5-(2,4-difluorophenyl)-4,5-diazatricyclo[5.2.1.0^{2,6}]deca-2(6),3-dien-3yl]-1,2,4-oxadiazole

The title compound was synthesized by a procedure similar to that described for Example 26. Intermediate 28 (590 mg, 1.52 mmol), sodium methoxide (90 mg, 1.67 mmol) and hydroxylamine hydrochloride (116 mg, 1.67 mmol) and dry methanol (10 ml) furnished the title compound as a white solid (490 mg, 92%). Enantiomeric Excess: 85.30%. M.P.: 89-90° C. ¹H-NMR (δ ppm, CDCl₃, 300 MHz): 7.84-7.72 (m, 1H); 7.02-6.96 (m, 2H); 3.71 (br. s, 1H); 3.49 (br. s, 1H); 2.12 (d, 8.7, 1H); 1.98 (d, J=7.8, 2H); 1.71 (d, J=7.8, 1H); 1.50 (s, 9H); 1.27 (d, J=7.8, 2H). IR (cm⁻¹, KBr): 3435 (s), 3071 (w), 2979 (s), 2949 (m), 2872 (m), 1607 (m), 1574 (m), 1559 (m), 1529 (s), 1491 (w), 1359 (m), 1272 (m), 1168 (m), 1144 (m), 1090 (m), 961 (m), 874 (m). MS (m/z): 371.22 ([M+H]⁺).

### Example 29

### Preparation of 3-[5-(2,4-Difluorophenyl)-4,5-diazatricyclo[5.2.1.0^{2,6}]deca-2(6),3-dien-3yl]-5-phenyl-1,2,4-oxadiazole

The title compound was synthesized by a procedure similar to that described for Example 26. Intermediate 29 (180 mg, 0.44 mmol), sodium methoxide (26 mg, 0.48 mmol), hydroxylamine hydrochloride (34 mg, 0.48 mmol) and dry methanol (5 ml) furnished the title compound as a white solid (117 mg, 68%). M.P.: 106-107° C. ¹H-NMR (δ ppm, CDCl₃, 300 MHz): 8.30-8.24 (m, 2H); 7.88-7.77 (m, 1H); 7.64-7.50 (m, 3H); 7.05-6.96 (m, 2H); 3.80 (br. s, 1H); 3.53 (br. s, 1H); 2.19 (d, 9.0, 1H); 2.05-1.85 (m, 2H); 1.75 (d, J=9.0, 1H); 1.38-1.22 (m, 2H). IR (cm⁻, KBr): 3444 (m), 3065 (w), 2989 (w), 2927 (w), 2874 (w), 1608 (s), 1559 (s), 1524 (m), 1495 (s), 1449 (m), 1431 (w), 1359 (s), 1329 (w), 1269 (s), 1252 (m), 1146 (m), 1115 (m), 974 (m), 958 (m), 859(m). MS (m/z): 391.54 ([M+H]⁺).

### Example 30

### Preparation of 3-(2,4-diflurophenyl)-5[(E)-3,3-dimethyl-1-butenyl}-3,4-diazatricyclo[5.2.1.0^{2,6}]deca-2(6),4-diene

Conc. H₂SO₄ (1.0 mL) was added to a solution of Example 5 (250 mg, 0.72 mmol) in 1,4-dioxane (0.5 mL) and stirred at RT for 1 h. The reaction mixture was distributed between water and ethyl acetate, organic layers were washed with water, brine and dried over Na₂SO₄. Purification of crude product by column chromatographic afforded the title compound as white solid (65 mg, 27%). M.P.: 98-101° C. ¹H-NMR (δ ppm, CDCl₃, 300 MHz): 7.65 (q, J=8.1, 1H); 6.95 (t, J=8.1, 2H); 6.40, 6.31 (AB, J=16.5, 2H); 3.53 (br. s, 1H); 3.42 (br. s, 1H); 2.06 (d, J=9.0, 1H); 1.93 (d, J=7.5, 2H); 1.65 (d, J=8.4, 1H), 1.24 (d, J=5.4, 2H); 1.12 (s, 9H). MS (m/z): 329.35 ([M+H]⁺).

### Example 31

### Preparation of 3-(2,4-Difluorophenyl)-5-(3,3-dimethylbutyl)-3,4-diazatricyclo[5.2.1.0^{2,6}]deca-2(6),4-diene

A solution of Example 5 (250 mg, 0.72 mmol) was dissolved in dichloromethane (5 ml), triethylsilane (345 μl, 2.16 mmol) was added and after stirring at RT for 15 min, boron trifluoride diethyl etherate (290 μl, 2.30 mmol) was added and stirred at RT overnight. The mixture was diluted with dichloromethane and washed successively with water, brine and dried. The solvent was removed under reduced pressure and the residue (103 mg) was dissolved in ethyl acetate (5 ml) and catalytic amount of 10% palladium carbon (10 mg) was added and hydrogenated at RT for 4 h. The mixture was filtered through celite and the solvent was evaporated. Purification of the crude product by SiO₂ column chromatography furnished the title compound as an off-white solid (27 mg, 11%). M.P.: 54-57° C. ¹H-NMR (δ ppm, CDCl₃, 300 MHz): 7.64 (br. q, J=8.4, 1H); 6.94 (t, J=8.4, 2H); 3.41 (br. s, 1H); 3.35 (br. s, 1H); 2.68-2.55 (m, 2H); 2.08-1.88 (m, 3H); 1.68-1.49 (m, 3H); 1.33-1.13 (m, 2H); 0.96 (s, 9H). IR (cm⁻¹, KBr): 3446 (w), 3044 (m), 2953 (s), 2870 (m), 1610 (m), 1532 (s), 1498 (m), 1463 (m), 1385 (m), 1364 (m), 1266 (m), 1237 (w), 1147 (m), 1109 (m), 1077 (m), 964 (m), 867 (m), 815 (m). MS (m/z): 331.43([M+H]⁺).

### Example 32

### Preparation of 3-(2,4-Difluorophenyl)-5-(3,3-dimethyl-1-butynyl)-3,4-diazatricyclo[5.2.1.0^{2,6}]deca-2(6),4-diene

To a solution of Intermediate 12 (200 mg, 0.53 mmol) and 3,3-dimethyl-4-butyne (98 μl, 0.80 mmol) in triethylamine (1 ml) were added catalytic amount of bis-[triphenylphosphine]palladium dichloride and copper(I)iodide and the mixture was stirred at RT for 3 h, solvent was removed and the residue subjected to SiO₂ column chromatography to furnish the title compound as yellow liquid (175 mg, 90%). ¹H-NMR (δ ppm, CDCl₃, 300 MHz): 7.69 (q, J=8.4, 1H); 6.95 (t, J=8.4, 2H); 3.43 (br. s, 2H); 2.03 (d, 6.3, 1H); 1.98-1.88 (m, 2H); 1.65 (d, J=3.6, 1H); 1.33 (s, 9H); 1.32-1.21 (m, 2H). IR (cm⁻¹, KBr): 3445 (m), 2967 (m), 1608 (w), 1521 (s), 1456 (m), 1361 (w), 1271 (m), 1141 (m), 1084 (m), 966 (m), 850 (m). MS (m/z): 327.38 ([M+H]⁺).

### Example 33

### Preparation of (1S,7R)-3-(2,4-Difluorophenyl)-5-(3,3-dimethyl-1-butynyl)-3,4-diazatricyclo[5.2.1.0^{2,6}]deca-2(6),4-diene

The title compound was synthesized by a procedure similar to that described for example 32. Intermediate 13 (200 mg, 0.53 mmol) and 3,3-dimethyl-4-butyne (98 μl, 0.80 mmol), Et₃N (2 ml), bis-[triphenylphosphine]palladium dichloride (25 mg) and copper(I)iodide (10 mg) furnished the title compound as light yellow solid (160 mg, 91%). Enantiomeric Excess: 89.95%. ¹H-NMR (δ ppm, CDCl₃, 300 MHz): 7.66 (q, J=8.4, 1H); 6.93 (t, J=8.1, 2H); 3.42 (br. s, 2H); 2.03 (d, J=8.4, 1H); 1.97-1.86 (m, 2H); 1.63 (d, J=8.7, 1H); 1.32 (s, 9H); 1.32-1.20 (m, 2H). IR (cm⁻¹, KBr): 3412 (w), 2971 (m), 2928 (m), 2872 (m), 1608 (w), 1523 (s), 1457 (m), 1363 (w), 1269 (m), 1215 (s), 1114 (m), 1086 (w), 966 (m), 756 (s). MS (m/z): 327.26 ([M+H]⁺).

### Example 34

### Preparation of (1R,7S)-3-(2,4-Difluorophenyl)-5-(3,3-dimethyl-1-butynyl)-3,4-diazatricyclo[5.2.1.0^{2,6}]deca-2(6),4-diene

The title compound was synthesized by a procedure similar to that described for example 32. Intermediate 14 (200 mg, 0.80 mmol), 3,3-dimethyl-4-butyne (147 μl, 1.21 mmol), Et₃N (3 ml), bis-[triphenylphosphine]palladium dichloride (37 mg) and copper(I)iodide (15 mg) yielded the product as a yellow solid (251 mg, 95%). Enantiomeric Excess: 90.62%. ¹H-NMR (δ ppm, CDCl₃, 300 MHz): 7.66 (q, J=8.4, 1H); 6.93 (t, J=8.7, 2H); 3.42 (br. s, 2H); 2.03 (d, J=8.7, 1H); 1.99-1.86 (m, 2H); 1.65 (d, J=8.7, 1H); 1.33 (s, 9H); 1.33-1.20 (m, 2H). IR (cm⁻¹, KBr): 2969 (s), 2929 (m), 2871 (m), 1607 (m), 1522 (s), 1456 (m), 1362 (w), 1268 (s), 1249 (w), 1143 (m), 1121 (w), 1083 (m), 965 (m), 848 (m). MS (m/z): 327.32 ([M+H]⁺).

### Example 35

### Preparation of 1-[5-(2,4-Difluorophenyl)-4,5-diazatricyclo[5.2.1.0^{2,6}]deca-2(6),3-dien-3-yl]-2-phenylacetylene

The title compound was synthesized by a procedure similar to that described for example 32. Intermediate 13 (100 mg, 0.26 mmol) and phenyl acetylene (35 μl, 0.32 mmol) furnished the title compound as a yellow solid (93 mg, 94%). M.P.: 101-104° C. ¹H-NMR (δ ppm, CDCl₃, 300 MHz): 7.73 (q, J=8.4, 1H); 7.58 (br. d., J=6.0, 2H); 7.34 (br. s, 3H); 6.99 (t, J=8.1, 2H); 3.53 (br. s, 1H); 3.49 (br. s, 1H); 2.09 (d, 7.8, 1H); 2.01-1.92 (m, 2H); 1.68 (d, J=8.7, 1H); 1.39-1.22 (m, 2H). IR (cm⁻¹, KBr): 3062 (w), 2975 (m), 2871 (m), 1607 (m), 1520 (s), 1442 (m), 1362 (w), 1269 (s), 1160 (w), 1144 (m), 1113 (m), 1085 (m), 965 (m), 848 (m), 756 (m). MS (m/z): 347.34 ([M+H]⁺).

### Example 36

### Preparation of N5-(3,3-Dimethyl-2-oxobutyl)-3-(2,4-difluorophenyl)-3,4-diazatricyclo[5.2.1.0^{2,6}]deca-2(6),4-dien-5-carboxamide

Intermediate 3 (175 mg, 0.60 mmol) was dissolved in dry dichloromethane (5 ml), cooled to 0° C., oxalyl chloride (63 μl, 0.72 mmol) was added dropwise followed by a catalytic amount of dry DMF and stirred at RT for 3 h. The solvent was removed completely under reduced pressure. To a stirred solution of solution of Intermediate 113 (91 mg, 0.60 mmol) in dry dichloromethane (5 ml) at 0° C., Et₃N (200 μl, 1.44 mmol) and catalytic amount of DMAP (5 mg) were added and stirred for 15 min, then a solution of above acid chloride in dry dichloromethane (5 ml) was added dropwise and the reaction was allowed to warm to room temperature and stirred overnight. The solution was distributed between water and dichloromethane and the layers were separated, organic layer washed with saturated solution of sodium bicarbonate and then with brine and dried over Na₂SO₄. After removal of the solvent, the residue was purified on SiO₂ column chromatography to furnish the title compound as white foam (180 mg 77%). M.P.: 42-43° C. ¹H-NMR (δ ppm, CDCl₃, 300 MHz): 7.77-7.67 (m, 1H); 7.53 (br. s, 1H); 7.04-6.96 (m, 2H); 4.44 (br. d., J=3.6, 2H); 3.72 (br. s, 1H); 3.47 (br. s, 1H); 2.09 (d, J=8.7, 1H); 1.96 (d, J=7.5, 2H); 1.71-1.63 (m, 1H); 1.25 (s, 9H); 1.28-1.23 (m, 2H). IR (cm⁻¹, KBr): 3402 (m), 2968 (s), 2872 (m), 1718 (m), 1667 (s), 1614 (m), 1523 (s), 1546 (s), 1493 (s), 1455 (m), 1365 (m), 1326 (w), 1270 (s), 1232 (m), 1145 (m), 1122 (m), 1093 (m), 963 (m), 850 (m). MS (m/z): 388.46 ([M+H]⁺).

### Example 37

### Preparation of N5-(3,3-Dimethyl-2-oxobutyl)-3-(1R,7S)-(2,4-Difluorophenyl)-3,4-diazatricyclo[5.2.1.0^{2,6}]deca-2(6),4-dien-5-carboxamide

The title compound was synthesized by a procedure similar to that described for example 36. Intermediate 5 (200 mg, 0.68 mmol), dry dichloromethane (20 ml), oxalyl chloride (71 μl, 0.82 mmol), Intermediate 113 (102 mg, 0.60 mmol), E₃N (225 μl, 1.63 mmol) and catalytic amount of DMAP (5 mg) furnished the title compound as white foam (217 mg, 82%). M.P.: 45-46° C. ¹H-NMR (δ ppm, CDCl₃, 300 MHz): 7.77-7.67 (m, 1H); 7.53 (br. s, 1H); 7.04-6.96 (m, 2H); 4.45 (br. d., J=3.0, 2H); 3.72 (br. s, 1H); 3.47 (br. s, 1H); 2.09 (d, J=8.7, 1H); 1.96 (d, J=7.5, 2H); 1.71-1.63 (m, 1H); 1.25 (s, 9H); 1.28-1.23 (m, 2H). MS (m/z): 388.46 ([M+H]⁺).

### Example 38

### Preparation of N5-(3,3-Dimethyl-2-oxobutyl)-3-(1S,7R)-(2,4-Difluorophenyl)-3,4-diazatricyclo[5.2.1.0^{2,6}]deca-2(6),4-dien-5-carboxamide

The title compound was synthesized by a procedure similar to that described for example 36. Intermediate 4 (230 mg, 0.79 mmol), dry dichloromethane (20 ml), oxalyl chloride (83 μl, 0.95 mmol), Intermediate 113 (120 mg, 0.79 mmol), E₃N (270 μl, 1.90 mmol) and catalytic amount of DMAP (5 mg) furnished the title compound as a white foam (150 mg 49%). M.P.: 46-48° C. ¹H-NMR (δ ppm, CDCl₃, 300 MHz): 7.77-7.67 (m, 1H); 7.53 (br. s, 1H); 7.04-6.96 (m, 2H); 4.45 (br. d., J=3.0, m, 2H); 3.72 (br. s, 1H); 3.47 (br. s, 1H); 2.09 (d, J=8.7, 1H); 1.96 (d, J=7.5, 2H); 1.71-1.63 (m, 1H); 1.25 (s, 9H); 1.28-1.23 (m, 2H). MS (m/z): 388.46 ([M+H]⁺).

### Example 39

### Preparation of N5-(3,3-Dimethyl-2-oxobutyl)-3-(4-chlorophenyl)-3,4-diazatricyclo[5.2.1.0^{2,6}]deca-2(6),4-dien-5-carboxamide

The title compound was synthesized by a procedure similar to that described for example 36. Intermediate 34 (200 mg, 0.69 mmol), dry dichloromethane (20 ml), oxalyl chloride (72 μl, 0.82 mmol), intermediate 113 (105 mg, 0.69 mmol), E₃N (230 μl, 1.65 mmol) and catalytic amount of DMAP (5 mg) furnished the title compound as yellow solid (169 mg 64%). ¹H-NMR (δ ppm, CDCl₃, 300 MHz): 7.67 (d, J=8.4, 2H); 7.59 (br. s, 1H); 7.43(d, J=8.7, 2H); 4.46 (br. s., 2H); 3.73 (br. s, 1H); 3.69 (br. s, 1H); 2.12 (d, J=7.8, 1H); 1.99 (d, J=7.5, 2H); 1.72 (d, J=8.7, 1H); 1.24 (s, 9H); 1.30-1.20 (m, 2H). MS (m/z): 386.52 ([M+H]⁺).

### Example 40

### Preparation of N5-(3,3-Dimethyl-2-oxobutyl)-3-(4-chloro-2-fluorophenyl)-3,4-diazatricyclo[5.2.1.0^{2,6}]deca-2(6),4-dien-5-carboxamide

The title compound was synthesized by a procedure similar to that described for example 36. Intermediate 38 (150 mg, 0.49 mmol), dry dichloromethane (15 ml), oxalyl chloride (51 μl, 0.58 mmol), intermediate 113 (74 mg, 0.49 mmol), Et₃N (165 μl, 1.17 mmol) and catalytic amount of DMAP (5 mg) furnished the title compound as a white foam (160 mg 81%). ¹H-NMR (δ ppm, CDCl₃, 300 MHz): 7.71 (t, J=9.0, 1H); 7.54 (br. s, 1H); 7.30-7.23 (m, 2H); 4.47-4.43 (m, 2H); 3.72 (br. s, 1H); 3.49 (br. s, 1H); 2.08 (d, J=6.0, 1H); 1.96 (d, J=6.0, 2H); 1.68 (d, J=9.0, 1H); 1.23 (s, 9H); 1.28-1.20 (m, 2H).

### Example 41

### Preparation of N5-(3,3-Dimethyl-2-oxobutyl)-3-(2,4,6-trifluorophenyl)-3,4-diazatricyclo[5.2.1.0^{2,6}]deca-2(6),4-dien-5-carboxamide

The title compound was synthesized by a procedure similar to that described for example 36. Intermediate 42 (150 mg, 0.48 mmol), dry dichloromethane (15 ml), oxalyl chloride (51 μl, 0.58 mmol), catalytic amount of dry DMF, Intermediate 113 (73 mg, 0.48 mmol), Et₃N (165 μl, 1.17 mmol) and catalytic amount of DMAP (5 mg) furnished the title compound as a white foam (170 mg 86%). ¹H-NMR (δ ppm, CDCl₃, 300 MHz): 7.48 (br. s, 1H); 6.91-6.82 (m, 2H); 4.43 (t, J=4.5, 2H); 3.74 (br. s, 1H); 3.29 (br. s, 1H); 2.19-2.08 (m, 1H); 1.96-1.85 (m, 2H); 1.68 (d, J=9.0, 1H); 1.22 (s, 9H); 1.28-1.20 (m, 2H).

### Example 42

### Preparation of Ethyl 4-[5-(2,4-difluorophenyl)-4,5-diazatricyclo[5.2.1.0^{2,6}]deca-2(6),3-dien-3-yl-carboxamido]-2,2-dimethyl-3-oxobutanoate

The title compound was synthesized by a procedure similar to that described for example 36. Intermediate 3 (300 mg, 1.03 mmol), dry dichloromethane (20 ml), oxalyl chloride (107 μl, 1.24 mmol), catalytic amount of dry DMF, Intermediate 117 (216 mg, 1.03 mmol), Et₃N (343 μl, 2.48 mmol) and catalytic amount of DMAP (10 mg) to furnish the title compound as yellow oil (300 mg 65%). ¹H-NMR (δ ppm, CDCl₃, 300 MHz): 7.76-7.64 (m, 1H); 7.44 (br. s, 1H); 7.08-6.92 (m, 2H); 4.45 (d, J=4.8, 2H); 4.21 (q, J=6.9, 2H); 3.72 (br. s, 1H); 3.46 (br. s, 1H); 2.08 (d, J=7.5, 1H); 1.96 (d, J=8.1, 2H); 1.68 (d, J=8.7, 1H); 1.46 (s, 6H); 1.27 (t, J=7.2, 3H); 1.32-1.20 (m, 2H). MS (m/z): 446.58 ([M+H]⁺).

### Example 43

### Preparation of Ethyl 4-[(1S,7R)-5-(2,4-difluorophenyl)-4,5-diazatricyclo[5.2.1.0^{2,6}]deca-2(6),3-dien-3-yl-carboxamido]-2,2-dimethyl-3-oxobutanoate

The title compound was synthesized by a procedure similar to that described for example 36. Intermediate 5 (400 mg, 1.37 mmol), dry dichloromethane (20 ml), oxalyl chloride (143 μl, 1.65 mmol), catalytic amount of dry DMF, intermediate 117 (346 mg, 1.65 mmol), Et₃N (458 μl, 3.31 mmol) and catalytic amount of DMAP (10 mg) afforded the title compound as a yellow oil (578 mg 94%). ¹H-NMR (δ ppm, CDCl₃, 300 MHz): 7.73-7.63 (m, 1H); 7.42 (br. s, 1H); 7.02-6.94 (m, 2H); 4.43 (d, J=6.0, 2H); 4.19 (q, J=6.9, 2H); 3.71 (br. s, 1H); 3.45 (br. s, 1H); 2.07 (d, J=9.0, 1H); 1.95 (d, J=9.0, 2H); 1.67 (d, J=9.0, 1H); 1.46 (s, 6H); 1.30-1.22 (m, 5H). MS (m/z): 446.72 ([M+H]⁺).

### Example 44

### Preparation of Ethyl 4-[(1R,7S) 5-(2,4-difluorophenyl)-4,5-diazatricyclo[5.2.1.0^{2,6}]deca-2(6),3-dien-3-yl-carboxamido]-2,2-dimethyl-3-oxobutanoate

The title compound was synthesized by a procedure similar to that described for example 36. Intermediate 4 (400 mg, 1.37 mmol), dry dichloromethane (20 ml), oxalyl chloride (143 μl, 1.65 mmol), catalytic amount of dry DMF, intermediate 117 (346 mg, 1.65 mmol), Et₃N (458 μl, 3.31 mmol) and catalytic amount of DMAP (10 mg) gave the title compound as an yellow oil (562 mg 92%). ¹H-NMR (δ ppm, CDCl₃, 300 MHz): 7.72-7.62 (m, 1H); 7.42 (br. s, 1H); 7.02-6.94 (m, 2H); 4.43 (d, J=4.2, 2H); 4.22 (q, J=6.9, 2H); 3.71 (br. s, 1H); 3.45 (br. s, 1H); 2.07 (d, J=8.1, 1H); 1.95 (d, J=7.8, 2H); 1.68 (d, J=8.1, 1H); 1.46 (s, 6H); 1.30-1.22 (m, 5H). MS (m/z): 446.65 ([M+H]⁺).

### Example 45

### Preparation of N5-(3,3-Dimethyl-2-oxobutyl)-3-(4-chloro-2-fluorophenyl)-3,4-diazatricyclo[5.2.1.0^{2,6}]deca-2(6),4-dien-5-carboxamide

The title compound was synthesized by a procedure similar to that described for example 36. Intermediate 38 (150 mg, 0.49 mmol), dry dichloromethane (15 ml), oxalyl chloride (51 μl, 0.58 mmol), intermediate 113 (74 mg, 0.49 mmol), Et₃N (165 μl, 1.17 mmol) and catalytic amount of DMAP (5 mg) furnished the title compound as a white foam (160 mg 81%). ¹H-NMR (δ ppm, CDCl₃, 300 MHz): 7.71 (t, J=9.0, 1H); 7.54 (br. s, 1H); 7.30-7.23 (m, 2H); 4.47-4.43 (m, 2H); 3.72 (br. s, 1H); 3.49 (br. s, 1H); 2.08 (d, J=6.0, 1H); 1.96 (d, J=6.0, 2H); 1.68 (d, J=9.0, 1H); 1.23 (s, 9H); 1.28-1.20 (m, 2H).

### Example 46a

### Preparation of (1S,7R)- or (1R,7S)-N5-(3,3-Dimethyl-2-oxobutyl)-3-(4-chloro-2-fluorophenyl)-3,4-diazatricyclo[5.2.1.0^{2,6}]deca-2(6),4-dien-5-carboxamide

The title compound was synthesized by a procedure similar to that described for example 17. Intermediate 39 (150 mg, 0.49 mmol), Et₃N (170 μl, 1.17 mmol), BOP reagent (227 mg, 1.05 mmol), dry DMF (1.0 ml) and Intermediate 113 (81 mg, 0.53 mmol) furnished the title compound as a white foam (160 mg 81%). ¹H-NMR (δ ppm, CDCl₃, 300 MHz): 7.69 (t, J=8.7, 1H); 7.50 (br. s, 1H); 7.32-7.20 (m, 2H); 4.48-4.42 (m, 2H); 3.71 (br. s, 1H); 3.48 (br. s, 1H); 2.07 (d, J=6.0, 1H); 1.95 (d, J=6.0, 2H); 1.67 (d, J=8.4, 1H); 1.23 (s, 11H).

### Example 46b

### Preparation of (1R,7S) or (1S,7R)-N5-(3,3-Dimethyl-2-oxobutyl)-3-(4-chloro-2-fluorophenyl)-3,4-diazatricyclo[5.2.1.0^{2,6}]deca-2(6),4-dien-5-carboxamide

The title compound was synthesized by a procedure similar to that described for example 17. Intermediate 40 (150 mg, 0.49 mmol), Et₃N (170 μl, 1.17 mmol), BOP reagent (227 mg, 1.05 mmol), dry DMF (1.0 ml) and intermediate 113 (81 mg, 0.53 mmol) furnished the title compound as white foam (160 mg 81%). ¹H-NMR (δ ppm, CDCl₃, 300 MHz): 7.68 (t, J=9.0, 1H); 7.50 (br. s, 1H); 7.30-7.20 (m, 2H); 4.46-4.43 (m, 2H); 3.71 (br. s, 1H); 3.48 (br. s, 1H); 2.07 (d, J=6.0, 1H); 1.96 (d, J=6.0, 2H); 1.67 (d, J=7.8, 1H); 1.23 (s, 11H).

### Example 47

### Preparation of N5-(3,3-Dimethyl-2-oxobutyl)-3-(4-methoxyphenyl)-3,4-diazatricyclo[5.2.1.0^{2,6}]deca-2(6),4-dien-5-carboxamide

The title compound was synthesized by a procedure similar to that described for example 17. Intermediate 44 (250 mg, 0.82 mmol), Et₃N (290 μl, 2.05 mmol), BOP reagent (382 mg, 0.86 mmol), dry DMF (2.5 ml) and intermediate 113 (185 mg, 1.23 mmol) furnished the title compound as an off-white solid (210 mg 65%). ¹H-NMR (δ ppm, CDCl₃, 300 MHz): 7.59 (d, J=8.7, 2H); 7.56 (br. s, 1H); 6.95 (d, J=8.7, 2H); 4.47-4.42 (m, 2H); 3.71 (br. s, 1H); 3.64 (br. s, 1H); 2.10 (d, J=8.7, 1H); 1.96 (d, J=7.8, 2H); 1.69 (d, J=9.0, 1H); 1.23 (s, 11H).

### Example 48

### Preparation of N5-(3,3-Dimethyl-2-oxobutyl)-3-(4-bromophenyl)-3,4-diazatricyclo[5.2.1.0^{2,6}]deca-2(6),4-dien-5-carboxamide

The title compound was synthesized by a procedure similar to that described for example 17. Intermediate 46 (250 mg, 0.75 mmol), Et₃N (265 μl, 1.88 mmol), BOP reagent (350 mg, 0.79 mmol), dry DMF (2.5 ml) and intermediate 113 (170 mg, 1.12 mmol) furnished the title compound as a yellow solid (290 mg 94%). ¹H-NMR (δ ppm, CDCl₃, 300 MHz): 7.62-7.52 (m, 4H); 4.46-4.42 (m, 2H); 3.72 (br. s, 1H); 3.68 (br. s, 1H); 2.12 (d, J=9.0, 1H); 1.98 (d, J=8.1, 2H); 1.71 (d, J=8.7, 1H); 1.24 (s, 9H); 0.89-0.80 (m, 2H).

### Example 49

### Preparation of N5-(3,3-Dimethyl-2-oxobutyl)-3-(4-nitrophenyl)-3,4-diazatricyclo[5.2.1.0^{2,6}]deca-2(6),4-dien-5-carboxamide

The title compound was synthesized by a procedure similar to that described for example 17. Intermediate 50 (237 mg, 0.79 mmol), Et₃N (270 μl, 1.98 mmol), BOP reagent (385 mg, 0.87 mmol), dry DMF (2.5 ml) and intermediate 113 (120 mg, 0.79 mmol) furnished the title compound as a pale yellow solid (306 mg 96%). ¹H-NMR (δ ppm, CDCl₃, 300 MHz): 8.32 (d, J=9.0, 2H); 7.90 (d, J=9.0, 2H); 7.58 (br. s, 1H); 4.47-4.44 (m, 2H); 3.78 (br. s, 1H); 3.75 (br. s, 1H); 2.14 (d, J=8.7, 1H); 2.03 (d, J=9.0, 2H); 1.75 (d, J=9.0, 1H); 1.24 (s, 11H).

### Example 50

### Preparation of N5-(3,3-Dimethyl-2-oxobutyl)-3-(4-fluorophenyl)-3,4-diazatricyclo[5.2.1.0^{2,6}]deca-2(6),4-dien-5-carboxamide

The title compound was synthesized by a procedure similar to that described for Example 36. Intermediate 32 (200 mg, 0.73 mmol), oxalyl chloride (76 μl, 0.88 mmol), intermediate 118 (121 mg, 0.80 mmol), E₃N (242 μl, 1.75 mmol), DMAP (5 mg) and dichloromethane (20 ml) were employed to obtain the title compound as a white foam (202 mg 75%). M.P.: 62-65 ° C. ¹H-NMR (δ ppm, CDCl₃, 300 MHz): 7.72-7.65 (m, 2H); 7.59 (br. s, 1H); 7.68 (t, J=8.7, 2H); 4.48-4.43 (m, 2H); 3.73 (br. s, 1H); 3.67 (br. s, 1H); 2.12 (d, J=8.4, 1H); 1.99 (d, J=8.1, 2H); 1.72 (d, J=8.7, 1H); 1.24 (s, 9H); 1.26-1.18 (m, 2H). IR (cm⁻¹, KBr): 3401 (m), 2968 (s), 2871 (m), 1720 (m), 1666 (s), 1550 (w), 1517 (s), 1491 (m), 1359 (m), 1276 (w), 1221 (m), 1154 (w), 1128 (w), 1093 (w), 836 (m). MS (m/z): 370.44 ([M+H]⁺).

### Example 51

### Preparation of N5-(3,3-Dimethyl-2-oxobutyl)-3-(2,4-difluorophenyl)-1,10,10-trimethyl-3,4-diazatricyclo[5.2.1.0^{2,6}]deca-2(6),4-dien-5-carboxamide

The title compound was synthesized by a procedure similar to that described for Example 36. Intermediate 73 (250 mg, 0.75 mmol), oxalyl chloride (78 μl, 0.90 mmol), Intermediate 113 (123 mg, 0.82 mmol), E₃N (249 μl, 1.80 mmol), catalytic amount of DMAP (5 mg) and dichloromethane (25 ml) furnished the title compound as a white foam (254 mg 78%). M.P.: 73-75° C. ¹H-NMR (δ ppm, CDCl₃, 300 MHz): 7.55-7.42 (m, 2H); 7.05-6.92 (m, 2H); 4.43 (d, J=4.8, 2H); 3.19 (br. s, 1H); 2.20-2.08 (m, 1H); 1.89-1.76 (m, 1H); 1.40-1.20 (m, 2H); 1.22 (s, 9H); 0.99 (s, 3H); 0.91 (s, 3H); 0.78 (s, 3H). IR (cm⁻¹, KBr): 3409 (m), 2968 (s), 2873 (m), 1717 (w), 1669 (s), 1614 (w), 1525 (s), 1494 (m), 1389 (w), 1367 (w), 1270 (m), 1225 (w), 1144 (m), 1118 (w), 1100 (w), 1017 (w), 851 (w). MS (m/z): 430.58 ([M+H]⁺).

### Example 52

### Preparation of N12-(3,3-Dimethyl-2-oxobutyl)-10-(2,4-difluorophenyl)-10,11-diazatetracyclo[6.5.2.1.0^{2,7}]pentadeca-2,4,6,9(13),11-pentaene-12-carboxamide

The title compound was synthesized by a procedure similar to that described for example 36. Intermediate 86 (100 mg, 0.28 mmol), dry dichloromethane (1.5 ml), oxalyl chloride (29 μl, 0.33 mmol), a catalytic amount of dry DMF and intermediate 113 (51 mg, 0.33 mmol) furnished the title compound as white foam (60 mg 47%). ¹H-NMR (δ ppm, CDCl₃, 300 MHz): 7.62-7.49 (m, 2H); 7.31 (d, J=6.3, 1H); 7.18-7.00 (m, 5H); 4.97 (s, 1H); 4.47-4.36 (m, 3H); 1.83-1.75 (m, 4H); 1.22 (s, 9H). MS (m/z): 450.60 ([M+H]⁺).

### Example 53

### Preparation of N7-(3,3-Dimethyl-2-oxobutyl)-5-(4-chlorophenyl)-5,6-diazatetracyclo[7.3.1.1^{3,11}.0^{4,8}]tetradeca-4(8),6-dien-7-carboxamide

The title compound was synthesized by a procedure similar to that described for example 36. Intermediate 89 (100 mg, 0.29 mmol), dry dichloromethane (2 ml), oxalyl chloride (30 μl, 0.34 mmol), a catalytic amount of dry DMF and intermediate 113(53 mg, 0.34 mmol) furnished the title compound as a white foam (72 mg 56%). ¹H-NMR (δ ppm, CDCl₃, 300 MHz): 7.57 (br. s, 1H); 7.42 (d, J=8.1, 2H); 7.29 (d, J=8.7, 2H); 4.40 (d, J=5.1, 2H); 3.89 (br. s, 1H); 3.01 (br. s, 1H); 2.19 (br. s, 2H); 2.04-1.78 (m, 8H); 1.30-1.19 (m, 2H); 1.21 (s, 9H). MS (m/z): 440.60 ([M+H]⁺).

### Example 54

### Preparation of 5-(tert-Butyl)-2-[5-(2,4-difluorophenyl)-4,5-diazatricyclo[5.2.1.0^{2,6}]deca-2(6),3-dien-3yl]-1,3-oxazole

A stirred solution of Example 36 (130 mg, 0.33 mmol) in dry toluene (4 ml) was treated with POCl₃ (92 μl, 1.00 mmol) and refluxed overnight. After cooling to room temperature, the mixture was diluted with ethyl acetate and washed with saturated solution of sodium bicarbonate and then with brine and dried over Na₂SO₄. Purification by SiO₂ column chromatography furnished the title compound as an off-white solid (110 mg 89%). M.P.: 58-60° C. ¹H-NMR (δ ppm, CDCl₃, 300 MHz): 7.85-7.60 (m, 1H); 7.04-6.94 (m, 2H); 6.82 (s, 1H); 3.73 (br. s, 1H); 3.50 (br. s, 1H); 2.14 (d, J=8.4, 1H); 2.05-1.92 (m, 2H); 1.72 (d, J=8.4, 1H); 1.36 (s, 9H); 1.32-1.21 (m, 2H). IR (cm⁻¹, KBr): 3444 (m), 2967 (s), 2872 (m), 1608 (m), 1525 (s), 1458 (w), 1366 (m), 1270 (s), 1234 (w), 1144 (m), 1119 (m), 1077 (m), 1038 (w), 964 (m), 849 (m). MS (m/z): 370.47 ([M+H]⁺).

### Example 55

### Preparation of 5-(tert-Butyl)-2-[(1S,7R)-5-(2,4-Difluorophenyl)-4,5-diazatricyclo[5.2.1.0^{2,6}]deca-2(6),3-dien-3yl]-1,3-oxazole

The title compound was synthesized by a procedure similar to that described for Example 54. Example 37 (200 mg, 0.36 mmol), dry toluene (4 ml) and POCl₃ (141 μl, 1.55 mmol) furnished the title compound as an off-white solid (159 mg 84%). M.P.: 93-96° C. ¹H-NMR (δ ppm, CDCl₃, 300 MHz): 7.85-7.60 (m, 1H); 7.04-6.94 (m, 2H); 6.82 (s, 1H); 3.73 (br. s, 1H); 3.50 (br. s, 1H); 2.14 (d, J=8.4, 1H); 2.05-1.92 (m, 2H); 1.72 (d, J=8.4, 1H); 1.36 (s, 9H); 1.32-1.21 (m, 2H). IR (cm⁻¹, KBr): 3436 (m), 2967 (s), 2871 (m), 1608 (m), 1581 (m), 1525 (s), 1496 (m), 1460 (m), 1388 (w), 1367 (m), 1270 (s), 1234 (w), 1159 (m), 1144 (s), 1119 (s), 1077 (s), 964 (s), 849 (m), 830 (m). MS (m/z): 370.47 ([M+H]⁺).

### Example 56

### Preparation of 5-(tert-Butyl)-2-[(1R,7S)-5-(2,4-Difluorophenyl)-4,5-diazatricyclo[5.2.1.0^{2,6}]deca-2(6),3-dien-3yl]-1,3-oxazole

The title compound was synthesized by a procedure similar to that described for Example 54. Example 38 (140 mg, 0.36 mmol), dry toluene (5 ml) and POCl₃ (92 μl, 1.00 mmol) furnished the title compound as an off-white solid (100 mg 75%). M.P.: 98-100° C. ¹H-NMR (δ ppm, CDCl₃, 300 MHz): 7.85-7.60 (m, 1H); 7.04-6.94 (m, 2H); 6.82 (s, 1H); 3.73 (br. s, 1H); 3.50 (br. s, 1H); 2.14 (d, J=8.4, 1H); 2.05-1.92 (m, 2H); 1.72 (d, J=8.4, 1H); 1.36 (s, 9H); 1.32-1.21 (m, 2H). IR (cm⁻¹, KBr): 3436 (m), 2967 (s), 2871 (m), 1608 (m), 1581 (m), 1525 (s), 1496 (m), 1460 (m), 1388 (w), 1367 (m), 1270 (s), 1234 (w), 1159 (m), 1144 (s), 1119 (s), 1077 (s), 964 (s), 849 (m), 830 (m). MS (m/z): 370.47 ([M+H]⁺).

### Example 57

### Preparation of Ethyl 2-{2-[5-(2,4-difluorophenyl)-4,5-diazatricyclo[5.2.1.0^{2,6}]deca-2(6),3-dien-3-yl]-1,3-oxazol-5-yl}-2-methyl propanoate

The title compound was synthesized by a procedure similar to that described for example 54. Example 42 (280 mg, 0.62 mmol), dry toluene (5 ml) and POCl₃ (172 μl, 1.87 mmol) were used to obtain the title compound as a yellow oil (185 mg, 69%). ¹H-NMR (δ ppm, CDCl₃, 300 MHz): 7.84-7.76 (m, 1H); 7.08-6.94 (m, 3H); 4.20-4.10 (m, 2H); 3.71 (br. s, 1H); 3.50 (br. s, 1H); 2.13 (d, J=9.0, 1H); 1.98 (d, J=6.9, 2H); 1.71 (d, J=9.0, 1H); 1.25 (s, 6H); 1.30-1.18 (m, 5H). IR (cm⁻¹, KBr): 3445 (m), 2983 (s), 2873 (m), 1734 (s), 1607 (m), 1524 (s), 1495 (m), 1386 (m), 1365 (m), 1270 (s), 1250 (m), 1147 (m), 1112 (m), 1077 (m), 1036 (w), 965 (m), 850 (m). MS (m/z): 428.75 ([M+H]⁺).

### Example 58

### Preparation of Ethyl 2-{2-[(1S,7R)-5-(2,4-difluorophenyl)-4,5-diazatricyclo[5.2.1.0^{2,6}]deca-2(6),3-dien-3-yl]-1,3-oxazol-5-yl}-2-methyl propanoate

The title compound was synthesized by a procedure similar to that described for example 54. Example 43 (550 mg, 1.23 mmol), dry toluene (10 ml) and POCl₃ (339 μl, 3.70 mmol) were used to obtain the title compound as a yellow oil (435 mg, 82%). ¹H-NMR (δ ppm, CDCl₃, 300 MHz): 7.83-7.70 (m, 1H); 7.02-6.92 (m, 3H); 4.14 (q, J=6.6, 2H); 3.70 (br. s, 1H); 3.49 (br. s, 1H); 2.13 (d, J=9.0, 1H); 1.98 (d, J=7.8, 2H); 1.71 (d, J=9.0, 1H); 1.24 (s, 6H); 1.30-1.20 (m, 5H). MS (m/z): 428.84 ([M+H]⁺).

### Example 59

### Preparation of Ethyl 2-{2-[(1R,7S)-5-(2,4-difluorophenyl)-4,5-diazatricyclo[5.2.1.0^{2,6}]deca-2(6),3-dien-3-yl]-1,3-oxazol-5-yl}-2-methyl propanoate

The title compound was synthesized by a procedure similar to that described for example 54. Example 44 (550 mg, 1.23 mmol), dry toluene (10 ml) and POCl₃ (339 μl, 3.70 mmol) were employed to get the title compound as an yellow oil (435 mg, 82%). ¹H-NMR (δ ppm, CDCl₃, 300 MHz): 7.84-7.72 (m, 1H); 7.02-6.92 (m, 3H); 4.14 (q, J=7.2, 2H); 3.70 (br. s, 1H); 3.49 (br. s, 1H); 2.13 (d, J=9.0, 1H); 1.98 (d, J=9.0, 2H); 1.71 (d, J=9.0, 1H); 1.24 (s, 6H); 1.30-1.22 (m, 5H). MS (m/z): 428.89 ([M+H]⁺).

### Example 60

### Preparation of 2-{2-[5-(2,4-Difurophenyl)-4,5-diazatricycyclo[5.2.1.0^{2,6}]deca-2(6),3-dien-3-yl]-1,3-oxazol-5-yl}-2-methylpropanoic acid

A procedure similar to that described for intermediate 3 was employed. Starting from example 57 (400 mg, 0.93 mol), isopropyl alcohol (3.5 ml), KOH (68 mg, 1.21 mmol) and water (1.0 ml) the title compound was obtained (300 mg, 80%). ¹H-NMR (δ ppm, CDCl₃, 300 MHz): 7.80-7.70 (m, 1H); 7.02-6.92 (m, 3H); 3.71 (br. s, 1H); 3.46 (br. s, 1H); 2.42-2.36 (m, 1H); 2.11 (d, J=7.8, 1H); 2.05-1.85 (m, 2H); 1.69 (d, J=9.0, 1H); 1.64 (s, 6H); 1.32-1.20 (m, 2H).

### Example 61

### Preparation of 2-{2-[(1R,7S)-5-(2,4-Difluorophenyl)-4,5-diazatricyclo[5.2.1.0^{2,6}]deca-2(6),3-dien-3-yl]-1,3-oxazol-5-yl}-2-methyl propanoic acid

This compound was prepared by a procedure similar to that described for intermediate 3. Example 59 (167 mg, 0.39 mol), isopropyl alcohol (1.3 ml), KOH (28 mg, 0.50 mmol) and water (0.5 ml) yielded the title compound as white solid (105 mg, 67%). Enantiomeric Excess: 93.19% ¹H-NMR (δ ppm, DMSO-d₆, 300 MHz): 12.76 (br. s, 1H); 7.82-7.72 (m, 1H); 7.63-7.54 (m, 1H); 7.26 (t, J=7.8, 1H); 7.15 (s, 1H); 3.59 (br. s, 1H); 3.49 (br. s, 1H); 2.03 (d, J=8.1, 1H); 1.96 (d, J=6.3, 2H); 1.69 (d, J=8.7, 1H); 1.52 (s, 6H); 1.22-1.05 (m, 2H). IR (cm⁻¹, KBr): 3446 (w), 3079 (m), 3008 (m), 2957 (m), 2939 (m), 2869 (m), 1703 (s), 1604 (w), 1518 (s), 1496 (m), 1454 (w), 1408 (w), 1363 (w), 1273 (s), 1142 (m), 1112 (m), 964 (m), 834 (m). MS (m/z): 400.27 ([M+H]⁺).

### Example 62

### Preparation of 2-{2-[(1S,7R)-5-(2,4-Difluorophenyl)-4,5-diazatricyclo[5.2.1.0^{2,6}]deca-2(6),3-dien-3-yl]-1,3-oxazol-5-yl}-2-methyl propanoic acid

This compound was prepared by a procedure similar to that described for intermediate 3. Example 58 (197 mg, 0.46 mol), isopropyl alcohol (1.5 ml), KOH (33 mg, 0.59 mmol) and water (0.5 ml) yielded the title compound as a white solid (120 mg, 65%). Enantiomeric Excess: 90.61%. ¹H-NMR (δ ppm, DMSO-d₆, 300 MHz): 12.76 (br. s, 1H); 7.82-7.72 (m, 1H); 7.57 (q, J=8.7, 1H); 7.26 (t, J=7.8, 1H); 7.15 (s, 1H); 3.59 (br. s, 1H); 3.49 (br. s, 1H); 2.03 (d, J=8.1, 1H); 1.96 (d, J=6.0, 2H); 1.69 (d, J=9.0, 1H); 1.52 (s, 6H); 1.24-1.05 (m, 2H). IR (cm⁻, KBr): 3445 (w), 3079 (m), 3008 (m), 2957 (m), 2939 (m), 2869 (m), 1700 (s), 1604 (w), 1518 (s), 1496 (m), 1454 (w), 1408 (w), 1363 (w), 1273 (s), 1142 (m), 1112 (m), 964 (m), 834 (m). MS (m/z): 400.25 ([M+H]⁺).

### Example 63

### Preparation of 2-{2-[5-(2,4-Difluorophenyl)-4,5-diazatricyclo[5.2.1.0^{2,6}]deca-2(6),3-dien-3yl]-1,3-oxazol-5-yl}-2-methyl-1-propanol

Example 57 (140 mg, 0.32 mmol) and lithium borohydride (14 mg, 0.65 mmol) were refluxed in dry THF for 4 h. The reaction mixture was concentrated, the residue obtained was diluted with water, acidified with 1N HCl and extracted with ethyl acetate. The combined organic layers were washed with brine and dried over Na₂SO₄. Purification of the crude product by SiO₂ column chromatography furnished the title compound as an off-white solid (102 mg 80%). M.P.: 66-68° C. ¹H-NMR (δ ppm, CDCl₃, 300 MHz): 7.84-7.72 (m, 1H); 7.04-6.95 (m, 2H); 6.94 (s, 1H); 3.72 (br. s, 1H); 3.68 (s, 2H); 3.50 (br. s, 1H); 2.14 (d, J=9.0, 1H); 1.99 (d, J=8.7, 2H); 1.72 (d, J=8.4, 1H); 1.36 (s, 6H); 1.29-1.23 (m, 2H). IR (cm⁻¹, KBr): 3430 (s), 2928 (m), 2872 (m), 1607 (m), 1522 (s), 1442 (m), 1388 (m), 1365 (m), 1327 (w), 1271 (s), 1234 (m), 1144 (m), 1077 (m), 1054 (m), 964 (m), 848 (m). MS (m/z): 386.59 ([M+H]⁺).

### Example 64

### Preparation of (1S,7R)-2-{2-[5-(2,4-Difluorophenyl)-4,5-diazatricyclo[5.2.1.0^{2,6}]deca-2(6),3-dien-3yl]-1,3-oxazol-5-yl}-2-methyl-1-propanol

The title compound was synthesized by a procedure similar to that described for Example 63. Example 58 (150 mg, 0.35 mmol), lithium borohydride (15 mg, 0.70 mmol) and dry THF (5 mL) furnished the title compound as an off-white solid (123 mg 91%). M.P.: 68-69° C. ¹H-NMR (δ ppm, CDCl₃, 300 MHz): 7.82-7.72 (m, 1H); 7.02-6.90 (m, 2H); 6.92 (s, 1H); 3.71 (br. s, 1H); 3.67 (s, 2H); 3.49 (br. s, 1H); 2.13 (d, J=8.1, 1H); 1.98 (d, J=9.0, 2H); 1.72 (d, J=8.7, 1H); 1.36 (s, 6H); 1.30-1.22 (m, 2H). IR (cm⁻¹, KBr): 3399 (s), 3080 (w), 2967 (s), 2871 (s), 1724 (w), 1608 (m), 1524 (s), 1497 (m), 1442 (m), 1389 (w), 1364 (m), 1271 (s), 1234 (w), 1144 (m), 1122 (m), 1078 (m), 964 (s), 848 (m), 831 (m). MS (m/z): 386.59 ([M+H]⁺).

### Example 65

### Preparation of (1R,7S)-2-{2-[5-(2,4-Difluorophenyl)-4,5-diazatricyclo[5.2.1.0^{2,6}]deca-2(6),3-dien-3yl]-1,3-oxazol-5-yl}-2-methyl-1-propanol

The title compound was synthesized by a procedure similar to that described for Example 63. Example 59 (150 mg, 0.35 mmol), lithium borohydride (15 mg, 0.70 mmol) and dry THF (5 mL) furnished the title compound as an off-white solid (129 mg 95%). M.P.: 70-72° C. ¹H-NMR (δ ppm, CDCl₃, 300 MHz): 7.81-7.70 (m, 1H); 7.02-6.93 (m, 2H); 6.92 (s, 1H); 3.70 (br. s, 1H); 3.67 (s, 2H); 3.49 (br. s, 1H); 2.13 (d, J=9.0, 1H); 1.98 (d, J=9.0, 2H); 1.71 (d, J=8.7, 1H); 1.35 (s, 6H); 1.30-1.22 (m, 2H). IR (cm⁻¹, KBr): 3399 (s), 2967 (s), 2871 (m), 1724 (m), 1608 (m), 1524 (s), 1442 (m), 1389 (w), 1364 (w), 1271 (s), 1234 (w), 1144 (m), 1078 (m), 964 (m), 848 (m), 831 (m). MS (m/z): 386.63 ([M+H]⁺).

### Example 66

### Preparation of 2-{2-[5-(2,4-Difurophenyl)-4,5-diazatricycyclo[5.2.1.0^{2,6}]deca-2(6),3-dien-3-yl]-1,3-oxazol-5-yl}-2-methylpropanamide

The title compound was synthesized by a procedure similar to that described for Intermediate 16. Example 60 (100 mg, 0.25 mmol), dry dichloromethane (1.5 ml), oxalyl chloride (26 μl, 0.30 mmol), a catalytic amount of dry DMF, dry acetone (5 ml) and aqueous ammonia (15 ml) furnished the title compound as a white solid (90 mg, 90%). M.P.: 221-223° C. ¹H-NMR (δ ppm, CDCl₃, 300 MHz): 7.80-7.70 (m, 1H); 7.06-6.94 (m, 3H); 5.67 (br. s, 1H); 5.34 (br. s, 1H); 3.70 (br. s, 1H); 3.50 (br. s, 1H); 2.14 (d, J=8.7, 1H); 2.00 (d, J=8.7, 2H); 1.73 (d, J=8.7, 1H); 1.64 (s, 6H); 1.32-1.20 (m, 2H). IR (cm⁻¹, KBr): 3470 (m), 3162 (m), 1691 (s), 1607 (m), 1516 (s), 1494 (m), 1454 (w), 1393 (w), 1362 (w), 1270 (m), 1140 (w), 1111 (w), 1078 (m), 961 (w), 845 (m), 835 (w). MS (m/z): 399.72 ([M+H]⁺).

### Example 67

### Preparation of 5-(tert-Butyl)-2-[5-(4-fluorophenyl)-4,5-diazatricyclo[5.2.1.0^{2,6}]deca-2(6),3-dien-3yl]-1,3-oxazole

The title compound was synthesized by a procedure similar to that described for Example 54. Example 50 (140 mg, 0.37 mmol), dry toluene (4.0 ml) and POCl₃ (104 μl, 1.13 mmol) gave the title compound as an off-white foam (113 mg 85%). M.P.: 64-65° C. ¹H-NMR (δ ppm, CDCl₃, 300 MHz): 7.75-7.69 (m, 2H); 7.15 (t, J=8.7, 2H); 6.81 (s, 1H); 3.73 (br. s, 1H); 3.70 (br. s, 1H); 2.17 (d, J=8.7, 1H); 2.04 (d, J=5.4, 2H); 1.75 (d, J=9.0, 1H); 1.36 (s, 9H); 1.32-1.10 (m, 2H). IR (cm⁻¹, KBr): 3436 (m), 2966 (s), 2870 (m), 1601 (w), 1581 (w), 1516 (s), 1493 (m), 1386 (w), 1366 (m), 1288 (m), 1229 (m), 1159 (w), 1125 (m), 1089 (w), 1076 (w), 970 (m), 834 (m). MS (m/z): 352.56 ([M+H]⁺).

### Example 68

### Preparation of 5-(tert-Butyl)-2-[-5-(4-chlorophenyl)-4,5-diazatricyclo[5.2.1.0^{2,6}]deca-2(6),3-dien-3yl]-1,3-oxazole

The title compound was synthesized by a procedure similar to that described for Example 54. To a stirred solution of example 39 (150 mg, 0.38 mmol), dry toluene (3 ml) and POCl₃ (107 μl, 1.16 mmol) furnished the title compound as pale yellow solid (107 mg 75%). M.P.: 133-135° C. ¹H-NMR (δ ppm, CDCl₃, 300 MHz): 7.72 (d, J=9.0, 2H); 7.42 (d, J=8.7, 2H); 6.82 (s, 1H); 3.73 (br. s, 2H); 2.18 (d, J=8.4, 1H); 2.01 (d, J=5.7, 2H); 1.75 (d, J=8.7, 1H); 1.37 (s, 9H); 1.31-1.21 (m, 2H). IR (cm⁻, KBr): 3413 (w), 2996 (m), 2967 (s), 2937 (m), 2867 (m), 1642 (w), 1594 (m), 1581 (m), 1504 (s), 1498 (s), 1446 (m), 1405 (w), 1366 (m), 1285 (m), 1252 (w), 1223 (w), 1206 (w), 1155 (m), 1124 (m), 1089 (s), 1077 (m), 1040 (m), 1007 (m), 969 (m), 829 (s), 806 (m). MS (m/z): 368.49 ([M+H]⁺).

### Example 69

### Preparation of 5-(tert-Butyl)-2-[5-(4-chloro-2-fluorophenyl)-4,5-diazatricyclo[5.2.1.0^{2,6}]deca-2(6),3-dien-3yl]-1,3-oxazole

The title compound was synthesized by a procedure similar to that described for Example 54. Example 45 (150 mg, 0.37 mmol), dry toluene (4 ml) and POCl₃ (110 μl, 1.13 mmol) furnished the title compound as an off-white solid (110 mg 76%). M.P.: 102-104° C. ¹H-NMR (δ ppm, CDCl₃, 300 MHz): 7.83-7.76 (m, 1H); 7.30-7.22 (m, 2H); 6.82 (s, 1H); 3.72 (br. s, 1H); 3.52 (br. s, 1H); 2.14 (d, J=9.0, 1H); 2.04-1.94 (m, 2H); 1.71 (d, J=9.0, 1H); 1.36 (s, 9H); 1.32-1.21 (m, 2H). IR (cm⁻¹, KBr): 3436 (m), 2966 (s), 2870 (s), 1589 (s), 1508 (s), 1491 (s), 1460 (m), 1446 (s), 1410(m), 1366 (m), 1326 (w), 1286 (m), 1223(m), 1159 (m), 1116 (m), 1078 (s), 1037 (m), 970 (m), 893 (m). MS (m/z): 386.61 ([M+H]⁺).

### Example 70

### Preparation of 5-(tert-Butyl)-2-[(1R,7S)- or (1S,7R)-5-(4-chloro-2-fluorophenyl)-4,5-diazatricyclo[5.2.1.0^{2,6}]deca-2(6),3-dien-3yl]-1,3-oxazole

The title compound was synthesized by a procedure similar to that described for Example 54. Example 46a (150 mg, 0.37 mmol), dry toluene (4 ml) and POCl₃ (102 μl, 1.11 mmol) furnished the title compound as an off-white solid (90 mg 63%). Enantiomeric Excess: 90%. M.P.: 65-70° C. ¹H-NMR (δ ppm, CDCl₃, 300 MHz): 7.75 (t, J=7.5, 1H); 7.32-7.20 (m, 2H); 6.80 (s, 1H); 3.71 (br. s, 1H); 3.51 (br. s, 1H); 2.13 (d, J=9.0, 1H); 2.05-1.94 (m, 2H); 1.71 (d, J=8.7, 1H); 1.36 (s, 9H); 1.33-1.21 (m, 2H). IR (cm⁻¹, KBr): 3436 (s), 2954 (s), 2925 (s), 2870 (m), 1590 (s), 1508 (s), 1491 (s), 1460 (m), 1410(m), 1366 (m), 1324 (w), 1286 (m), 1223(m), 1159 (m), 1116 (m), 1077 (m), 1037 (m), 971 (m), 893 (m). MS (m/z): 386.38 ([M+H]⁺).

### Example 71

### Preparation of 5-(tert-Butyl)-2-[(1S,7R)- or (1R,7S)-5-(4-chloro-2-fluorophenyl)-4,5-diazatricyclo[5.2.1.0^{2,6}]deca-2(6),3-dien-3yl]-1,3-oxazole

The title compound was synthesized by a procedure similar to that described for Example 54. Example 46b (150 mg, 0.37 mmol), dry toluene (4 ml) and POCl₃ (102 μl, 1.11 mmol) furnished the title compound as an off-white solid (80 mg 55%). Enantiomeric Excess: 91.47%. M.P.: 65-70° C. ¹H-NMR (δ ppm, CDCl₃, 300 MHz): 7.77 (t, J=7.5, 1H); 7.31-7.20 (m, 2H); 6.80 (s, 1H); 3.72 (br. s, 1H); 3.52 (br. s, 1H); 2.13 (d, J=8.1, 1H); 2.05-1.92 (m, 2H); 1.71 (d, J=8.4, 1H); 1.36 (s, 9H); 1.32-1.21 (m, 2H). IR (cm⁻¹, KBr): 3564 (s), 2965 (s), 2870 (m), 1637 (s), 1458 (s), 1460 (m), 1387 (m), 1364 (m), 1286 (m), 1221 (m), 1160 (m), 1125 (m), 1076 (m), 1049 (m), 971 (m), 893 (m). MS (m/z): 386.33 ([M+H]⁺).

### Example 72

### Preparation of 5-(tert-Butyl)-2-[5-(2,4,6-trifluorophenyl)-4,5-diazatricyclo[5.2.1.0^{2,6}]deca-2(6),3-dien-3yl]-1,3-oxazole

The title compound was synthesized by a procedure similar to that described for example 54. Example 41 (170 mg, 0.41 mmol), dry toluene (4 ml) and POCl₃ (170 μl, 1.74 mmol) furnished the title compound as an off-white solid (50 mg 30%). M.P.: 176-178° C. ¹H-NMR (δ ppm, CDCl₃, 300 MHz): 6.92-6.80 (m, 2H); 6.81 (s, 1H); 3.76 (br. s, 1H); 3.32 (br. s, 1H); 2.15 (d, J=9.0, 1H); 2.06-1.92 (m, 2H); 1.72 (d, J=7.5, 1H); 1.35 (s, 9H); 1.32-1.22 (m, 2H). IR (cm⁻¹, KBr): 3424 (m), 3060 (m), 2974 (s), 2929 (m), 2870 (m), 1644 (m), 1602 (s), 1537 (s), 1491(m), 1453 (m), 1370 (m), 1348 (m), 1287 (m), 1184 (m), 1131 (m), 1115 (s), 1094 (m), 1079 (m), 1035 (m), 1001 (m), 970 (m), 865 (m). MS (m/z): 388.60 ([M+H]⁺).

### Example 73

### Preparation of 5-(tert-Butyl)-2-[5-(4-methoxyphenyl)-4,5-diazatricyclo[5.2.1.0^{2,6}]deca-2(6),3-dien-3yl]-1,3-oxazole

The title compound was synthesized by a procedure similar to that described for Example 54. Example 47 (210 mg, 0.55 mmol), dry toluene (4 ml) and POCl₃ (153 μl, 1.65 mmol) furnished the title compound as an light yellow solid (151 mg 68%). M.P.: 110-113° C. ¹H-NMR (δ ppm, CDCl₃, 300 MHz): 7.63 (d, J=8.7, 2H); 6.95 (d, J=8.7, 2H); 6.78 (s, 1H); 3.83 (s, 3H); 3.71 (br. s, 1H); 3.66 (br. s, 1H); 2.15 (d, J=8.7, 1H); 2.01 (d, J=9.0, 2H); 1.72 (d, J=8.7, 1H); 1.36 (s, 9H); 1.33-1.21 (m, 2H). IR (cm⁻¹, KBr): 3431 (m), 2965 (s), 2986 (m), 2881 (m), 2907 (m), 1610 (w), 1582 (w), 1519 (s), 1492 (w), 1460 (w), 1474 (m), 1366 (m), 1291 (m), 1252 (s), 1212 (w), 1162 (m), 1122 (m), 1112 (m), 1097 (m), 1046 (m), 973 (m), 837 9m). MS (m/z): 364.33 ([M+H]⁺).

### Example 74

### Preparation of 5-(tert-Butyl)-2-[5-(4-bromophenyl)-4,5-diazatricyclo[5.2.1.0^{2,6}]deca-2(6),3-dien-3yl]-1,3-oxazole

The title compound was synthesized by a procedure similar to that described for Example 54. Example 48 (150 mg, 0.34 mmol), dry toluene (1.5 ml) and POCl₃ (97 μl, 1.44 mmol) furnished the title compound as yellow solid (109 mg 74%). M.P.: 118-121° C. ¹H-NMR (δ ppm, CDCl₃, 300 MHz): 7.63 (d, J=8.7, 2H); 7.54 (d, J=8.7, 2H); 6.79 (s, 1H); 3.83 (s, 3H); 3.71 (br. s, 2H); 2.17 (d, J=8.7, 1H); 2.00 (d, J=5.4, 2H); 1.74 (d, J=8.7, 1H); 1.36 (s, 9H); 1.34-1.20 (m, 2H). IR (cm⁻¹, KBr): 3423 (m), 2991 (m), 2967 (s), 2934 (m), 2866 (m), 1618 (w), 1589 (m), 1499 (s), 1488 (s), 1450 (m), 1400 (w), 1366 (s), 1320 (w), 1285 (m), 1258 (w), 1154 (m), 1124 (m), 1077 (m), 1069 (m), 1090 (m), 1040 (m), 1020 (m), 1005 (m), 970 (m), 947 (m), 826 (s). MS (m/z): 414.38 ([M+2H]⁺).

### Example 75

### Preparation of 5-(tert-Butyl)-2-[5-(4-nitrophenyl)-4,5-diazatricyclo[5.2.1.0^{2,6}]deca-2(6),3-dien-3yl]-1,3-oxazole

The title compound was synthesized by a procedure similar to that described for Example 54. Example 49 (295 mg, 0.74 mmol), dry toluene (3.0 ml) and POCl₃ (208 μl, 2.23 mmol) furnished the title compound as a yellow solid (162 mg 58%). M.P.: 145-148° C. ¹H-NMR (δ ppm, CDCl₃, 300 MHz): 8.32 (d, J=8.4, 2H); 7.95 (d, J=8.7, 2H); 6.83 (s, 1H); 3.83 (br. s, 1H); 3.74 (br. s, 1H); 2.18 (d, J=7.8, 1H); 2.05 (d, J=7.8, 2H); 1.79 (d, J=8.7, 1H); 1.38 (s, 9H); 1.33-1.18 (m, 2H). IR (cm⁻¹, KBr): 3444 (w), 3121 (w), 3078 (w), 2968 (m), 2869 (m), 1596 (s), 1517 (s), 1508 (s), 1490 (m), 1442 (m), 1415 (w), 1368 (w), 1334 (s), 1285 (m), 1157 (w), 1125 (m), 1088 (w), 1077 (w), 1040 (w), 1011 (w), 969 (w), 852 9m). MS (m/z): 379.25 ([M+H]⁺).

### Example 76

### Preparation of 5-(tert-Butyl)-2-[5-(2,4-difluorophenyl)-7,10,10-trimethyl-4,5-diazatricyclo[5.2.1.0^{2,6}]deca-2(6),3-dien-3yl]-1,3-oxazole

The title compound was synthesized by a procedure similar to that described for Example 54. The Example 51 (190 mg, 0.57 mmol), dry toluene (4 ml) and POCl₃ (158 μl, 1.73 mmol) furnished the title compound as an off-white foam (140 mg 71%). M.P.: 78-81° C. ¹H-NMR (δ ppm, CDCl₃, 300 MHz): 7.62-7.46 (m, 1H); 7.22-6.95 (m, 2H); 6.80 (s, 1H); 3.20 (br. s, 1H); 2.21-2.05 (m, 1H); 1.85-1.68 (m, 1H); 1.45-1.20 (m, 2H); 1.34 (s, 9H); 1.06 (s, 3H); 0.94 (s, 3H); 0.84 (s, 3H). IR (cm⁻¹, KBr): 3435 (m), 2966 (s), 2872 (m), 1608 (m), 1526 (s), 1498 (m), 1461 (w), 1367 (w), 1270 (s), 1233 (w), 1204 (w), 1143 (m), 1121 (m), 1090 (m), 1061 (w), 1012 (w), 963 (m). MS (m/z): 412.50 ([M+H]⁺).

### Example 77

### Preparation of 5-(tert-Butyl)-2-[12-(2,4-difluorophenyl)-11,12-diazatetracyclo[6.5.2.0^{2,7}.0^{9,13}]pentadeca-2(7),3,5,9(13),10-pentaen-10-yl]-1,3-oxazole

The title compound was synthesized by a procedure similar to that described for Example 54. Example 52 (50 mg, 0.11 mmol), dry toluene (1 ml) and POCl₃ (31 μl, 0.33 mmol) gave the title compound as waxy material (38 mg 81%). ¹H-NMR (δ ppm, CDCl₃, 300 MHz): 7.72-7.64 (m, 1H); 7.33 (d, J=6.3, 1H); 7.17 (d, 6.9, 1H); 7.12-6.98 (m, 4H); 6.82 (s, 1H); 4.94 (br. s, 1H); 4.40 (br. s, 1H); 1.89-1.80 (m, 4H); 1.37 (s, 9H). IR (cm⁻¹, KBr): 3850 (w), 3564 (m), 3444 (m), 3070 (w), 2963 (s), 2930 (s), 2869 (m), 1609 (m), 1523 (m), 1504 (m), 1459 (m), 1367 (m), 1269 (s), 1204 (w), 1143 (s), 1118 (s), 1086 (s), 1036 (m), 966 (m), 848 (m), 810 (m). MS (m/z): 432.70 ([M+H]⁺).

### Example 78

### Preparation of 5-(tert-Butyl)-2-[5-(4-chlorophenyl)-5,6-diazatetracyclo[7.3.1.1^{3,11}.0^{4,8}]tetradeca-4(8),6-dien-3-yl]-1,3-oxazole

The title compound was synthesized by a procedure similar to that described for Example 54. Example 53 (50 mg, 0.11 mmol), dry toluene (1.0 ml) and POCl₃ (51 μl, 0.34 mmol) furnished the title compound as an off-white solid (32 mg 68%). M.P.: 178-180° C. ¹H-NMR (δ ppm, CDCl₃, 300 MHz): 7.42 (d, J=7.2, 2H); 7.33 (d, J=8.1, 2H); 6.77 (s, 1H); 3.80 (br. s, 1H); 3.03 (br. s, 1H); 2.22 (br. s, 2H); 2.12-1.82 (m, 8H); 1.34 (s, 9H); 1.32-1.20 (m, 2H). IR (cm⁻¹, KBr): 3840 (w), 3503 (w), 2951 (s), 2928 (s), 2906 (s), 2847 (m), 1596 (w), 1583 (w), 1497 (s), 1459 (m), 1405 (w), 1364 (m), 1289 (m), 1236 (m), 1088 (m), 1008 (m), 972 (w), 839 (m), 824 (m). MS (m/z): 422.85 ([M+H]⁺).

### Example 79

### Preparation of 2-[5-(2,4-Difluorophenyl)-4,5-diazatricyclo[5.2.1.0^{2,6}]deca-2(6),3-dien-3yl]-4-phenyl-1,3-thiazole

Intermediate 19 (100 mg, 0.32 mmol) and phenacyl bromide (78 mg, 0.39 mmol) were mixed in dry THF (5.0 ml) and the mixture was stirred at RT for 4 h, and heated at reflux for 2 h. The solvent was removed under reduced pressure, treated with a saturated solution of sodium bicarbonate and extracted into dichloromethane and the combined organic layers were washed with water, brine and dried over Na₂SO₄. Purification of crude by SiO₂ column chromatography furnished the title compound as a white solid (118 mg, 89%). M.P.: 128-130° C. ¹H-NMR (δ ppm, CDCl₃, 300 MHz): 8.02 (d, J=6.9, 2H); 7.84-7.74 (m, 1H); 7.50-7.32 (m, 4H); 7.08-6.95 (m, 2H); 3.89 (br. s, 1H); 3.52 (br. s, 4H); 2.18 (d, J=7.8, 1H); 2.11-1.94 (m, 2H); 1.74 (d, 8.7, 2H); 1.41-1.24 (m, 2H). IR (cm⁻¹, KBr): 3436 (m), 2947 (w), 2869 (w), 1610 (m), 1521 (s), 1484 (w), 1473 (w), 1441 (m), 1270 (s), 1143 (m), 983 (m). MS (m/z): 406.36 ([M+H]⁺).

### Example 80

### Preparation of 4-(tert-Butyl)-2-[5-(2,4-difluorophenyl)-4,5-diazatricyclo[5.2.1.0^{2,6}]deca-2(6),3-dien-3yl]-1,3-thiazole

The title compound was synthesized by a procedure similar to that described for Example 79. Intermediate 19 (140 mg, 0.45 mmol), 1-bromopinacolone (63 μl, 0.47 mmol) and THF (5.0 mL) furnished the title compound as white solid (125 mg, 71%). M.P.: 102-103° C. ¹H-NMR (δ ppm, CDCl₃, 300 MHz): 7.76 (q, J=8.4, 1H); 7.00 (t, J=8.4, 2H); 6.85 (s, 1H); 3.78 (br. s, 1H); 3.49 (br. s, 1H); 2.15 (d, 8.1, 1H); 2.08-1.89 (m, 2H); 1.71 (d, J=9.0, 1H); 1.40 (s, 9H); 1.35-1.22 (m, 2H). IR (cm⁻¹, KBr): 3444 (m), 3124 (w), 3080 (w), 3055 (w), 3013 (m), 2958 (s), 2926 (s), 2867 (m), 1608 (m), 1528 (s), 1505 (s), 1456 (w), 1438 (m), 1358 (m), 1350 (m), 1267 (m), 1236 (m), 1143 (m), 991 (m). MS (m/z): 386.41 ([M+H]⁺).

### Example 81

### Preparation of 5-(tert-Butyl)-2-[5-(2,4-difluorophenyl)-4,5-diazatricyclo[5.2.1.0^{2,6}]deca-2(6),3-dien-3yl]-1,3-thiazole

The title compound was synthesized by a procedure similar to that described for Example 24. Example 36 (162 mg, 0.41 mmol) and P₂S₅ (139 mg, 0.62 mmol) furnished the title compound as a white solid (132 mg 82%). M.P.: 59-60° C. ¹H-NMR (δ ppm, CDCl₃, 300 MHz): 7.81-7.20 (m, 1H); 7.55 (s, 1H); 7.03-6.96 (m, 2H); 3.77 (br. s, 1H); 3.49 (br. s, 1H); 2.13 (d, J=6.6, 1H); 2.05-1.85 (m, 2H); 1.71 (d, J=9.0, 1H); 1.42 (s, 9H); 1.38-1.22 (m, 2H). IR (cm⁻¹, KBr): 3445 (m), 2962 (s), 2869 (m), 1607 (m), 1523 (s), 1459 (w), 1437 (w), 1364 (w), 1269 (m), 1143 (m), 1121 (w), 1086 (w), 1056 (w), 981 (m), 950 (w), 848 (m). MS (m/z): 386.58 ([M+H]⁺).

### Example 82

### Preparation of 5-(tert-Butyl)-2-[(1S,7R)-5-(2,4-difluorophenyl)-4,5-diazatricyclo[5.2.1.0^{2,6}]deca-2(6),3-dien-3yl]-1,3-thiazole

The title compound was synthesized by a procedure similar to that described for Example 24. Example 37 (310 mg, 0.80 mmol) and P₂S₅ (266 mg, 1.20 mmol) furnished the title compound as a white solid (250 mg 81%). Enantiomeric Excess: 87.68%. M.P.: 59-61° C. ¹H-NMR (δ ppm, CDCl₃, 300 MHz): 7.82-7.68 (m, 1H); 7.53 (s, 1H); 7.02-6.92 (m, 2H); 3.75 (br. s, 1H); 3.48 (br. s, 1H); 2.13 (d, J=8.4, 1H); 2.06-1.85 (m, 2H); 1.70 (d, J=8.7, 1H); 1.41 (s, 9H); 1.30 (d, J=6.0, 2H). IR (cm⁻¹, KBr): 3434 (m), 2962 (s), 2869 (m), 1607 (m), 1523 (s), 1460 (w), 1437 (w), 1364 (w), 1269 (m), 1143 (m), 1121 (w), 1086 (w), 1056 (w), 981 (m), 962 (m), 950 (w), 848 (m). MS (m/z): 386.20 ([M+H]⁺).

### Example 83

### Preparation of 5-(tert-Butyl)-2-[(1R,7S)-5-(2,4-difluorophenyl)-4,5-diazatricyclo[5.2.1.0^{2,6}]deca-2(6),3-dien-3yl]-1,3-thiazole

The title compound was synthesized by a procedure similar to that described for Example 24. The Intermediate 38 (340 mg, 0.87 mmol) and P₂S₅ (292 mg, 1.31 mmol) furnished the title compound as a white solid (265 mg 78%). Enantiomeric Excess: 88.7%. M.P.: 61-63° C. ¹H-NMR (δ ppm, CDCl₃, 300 MHz): 7.78-7.67 (m, 1H); 7.52 (s, 1H); 6.96 (t, J=8.4, 2H); 3.75 (br. s, 1H); 3.48 (br. s, 1H); 2.12 (d, J=8.7, 1H); 2.06-1.85 (m, 2H); 1.74-1.69 (m, 1H); 1.41 (s, 9H); 1.31 (d, J=6.6, 2H). IR (cm⁻¹, KBr): 3441 (s), 3086 (w), 2962 (s), 2869 (m), 1608 (m), 1522 (s), 1459 (w), 1437 (w), 1364 (w), 1269 (m), 1143 (m), 1121 (w), 1086 (w), 1056 (w), 981 (m), 962 (m), 950 (w), 848 (m). MS (m/z): 386.28 ([M+H]⁺).

### Example 84

### Preparation of 5-[5-(tert-Butyl)-1H-2-imidozolyl]-3-(2,4-difluorophenyl)-3,4-diazatricyclo[5.2.1.0^{2,6}]deca-2(6),4-diene

A mixture of Intermediate 30 (120 mg, 0.41 mmol) and 1-chloro-3,3-dimethylbutanone (55 μl, 0.41 mmol) in THF-water (5 mL) (4:1) was refluxed overnight. The solvent was evaporated, diluted with water, the residue extracted with dichloromethane and the organic layers dried over Na₂SO₄. Purification of the crude product by SiO₂ column chromatography furnished the title compound as an off-white solid (106 mg 69%). M.P.: 165-167° C. ¹H-NMR (δ ppm, CDCl₃, 300 MHz): 9.52 (br. s, 1H); 7.68-7.60 (m, 1H); 6.99 (t, J=9.0, 2H); 6.85 (s, 1H); 3.81 (br. s, 1H); 3.45 (br. s, 1H); 2.11 (d, J=7.8, 1H); 1.99-1.94 (m, 2H); 1.72-1.66 (m, 1H); 1.32 (s, 9H); 1.34-1.28 (m, 2H). IR (cm^{31 1}, KBr): 3435 (m), 2959 (s), 2869 (m), 1608 (m), 1520 (s), 1443 (w), 1423 (w), 1366 (s), 1269 (m), 1234 (w), 1110 (m), 1142 (m), 1082 (w), 969 (w), 845 (w). MS (m/z): 369.71 ([M+H]⁺).

### Example 85

### Preparation of 5-[4-(tert-Butyl)-1-methyl-1H-2-imidozolyl]-3-(2,4-difluorophenyl)-3,4-diazatricyclo[5.2.1.0^{2,6}]deca-2(6),4-diene or 5-[5-(tert-Butyl)-1-methyl-1H-2-imidozolyl]-3-(2,4-difluorophenyl)-3,4-diazatricyclo[5.2.1.0^{2,6}]deca-2(6),4-diene

Example 84 (50 mg, 0.41 mmol) and methyl iodide (55 μl, 0.41 mmol) were mixed in THF-water (5 mL) (4:1) and heated to reflux for overnight. The solvent evaporated and diluted with water and extracted with dichloromethane and dried over Na₂SO₄. The purification of crude product by SiO₂ column chromatography furnished a single isomer as an off-white solid (40 mg 78%). M.P.: 115-117° C. ¹H-NMR (δ ppm, CDCl₃, 300 MHz): 7.69 (q, J=8.1, 1H); 6.95 (t, J=8.1, 2H); 6.59 (s, 1H); 3.86 (s, 3H); 3.70 (br. s, 1H); 3.47 (br. s, 1H); 2.14 (d, J=8.1, 1H); 1.98-1.85 (m, 2H); 1.39 (s, 9H); 1.30-1.22 (m, 3H). IR (cm⁻¹, KBr): 3442 (m), 2948 (s), 2924 (s), 2866 (m), 1605 (w), 1545 (m), 1518 (s), 1458 (m), 1435 (m), 1360 (w), 1269 (m), 1233 (w), 1211 (w), 1143 (m), 1080 (m), 1021 (m), 963 (m), 841 (m). MS (m/z): 383.53 ([M+H]⁺).

### Example 86

### Preparation of E or Z-1-{5-(2,4-Diflurophenyl)-4,5-diazatricyclo[5.2.1.0^{2,6}.]deca-2(6),3-dien-3-yl]}-3,3,-dimethyl-1-butanone-O-methyl-oxime

To a solution of Example 6 (75 mg, 0.21 mmol) in dry ethanol, pyridine (200 μl, 2.35 mmol) and methoxyamine hydrochloride were added and the reaction was stirred at RT for 5 h and then refluxed overnight. The solvent was evaporated, the residue diluted with water and extracted into ethylacetate. Organic extracts were washed with brine and dried over Na₂SO₄. Purification by chromatography furnished the title compound (58 mg, 71%) as an oil. ¹H-NMR (δ ppm, CDCl₃, 300 MHz): 7.63-7.56 (m, 1H); 6.94 (t, J=8.4, 2H); 3.93 (s, 3H), 3.57 (br. s, 1H); 3.42 (br. s, 1H); 2.82, 2.71 (AB, J=12.2, 2H); 2.06 (d, J=8.4, 2H); 1.90 (d, J=7.8, 2H); 1.62 (d, J=8.7, 1H); 1.28-1.16 (m, 2H); 0.96 (s, 9H). MS (m/z): 374.28 ([M+H]⁺).

### Example 87

### Preparation of 5-[4-(tert-Butyl)phenyl]-3-(2,4-difluorophenyl)-3,4-diazatricyclo[5.2.1.0^{2,6}]deca-2(6),4-diene

The intermediate 12 (200 mg, 0.53 mmol), 4-tert-butylphenyl boronic acid (144 mg, 0.80 mmol) and sodium carbonate (284 mg, 2.68 mmol) in dioxane (5 mL)-water (2.5 mL) was degassed with nitrogen for 10 min. Tetrakis(triphenylphosphine)palladium(0) (62 mg, 0.05 mmol) was added and the reaction was heated at 100° C. overnight. Dioxane was evaporated and diluted with water and extracted with ethyl acetate and the combined organic layers were washed with brine and dried over Na₂SO₄. The solvent was removed and the residue subjected to SiO₂ column chromatography to furnish the title compound as pale yellow solid (120 mg, 59%). M.P.: 130-132° C. ¹H-NMR (δ ppm, CDCl₃, 300 MHz): 7.77 (d, J=8.4, 2H); 7.74-7.70 (m, 1H); 7.41 (d, J=9.0, 2H); 7.00-6.94 (m, 2H); 3.65 (br. s, 1H); 3.47 (br. s, 1H); 2.11 (d, J=7.5, 1H); 1.96 (d, J=8.4, 2H); 1.69 (d, J=8.7, 1H); 1.34 (s, 9H); 1.28 (d, J=7.8, 2H). IR (cm⁻¹, KBr): 3445 (m), 3074 (m), 3029 (m), 2961 (s), 2869 (s), 1606 (s), 1514 (s), 1455 (s), 1365 (s), 1321 (w), 1265 (s), 1233 (m), 1142 (s), 1086 (s), 1061 (m), 1024 (m), 1005 (m), 960 (m), 845 (m), 829 (m). MS (m/z): 379.30 ([M+H]⁺).

### Example 88

### Preparation of 3-[5-(2,4-Difluorophenyl)-4,5-diazatricyclo[5.2.1.0^{2,6}]deca-2(6),3-dien-3-yl]benzaldehyde

The title compound was synthesized by a procedure similar to that described for Example 87. The Intermediate 12 (200 mg, 0.53 mmol), 3-formylphenyl boronic acid (121 mg, 0.80 mmol) and sodium carbonate (284 mg, 2.68 mmol), dioxane (5 mL)-water (2.5 mL) and tetrakis(triphenyl)palladium (0) (31 mg, 0.05 mmol) furnished the title compound as off-white solid (120 mg, 63%). M.P.: 98-100° C. ¹H-NMR (δ ppm, CDCl₃, 300 MHz): 10.05 (br. s, 1H); 8.34 (br. s, 1H); 8.12 (d, J=7.5, 1H); 7.83-7.73 (m, 2H); 7.56 (t, J=7.8, 1H); 7.02-6.96 (m, 2H); 3.72 (br. s, 1H); 3.51 (br. s, 1H); 2.14 (d, J=8.4, 1H); 2.04-1.85 (m, 2H); 1.74 (d, J=8.4, 1H); 1.30 (d, J=8.4, 2H). IR (cm⁻¹, KBr): 3072 (m), 2970 (m), 2953 (w), 2871 (m), 1699 (s), 1606 (m), 1523 (s), 1444 (s), 1397 (m), 1397 (m), 1269 (m), 1167 (m), 1121 (m), 1087 (m), 1060 (m), 963 (m), 861 (m). MS (m/z): 351.27 ([M+H]⁺).

### Example 89

### Preparation of 3-[5-(2,4-Difluorophenyl)-4,5-diazatricyclo[5.2.1.0^{2,6}]deca-2(6),3-dien-3-yl]phenylmethanol

Example 88 (120 mg, 0.34 mmol) and sodium borohydride (26 mg, 0.68 mmol) were refluxed in dry THF for 1 h. The reaction mixture was concentrated, the residue obtained was diluted with water, acidified with 1N HCl and extracted with ethyl acetate. The combined organic layers were washed with brine and dried over Na₂SO₄. Purification of the crude product by SiO₂ column chromatography furnished the title compound as an off-white solid (70 mg 58%). M.P.: 113-115° C. ¹H-NMR (δ ppm, CDCl₃, 300 MHz): 7.86 (br. s, 1H); 7.80-7.64 (m, 2H); 7.42-7.23 (m, 2H); 7.02-6.95 (m, 2H); 4.74 (br. s, 2H); 3.68 (br. s, 1H); 3.49 (br. s, 1H); 2.12 (d, J=8.4, 1H); 1.98 (d, J=9.0, 2H); 1.72 (d, J=8.1, 1H); 1.30 (d, J=7.5, 2H). IR (cm⁻¹, KBr): 3376 (m), 2975 (m), 2875 (m), 1677 (s), 1603 (w), 1557 (m), 1508 (s), 1453 (m), 1386 (m), 1368 (m), 1253 (w), 1224 (m), 1100 (m), 945 (m). MS (m/z): 353.39 ([M+H]⁺).

### Example 90

### Preparation of N1-(tert-Butyl)-3-[4-(2,4-Difluorophenyl)-4,5-diazatricyclo[5.2.1.0^{2,6}]deca-2(6),3-dien-3-yl]propanamide or N1-(tert-Butyl)-3-[5-(2,4-Difluorophenyl)-4,5-diazatricyclo[5.2.1.0^{2,6}]deca-2(6),3-dien-3-yl]propanamide

Triethylamine (40 μl, 0.28 mmol) and BOP reagent (117 mg, 0.26 mmol) were added to intermediate 81 (77 mg, 0.24 mmol) in dry DMF (1.0 ml). After stirring at RT for 30 min, tert-butylamine (25 μl, 0.24 mmol) was added and the mixture was stirred overnight, diluted with water, extracted with ethyl acetate, organic extracts washed with brine and dried over Na₂SO₄. Evaporation of the solvent and purification of the crude product by SiO₂ column chromatography furnished the title compound as a thick paste (78 mg, 86%). ¹H-NMR (δ ppm, DMSO-d₆, 300 MHz): 7.65 (q, J=9.0, 1H); 7.56-7.44 (m, 2H); 7.21 (t, J=8.4, 1H); 3.41-3.28 (m, 2H, under H₂O signal); 2.73 (t, J=7.2, 2H); 2.37 (t, J=7.2, 2H); 1.87 (d, J=6.9, 3H); 1.59 (d, J=7.8, 1H); 1.24 (s, 9H); 1.06 (d, J=6.9, 2H). IR (cm⁻¹, KBr): 3435 (m), 3078 (w), 2967 (m), 2871 (m), 1647 (s), 1530 (s), 1454 (s), 1364 (s), 1269 (m), 1224 (m), 1143 (m), 1110 (m), 1121 (m), 1080 (m), 965 (m), 850 (m). MS (m/z): 374.45 ([M+H]⁺).

### Example 91

### Preparation of N1-(tert-Butyl)-3-[5-(2,4-Difluorophenyl)-4,5-diazatricyclo[5.2.1.0^{2,6}]deca-2(6),3-dien-3-yl]propanamide or N1-(tert-Butyl)-3-[4-(2,4-Difluorophenyl)-4,5-diazatricyclo[5.2.1.0^{2,6}]deca-2(6),3-dien-3-yl]propanamide

The title compound was synthesized by a procedure similar to that described for Example 90. Intermediate 82 (70 mg, 0.21 mmol), triethylamine (36 μl, 0.28 mmol), BOP reagent (106 mg, 0.24 mmol), dry DMF (1.0 ml) and tert-butylamine (23 μl, 0.21 mmol) furnished the title compound as a thick paste (60 mg, 73%). ¹H-NMR (δ ppm, CDCl₃, 300 MHz): 7.46-7.38 (m, 1H); 6.95 (t, J=8.1, 2H); 5.21 (br. s, 1H); 3.36 (br. s, 2H); 2.75 (t, J=7.8, 2H); 2.43 (t, J=7.2, 2H); 1.99-1.91 (m, 3H); 1.68 (d, J=8.7, 1H); 1.32 (s, 9H); 1.33-1.20 (m, 2H). IR (cm⁻¹, KBr): 3360 (m), 3088 (w), 2978 (m), 2928 (m), 2871 (m), 1670 (s), 1599 (m), 1520 (s), 1487 (m), 1455 (w), 1415 (w), 1376 (w), 1360 (w), 1271 (m), 1252 (m), 1220 (m), 1141 (m), 1109 (m), 1091 (m), 962 (m), 850 (m). MS (m/z): 374.35 ([M+H]⁺).

### Example 92a

### Preparation of Methyl (2S)-2-[5-(2,4-difluorophenyl)-4,5-diazatricyclo[5.2.1.0.^{2,6}]deca-2(6),3-dien-3-ylcarboxamido]-2-(4-fluorophenyl)ethanoate

The title compound was synthesized by a procedure similar to that described for example 17. Intermediate 3 (400 mg, 1.37 mmol), DMF (4.0 ml), Et₃N (450 μl, 3.31 mmol), BOP reagent (670 mg, 1.51 mmol) and (S)-(−)-2-(4-flurophenyl)glycine methyl ester hydrochloride (302 mg, 1.37 mmol) furnished the title compound (543 mg, 86%). M.P.: 51-52° C. ¹H-NMR (δ ppm, CDCl₃, 300 MHz): 7.78-7.68 (m, 2H); 7.44-7.41 (m, 2H); 7.06-6.95 (m, 4H); 5.63 (d, J=6.9, 1H); 3.77, 3.76 (2s, 3H); 3.70 (br. s, 1H); 3.44 (br. s, 1H); 2.10-2.02 (m, 1H); 2.00-1.90 (m, 2H); 1.67 (d, J=7.8, 1H); 1.30-1.19 (m, 2H).

### Example 92b

### Preparation of N5-[(1S)-2-Hydroxy-1-(4-fluorophenyl)ethyl 3-(2,4-difluorophenyl)-3,4-diazatricyclo[5.2.1.0.^{2,6}]deca-2(6),4-diene-5-carboxamide

The title compound was synthesized by a procedure similar to that described for example 63. Example 92a (400 mg, 0.87 mmol), THF (6 ml) and LiBH₄ (38 mg, 1.75 mmol) furnished the title compound (288 mg, 76%). M.P.: 116-119° C. ¹H-NMR (δ ppm, CDCl₃, 300 MHz): 7.65 (q, J=8.4, 1H); 7.49-7.41 (m, 1H); 7.40-7.32 (m, 2H); 7.08-6.96 (m, 4H); 5.24-5.18 (m, 1H), 3.96 (d, J=5.1, 2H); 3.72 (br. s, 1H); 3.44 (br. s, 1H); 2.12-2.02 (m, 2H); 1.99-1.92 (m, 2H); 1.68 (d, J=8.7, 1H); 1.28-1.20 (m, 2H).

### Example 93

### Preparation of N5-(2-hydroxy-1,1-dimethylethyl)-3-(2,4-diflurophenyl)-3,4-diazatricyclo[5.2.1.0^{2,6}]deca-2(6),4-diene-5-carboxamide

The Intermediate 3 (500 mg, 1.72 mmol) was dissolved in dry DMF (5.0 ml), Et₃N (267 μl, 2.58 mmol) and BOP reagent (800 mg, 1.81 mmol) were added to the solution. After stirring at RT for 30 min., 2-amino-2-methyl propanol (247 μl, 2.58 mmol) was added and the mixture was was stirred at RT overnight. The reaction was then diluted with water, extracted with ethyl acetate and combined organic extracts were washed with brine and dried over Na₂SO₄. Purification of the crude product by column chromatography furnished the title compound as an off-white solid (410 mg, 65%). M.P.: 127-129° C. ¹H-NMR (δ ppm, CDCl₃, 300 MHz): 7.70-7.61 (m, 1H); 7.06-6.96 (m, 2H); 6.93 (br. s, 1H); 5.01 (br. s, 1H); 3.69 (br. s, 3H); 3.44 (br. s, 1H); 2.08 (d, J=9.0, 1H); 2.04-1.94 (m, 2H); 1.69 (d, J=9.0, 1H); 1.39 (s, 6H); 1.30-1.20 (m, 2H). IR (cm⁻¹, KBr): 3405 (s), 3377 (s), 3054 (w), 2976 (m), 2936 (m), 2870 (m), 1652 (s), 1608 (m), 1549 (s), 1523 (s), 1493 (s), 1447 (s), 1394 (w), 1372 (w), 1360 (w), 1273 (s), 1255 (m), 1236 (w), 1146 (m), 1093 (m), 1062 (m), 965 (m), 827 (w). MS (m/z): 362.33 ([M+H]⁺).

### Example 94

### Preparation of (1R,7S)-N5-(2-hydroxy-1,1-dimethylethyl)-3-(2,4-difluorophenyl)-3,4-diazatricyclo[5.2.1.0^{2,6}]deca-2(6),4-diene-5-carboxamide

This compound was prepared from intermediate 5 (250 mg, 0.86 mmol), dry dimethyl formamide (1-5 mL), triethylamine (134 μL, 0.94 mmol), benzotriazol-1-yloxytris(dimethylamino)-phosphonium hexafluorophosphate (400 mg, 0.90 mmol) and 2-amino-2-methyl propanol (99 μL, 1.03 mmol) according to a procedure described in Example 93 and the title compound was obtained as an off-white solid. M.P.: 148-152° C. ¹H-NMR (δ ppm, CDCl₃, 300 MHz): 7.66 (q, J=9.0, 1H); 7.03 (t, J=9.0, 2H); 6.96 (br. s, 1H); 3.72 (br. s, 1H); 3.70 (br. s, 2H); 3.45 (br. s, 1H); 2.09 (d, J=9.0, 1H); 2.04-1.92 (m, 2H); 1.70 (d, J=8.7, 1H); 1.40 (s, 6H), 1.32-1.20 (m, 2H). IR (cm⁻¹, KBr): 3421 (m), 3379 (s), 3054 (w), 2975 (m), 2870 (m), 1652 (s), 1606 (w), 1548 (m), 1522 (s), 1492 (m), 1447 (m), 1396 (w), 1273 (s), 1145 (m), 1060 (m), 965 (m). MS (m/z): 362.28 ([M+H]⁺).

### Example 95

### Preparation of (1S,7R)-N5-(2-Hydroxy-1,1-dimethylethyl)-3-(2,4-difluorophenyl)-3,4-diazatricyclo[5.2.1.0^{2,6}]deca-2(6),4-diene-5-carboxamide

This compound was prepared from intermediate 4 (100 mg, 0.34 mmol), dry dimethyl formamide (1-5 mL), triethylamine (58 μL, 0.41 mmol), benzotriazol-1-yloxytris(dimethylamino)-phosphonium hexafluorophosphate (160 mg, 0.36 mmol) and 2-amino-2-methyl propanol (43 μL, 0.44 mmol) according to a procedure described in Example 93 and furnished the title compound as an off-white solid. M.P.: 142-144° C. ¹H-NMR (δ ppm, CDCl₃, 300 MHz): 7.66 (q, J=9.0, 1H); 7.02 (t, J=9.0, 2H); 6.95 (br. s, 1H); 3.70 (br. s, 3H); 3.44 (br. s, 1H); 2.09 (d, J=9.0, 1H); 2.04-1.92 (m, 2H); 1.69 (d, J=8.4, 1H); 1.40 (s, 6H), 1.33-1.22 (m, 2H). IR (cm⁻¹, KBr): 3415 (m), 3378 (s), 3055 (w), 2976 (m), 2870 (m), 1652 (s), 1608 (w), 1548 (m), 1523 (s), 1493 (m), 1447 (m), 1396 (w), 1273 (s), 1146 (m), 1061 (m), 965 (m). MS (m/z): 362.29 ([M+H]⁺).

### Example 96

### Preparation of N5-(2-Hydroxy-1,1-dimethylethyl)-3-(4-chlorophenyl)-3,4-diazatricyclo[5.2.1.0^{2,6}]deca-2(6),4-diene-5-carboxamide

This compound was prepared from intermediate 34 (100 mg, 0.34 mmol), dry dimethyl formamide (1-5 mL), triethylamine (52 μL, 0.38 mmol), benzotriazol-1-yloxytris(dimethylamino)-phosphonium hexafluorophosphate (159 mg, 0.35 mmol) and 2-amino-2-methyl-1-propanol (49 μL, 0.51 mmol) according to a procedure described in Example 93 and furnished the title compound as a white solid. M.P.: 181-184° C. ¹H-NMR (δ ppm, CDCl₃, 300 MHz): 7.62 (d, J=9.0, 1H); 7.44 (d, J=9.0, 2H); 6.98 (br. s, 1H); 5.03 (t, J=6.0, 1H); 3.71 (br. s, 2H); 3.69 (br. s, 2H); 2.12 (d, J=8.4, 1H); 2.06-1.95 (m, 2H); 1.73 (d, J=8.4, 1H); 1.41 (s, 6H); 1.29-1.20 (m, 2H). IR (cm⁻¹, KBr): 3325 (m), 3099 (w), 3001 (m), 2969 (m), 2950 (m), 2864 (m), 1635 (s), 1593 (m), 1551 (s), 1503 (s), 1491 (s), 1438 (w), 1404 (w), 1388 (w), 1354 (m), 1276 (m), 1257 (m), 1171 (m), 1122 (m), 1087 (m), 1072 (m), 1025 (w), 1006 (w), 869 (m) 836 (m). MS (m/z): 360.37 ([M+H]⁺).

### Example 97

### Preparation of (1R,7S)- or (1S,7R)-N5-(2-Hydroxy-1,1-dimethylethyl)-3-(4-chlorophenyl)-3,4-diazatricyclo[5.2.1.0^{2,6}]deca-2(6),4-diene-5-carboxamide

This compound was prepared from intermediate 35 (70 mg, 0.24 mmol), dry dimethyl formamide (1-5 mL), triethylamine (37 μL, 0.26 mmol), benzotriazol-1-yloxytris(dimethylamino)-phosphonium hexafluorophosphate (112 mg, 0.25 mmol) and 2-amino-2-methyl propanol (35 μL, 0.36 mmol) according to a procedure described in Example 93 and furnished the title compound as a white solid. M.P.: 167-169° C. ¹H-NMR (δ ppm, CDCl₃, 300 MHz): 7.63 (d, J=8.4, 2H); 7.45 (d, 8.4, 2H); 5.04 (t, J=6.6, 1H); 3.72 (m, 2H); 3.70 (br. s, 2H); 2.13 (d, J=7.8, 1H); 2.01 (d, J=7.2, 2H); 1.73 (d, J=9.0, 1H); 1.41 (s, 6H); 1.30-1.21 (m, 2H). IR (cm⁻¹, KBr): 3332 (s), 3097 (m), 2999 (m), 2967 (m), 2868 (m), 1640 (s), 1593 (w), 1551 (s), 1502 (s), 1449 (s), 1405 (w), 1388 (w), 1368 (w), 1353 (m), 1277 (m), 1256 (m), 1171 (m), 1122 (m), 1087 (m), 1072 (m), 1025 (m), 953 (m), 869 (m), 836 (m). MS (m/z): 360.25 ([M+H]⁺).

### Example 98

### Preparation of (1S,7R)- or (1R,7S)-N5-(2-Hydroxy-1,1-dimethylethyl)-3-(4-chlorophenyl)-3,4-diazatricyclo[5.2.1.0^{2,6}]deca-2(6),4-diene-5-carboxamide

This compound was prepared from intermediate 36 (70 mg, 0.32 mmol), dry dimethyl formamide (1-5 mL), triethylamine (37 μL, 0.26 mmol), benzotriazol-1-yloxytris(dimethylamino)-phosphonium hexafluorophosphate (113 mg, 0.25 mmol) and 2-amino-2-methyl propanol (35 μL, 0.36 mmol) according to a procedure described in Example 93 and furnished the title compound as a white solid. M.P.: 166-168° C. ¹H-NMR (δ ppm, CDCl₃, 300 MHz): 7.62 (d, J=8.4, 2H); 7.44 (d, 8.7, 2H); 5.04 (t, J=6.6, 1H); 3.71 (m, 2H); 3.69 (br. s, 2H); 2.12 (d, J=8.1, 1H); 2.01 (d, J=7.2, 2H); 1.72 (d, J=8.7, 1H); 1.41 (s, 6H); 1.30-1.21 (m, 2H). IR (cm⁻¹, KBr): 3331 (m), 3097 (w), 2999 (w), 2967 (m), 2869 (w), 1640 (s), 1593 (w), 1551 (s), 1503 (s), 1492 (s), 1449 (w), 1404 (w), 1388 (w), 1353 (m), 1277 (m), 1256 (m), 1171 (w), 1122 (w), 1087 (m), 1072 (m), 1007 (m), 869 (m), 836 (m). MS (m/z): 360.53 ([M+H]⁺).

### Example 99

### Preparation of N5-(2-Hydroxy-1,1-dimethylethyl)-3-(4-bromophenyl)-3,4-diazatricyclo[5.2.1.0^{2,6}]deca-2(6),4-diene-5-carboxamide

This compound was prepared from intermediate 46 (100 mg, 0.30 mmol), dry dimethyl formamide (1-5 mL), triethylamine (47 μL, 0.33 mmol), benzotriazol-1-yloxytris(dimethylamino)-phosphonium hexafluorophosphate (140 mg, 0.31 mmol) and 2-amino-2-methyl-1-propanol (43 μL, 0.45 mmol) according to a procedure described in Example 93 and furnished the title compound as white solid. M.P.: 184-186° C. ¹H-NMR (δ ppm, CDCl₃, 300 MHz): 7.62-7.52 (m, 4H); 6.98 (br. s, 1H); 5.05 (t, J=6.3, 1H); 3.71 (br. s, 2H); 3.69 (br. s, 2H); 2.12 (d, J=9.0, 1H); 2.06-1.95 (m, 2H); 1.72 (d, J=8.7, 1H); 1.41 (s, 6H); 1.30-1.20 (m, 2H). IR (cm⁻¹, KBr): 3407 (m), 3377 (m), 3054 (m), 2976 (m), 2937 (m), 2870 (w), 1651 (m), 1608 (w), 1548 (m), 1523 (s), 1492 (m), 1447 (m), 1372 (m), 1273 (w), 1146 (m), 1093 (w), 1061 (m), 965 (m), 827 (m). MS (m/z): 406.41 ([M+H]⁺).

### Example 100

### Preparation of N5-(2-Hydroxy-1,1-dimethylethyl)-3-(4-chloro-2-fluorophenyl)-3,4-diazatricyclo[5.2.1.0^{2,6}]deca-2(6),4-diene-5-carboxamide

This compound was prepared from intermediate 38 (100 mg, 0.32 mmol), dry dimethyl formamide (1-5 mL), triethylamine (51 μL, 0.35 mmol), benzotriazol-1-yloxytris(dimethylamino)-phosphonium hexafluorophosphate (150 mg, 0.34 mmol) and 2-amino-2-methyl propanol (40 μL, 0.42 mmol) according to a procedure described in Example 93 and furnished the title compound as a white solid. M.P.: 158-160° C. ¹H-NMR (δ ppm, CDCl₃, 300 MHz): 7.64 (t, J=8.1, 1H); 7.32-7.24 (m, 2H); 6.94 (br. s, 1H); 5.02 (t, J=6.0, 1H); 3.71 (br. s, 3H); 3.46 (br. s, 1H); 2.08 (d, J=9.0, 1H); 2.04-1.80 (m, 2H); 1.69 (d, J=9.0, 1H); 1.40 (s, 6H); 1.28-1.20 (m, 2H). IR (cm⁻¹, KBr): 3390 (s), 3350 (s), 3083 (w), 2968 (m), 2937 (m), 2866 (s), 1633 (s), 1590 (m), 1554 (m), 1511 (m), 1444 (m), 1406 (w), 1386 (w), 1356 (m), 1278 (m), 1225 (m), 1167 (m), 1108 (m), 1077 (m), 1068 (m), 984 (m). MS (m/z): 378.32 ([M+H]⁺).

### Example 101

### Preparation of N5-(2-Hydroxy-1,1-dimethylethyl)-3-(2,4,6-trifluorophenyl)-3,4diazatricyclo[5.2.1.0^{2,6}]deca-2(6),4-diene-5-carboxamide

This compound was prepared from intermediate 42 (100 mg, 0.32 mmol), dry dimethyl formamide (1-5 mL), triethylamine (51 μL, 0.35 mmol), benzotriazol-1-yloxytris(dimethylamino)-phosphonium hexafluorophosphate (150 mg, 0.34 mmol) and 2-amino-2-methyl propanol (37 μL, 0.38 mmol) according to a procedure described in Example 93 and furnished the title compound as a white solid. M.P.: 122-125° C. ¹H-NMR (δ ppm, CDCl₃, 300 MHz): 6.91-6.82 (m, 3H); 5.05 (t, J=6.0, 1H); 3.74 (br. s, 1H); 3.69 (d, J=6.0, 2H); 3.28 (br. s, 1H); 2.11 (d, J=7.5, 1H); 2.06-1.85 (m, 2H); 1.69 (d, J=8.4, 1H); 1.38 (s, 6H); 1.30-1.21 (m, 2H). IR (cm⁻¹, KBr): 3435 (s), 3390 (s), 3071 (w), 2968 (m), 2949 (m), 2870 (s), 1654 (s), 1607 (m), 1551 (m), 1537 (s), 1496 (m), 1450 (m), 1372 (w), 1343 (w), 1278 (w), 1253 (m), 1182 (m), 1163 (m), 1133 (m), 1119 (m), 1063 (m), 1039 (m), 998 (m), 871 (m), 844 (m). MS (m/z): 380.45 ([M+H]⁺).

### Example 102

### Preparation of (1R,7S)- or (1S,7R)-N5-(tert-Butyl)-3-(4-bromophenyl)-3,4-diazatricyclo[5.2.1.0^{2,6}]deca-2(6),4-diene-5-carboxamide

This compound was prepared from intermediate 47 (75 mg, 0.22 mmol), dry dimethyl formamide (1-5 mL), triethylamine (34 μL, 0.24 mmol), benzotriazol-1-yloxytris(dimethylamino)-phosphonium hexafluorophosphate (102 mg, 0.24 mmol) and tert-butyl amine (36 μL, 0.33 mmol) according to a procedure described in Example 93 and furnished the title compound as a white solid. M.P.: 148-149° C. ¹H-NMR (δ ppm, CDCl₃, 300 MHz): 7.57 (s, 4H); 6.78 (br. s, 1H); 3.75 (br. s, 1H); 3.66 (br. s, 1H); 2.12 (d, J=8.4, 1H); 1.98 (d, J=8.1, 2H); 1.71 (d, J=9.0, 1H); 1.48 (s, 9H); 1.30-1.18 (m, 2H). IR (cm⁻¹, KBr): 3331 (m), 3070 (m), 2968 (m), 2955 (w), 2922 (m), 2868 (m), 1655 (s), 1590 (w), 1547 (s), 1499 (s), 1453 (w), 1437 (w), 1400 (w), 1351 (m), 1276 (m), 1253 (m), 1214 (m), 1174 (m), 1005 (m), 834 (m). MS (m/z): 388.49 ([M+H]⁺).

### Example 103

### Preparation of (1S,7R)- or (1R,7S)-N5-(tert-Butyl)-3-(4-bromophenyl)-3,4-diazatricyclo[5.2.1.0^{2,6}]deca-2(6),4-diene-5-carboxamide

This compound was prepared from intermediate 48 (100 mg, 0.30 mmol), dry dimethyl formamide (1-5 mL), triethylamine (47 μL, 0.33 mmol), benzotriazol-1-yloxytris(dimethylamino)-phosphonium hexafluorophosphate (140 mg, 0.31 mmol) and tert-butyl amine (47 μL, 0.45 mmol) according to a procedure described in Example 93 and furnished the title compound as a white solid. M.P.: 149-151° C. ¹H-NMR (δ ppm, CDCl₃, 300 MHz): 7.57 (s, 4H); 6.78 (br. s, 1H); 3.75 (br. s, 1H); 3.66 (br. s, 1H); 2.11 (d, J=7.2, 1H); 1.96 (d, J=8.1, 2H); 1.70 (d, J=8.7, 1H); 1.48 (s, 9H); 1.30-1.18 (m, 2H). IR (cm⁻¹, KBr): 3331 (m), 3070 (w), 2968 (m), 2955 (m), 2869 (w), 1656 (s), 1590 (w), 1547 (m), 1499 (s), 1489 (s), 1400 (w), 1351 (m), 1310 (w), 1276 (m), 1254 (m), 1214 (m), 1162 (w), 1074 (w), 1005 (w), 868 (w), 833 (m). MS (m/z): 388.49 ([M+H]⁺).

### Example 104

### Preparation of (1R,7S)- or (1S,7R)-N5-(tert-Butyl)-3-(4-chlorophenyl)-3,4-diazatricyclo[5.2.1.0^{2,6}]deca-2(6),4-diene-5-carboxamide

This compound was prepared from intermediate 35 (100 mg, 0.34 mmol), dry dimethyl formamide (1-5 mL), triethylamine (53 μL, 0.37 mmol), benzotriazol-1-yloxytris(dimethylamino)-phosphonium hexafluorophosphate (161 mg, 0.35 mmol) and tert-butyl amine (55 μL, 0.51 mmol) according to a procedure described in Example 93 and furnished the title compound as a white solid. M.P.: 159-162° C. ¹H-NMR (δ ppm, CDCl₃, 300 MHz): 7.62 (d, J=8.4, 2H); 7.43 (d, J=8.4, 2H); 6.79 (br. s, 1H); 3.75 (br. s, 1H); 3.65 (br. s, 1H); 2.11 (d, J=8.7, 1H); 1.98 (d, J=8.4, 2H); 1.70 (d, J=8.7, 1H); 1.48 (s, 9H); 1.30-1.16 (m, 2H). IR (cm⁻¹, KBr): 3333 (m), 3073 (w), 2966 (m), 2968 (m), 1655 (s), 1594 (s), 1547 (m), 1502 (m), 1489 (s), 1443 (m), 1406 (w), 1390 (w), 1359 (m), 1276 (m), 1256 (m), 1218 (m), 1120 (m), 1087 (m), 836 (m). MS (m/z): 344.42 ([M+H]⁺).

### Example 105

### Preparation of (1S,7R)- or (1R,7S)-N5-(tert-Butyl)-3-(4-chlorophenyl)-3,4-diazatricyclo[5.2.1.0^{2,6}]deca-2(6),4-diene-5-carboxamide

This compound was prepared from intermediate 36 (87 mg, 0.30 mmol), dry dimethyl formamide (1-5 mL), triethylamine (47 μL, 0.33 mmol), benzotriazol-1-yloxytris(dimethylamino)-phosphonium hexafluorophosphate (140 mg, 0.31 mmol) and tert-butyl amine (48 μL, 0.45 mmol) according to a procedure described in Example 93 and furnished the title compound as a white solid. M.P.: 158-160° C. ¹H-NMR (δ ppm, CDCl₃, 300 MHz): 7.62 (d, J=8.4, 2H); 7.43 (d, J=8.7, 2H); 6.78 (br. s, 1H); 3.75 (br. s, 1H); 3.66 (br. s, 1H); 2.11 (d, J=8.1, 1H); 1.98 (d, J=7.8, 2H); 1.71 (d, J=8.7, 1H); 1.48 (s, 9H); 1.32-1.15 (m, 2H). IR (cm⁻¹, KBr): 3333 (m), 3073 (w), 2966 (m), 2968 (m), 1655 (s), 1594 (s), 1547 (m), 1502 (m), 1489 (s), 1443 (m), 1406 (w), 1390 (w), 1359 (m), 1276 (m), 1256 (m), 1218 (m), 1120 (m), 1087 (m), 836 (m). MS (m/z): 344.45 ([M+H]⁺).

### Example 106

### Preparation of N5-(tert-Butyl)-3-[2,4-difluorophenyl]-3,4-diazatricyclo[5.2.1.0^{2,6}]deca-2(6),4-diene-5-carboxamide

The title compound was prepared from intermediate 3 (100 mg, 0.34 mmol), dimethylformamide (1-5 mL), triethylamine (57 μL, 0.40 mmol), benzotriazol-1-yloxytris(dimethylamino)-phosphonium hexafluorophosphate (165 mg, 0.37 mmol) and 2-amino-2-methylpropane (36 μL, 0.34 mmol) according to a procedure described in Example 93 and furnished the title compound. M.P.: 109-111° C. ¹H-NMR (δ ppm, CDCl₃, 300 MHz): 7.71-7.62 (m, 1H), 7.04-6.97 (m, 2H); 6.74 (br. s, 1H); 3.75 (br. s, 1H); 3.43 (br. s, 1H); 2.10-2.04 (m, 1H); 2.01-1.90 (m, 2H); 1.67 (d, J=8.4, 1H); 1.46 (s, 9H); 1.33-1.19 (m, 2H). IR (cm⁻¹, KBr): 3323 (m), 2968 (m), 1652 (s), 1609 (s), 1547 (s), 1522 (s), 1495 (w), 1448 (m), 1391 (w), 1360 (m), 1272 (m) 1258 (w), 1145 (w), 1109 (m), 965 (m), 849 (m). MS (m/z): 346.0(M+H⁺).

### Example 107

### Preparation of N5-(tert-Butyl)-3-[4-Nitrophenyl]-3,4-diazatricyclo[5.2.1.0^{2,6}]deca-2(6),4-diene-5-carboxamide

This compound was prepared from intermediate 50 (120 mg, 0.40 mmol), dry dimethyl formamide (1-5 mL), triethylamine (66 μL, 0.48 mmol), benzotriazol-1-yloxytris(dimethylamino)-phosphonium hexafluorophosphate (194 mg, 0.44 mmol) and tert-butyl amine (46 μL, 0.44 mmol) according to a procedure described in Example 93 and furnished the title compound as a yellow solid. M.P.: 224-226° C. ¹H-NMR (δ ppm, CDCl₃, 300 MHz): 8.35 (d, J=8.7, 2H); 7.88 (d, J=9.0, 2H); 6.78 (br. s, 1H); 3.77 (br. s, 2H); 2.15 (d, J=8.4, 1H); 2.03 (d, J=8.1, 2H); 1.74 (d, J=9.0, 1H); 1.49 (s, 9H); 1.24 (d, J=7.8, 2H). IR (cm⁻¹, KBr): 3365 (m), 3080 (w), 2941 (m), 2965 (m), 1660 (s), 1594 (s), 1547 (m), 1522 (m), 1487 (s), 1455 (w), 1332 (s), 1276 (m), 1257 (m), 1216 (w), 1104 (m), 853 (m). MS (m/z): 355.26 ([M+H]⁺).

### Example 108

### Preparation of N5-(tert-Butyl)-3-[4-aminophenyl]-3,4-diazatricyclo[5.2.1.0^{2,6}]deca-2(6),4-diene-5-carboxamide

Example 107 (340 mg, 0.96 mmol) and 10% palladium/carbon (40-50 mg) were taken in methanol and hydrogenated for about 90 minutes at about 40 PSI. The reaction mixture was then filtered through celite and washed with methanol and concentrated under reduced pressure and the crude product was used in the next step. ¹H-NMR (δ ppm, CDCl₃, 300 MHz): 7.42 (d, J=8.4, 2H); 6.80 (br. s, 1H); 6.74 (d, J=9.0, 2H); 3.73 (br. s, 1H); 3.57 (br. s, 1H); 2.08 (d, J=8.7, 1H); 2.00-1.90 (m, 2H); 1.67 (d, J=8.4, 1H); 1.46 (s, 9H); 1.28-1.16 (m, 2H).

### Example 109

### Preparation of N1-{4-[5-(tert-Butylcarbamoyl)-3,4-diazatricyclo[5.2.1.0^{2,6}]deca-2(6),4-dien-3-yl]phenyl}acetamide

Example 108 (75 mg, 0.23 mmol) in dry pyridine (1-5 mL) was treated at about 0° C. with acetic anhydride (24 μL, 0.25 mmol) and catalytic amount of 4-Dimethylaminopyridine (1 mg) and the reaction mixture was stirred at an ambient temperature for about 1 hour. The reaction mixture was poured into 1N hydrogen chloride and extracted with ethyl acetate and the combined organic layers were washed with water, brine and dried over sodium sulphate. After removal of the solvent, the residue was purified on silicondioxide column chromatography to furnish the title compound as pale yellow solid. M.P.: 122-124° C. ¹H-NMR (δ ppm, CDCl₃, 300 MHz): 7.62 (s, 4H); 7.38 (br. s, 1H); 6.81 (br. s, 1H); 3.74 (br. s, 1H); 3.65 (br. s, 1H); 2.21 (s, 3H); 2.09 (d, J=9.0, 1H); 2.02-1.90 (m, 2H); 1.69 (d, J=8.4, 1H); 1.47 (s, 9H); 1.29-1.15 (m, 2H). IR (cm⁻¹, KBr): 3401 (w), 3277 (m), 3138 (w), 3076 (w), 2966 (m), 2870 (m), 1656 (s), 1608 (w), 1547 (s), 1519 (s), 1445 (w), 1409 (w), 1365 (m), 1310 (m), 1256 (m), 1217 (m), 1108 (m), 833 (m). MS (m/z): 367.10 ([M+H]⁺).

### Example 110

### Preparation of N1-{4-[5-(tert-Butylcarbamoyl)-3,4-diazatricyclo[5.2.1.0^{2,6}]deca-2(6),4-dien-3-yl]phenyl}carbamic acid ethyl ester

This compound was prepared from Example 108 (75 mg, 0.30 mmol), dry pyridine (1 ml) and ethyl chloroformate (30 μL, 0.32 mmol) according to a procedure described in example 109 and furnished the title compound as yellow solid. M.P.: 103-104° C. ¹H-NMR (δ ppm, CDCl₃, 300 MHz): 7.60 (d, J=8.7, 2H); 7.48 (d, J=8.7, 2H); 6.80 (br. s, 1H); 6.68 (br. s, 1H); 4.22 (q, J=7.2, 2H); 3.76 (br. s, 1H); 3.65 (br. s, 1H); 2.08 (d, J=8.7, 1H); 2.02-1.90 (m, 2H); 1.69 (d, J=8.7, 1H); 1.46 (s, 9H); 1.26 (s, 3H); 1.29-1.20 (m, 2H). IR (cm⁻¹, KBr): 3397 (w), 3288 (s), 3137 (w), 3072 (w), 2966 (m), 1731 (m), 1658 (s), 1544 (m), 1524 (s), 1445 (s), 1311 (s), 1277 (m), 1222 (s), 1065 (m), 838 (m). MS (m/z): 397.41 ([M+H]⁺).

### Example 111

### Preparation of N5,N5-Diisopropyl-3-(2,4-difluorophenyl)-3,4-diazatricyclo[5.2.1.0^{2,6}]deca-2(6),4-diene-5-carboxamide

The intermediate 3 (150 mg, 0.46 mmol) in dichloromethane (10-20 mL) was treated with catalytic amount of dry dimethyl formamide and cooled in an ice bath. Oxalyl chloride (179 μL, 2.06 mmol) was added dropwise and the reaction mixture was stirred at an ambient temperature for about 3 hours. The solvent was evaporated and the residue dissolved in dichloromethane (5-10 mL) and added dropwise to a cold solution of diisopropyl amine (480 μL, 3.44 mmol) and triethylamine (285 μL, 2.06 mmol) in dichloromethane (5-10 mL) and the reaction mixture was stirred at an ambient temperature for about 1 hour. The reaction mixture was washed with water, brine and dried over sodium sulphate. The purification of the crude product by silicondioxide column chromatography furnished the title compound as an off-white solid. M.P.: 107-109° C. ¹H-NMR (δ ppm, CDCl₃, 300 MHz): 7.67 (q, J=9.0, 1H); 7.97 (t, J=9.0, 2H); 4.70 (br. s, 1H); 3.53 (br. s, 2H); 3.47 (br. s, 1H); 2.07 (d, J=9.0, 1H); 2.04-1.89 (m, 2H); 1.68-1.60 (m, 1H), 1.53 (s, 12H), 1.32-1.20 (m, 2H). IR (cm⁻¹, KBr): 3395 (m), 3084 (w), 2962 (s), 2847 (s), 2902 (s), 1671 (s), 1607 (w), 1513 (s), 1486 (m), 1454 (m), 1386 (m), 1361 (m), 1258 (m), 1220 (m), 1162 (m), 1096 (m), 1085 (s), 1033 (m), 1013 (m), 869 (m), 837 (m). MS (m/z): 374.33 ([M+H]⁺).

### Example 112

### Preparation of N5-(1,1-Dimethylhexyl)-3-(2,4-difluorophenyl)-3,4-diazatricyclo[5.2.1.0^{2,6}]deca-2(6),4-diene-5-carboxamide

Triethylamine (114 μL, 0.81 mmol) and benzotriazol-1-yloxytris(dimethylamino)-phosphonium hexafluorophosphate (160 mg, 0.36 mmol) were added to intermediate 3 (100 mg, 0.34 mmol) in dry dimethyl formamide (1.0-5.0 mL). After stirring at an ambient temperature for about 30 minutes and 1,1-dimethyl-n-hexylamine (86 mg, 0.51 mmol) was added. The reaction mixture was stirred overnight, diluted with water, extracted with ethyl acetate, washed with brine and dried over sodium sulphate. Evaporation of the solvent and purification of the crude product by silicon dioxide column chromatography to furnished the N5-(1,1-Dimethylhexyl)-3-(2,4-difluorophenyl)-3,4-diazatricyclo[5.2.1.0^{2,6}]deca-2(6),4-diene-5-carboxamide as an off-white solid. M.P.: 76-78° C. ¹H-NMR (δ ppm, CDCl₃, 300 MHz): 7.67 (q, J=9.0, 1H); 7.05-6.92 (m, 2H); 6.67 (br. s, 1H); 3.74 (br. s, 1H); 3.43 (br. s, 1H); 2.07 (d, J=9.0, 1H); 2.04-1.92 (m, 2H); 1.67 (d, J=8.1, 1H); 1.42 (s, 6H); 1.41-1.20 (m, 8H). IR (cm⁻¹, KBr): 3331 (m), 3092 (w), 2953 (m), 2933 (m), 2859 (m), 1656 (s), 1609 (m), 1520 (s), 1495 (s), 1469 (m), 1455 (m), 1386 (w), 1360 (m), 1271 (m), 1256 (m), 1239 (w), 1146 (m), 1121 (m), 1110 (m), 1092 (m), 963 (m), 857 (m). MS (m/z): 402.18 ([M+H]⁺).

### Example 113

### Preparation of N5-(2,2,2-trifluoroethyl)-3-(2,4-diflurophenyl)-3,4-diazatricyclo[5.2.1.0^{2,6}]deca-2(6),4-diene-5-carboxamide

This compound was prepared from intermediate 3 (100 mg, 0.34 mmol), dry dimethyl formamide (1-5 mL), triethylamine (52 μL, 0.37 mmol), benzotriazol-1-yloxytris(dimethylamino)-phosphonium hexafluorophosphate (159 mg, 0.35 mmol) and 2,2,2-trifluroethylamine (41 μL, 0.51 mmol) according to a procedure described in Example 93 and furnished the title compound as white solid. M.P.: 149-152° C. ¹H-NMR (δ ppm, CDCl₃, 300 MHz): 7.66 (q, J=8.4, 1H); 7.13 (br. s, 1H); 7.03 (t, J=8.1, 2H); 4.08 (q, J=6.9, 2H); 3.75 (br. s, 2H); 3.46 (br. s, 1H); 2.09 (d, J=8.4, 1H); 1.96 (d, J=8.4, 2H); 1.69 (d, J=9.0, 1H); 1.29-1.20 (m, 2H). IR (cm⁻¹, KBr): 3341 (m), 3087 (m), 2964 (m), 2872 (w), 1663 (s), 1609 (w), 1560 (s), 1520 (s), 1500 (m), 1401 (w), 1356 (w), 1284 (m), 1270 (m), 1234 (m), 1159 (s), 1124 (m), 1092 (m), 1063 (w), 1038 (w), 962 (m), 868 (m). MS (m/z): 372.30 ([M+H]⁺).

### Example 114

### Preparation of N5-(tert-Butyl)-3-(4-methylphenyl)-3,4-diazatricyclo[5.2.1.0^{2,6}]deca-2(6),4-diene-5-carboxamide

This compound was prepared from intermediate 52 (100 mg, 0.41 mmol), dry dimethyl formamide (1-5 mL) and triethylamine (65 μL, 0.45 mmol) and benzotriazol-1-yloxytris(dimethylamino)-phosphonium hexafluorophosphate (193 mg, 0.43 mmol) and tert-butyl amine (66 μL, 0.62 mmol) according to a procedure described in Example 93 and furnished the title compound as a white solid. M.P.: 135-137° C. ¹H-NMR (δ ppm, CDCl₃, 300 MHz): 7.54 (d, J=8.1, 2H); 7.26 (br. s, 2H); 6.82 (br. s, 1H); 3.75 (br. s, 1H); 3.65 (br. s, 1H); 2.39 (s, 3H); 2.07 (d, J=9.0, 1H); 2.02-1.90 (m, 2H); 1.69 (d, J=9.0, 1H); 1.47 (s, 9H); 1.32-1.20 (m, 2H). IR (cm⁻¹, KBr): 3324 (m), 2992 (m), 2966 (m), 2923 (w), 1651 (s), 1613 (w), 1543 (m), 1521 (s), 1493 (m), 1445 (m), 1355 (m), 1275 (m), 1256 (w), 1225 (w), 1106 (w), 868 (w), 811 (w).

### Example 115

### Preparation of N5-(tert-Butyl)-3-(4-trifluoromethyphenyl)-3,4-diazatricyclo[5.2.1.0^{2,6}]deca-2(6),4-diene-5-carboxamide

This compound was prepared from intermediate 54 (100 mg, 0.31 mmol), dry dimethyl formamide (1-5 mL), triethylamine (48 μL, 0.34 mmol), benzotriazol-1-yloxytris(dimethylamino)-phosphonium hexafluorophosphate (144 mg, 0.32 mmol) and tert-butyl amine (49 μL, 0.46 mmol) according to a procedure described in Example 93 and furnished the title compound as an off-white solid. M.P.: 174-176° C. ¹H-NMR (δ ppm, CDCl₃, 300 MHz): 7.82 (d, J=8.4, 2H); 7.72 (d, J=8.4, 2H); 6.80 (br. s, 1H); 3.76 (br. s, 1H); 3.72 (br. s, 1H); 2.13 (d, J=9.0, 1H); 2.00 (d, J=9.0, 2H); 1.72 (d, J=9.0, 1H); 1.48 (s, 9H); 1.32-1.20 (m, 2H). IR (cm⁻¹, KBr): 3344 (m), 3080 (w), 2967 (m), 2950 (m), 2933 (m), 2868 (m), 1664 (s), 1614 (m), 1525 (m), 1494 (m), 1443 (m), 1325 (s), 1276 (m), 1163 (s), 1129 (s), 1106 (m), 1068 (s), 869 (m), 857 (m). MS (m/z): 378.49 ([M+H]⁺).

### Example 116

### Preparation of N5-(tert-Butyl)-3-[4-(tert-butyl)phenyl]-3,4-diazatricyclo[5.2.1.0^{2,6}]deca-2(6),4-diene-5-carboxamide

This compound was prepared from intermediate 56 (120 mg, 0.38 mmol), dry dimethyl formamide (1-5 mL), triethylamine (64 μL, 0.46 mmol), benzotriazol-1-yloxytris(dimethylamino)-phosphonium hexafluorophosphate (188 mg, 0.42 mmol) and tert-butylamine (44 μL, 0.42 mmol) according to a procedure described in Example 93 and furnished the title compound as an off-white solid. M.P.: 148-150° C. ¹H-NMR (δ ppm, CDCl₃, 300 MHz): 7.58 (d, J=8.7, 2H); 7.47 (d, J=9.0, 2H); 6.83 (br. s, 1H); 3.75 (br. s, 1H); 3.65 (br. s, 1H); 2.15-2.06 (m, 1H); 2.00-1.92 (m, 2H); 1.69 (d, J=8.1, 1H); 1.47 (s, 9H); 1.35 (s, 9H); 1.30-1.20 (m, 2H). IR (cm⁻¹, KBr): 3397 (w), 2960 (m), 2871 (m), 1673 (s), 1609 (w), 1547 (s), 1523 (s), 1497 (m), 1447 (m), 1364 (m), 1346 (m), 1163 (w), 1277 (m), 1254 (m), 1228 (m), 1219 (m), 1107 (m), 842 (m). MS (m/z): 366.26 ([M+H]⁺).

### Example 117

### Preparation of N5-(tert-Butyl)-3-(phenyl)-3,4-diazatricyclo[5.2.1.0^{2,6}]deca-2(6),4-diene-5-carboxamide

This compound was prepared from intermediate 119 (51 mg, 0.20 mmol), dry dimethyl formamide (1-5 mL), triethylamine (31 μL, 0.22 mmol), benzotriazol-1-yloxytris(dimethylamino)-phosphonium hexafluorophosphate (93 mg, 0.21 mmol) and tert-butyl amine (32 μL, 0.30 mmol) according to a procedure described in Example 93 and furnished the title compound as an off-white solid. M.P.: 101-103° C. ¹H-NMR (δ ppm, CDCl₃, 300 MHz): 7.67 (d, J=9.0, 2H); 7.47 (t, J=6.0, 2H); 7.34-7.27 (m, 1H); 6.83 (br. s, 1H); 3.76 (br. s, 1H); 3.69 (br. s, 1H); 2.11 (d, J=9.0, 1H); 1.97 (d, J=9.0, 2H); 1.70 (d, J=9.0, 1H); 1.48 (s, 9H); 1.32-1.20 (m, 2H). IR (cm⁻¹, KBr): 3325 (m), 3065 (w), 2966 (m), 2958 (m), 2924 (m), 2861 (w), 1654 (s), 1599 (m), 1547 (m), 1509 (m), 1491 (m), 1450 (m), 1357 (m), 1276 (m), 1257 (m), 1228 (m), 1120 (w), 869 (w). MS (m/z): 310.36 ([M+H]⁺).

### Example 118

### Preparation of N5-(tert-Butyl)-3-[4-methoxyphenyl]-3,4-diazatricyclo[5.2.1.0^{2,6}]deca-2(6),4-diene-5-carboxamide

This compound was pre[pared from intermediate 44 (100 mg, 0.35 mmol), dry dimethyl formamide (1-5 mL), triethylamine (53 μL, 0.38 mmol), benzotriazol-1-yloxytris(dimethylamino)-phosphonium hexafluorophosphate (162 mg, 0.36 mmol) and tert-butylamine (55 μL, 0.52 mmol) according to a procedure described in Example 93 and furnished the title compound as an off-white solid. M.P.: 145-147° C. ¹H-NMR (δ ppm, CDCl₃, 300 MHz): 7.57 (d, J=8.7, 2H); 6.98 (d, J=8.7, 2H); 6.81 (br. s, 1H); 3.85 (s, 3H); 3.75 (br. s, 1H); 3.61 (br. s, 1H); 2.14-2.06 (m, 1H); 2.04-1.90 (m, 2H); 1.69 (d, J=8.1, 1H); 1.47 (s, 9H); 1.28-1.20 (m, 2H). IR (cm⁻¹, KBr): 3409 (m), 3077 (w), 2983 (m), 2863 (m), 2866 (m), 1675 (m), 1610 (w), 1590 (w), 1549 (m), 1518 (s), 1491 (s), 1445 (m), 1389 (w), 1363 (w), 1354 (w), 1276 (w), 1251 (m), 1214 (m), 1168 (m), 1101 (w), 868 (w), 834 (m).

### Example 119

### Preparation of N5-(tert-Butyl)-3-(4-chloro-2-fluorophenyl)-3,4-diazatricyclo[5.2.1.0^{2,6}]deca-2(6),4-diene-5-carboxamide

This compound was prepared from intermediate 38 (100 mg, 0.32 mmol), dry dimethyl formamide (1-5 mL), triethylamine (50 μL, 0.35 mmol), benzotriazol-1-yloxytris(dimethylamino)-phosphonium hexafluorophosphate (151 mg, 0.34 mmol) and tert-butyl amine (41 μL, 0.39 mmol) according to a procedure described in Example 93 and furnished the title compound as a white solid. M.P.: 170-173° C. ¹H-NMR (δ ppm, CDCl₃, 300 MHz): 7.65 (t, J=8.1, 1H); 7.32-7.24 (m, 2H); 6.74 (br. s, 1H); 3.74 (br. s, 1H); 3.45 (br. s, 1H); 2.07 (d, J=8.4, 1H); 2.02-1.85 (m, 2H); 1.67 (d, J=8.4, 1H); 1.46 (s, 9H); 1.34-1.20 (m, 2H). IR (cm⁻¹, KBr): 3447 (w), 3325 (m), 3028 (w), 2970 (m), 2928 (m), 2872 (m), 1651 (s), 1612 (w), 1589 (m), 1545 (m), 1513 (s), 1505 (s), 1446 (m), 1358 (m), 1278 (m), 1227 (m), 1078 (m), 894 (m). MS (m/z): 362.30 ([M+H]⁺).

### Example 120

### Preparation of (1R,7S)- or (1S,7R)-N5-(tert-Butyl)-3-(4-chloro-2-fluorophenyl)-3,4-diazatricyclo[5.2.1.0^{2,6}]deca-2(6),4-diene-5-carboxamide

This compound was prepared from intermediate 39 (100 mg, 0.32 mmol), dry dimethyl formamide (1-5 mL), triethylamine (42 μL, 0.35 mmol), benzotriazol-1-yloxytris(dimethylamino)-phosphonium hexafluorophosphate (152 mg, 0.34 mmol) and tert-butyl amine (42 μL, 0.39 mmol) according to a procedure described in Example 93 and furnished the title compound as a white solid. M.P.: 148-152° C. ¹H-NMR (δ ppm, CDCl₃, 300 MHz): 7.65 (t, J=8.4, 1H); 7.33-7.24 (m, 2H); 6.74 (br. s, 1H); 3.74 (br. s, 1H); 3.45 (br. s, 1H); 2.07 (d, J=7.8, 1H); 2.04-1.85 (m, 2H); 1.67 (d, J=8.7, 1H); 1.47 (s, 9H); 1.30-1.20 (m, 2H). IR (cm⁻¹, KBr): 3435 (w), 3334 (m), 3080 (w), 2968 (m), 2874 (m), 1653 (s), 1612 (w), 1590 (m), 1545 (m), 1506 (s), 1490 (m), 1359 (m), 1278 (m), 1227 (m), 1078 (m), 894 (m). MS (m/z): 362.30 ([M+H]⁺).

### Example 121

### Preparation of (1S,7R)- or (1R,7S)-N5-(tert-Butyl)-3-(4-chloro-2-fluorophenyl)-3,4-diazatricyclo[5.2.1.0^{2,6}]deca-2(6),4-diene-5-carboxamide

This compound was prepared from intermediate 40 (100 mg, 0.32 mmol), dry dimethyl formamide (1-5 mL), triethylamine (42 μL, 0.35 mmol), benzotriazol-1-yloxytris(dimethylamino)-phosphonium hexafluorophosphate (152 mg, 0.34 mmol) and tert-butyl amine (42 μL, 0.39 mmol) according to a procedure described in Example 93 and furnished the title compound as a white solid. M.P.: 148-151° C. ¹H-NMR (δ ppm, CDCl₃, 300 MHz): 7.63 (t, J=8.7, 1H); 7.32-7.24 (m, 2H); 6.72 (br. s, 1H); 3.73 (br. s, 1H); 3.44 (br. s, 1H); 2.06 (d, J=9.0, 1H); 2.02-1.85 (m, 2H); 1.70-1.62 (m, 1H); 1.46 (s, 9H); 1.34-1.20 (m, 2H). IR (cm⁻¹, KBr): 3437 (w), 3335 (m), 3081 (w), 2967 (m), 2927 (m), 2870 (m), 1653 (s), 1545 (m), 1506 (m), 1491 (m), 1446 (m), 1359 (m), 1278 (m), 1227 (m), 1078 (m), 894 (m). MS (m/z): 362.35 ([M+H]⁺).

### Example 122

### Preparation of N5-(tert-Butyl)-3-(2-chloro-4-fluorophenyl)-3,4-diazatricyclo[5.2.1.0^{2,6}]deca-2(6),4-diene-5-carboxamide

This compound was prepared from intermediate 58 (100 mg, 0.32 mmol), dry dimethyl formamide (1-5 mL), triethylamine (51 μL, 0.35 mmol), benzotriazol-1-yloxytris(dimethylamino)-phosphonium hexafluorophosphate (151 mg, 0.34 mmol) and tert-butylamine (42 μL, 0.39 mmol) according to a procedure described in Example 93 and furnished the title compound as a white solid. M.P.: 122-124° C. ¹H-NMR (δ ppm, CDCl₃, 300 MHz): 7.47 (dd, J=8.7, 5.4, 1H); 7.32-7.25 (dd, J=8.1, 2.7, 1H); 7.15-7.06 (td, J=8.7, 2.7 1H); 6.71 (br. s, 1H); 3.75 (br. s, 1H); 3.32 (br. s, 1H); 2.11 (d, J=9.0, 1H); 2.00-1.82 (m, 2H); 1.68 (d, J=8.7, 1H); 1.45 (s, 9H); 1.34-1.20 (m, 2H). IR (cm⁻¹, KBr): 3402 (m), 3065 (w), 2968 (m), 2925 (m), 2870 (m), 1668 (s), 1605 (w), 1544 (m), 1521 (s), 1492 (s), 1446 (m), 1390 (m), 1363 (m), 1255 (m), 1206 (m), 1159 (m), 1119 (m), 1069 (m), 859 (m). MS (m/z): 362.45 ([M+H]⁺).

### Example 123

### Preparation of N5-(tert-Butyl)-3-(2,4,6-trifluorophenyl)-3,4-diazatricyclo[5.2.1.0^{2,6}]deca-2(6),4-diene-5-carboxamide

This compound was prepared from intermediate 42 (100 mg, 0.32 mmol), dry dimethyl formamide (1-5 mL), triethylamine (51 μL, 0.35 mmol), benzotriazol-1-yloxytris(dimethylamino)-phosphonium hexafluorophosphate (150 mg, 0.34 mmol) and tert-butyl amine (41 μL, 0.39 mmol) according to a procedure described in Example 93 and furnished the title compound as a white solid. M.P.: 142-146° C. ¹H-NMR (δ ppm, CDCl₃, 300 MHz): 6.86 (t, J=7.8, 2H); 6.89 (br. s, 1H); 3.76 (br. s, 1H); 3.26 (br. s, 1H); 2.09 (d, J=9.0, 1H); 2.00-1.85 (m, 2H); 1.67 (d, J=8.7, 1H); 1.45 (s, 9H); 1.27-1.20 (m, 2H). IR (cm⁻¹, KBr): 3412 (m), 3060 (m), 2967 (m), 2928 (m), 2871 (m), 1677 (s), 1643 (m), 1608 (s), 1538 (s), 1497 (m), 1450 (m), 1389 (w), 1341 (m), 1363 (m), 1277 (m), 1255 (m), 1221 (m), 1185 (m), 1161 (m), 1133 (m), 1117 (m), 1041 (m), 997 (m), 867 (m), 838 (m). MS (m/z): 364.37 ([M+H]⁺).

### Example 124

### Preparation of N5-(tert-Butyl)-3-(2,4,5-trifluorophenyl)-3,4-diazatricyclo[5.2.1.0^{2,6}]deca-2(6),4-diene-5-carboxamide

This compound was prepared from intermediate 60 (100 mg, 0.32 mmol), dry dimethyl formamide (1-5 mL), triethylamine (48 μL, 0.35 mmol), benzotriazol-1-yloxytris(dimethylamino)-phosphonium hexafluorophosphate (148 mg, 0.33 mmol) and tert-butyl amine (51 μL, 0.48 mmol) according to a procedure described in Example 93 and furnished the title compound as a white solid. M.P.: 127-130° C. ¹H-NMR (δ ppm, CDCl₃, 300 MHz): 7.66-7.55 (m, 1H); 7.18-7.06 (m, 1H); 6.72 (br. s, 1H); 3.74 (br. s, 1H); 3.46 (br. s, 1H); 2.07 (d, J=8.1, 1H); 2.04-1.82 (m, 2H); 1.67 (d, J=8.4, 1H); 1.47 (s, 9H); 1.32-1.18 (m, 2H). IR (cm⁻¹, KBr): 3471 (w), 3330 (w), 2965 (m), 2974 (m), 1654 (s), 1527 (m), 1452 (m), 1414 (w), 1365 (w), 1345 (w), 1247 (m), 1189 (m), 1138 (w), 826 (m). MS (m/z): 364.44 ([M+H]⁺).

### Example 125

### Preparation of N5-(tert-Butyl)-3-(3,5-difluorophenyl)-3,4-diazatricyclo[5.2.1.0^{2.6}]deca-2(6),4-diene-5-carboxamide

This compound was prepared from intermediate 62 (100 mg, 0.34 mmol), dry dimethyl formamide (1-5 mL), triethylamine (54 μL, 0.37 mmol), benzotriazol-1-yloxytris(dimethylamino)-phosphonium hexafluorophosphate (160 mg, 0.36 mmol) and tert-butyl amine (44 μL, 0.41 mmol) according to a procedure described in Example 93 and furnished the title compound as a white solid. M.P.: 103-107° C. ¹H-NMR (δ ppm, CDCl₃, 300 MHz): 7.26-7.22 (m, 2H); 6.75-6.68 (m, 2H); 3.73 (br. s, 1H); 3.70 (br. s, 1H); 2.10 (d, J=9.0, 1H); 1.99 (d, J=7.8, 2H); 1.71 (d, J=8.7, 1H); 1.48 (s, 9H); 1.26-1.18 (m, 2H). IR (cm⁻¹, KBr): 3325 (w), 3058 (m), 2971 (m), 2873 (m), 1661 (m), 1625 (s), 1548 (s), 1510 (s), 1482 (m), 1450 (m), 1390 (w), 1364 (m), 1339 (m), 1230 (m), 1205 (m), 1156 (m), 1126 (m), 1113 (m), 889 (m), 865 (m), 831 (m). MS (m/z): 346.40 ([M+H]⁺).

### Example 126

### N5-(tert-Butyl)-4-(2,4-difluorophenyl)-3,4-Diazatricyclo[5.2.1.0^{2,6}]deca-2,5-diene-5-carboxamide

The title compound was synthesized by a procedure similar to that described for example 93. Intermediate 70 (100 mg, 0.34 mmol), dry DMF (1.0 ml), Et₃N (52 μl, 0.37 mmol), BOP reagent (159 mg, 0.36 mmol) and tert-butylamine (54 μl, 0.51 mmol) furnished the title compound as an off-white solid (94 mg, 80%). M.P.: 171-173° C. ¹H-NMR (δ ppm, CDCl₃, 300 MHz): 7.51 (q, J=8.7, 1H); 7.00-6.85 (m, 2H); 5.70 (br. s, 1H); 3.45 (br. s, 1H); 3.42 (br. s, 1H); 2.08 (d, J=7.5, 1H); 2.01 (d, J=7.5, 2H); 1.77 (d, J=9.0, 1H); 1.55 (d, J=8.7, 2H); 1.40 (s, 9H). IR (cm⁻¹, KBr): 3316 (m), 3086 (m), 2977 (m), 2907 (w), 2874 (m), 1655 (s), 1605 (m), 1588 (w), 1519 (s), 1458 (w), 1395 (w), 1367 (m), 1329 (m), 1234 (m), 1290 (m), 1272 (m), 1204 (w), 1143 (m), 1114 (m), 1061 (m), 962 (m), 855 (m). MS (m/z): 346.30 ([M+H]⁺).

### Example 127

### Preparation of 5-(2,4-Difluorophenyl)-4,5-diazatricyclo[5.2.1.0^{2,6}]deca-2(6),3-dien-3-yl 1,2,3,4-tetrahydro-2-isoquinolinyl methanone

This compound was prepared from intermediate 3 (100 mg, 0.34 mmol), dry DMF dimethyl formamide (1-5 mL), triethylamine (57 μL, 0.41 mmol), benzotriazol-1-yloxytris(dimethylamino)-phosphonium hexafluorophosphate (167 mg, 0.37 mmol) and 1,2,3,4-tetrahydro isoquinone (43 μL, 0.34 mmol) according to a procedure described in Example 93 and furnished the title compound as a white solid. M.P.: 134-138° C. ¹H-NMR (δ ppm, CDCl₃, 300 MHz): 7.74-7.62 (m, 1H); 7.18-7.11 (m, 4H); 7.10-6.90 (m, 2H); 5.21 (br. s, 1H); 4.91 (br. s, 1H); 4.19 (br. s, 1H); 4.00 (br. s, 1H); 3.60 (br. s, 1H); 3.48 (br. s, 1H); 2.96 (br. s, 2H); 2.15-2.05 (m, 1H); 2.02-1.90 (m, 2H); 1.68 (d, J=9.0, 1H); 1.35-1.20 (m, 2H). IR (cm⁻¹, KBr): 3436 (m), 3044 (w), 2962 (m), 2940 (m), 1621 (s), 1583 (w), 1524 (m), 1496 (w), 1434 (s), 1268 (m), 1252 (w), 1219 (m), 1142 (m), 1102 (m), 1085 (m), 963 (m). MS (m/z): 406.39 ([M+H]⁺).

### Example 128

### Preparation of 5-(2,4-Difluorophenyl)-4,5-diazatricyclo[5.2.1.0^{2,6}]deca-2(6),3-dien-3-yl 1,2,3,4-tetrahydro-1-quinolinyl methanone

This compound was prepared from intermediate 3 (100 mg, 0.34 mmol), dry DMF dimethyl formamide (1-5 mL), triethylamine (57 μL, 0.41 mmol), benzotriazol-1-yloxytris(dimethylamino)-phosphonium hexafluorophosphate (167 mg, 0.37 mmol) and 1,2,3,4-tetrahydro quinoline (43 μL, 0.34 mmol) according to a procedure described in Example 93 and furnished the title compound as an off-white solid. M.P.: 103-107° C. ¹H-NMR (δ ppm, CDCl₃, 300 MHz): 7.75-7.62 (m, 1H); 7.16 (d, J=6.6, 1H); 7.10-6.90 (m, 5H); 4.15-4.02 (m, 1H); 4.01-3.90 (m, 1H); 3.36 (br. s, 1H); 2.92-2.68 (m, 3H); 2.15-1.85 (m, 2H); 1.80-1.72 (m, 2H); 1.50-1.40 (m, 1H); 1.26-1.20 (m, 1H). IR (cm⁻¹, KBr): 3435 (m), 3016 (w), 3075 (w), 2985 (m), 2926 (m), 2951 (m), 2865 (m), 1639 (s), 1604 (m), 1519 (s), 1493 (s), 1453 (m), 1427 (m), 1385 (s), 1345 (w), 1271 (m), 1206 (m), 1145 (m), 1088 (m), 1078 (m), 966 (m). MS (m/z): 406.37 ([M+H]⁺).

### Example 129

### Preparation of N5-[(1R)-Indan-1-yl]-3-(2,4-difluorophenyl)-3,4-diazatricyclo[5.2.1.0^{2,6}]deca-2(6),4-diene-5-carboxamide

This compound was prepared from intermediate 3 (100 mg, 0.34 mmol), dry dimethyl formamide (1-5 mL), triethylamine (57 μL, 0.41 mmol), benzotriazol-1-yloxytris(dimethylamino)-phosphonium hexafluorophosphate (167 mg, 0.37 mmol) and (R)-(−)-1-aminoindane (44 μL, 0.34 mmol) according to a procedure described in Example 93 and furnished the title compound as an off-white solid. M.P.: 158-161° C. ¹H-NMR (δ ppm, CDCl₃, 300 MHz): 7.69-7.58 (m, 1H); 7.42-7.32 (m, 1H); 7.30-7.15 (m, 2H); 7.09 (d, J=7.2, 1H); 7.05-6.92 (m, 2H); 5.75-5.62 (m, 1H); 3.76 (br. s, 1H); 3.46 (br. s, 1H); 3.10-2.85 (m, 2H); 2.75-2.60 (m, 1H); 2.12-2.05 (m, 1H); 2.02-1.82 (m, 3H); 1.70 (d, J=9.0, 1H), 1.68-1.65 (m, 1H); 1.40-1.20 (m, 2H). IR (cm⁻¹, KBr): 3412 (w), 3285 (m), 3076 (w), 3005 (w), 2960 (m), 2872 (m), 1644 (s), 1609 (w), 1547 (s), 1524 (s), 1495 (m), 1458 (m), 1361 (m), 1272 (m), 1163 (w), 1141 (m), 1090 (m), 964 (m). MS (m/z): 406.14 ([M+H]⁺).

### Example 130

### Preparation of 3-(2,4-Difluorophenyl)-3,4-diazatricyclo[5.2.1.0^{2,6}]deca-2(6),4-dien-5-carboxylic N′-tert-butylhydrazide

This compound was prepared intermediate 3 (100 mg, 0.34 mmol), dry dimethyl formamide (1-5 mL), triethylamine (100 μL, 0.75 mmol), benzotriazol-1-yloxytris(dimethylamino)-phosphonium hexafluorophosphate (160 mg, 0.36 mmol) and tert-butylhydrazine hydrochloride (47 mg, 0.37 mmol) according to a procedure described in Example 93 and furnished the title compound as an off-white solid. M.P.: 140-142° C. ¹H-NMR (δ ppm, CDCl₃, 300 MHz): 8.01 (br. s, 1H); 7.66 (d, J=9.0, 1H); 7.05-6.96 (m, 2H); 4.78 (br. s, 1H); 3.74 (br. s, 1H); 3.46 (br. s, 1H); 2.09 (d, J=9.0, 1H); 1.97 (d, J=7.8, 2H); 1.69 (d, J=8.7, 1H); 1.27 (d, J=7.2, 2H); 1.16 (s, 9H); IR (cm⁻¹, KBr): 3345 (w), 3304 (w), 2971 (m), 2872 (w), 1664 (m), 1651 (m), 1523 (s), 1470 (w), 1436 (m), 1389 (w), 1363 (m), 1271 (m), 1255 (m), 1148 (m), 1089 (m), 1021 (w), 968 (m), 870 (w). MS (m/z): 361.29 ([M+H]⁺).

### Example 131

### Preparation of N5-[(1S)-1-(4-fluorophenyl)-2-methoxyethyl]-3-(2,4-difluorophenyl)-3,4-diazatricyclo[5.2.1.0^{2,6}]deca-2(6),4-diene-5-carboxamide

Methyl iodide (109 μL, 1.74 mmol) was added to a suspension of intermediate 92 (150 mg, 0.35 mmol) and silver oxide (202 mg, 0.87 mmol) in dry toluene (3.0-5.0 mL), and the reaction flask was covered with aluminum foil and heated at about 40° C. for overnight. The reaction mixture was allowed to cool and filtered through celite and evaporated. Purification of the crude product by silicondioxide column chromatography and furnished the title compound as an off-white solid. M.P.: 109-111° C. ¹H-NMR (δ ppm, CDCl₃, 300 MHz): 7.71 (q, J=9.0, 1H); 7.50-7.30 (m, 3H); 7.09-6.92 (m, 4H); 5.38-5.25 (m, 1H); 3.71 (s, 3H); 3.44 (br. s, 1H); 3.38 (br. s, 3H); 2.10-2.01 (m, 1H); 2.00-1.88 (m, 2H); 1.67 (d, J=8.7, 1H); 1.30-1.19 (m, 2H). IR (cm⁻¹, KBr): 3403 (m), 3332 (m), 2966 (w), 2973 (m), 2863 (w), 1634(s), 1588 (w), 1552 (s), 1500 (s), 1453 (w), 1400 (m), 1385 (w), 1354 (m), 1276 (m), 1256 (w), 1171 (m), 1069 (s), 1004 (w), 834 (m).

### Example 132

### Preparation of N5-(2-Methoxy-1,1-dimethylethyl)-3-(2,4-difluorophenyl)-3,4-diazatricyclo[5.2.1.0^{2,6}]deca-2(6),4-dien-5-carboxamide

The title compound was prepared from Example 93 (200 mg, 0.55 mmol), silver oxide (317 mg, 1.37 mmol), dry toluene (4.0 ml) and methyl iodide (172 μL, 2.77 mmol) according to a procedure described in Example 131 and furnished the title compound as a yellow oil. ¹H-NMR (δ ppm, CDCl₃, 300 MHz): 7.72-7.66 (m, 1H); 7.04-6.92 (m, 3H); 3.73 (br. s, 1H); 3.51 (br. s, 2H); 3.48-3.35 (m, 4H); 2.07 (d, J=8.7, 1H); 2.00-1.88 (m, 2H); 1.67 (d, J=8.7, 1H); 1.46 (s, 6H); 1.32-1.19 (m, 2H). IR (cm⁻¹, KBr): 3406 (m), 2974 (m), 2930 (m), 2874 (m), 1672 (s), 1611 (w), 1525 (s), 1495 (s), 1447 (m), 1392 (m), 1361 (m), 1271 (s), 1232 (m), 1145 (m), 1110 (m), 966 (m), 870 (m), 851 (m). MS (m/z): 376.59 ([M+H]⁺).

### Example 133

### Preparation of tert-Butylamino 5-(2,4-difluorophenyl)-4,5-diazatricyclo[5.2.1.0^{2,6}]deca-2(6),3-diene-3-yl methanethione

The example 106 (300 mg, 0.86 mmol) and phosphorous pentasulphide (464 mg, 2.08 mmol) were refluxed in pyridine for about 24 hours. The reaction mixture was then poured into 10% sodium hydroxide and extracted with ethyl acetate. The combined organic layers were washed with water, brine and dried over sodium sulphate. Purification of the crude by silicon dioxide column chromatography furnished the titled compound as a yellow solid. M.P.: 148-150° C. ¹H-NMR (δ ppm, CDCl₃, 300 MHz): 8.61 (br. s, 1H); 7.64 (q, J=9.0, 1H); 7.00 (t, J=9.0, 2H); 3.96 (br. s, 1H); 3.41 (br. s, 1H); 2.07 (d, J=7.2, 1H); 2.00-1.92 (m, 2H); 1.64 (s, 10H); 1.36-1.18 (m, 2H). IR (cm⁻¹, KBr): 3441 (w), 3338 (m), 2965 (m), 2871 (m), 1612 (m), 1523 (s), 1450 (w), 1401 (s), 1363 (m), 1270 (m), 1211 (m), 1144 (m), 1121 (m), 1095 (m), 1062 (m), 1013 (m), 964 (m). MS (m/z): 362.08 ([M+H]⁺).

### Example 134

### Preparation of N5-(2-Hydroxy-1,1-dimethylethyl)-3-(2,4-difluorophenyl)-3,4-diazatricyclo[5.2.2.0^{2,6}]undeca-2(6),4-diene-5-carboxamide

This compound was prepared from intermediate 104 (77 mg, 0.25 mmol), dry dimethyl formamide (1-5 mL), triethylamine (42 μL, 0.30 mmol), benzotriazol-1-yloxytris(dimethylamino)-phosphonium hexafluorophosphate (122 mg, 0.27 mmol) and 2-amino-2-methyl propanol (24 μL, 0.259 mmol) according to a procedure described in Example 93 and furnished the title compound as a white solid. M.P.: 128-131° C.

¹H-NMR (δ ppm, CDCl₃, 300 MHz): 7.64-7.53 (m, 1H); 7.12-6.94 (m, 3H); 3.78 (br. s, 1H); 3.70 (br. s, 2H); 3.13 (br. s, 1H); 1.77 (d, J=6.6, 4H); 1.52-1.24 (m, 4H); 1.39 (s, 6H). IR (cm⁻¹, KBr): 3390 (m), 3344 (m), 3279 (m), 3045 (w), 2965 (m), 2950 (m), 2871 (m), 1643 (s), 1612 (w), 1558 (s), 1522 (s), 1503 (m), 1449 (m), 1389 (w), 1367 (w), 1263 (m), 1274 (m), 1174 (s), 1151 (m), 1097 (m), 1073 (m), 957 (m), 857 (m). MS (m/z): 376.27 ([M+H]⁺).

### Example 135

### Preparation of N5-(2-Hydroxy-1,1-dimethylethyl)-3-(4-fluorophenyl)-3,4-diazatricyclo[5.2.2.0^{2,6}]undeca-2(6),4-diene-5-carboxamide

This compound was prepared from intermediate 102 (90 mg, 0.31 mmol), dry dimethyl formamide (1-5 mL), triethylamine (52 μL, 0.37 mmol), benzotriazol-1-yloxytris(dimethylamino)-phosphonium hexafluorophosphate (152 mg, 0.34 mmol) and 2-amino-2-methyl propanol (30 μL, 0.31 mmol) according to a procedure described in Example 93 and furnished the title compound as a white solid. M.P.: 165-168° C. ¹H-NMR (δ ppm, CDCl₃, 300 MHz): 7.56-7.44 (m, 2H); 7.20 (d, J=9.0, 2H); 7.08 (br. s, 1H); 3.79 (br. s, 1H); 3.70 (br. s, 2H); 3.39 (br. s, 1H); 1.80 (d, J=6.0, 4H); 1.54-1.28 (m, 4H); 1.41 (s, 6H). IR (cm⁻¹, KBr): 3336 (m), 3295 (m), 3082 (w), 2936 (m), 2957 (m), 2936 (m), 2866 (m), 1639 (s), 1535 (m), 1556 (m), 1515 (s), 1500 (m), 1451 (m), 1364 (w), 1262 (m), 1218 (m), 1173 (m), 1071 (m), 844 (m). MS (m/z): 358.31 ([M+H]⁺).

### Example 136

### Preparation of N5-(tert-Butyl)-3-(4-chlorophenyl)-3,4-diazatricyclo[5.2.2.0^{2,6}]undeca-2(6),4-diene-5-carboxamide

This compound was prepared from intermediate 96 (100 mg, 0.33 mmol), dry dimethyl formamide (1-5 mL), triethylamine (55 μL, 0.39 mmol), benzotriazol-1-yloxytris(dimethylamino)-phosphonium hexafluorophosphate (160 mg, 0.36 mmol) and tert-butyl amine (34 μL, 0.33 mmol) according to a procedure described in Example 93 and furnished the title compound as a white solid. M.P.: 198-200° C. ¹H-NMR (δ ppm, CDCl₃, 300 MHz): 7.47 (br. s, 4H); 6.87 (br. s, 1H); 3.83 (br. s, 1H); 3.42 (br. s, 1H); 1.78 (d, J=8.4, 4H); 1.60-1.52 (m, 4H); 1.48 (s, 9H). IR (cm⁻¹, KBr): 3397 (m), 3059 (w), 2952 (m), 2932 (m), 2866 (m), 1664 (s), 1597 (w), 1552 (m), 1534 (m), 1507 (s), 1489 (s), 1439 (m), 1363 (m), 1261 (m), 1226 (m), 1089 (m), 838 (m). MS (m/z): 358.16 ([M+H]⁺).

### Example 137

### Preparation of N5-(tert-Butyl)-3-(4-trifluoromethylphenyl)-3,4-diazatricyclo[5.2.2.0^{2,6}]undeca-2(6),4-diene-5-carboxamide

This compound was prepared from intermediate 98 (100 mg, 0.29 mmol), dry dimethyl formamide (1-5 mL), triethylamine (49 μL, 0.35 mmol), benzotriazol-1-yloxytris(dimethylamino)-phosphonium hexafluorophosphate (144 mg, 0.32 mmol) and tert-butyl amine (31 μL, 0.29 mmol) according to a procedure described in Example 93 and furnished the title compound as a white solid. M.P.: 174-176° C. ¹H-NMR (δ ppm, CDCl₃, 300 MHz): 7.76 (d, J=9.0, 2H); 7.68 (d, J=9.0, 2H); 6.88 (br. s, 1H); 3.85 (br. s, 1H); 3.49 (br. s, 1H); 1.79 (d, J=9.0, 4H); 1.62-1.52 (m, 4H); 1.48 (s, 9H). IR (cm⁻¹, KBr): 3403 (m), 3068 (w), 2964 (m), 2932 (m), 2872 (m), 1666 (s), 1617 (w), 1593 (m), 1617 (m), 1539 (s), 1497 (s), 1443 (m), 1362 (m), 1324 (m), 1263 (m), 1220 (m), 1162 (s), 1126 (s), 1102 (s), 1065 (s), 1014 (m), 856 (m), 846(m).

### Example 138

### Preparation of N5-(tert-Butyl)-3-(4-bromophenyl)-3,4-diazatricyclo[5.2.2.0^{2,6}]undeca-2(6),4-diene-5-carboxamide

This compound was prepared from intermediate 100 (100 mg, 0.28 mmol), dry dimethyl formamide (1-5 mL), triethylamine (47 μL, 0.34 mmol), benzotriazol-1-yloxytris(dimethylamino)-phosphonium hexafluorophosphate (139 mg, 0.31 mmol) and tert-butylamine (30 μL, 0.28 mmol) according to a procedure described in Example 93 and furnished the title compound as an off-white solid. M.P.: 191-193° C. ¹H-NMR (δ ppm, CDCl₃, 300 MHz): 7.61 (d, J=9.0, 2H); 7.42 (d, J=9.0, 2H); 6.87 (br. s, 1H); 3.83 (br. s, 1H); 3.42 (br. s, 1H); 1.77 (d, J=9.0, 4H); 1.62-1.52 (m, 4H); 1.48 (s, 9H). IR (cm⁻¹, KBr): 3396 (m), 3351 (w), 2958 (m), 2932 (m), 2865 (m), 1662 (s), 1591 (m), 1535 (m), 1505 (m), 1487 (s), 1444 (m), 1363 (m), 1316 (w), 1261 (m), 1227 (m), 1148 (m), 1068 (m), 1009 (m), 855 (m), 835 (m).

### Example 139

### Preparation of N5-(tert-Butyl)-3-(4-fluorophenyl)-3,4-diazatricyclo[5.2.2.0^{2,6}]undeca-2(6),4-diene-5-carboxamide

This compound was prepared from intermediate 102 (90 mg, 0.33 mmol), dry dimethyl formamide (1-5 mL), triethylamine (52 μL, 0.37 mmol), benzotriazol-1-yloxytris(dimethylamino)-phosphonium hexafluorophosphate (152 mg, 0.35 mmol) and tert-butyl amine (32 μL, 0.31 mmol) according to a procedure described in Example 93 and furnished the title compound as an off-white solid. M.P.: 190-193° C. ¹H-NMR (δ ppm, CDCl₃, 300 MHz): 7.53-7.47 (m, 2H); 7.22-7.15 (m, 2H); 6.88 (br. s, 1H); 3.83 (br. s, 1H); 3.38 (br. s, 1H); 1.78 (d, J=8.4, 4H); 1.54-1.25 (m, 4H); 1.48 (s, 9H). IR (cm⁻¹, KBr): 3401 (m), 3059 (w), 2966 (m), 2944 (m), 2864 (m), 1664 (s), 1606 (w), 1554 (m), 1536 (m), 1517 (s), 1496 (s), 1442 (m), 1364 (m), 1263 (m), 1222 (m), 1170 (m), 1150 (m), 856 (m), 845 (m). MS (m/z): 342.24 ([M+H]⁺).

### Example 140

### Preparation of (1S,7R)-N5-(tert-Butyl)-3-(2,4-difluorophenyl)-1,10,10-trimethyl-3,4-diazatricyclo[5.2.1.0^{2,6}]deca-2(6),4-diene-5-carboxamide

This compound was prepared from the Intermediate 79 (100 mg, 0.30 mmol), dry dimethyl formamide (1-5 mL) and triethylamine (47 μL, 0.33 mmol) and benzotriazol-1-yloxytris(dimethylamino)-phosphonium hexafluorophosphate (140 mg, 0.31 mmol) and tert-butyl amine (48 μL, 0.62 mmol) according to a procedure described in Example 93 and furnished the title compound as a white solid. M.P.: 161-163° C. ¹H-NMR (δ ppm, CDCl₃, 300 MHz): 7.53-7.43 (m, 1H); 7.04-6.94 (m, 2H); 6.71 (br. s, 1H); 3.22 (d, J=3, 1H); 2.18-2.07 (m, 1H); 1.85-1.73 (m, 1H); 1.45 (s, 9H); 1.36-1.24 (m, 2H); 0.97 (s, 3H); 0.90 (s, 3H); 0.78 (s, 3H). IR (cm⁻¹, KBr): 3406 (m), 3088 (m), 3043 (w), 2917 (s), 1671 (s), 1616 (w), 1555 (s), 1523 (s), 1492 (s), 1444 (s), 1392 (m), 1365 (m), 1272 (s), 1217 (m), 1163 (w), 1150 (m), 1100 (m), 968 (w), 861 (m). MS (m/z): 388.42 ([M+H]⁺).

### Example 141

### Preparation of N5-(2-Hydroxy-1,1-dimethyl)-3-(2,4-difluorophenyl)-1,10,10-trimethyl-3,4-diazatricyclo[5.2.1.0^{2,6}]deca-2(6),4-diene-5-carboxamide

This compound was prepared from the intermediate 79 (100 mg, 0.30 mmol), dry dimethyl formamide (1-5 mL), triethylamine (46 μL, 0.33 mmol) and benzotriazol-1-yloxytris(dimethylamino)-phosphonium hexafluorophosphate (140 mg, 0.35 mmol) and 2-amino-2-methyl propanol (43 μL, 0.45 mmol) according to a procedure described in Example 93 and furnished the title compound as white solid M.P.: 130-132° C. ¹H-NMR (δ ppm, DMSO-d₆, 300 MHz): 7.73 (q, J=9.0, 1H); 7.64-7.58 (m, 1H); 7.32-7.22 (m, 1H); 7.11 (br. s, 1H); 3.45-3.30 (2H, under H2O signal); 3.02 (br. s, 1H); 2.12-1.95 (m, 2H); 1.90-1.78 (m, 1H); 1.29 (s, 6H); 1.08-0.99 (m, 1H); 0.88 (s, 6H); 0.73 (s, 3H). IR (cm⁻¹, KBr): 3381 (m), 3086 (w), 2972 (m), 2874 (w), 1638 (m), 1650 (m), 1614 (w), 1551 (m), 1524 (s), 1499 (m), 1448 (w), 1389 (w), 1324 (w), 1269 (m), 1225 (w), 1142 (w), 1074 (w), 1018 (m), 963 (w), 846 (w).

### Example 142

### Preparation of N(7)-(tert-Butyl)-5-(2,4-difluorophenyl)-5,6-diazatetracyclo[7.3.1.1^{3,11}.0^{4,8}]tetradeca-4(8),6-diene-7-carboxamide

This compound was from intermediate 91 (150 mg, 0.43 mmol), dry dimethyl formamide (1-5 mL), triethylamine (72 μL, 0.52 mmol), benzotriazol-1-yloxytris(dimethylamino)-phosphonium hexafluorophosphate (211 mg, 0.47 mmol) and tert-butyl amine (55 μL, 0.52 mmol) according to a procedure described in Example 93 and furnished the title compound as an off-white solid. M.P.: 188-190° C. ¹H-NMR (δ ppm, CDCl₃, 300 MHz): 7.49-7.39 (m, 1H); 7.05-6.94 (m, 2H); 6.79 (br. s, 1H); 3.96 (br. s, 1H); 2.64 (br. s, 1H); 2.17 (br. s, 2H); 2.04-1.72 (m, 10H); 1.41 (s, 9H). IR (cm⁻¹, KBr): 3398 (m), 3089 (w), 2963 (m), 2922 (m), 1672 (s), 1613 (m), 1570 (w), 1520 (s), 1525 (m), 1485 (m), 1442 (m), 1389 (m), 1362 (m), 1261 (m), 1228 (m), 1144 (m), 1099 (s), 1083 (m), 1030 (m), 849 (m). MS (m/z): 400.35 ([M+H]⁺).

### Example 143

### Preparation of N(7)-(tert-Butyl)-5-(4-fluorophenyl)-5,6-diazatetracyclo[7.3.1.1^{3,11}.0^{4,8}]tetradeca-4(8),6-diene-7-carboxamide

This compound was prepared from intermediate 93 (150 mg, 0.46 mmol), dry dimethyl formamide (1-5 mL), triethylamine (76 μL, 0.55 mmol), benzotriazol-1-yloxytris(dimethylamino)-phosphonium hexafluorophosphate (223 mg, 0.50 mmol) and tert-butylamine (58 μL, 0.55 mmol) according to a procedure described in Example 93 and furnished the title compound as an off-white solid. M.P.: 214-216° C. ¹H-NMR (δ ppm, CDCl₃, 300 MHz): 7.34-7.20 (m, 2H); 7.16 (t, J=9.0, 2H); 6.84 (br. s, 1H); 3.98 (br. s, 1H); 2.94 (br. s, 1H); 2.18 (br. s, 2H); 2.04-1.73 (m, 10H); 1.44 (s, 9H). IR (cm⁻¹, KBr): 3395 (m), 3084 (w), 2962 (s), 2847 (s), 2902 (s), 1671 (s), 1607 (w), 1513 (s), 1486 (m), 1454 (m), 1386 (m), 1361 (m), 1258 (m), 1220 (m), 1162 (m), 1096 (m), 1085 (s), 1033 (m), 1013 (m), 869 (m), 837 (m). MS (m/z): 382.29 ([M+H]⁺).

### Example 144

### Preparation of (1R,8R)-N5-(tert-Butyl)-3-(2,4-diflurophenyl)-9,9-dimethyl-3,4-diazatricyclo[6.1.1.0^{2,6}]deca-2(6),4-diene-5-carboxamide

This compound was prepared from intermediate 111 (100 mg, 0.31 mmol), dry dimethyl formamide (1-5 mL), triethylamine (52 μL, 0.37 mmol) and benzotriazol-1-yloxytris(dimethylamino)-phosphonium hexafluorophosphate (143 mg, 0.32 mmol) and tert-butylamine (49 μL, 0.46 mmol) according to a procedure described in Example 93 to furnish the title compound as an off-white solid. M.P.: 168-171° C. ¹H-NMR (δ ppm, CDCl₃, 300 MHz): 7.50 (q, J=9.0, 1H); 7.04-6.92 (m, 2H); 6.78 (br. s, 1H); 3.04-2.98 (m, 2H); 2.66 (br. s, 2H); 2.33 (br. s, 1H); 1.47 (s, 9H); 1.38 (s, 4H); 0.75 (s, 3H). IR (cm⁻¹, KBr): 3406 (m), 3088 (m), 3043 (w), 2917 (s), 1671 (s), 1616 (w), 1555 (s), 1523 (s), 1492 (s), 1444 (s), 1392 (m), 1365 (m), 1272 (s), 1217 (m), 1163 (w), 1150 (m), 1100 (m), 968 (w), 861 (m). MS (m/z): 374.22 ([M+H]⁺).

### Example 145

### Preparation of (1S,8S)-N5-(tert-Butyl)-3-(2,4-diflurophenyl)-9,9-dimethyl-3,4-diazatricyclo[6.1.1.0^{2,6}]deca-2(6),4-diene-5-carboxamide

This compound was prepared from intermediate 107 (100 mg, 0.31 mmol), dry dimethyl formamide (1-5 mL), triethylamine (54 μL, 0.37 mmol), benzotriazol-1-yloxytris(dimethylamino)-phosphonium hexafluorophosphate (145 mg, 0.32 mmol) and tert-butyl amine (50 μL, 0.46 mmol) according to a procedure described in Example 93 and furnished the title compound as a white solid. M.P.: 167-170° C. ¹H-NMR (δ ppm, CDCl₃, 300 MHz): 7.56-7.44 (m, 1H); 7.08-6.92 (m, 2H); 3.06-2.97 (m, 2H); 2.72-2.62 (m, 2H); 2.33 (br. s, 1H); 1.53-1.42 (m, 9H); 1.38 (br. s, 4H); 0.75 (br. s, 3H). IR (cm⁻¹, KBr): 3406 (m), 3088 (m), 3043 (w), 2917 (s), 1671 (s), 1616 (w), 1555 (s), 1523 (s), 1492 (s), 1444 (s), 1392 (m), 1365 (m), 1272 (s), 1217 (m), 1163 (w), 1150 (m), 1100 (m), 968 (w), 861 (m). MS (m/z): 374.20 ([M+H]⁺).

### Example 146

### Preparation of 2-[5-(2,4-diflurophenyl)-4,5-diazatricyclo[5.2.1.0^{2,6}]deca-2(6),3-dien-3-yl]-4,4-dimethyl-4,5-dihydro-1,3-oxazole

A solution of Example 93 (100 mg, 0.277 mmol) in ethyl acetate (2 ml) was treated with thionyl chloride (40 μl, 0.55 mmol) and the mixture was stirred at RT for 2 h. After diluting with water, the mixture was extracted into ethyl acetate and combined organic layers were washed with brine, dried over Na₂SO₄ and the solvent evaporated. Purification by column chromatography furnished the title compound as off white solid (60 mg, 63%). M.P.: 134-137° C. ¹H-NMR (δ ppm, CDCl₃, 300 MHz): 7.77 (q, J=8.4, 1H), 6.96 (t, J=8.4, 2H); 4.10 (s, 2H); 3.65 (br. s, 1H); 3.47 (br. s, 1H); 2.09 (d, J=8.7, 1H); 1.95 (d, J=7.8, 2H); 1.68 (d, J=9.0, 1H); 1.40, 1.39 (2s, 6H); 1.33-1.24 (m, 2H). MS (m/z): 344.43(M+H⁺).

### Pharmacological Activity

The compounds described herein can be tested for their activity for cannabinoid receptors following any procedures known to a person ordinary skill in the art. For example, the following protocols can be employed for testing the compounds. These protocols are illustrative and do not limit to the scope of the present invention.

### Example 147

### In-vitro Protocol for Rat CB1 Receptor Binding Using Brain Membrane

In this assay, [³H]SR141716A was used to bind the CB1 receptor present in a rat brain membrane preparation which can be displaced by unlabeled ligands having affinity to the CB1 receptor.

The assay was performed according to the modified method of Thomas et al., JPET 285: 285-292 (1998). The total reaction mixture (250 ml) contains Tris-BSA buffer (50 mM Tris, pH 7.4 with 1.5% BSA) or unlabeled SR141716A (1 mM) or test samples (1 mM), [³H] SR141716A (2 nM) and 100 mg of rat brain membrane. The non-specific binding was defined by 1 mM of SR141716A. The assay mixture was incubated at 37° C. for 1 hour. The reaction was then stopped by rapid filtration under vaccum using Whatman GF/B-96 micro filter plate. A scintillation cocktail was added and radioactive counts were measured using Topcount beta scintillation counter.

The standard and test sample dilutions were made in an assay buffer containing either ethanol or DMSO at a final concentration of 1%. The percent (%) displacement by a test ligand was calculated by comparing the specific bound values.

### Example 148

### Protocol for in-vitro Assay Using hCB1-CHO Membranes

In this assay, [³H]-CP-55, 940 was used as the radioligand to bind human CB1 receptors expressed on the membranes from CHO cells (the hCB1-CHO cell line was generated in-house) which can be displaced by unlabeled ligands having affinity to the CB1 receptor.

The assay was performed according to the modified method of Ross et al., Br. J. Pharmacol. 128, 735-743 (1999). The reaction was set up in a total volume of 200 μl in PEI (Poly(ethyleneimine)) (0.2%) precoated Millipore GFB (Glass Fibre-B) filter plates. 1 mM stocks of test compounds were prepared in DMSO and tested at a final concentration of 300 nM. The non-specific binding was determined by 0.5 μM CP-55, 940. The total reaction mixture contained Tris-BSA buffer (50 mM Tris, 5 mM MgCl₂, 1 mM EDTA, pH 7.4 with 0.1% BSA), unlabelled CP-55, 940 (0.5 μM) or test samples, [³H]-CP-55, 940 (0.75 nM) and 50 μg of human CB1 receptor preparation. The assay mixture (with or without the test compound) was incubated at 37° C. for 1 hour. The reaction was stopped by rapid filtration under vacuum and the radioactivity on the filters was measured by liquid scintillation counting.

### Example 149

### In-vitro Protocol for Rat CB2 Receptor Binding Using Spleen Membrane

In this assay, [³H]CP55,940 was used to bind the CB2 receptor present in a rat spleen membrane preparation which can be displaced by unlabeled ligands having affinity to the CB2 receptor.

The assay was performed according to the modified method of Rinaldi-Carmona et al., JPET, 284: 644-650 (1998). The total reaction mixture (250 ml) contains Tris-BSA buffer (50 mM Tris, pH 7.4 with 1.5% BSA) or unlabeled SR144528 (1 mM) or test samples (300 nM), [³H]CP55,940 (1 nM) and 100 mg of rat brain membrane. The non-specific binding was defined by 1 mM of SR144528. The assay mixture was incubated at 37° C. for 1 hour. The reaction was then stopped by rapid filtration under vaccum using Whatman GF/B-96 micro filter plate. A scintillation cocktail was added and radioactive counts were measured using Topcount beta scintillation counter. The standard and test sample dilutions were made in an assay buffer containing either ethanol or DMSO at a final concentration of 1%. The percent (%) displacement by a test ligand was calculated by comparing the specific bound values.

### Example 150

### Protocol for in-vitro Radioligand Binding Assay Using CHO-hCB2 Membranes

In this assay, [³H]-CP-55, 940 was used as the radioligand to bind human CB2 receptor expressed on the membranes from CHO cells (hCB2-CHO cell line was procured form Euroscreen) which can be displaced by unlabeled ligands having affinity to the CB2 receptor.

The assay was performed according to the modified method of Ross et al., Br. Jrnl. Pharmacol. 128, 735-743 (1999). The reaction was set up in a total volume of 200 μl in PEI (0.2%) precoated Millipore filter plates. Tris-BSA buffer containing 50 mM Tris, 5 mM MgCl₂, 1 mM EDTA pH 7.4 with 0.1% BSA was used as the assay buffer. 0.75 nM [³H]-CP-55, 940 was used as the radioligand. The non-specific binding was determined by cold 0.5 μM Win-55, 212-2. 1 mM stocks of the test compounds were prepared in DMSO. Binding was initiated by the addition of 0.25 μg/100 μl hCB2-CHO membranes, with or without the addition of test compounds and incubation for 1 hour at 30° C. The reaction was stopped by rapid filtration under vacuum. Radioactivity on the filters was measured by liquid scintillation counting using microscint PS. Data from binding curves was used to calculate IC₅₀/Ki values for the test compounds using Graph Pad Prism software.

Reagents and buffer composition: Assay buffer: 50 mM Tris, 5 mM MgCl₂, 1 mM EDTA, 0.1% FAF-BSA and pH 7.4; Washing buffer: 50 mM Tris, 0.5% FAF-BSA and pH 7.4

The compounds described herein were screened at 300 nM for activity using Chinese Hamster Ovarian (CHO) cell membranes overexpressing human CB₂ cannabinoid receptor in a competitive binding experiment using [³H]-CP55940 as the radioligand. IC₅₀ values were determined for compounds which displaced >50% of [³H]-CP55940 from binding hCB2 receptors. These values were in the range from about 350 nM to about 1.5 nM, for example from about 200 nM to about 1.5 nM, or for example, from about 100 nM to about 1.5 nM, or for example, from about 30 nM to about 1.5 nM.

Some Compounds described herein showed activity towards CB1 receptors in the range of from about 12.3 μM to about 43 nM, for example from about 5 μM to about 43 nM, or for example, from about 1 μM to about 43 nM, or for example, from about 0.5 μM to about 43 nM.

### Example 151

### Pharmacokinetic Study

Male Sprague-Dawly rats were administered by oral gavage a single oral dose of 10 mg/kg b.wt. test compound in 0.5% methylcellulose suspension in overnight fasted (12 hrs before dose) condition. Fasting was continued till 4 hr post dose. Blood samples were collected predose and at 15 and 30 minutes and 1, 2, 3, 4, 6, 8, 12 and 24 hrs post-dose and were kept on an ice bath till further processing. These samples were separated for plasma by centrifuging at 4° C. for 10 minutes at 1000×g and then stored at −70° C. till further analysis. These samples were analyzed for the levels of the test compound by HPLC-UV and the pharmacokinetic parameters were calculated using these concentrations by using WinNonlin software (Pharsight Inc., USA, Version 5.1). The results are shown in the table below.

Mean Pharmacokinetic Parameters

| | | Compound No | |
|---|---|---|---|
| | | 321 | Compound No: |
| | | (WO06/129178) | 98 |
| No of animals | | 4 | 3 |
| C max | ng/ml | 131.30 | 1948.91 |
| AUC 0-t | ng · hr/ml | 1215.62 | 11665.79 |
| AUC 0-inf | ng · hr/ml | 1552.60 | 11969.78 |
| *T max | hr | 0.50 | 0.50 |
| ^{#}t_{1/2} | hr | 7.28 | 4.77 |

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above.

All publications and patent applications cited in this application are herein incorporated by reference to the same extent as if each individual publication or patent application was specifically and individually indicated to be incorporated herein by reference.

1. A compound of formula (I) 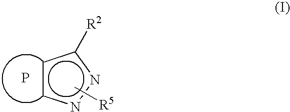
or a pharmaceutically acceptable salt thereof, a pharmaceutically acceptable solvate thereof, a regioisomer thereof, a stereoisomer thereof, a prodrug thereof, or a N-oxide thereof,
wherein
ring P is a bridged bicyclic ring system having 0-2 double bonds, which is optionally substituted with up to 10 R¹ groups;
each occurrence of R¹ is independently hydrogen, nitro, cyano, halogen, alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclyl, heterocyclylalkyl, NR³R⁴, C(═B)R⁴, C(O)OR⁴, C(O)NR³R⁴, S(O)ₘR⁴, S(O)ₘNR³R⁴, OR⁴, SR⁴ or a protecting group;
alternatively, two R¹ groups, together with the atoms to which they are attached, form an aryl or heteroaryl group;
each occurrence of R³ and R⁴ is independently hydrogen, nitro, halo, alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclyl, heterocyclylalkyl, NR^{a}R^{b}, C(═B)R^{b}, C(O)OR^{b}, C(O)NR^{a}R^{b}, S(O)ₘR^{b}, S(O)ₘNR^{a}R^{b}, OR^{b}, or SR^{b}, or R^{a} and R^{b}, when bound to a common atom, are joined to form a 3-7 membered cyclic ring containing one or more heteroatoms or groups selected from N, O, S, C(O) or SO₂, wherein the 3-7 membered cyclic ring is optionally substituted with one or more R^{c} groups;
each occurrence of R^{a} and R^{b} is independently hydrogen, alkyl, alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclyl, or heterocyclylalkyl;
each occurrence of R^{c} is independently hydrogen, alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclyl, or heterocyclylalkyl;
R² is
i) an optionally mono-, di-, or tri-substituted group selected from alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclyl, or heterocyclylalkyl, wherein the optional substituents are independently selected from —C(O)H, alkyl, aryl, and cycloalkyl which are unsubstituted or substituted with one or more of hydroxy, halogen, nitro, alkyl, alkoxy, COOR″ (wherein R″ is hydrogen or alkyl), or CONR^{3a}R^{4a}; or
ii) C(O)NHNHR^{3a}, C(O)NHNHC(O)R^{4a}, C(═S)NH₂, C(═NR^{3a})R^{4a}, (CH₂)ₚNR^{3a}R^{4a}, CH═CR^{3a}R^{4a}, CF₂R^{4a}, CHFR^{4a}, (CH₂)ₚOR^{3a}, C(═B)R^{3a}, C(O)OR^{3a}, NR^{3aa}CONR^{3a}R^{4a}, S(O)ₘR^{3a}, S(O)ₘNR^{3a}R^{4a}, NR^{3a}COR^{4a}, NR^{3a}CSR^{4a}, NR^{3a}SO₂R^{4a}, C(═NR^{3aa})NR^{3a}R^{4a}, C(═NOR^{3a})R^{4a}, C(═NNR^{3a})R^{4a}, (CH₂)ₚ—CONHR^{3a}, C≡C—R^{3a}, C(O)NH(CH₂)ₚC(O)R^{3a}, (CH₂)ₚ—CONR^{3a}R^{4a}, or C(OR^{3a})R^{4a},
with the proviso that R² is not 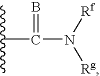
where B is O, S, or NR^{h}, and R^{f}, R^{g}, and R^{h} are independently any atom or group;
each occurrence of R^{3a}, R^{3aa}, and R^{4a} is independently i) hydrogen, nitro, or halogen, or ii) an optionally substituted group selected from alkyl, alkenyl, alkynyl, cycloalkyl, cycloalkylalkyl, cycloalkenyl, cycloalkenylalkyl, aryl, arylalkyl, heteroaryl, heteroarylalkyl, heterocyclyl, heterocyclylalkyl, NR^{a}R^{b}, C(═B)R^{b}, C(O)OR^{b}, C(O)NR^{a}R^{b}, S(O)ₘR^{b}, S(O)ₘNR^{a}R^{b}, OR^{b}, or SR^{b}, or R^{3a} and R^{4a}, when bound to a common atom, are joined to form a 3-7 membered cyclic ring containing one or more heteroatoms or groups selected from N, O, S, C(O) or SO₂, wherein the 3-7 membered cyclic ring is optionally substituted with one or more R^{c} groups;
R⁵ is hydrogen, alkyl, aryl, heteroaryl, or heterocyclyl, and R⁵ is optionally mono-, di- or tri-substituted with substituents selected from nitro, cyano, acyl, halogen, alkyl, haloalkyl, alkenyl, alkynyl, cycloalkyl, cycloalkylalkyl, cycloalkenyl, cycloalkenylalkyl, aryl, arylalkyl, heteroaryl, heteroarylalkyl, heterocyclic, heterocyclylalkyl, NR³R⁴, C(═B)R⁴, C(O)OR⁴, C(O)NR³R⁴, S(O)ₘR⁴, S(O)ₘNR³R⁴, OR⁴, SR⁴ or a protecting group;
each occurrence of m and p is independently 0, 1 or 2; and
each occurrence of B is O, S or NR^{b}.

2. The compound of claim 1, wherein the compound is selected from

| Compound | |
|---|---|
| No | Compound Name |
| 1. | 5-(2,4-difluorophenyl)-4,5-diazatricyclo[5.2.1.0^{2,6}.]deca-2(6),3-dien-3-yl- |
| | phenylmethanone; |
| 2. | 1-[5-(2,4-diflurophenyl)-4,5-diazatricyclo[5.2.1.0^{2,6}]deca-2(6),3-dien-3- |
| | yl]-1-hexanone; |
| 3. | 5-(2,4-diflurophenyl)-4,5-diazatricyclo[5.2.1.0^{2,6}.]deca-2(6),3-dien-3-yl- |
| | 1-naphthylmethanone; |
| 4. | 4-(2,4-Dichlorophenyl)-4,5-diazatricyclo[5.2.1.0^{2,6}]deca-2,5-dien-3-yl- |
| | phenylmethanone; |
| 5. | 1-[5-(2,4-diflurophenyl)-4,5-diazatricyclo[5.2.1.0^{2,6}]deca-2(6),3-dien-3- |
| | yl]-3,3,-dimethyl-1-butanol; |
| 6. | 1-{5-(2,4-diflurophenyl)-4,5-diazatricyclo[5.2.1.0^{2,6}.]deca-2(6),3-dien-3- |
| | yl]}-3,3,-dimethyl-1-butanone; |
| 7. | N2-[5-(2,4-diflurophenyl)-4,5-diazatricyclo[5.2.1.0^{2,6]}deca-2(6),3-dien-3- |
| | ylmethyl]-2-methyhyl-2-propanamine; |
| 8. | N2-[5-(2,4-diflurophenyl)-4,5-diazatricyclo[5.2.1.0^{2,6]}deca-2(6),3-dien-3- |
| | ylmethyl]-2-phenyl-2-propanamine hydrochloride; |
| 9. | N1-[5-(2,4-Difluorophenyl)-4,5-diazatricyclo[5.2.1.0^{2,6}]deca-2(6),3-dien- |
| | 3-yl]-2,2-dimethylpropanamide; |
| 10. | 3-(2,4-Difluorophenyl)-5-sulfonamide-3,4-diazatricyclo[5.2.1.0^{2,6}]deca- |
| | 2(6),4-diene; |
| 11. | N5,N5-Dibenzyl-3-(2,4-difluorophenyl)-3,4-diazatricyclo[5.2.1.0^{2,6}]deca- |
| | 2(6),4-dien-5-amine; |
| 12. | (1S,8S)-N1-[5-(2,4-difluorophenyl)-9,9-dimethyl-4,5-diazatricyclo |
| | (6,1,1,0^{2,6})-deac-2(6), 3-dien-3-yl]-2,2-dimethyl propanamide; |
| 13. | 5-benzyloxymethyl-3-(2,4-diflurophenyl)-3,4-diazatricylo |
| | [5.2.1.0^{2,6}.]deca-2(6),4diene; |
| 14. | tert-Butyl-3-(2,4-difluorophenyl)-3,4-diazatricyclo[5.2.1.0^{2,6}]deca-2(6),4- |
| | diene-5-carboxylate; |
| 15. | 3-(2,4-Difluorophenyl)-5-(1-fluro-3,3-dimethylbutyl)-3,4- |
| | diazatricyclo[5.2.1.0^{2,6}]deca-2(6),4-diene; |
| 20. | 2-(tert-Butyl)-5-[5-(2,4-difluorophenyl)-4,5-diazatricyclo[5.2.1.0^{2,6}]deca- |
| | 2(6),3-dien-3yl]-1,3,4-oxadiazole; |
| 21. | 2-[5-(2,4-Difluorophenyl)-4,5-diazatricyclo[5.2.1.0^{2,6}]deca-2(6),3-dien- |
| | 3yl]-5-Pentyl-1,3,4-oxadiazole; |
| 22. | 2-(1-Adamantly)-5-[5-(2,4-difluorophenyl)-4,5-diazatricyclo[5.2.1.0^{2,6}] |
| | deca-2(6),3-dien-3yl]-1,3,4-oxadiazole; |
| 23. | 2-(Cyclohexyl)-5-[5-(2,4-difluorophenyl)-4,5-diazatricyclo[5.2.1.0^{2,6}] |
| | deca-2(6),3-dien-3yl]-1,3,4-oxadiazole; |
| 24. | 2-(tert-Butyl)-5-[5-(2,4-difluorophenyl)-4,5-diazatricyclo[5.2.1.0^{2,6}]deca- |
| | 2(6),3-dien-3yl]-1,3,4-thiadiazole; |
| 25. | 5-[5-(tert-Butyl)-1H-1,2,4-triazol-3-yl]-3-(2,4-difluorophenyl)-3,4- |
| | diazatricyclo [5.2.1.0^{2,6}]deca-2(6),4-diene; |
| 26. | 5-(tert-Butyl)-3-[5-(2,4-difluorophenyl)-4,5-diazatricyclo[5.2.1.0^{2,6}]deca- |
| | 2(6),3-dien-3yl]-1,2,4-oxadiazole; |
| 27. | 5-(tert-Butyl)-3-[(1S,7R)-5-(2,4-difluorophenyl)-4,5- |
| | diazatricyclo[5.2.1.0^{2,6}]deca-2(6),3-dien-3yl]-1,2,4-oxadiazole; |
| 28. | 5-(tert-Butyl)-3-[(1R,7S)-5-(2,4-difluorophenyl)-4,5-diazatricyclo[5.2.1.0^{2,6}] |
| | deca-2(6),3-dien-3yl]-1,2,4-oxadiazole; |
| 29. | 3-[5-(2,4-Difluorophenyl)-4,5-diazatricyclo [5.2.1.0^{2,6}]deca-2(6),3-dien- |
| | 3yl]-5-phenyl-1,2,4-oxadiazole; |
| 30. | 3-(2,4-diflurophenyl)-5[(E)-3,3-dimethyl-1-butenyl}-3,4- |
| | diazatricyclo[5.2.1.0^{2,6}]deca-2(6),4-diene; |
| 31. | 3-(2,4-Difluorophenyl)-5-(3,3-dimethylbutyl)-3,4-diazatricyclo[5.2.1.0^{2,6}] |
| | deca-2(6),4-diene; |
| 32. | 3-(2,4-Difluorophenyl)-5-(3,3-dimethyl-1-butynyl)-3,4- |
| | diazatricyclo[5.2.1.0^{2,6}]deca-2(6),4-diene; |
| 33. | (1S,7R)-3-(2,4-Difluorophenyl)-5-(3,3-dimethyl-1-butynyl)-3,4- |
| | diazatricyclo[5.2.1.0^{2,6}]deca-2(6),4-diene; |
| 34. | (1R,7S)-3-(2,4-Difluorophenyl)-5-(3,3-dimethyl-1-butynyl)-3,4- |
| | diazatricyclo[5.2.1.0^{2,6}]deca-2(6),4-diene; |
| 35. | 1-[5-(2,4-Difluorophenyl)-4,5-diazatricyclo[5.2.1.0^{2,6}]deca-2(6),3-dien-3- |
| | yl]-2-phenylacetylene; |
| 54. | 5-(tert-Butyl)-2-[5-(2,4-difluorophenyl)-4,5-diazatricyclo[5.2.1.0^{2,6}]deca- |
| | 2(6),3-dien-3yl]-1,3-oxazole; |
| 55. | 5-(tert-Butyl)-2-[(1S,7R)-5-(2,4-Difluorophenyl)-4,5- |
| | diazatricyclo[5.2.1.0^{2,6}]deca-2(6),3-dien-3yl]-1,3-oxazole; |
| 56. | 5-(tert-Butyl)-2-[(1R,7S)-5-(2,4-Difluorophenyl)-4,5- |
| | diazatricyclo[5.2.1.0^{2,6}]deca-2(6),3-dien-3yl]-1,3-oxazole; |
| 57. | Ethyl 2-{2-[5-(2,4-difluorophenyl)-4,5-diazatricyclo [5.2.1.0^{2,6}]deca- |
| | 2(6),3-dien-3-yl]-1,3-oxazol-5-yl}-2-methyl propanoate; |
| 58. | Ethyl 2-{2-[(1S,7R)-5-(2,4-difluorophenyl)-4,5-diazatricyclo [5.2.1.0^{2,6}] |
| | deca-2(6),3-dien-3-yl]-1,3-oxazol-5-yl}-2-methyl propanoate; |
| 59. | Ethyl 2-{2-[(1R,7S)-5-(2,4-difluorophenyl)-4,5-diazatricyclo[5.2.1.0^{2,6}] |
| | deca-2(6),3-dien-3-yl]-1,3-oxazol-5-yl}-2-methyl propanoate; |
| 60. | 2-{2-[5-(2,4-Difurophenyl)-4,5-diazatricycyclo[5.2.1.0^{2,6}]deca-2(6),3- |
| | dien-3-yl]-1,3-oxazol-5-yl}-2-methylpropanoic acid; |
| 61. | 2-{2-[(1R,7S)-5-(2,4-Difluorophenyl)-4,5-diazatricyclo [5.2.1.0^{2,6}]deca- |
| | 2(6),3-dien-3-yl]-1,3-oxazol-5-yl}-2-methyl propanoic acid; |
| 62. | 2-{2-[(1S,7R)-5-(2,4-Difluorophenyl)-4,5-diazatricyclo [5.2.1.0^{2,6}]deca- |
| | 2(6),3-dien-3-yl]-1,3-oxazol-5-yl}-2-methyl propanoic acid; |
| 63. | 2-{2-[5-(2,4-Difluorophenyl)-4,5-diazatricyclo [5.2.1.0^{2,6}]deca-2(6),3- |
| | dien-3yl]-1,3-oxazol-5-yl}-2-methyl-1-propanol; |
| 64. | (1S,7R)-2-{2-[5-(2,4-Difluorophenyl)-4,5-diazatricyclo[5.2.1.0^{2,6}]deca- |
| | 2(6),3-dien-3yl]-1,3-oxazol-5-yl}-2-methyl-1-propanol; |
| 65. | (1R,7S)-2-{2-[5-(2,4-Difluorophenyl)-4,5-diazatricyclo [5.2.1.0^{2,6}]deca- |
| | 2(6),3-dien-3yl]-1,3-oxazol-5-yl}-2-methyl-1-propanol; |
| 66. | 2-{2-[5-(2,4-Difurophenyl)-4,5-diazatricycyclo[5.2.1.0^{2,6}]deca-2(6),3- |
| | dien-3-yl]-1,3-oxazol-5-yl}-2-methylpropanamide; |
| 67. | 5-(tert-Butyl)-2-[5-(4-fluorophenyl)-4,5-diazatricyclo [5.2.1.0^{2,6}]deca- |
| | 2(6),3-dien-3yl]-1,3-oxazole; |
| 68. | 5-(tert-Butyl)-2-[-5-(4-chlorophenyl)-4,5-diazatricyclo[5.2.1.0^{2,6}]deca- |
| | 2(6),3-dien-3yl]-1,3-oxazole; |
| 69. | 5-(tert-Butyl)-2-[5-(4-chloro-2-fluorophenyl)-4,5-diazatricyclo[5.2.1.0^{2,6}] |
| | deca-2(6),3-dien-3yl]-1,3-oxazole; |
| 70. | 5-(tert-Butyl)-2-[(1R,7S)- or (1S,7R)-5-(4-chloro-2-fluorophenyl)-4,5- |
| | diazatricyclo[5.2.1.0^{2,6}]deca-2(6),3-dien-3yl]-1,3-oxazole; |
| 71. | 5-(tert-Butyl)-2-[(1S,7R)- or (1R,7S)-5-(4-chloro-2-fluorophenyl)-4,5- |
| | diazatricyclo[5.2.1.0^{2,6}]deca-2(6),3-dien-3yl]-1,3-oxazole; |
| 72. | 5-(tert-Butyl)-2-[5-(2,4,6-trifluorophenyl)-4,5-diazatricyclo[5.2.1.0^{2,6}] |
| | deca-2(6),3-dien-3yl]-1,3-oxazole; |
| 73. | 5-(tert-Butyl)-2-[5-(4-methoxyphenyl)-4,5-diazatricyclo[5.2.1.0^{2,6}]deca- |
| | 2(6),3-dien-3yl]-1,3-oxazole; |
| 74. | 5-(tert-Butyl)-2-[5-(4-bromophenyl)-4,5-diazatricyclo[5.2.1.0^{2,6}]deca- |
| | 2(6),3-dien-3yl]-1,3-oxazole; |
| 75. | 5-(tert-Butyl)-2-[5-(4-nitrophenyl)-4,5-diazatricyclo[5.2.1.0^{2,6}]deca- |
| | 2(6),3-dien-3yl]-1,3-oxazole; |
| 76. | 5-(tert-Butyl)-2-[5-(2,4-difluorophenyl)-7,10,10-trimethyl-4,5- |
| | diazatricyclo [5.2.1.0^{2,6}]deca-2(6),3-dien-3yl]-1,3-oxazole; |
| 77. | 5-(tert-Butyl)-2-[12-(2,4-difluorophenyl)-11,12-diazatetracyclo [6.5.2.0^{2,7}.0^{9,13}] |
| | pentadeca-2(7),3,5,9(13),10-pentaen-10-yl]-1,3-oxazole; |
| 78. | 5-(tert-Butyl)-2-[5-(4-chlorophenyl)-5,6-diazatetracyclo [7.3.1.1^{3,11}.0^{4,8}] |
| | tetradeca-4(8),6-dien-3-yl]-1,3-oxazole; |
| 79. | 2-[5-(2,4-Difluorophenyl)-4,5-diazatricyclo [5.2.1.0^{2,6}]deca-2(6),3-dien- |
| | 3yl]-4-phenyl-1,3-thiazole; |
| 80. | 4-(tert-Butyl)-2-[5-(2,4-difluorophenyl)-4,5-diazatricyclo[5.2.1.0^{2,6}]deca- |
| | 2(6),3-dien-3yl]-1,3-thiazole; |
| 81. | 5-(tert-Butyl)-2-[5-(2,4-difluorophenyl)-4,5-diazatricyclo[5.2.1.0^{2,6}]deca- |
| | 2(6),3-dien-3yl]-1,3-thiazole; |
| 82. | 5-(tert-Butyl)-2-[(1S,7R)-5-(2,4-difluorophenyl)-4,5- |
| | diazatricyclo[5.2.1.0^{2,6}]deca-2(6),3-dien-3yl]-1,3-thiazole; |
| 83. | 5-(tert-Butyl)-2-[(1R,7S)-5-(2,4-difluorophenyl)-4,5-diazatricyclo[5.2.1.0^{2,6}] |
| | deca-2(6),3-dien-3yl]-1,3-thiazole; |
| 84. | 5-[5-(tert-Butyl)-1H-2-imidozolyl]-3-(2,4-difluorophenyl)-3,4- |
| | diazatricyclo[5.2.1.0^{2,6}]deca-2(6),4-diene; |
| 85. | 5-[4-(tert-Butyl)-1-methyl-1H-2-imidozolyl]-3-(2,4-difluorophenyl)-3,4- |
| | diazatricyclo[5.2.1.0^{2,6}]deca-2(6),4-diene or 5-[5-(tert-Butyl)-1-methyl- |
| | 1H-2-imidozolyl]-3-(2,4-difluorophenyl)-3,4-diazatricyclo[5.2.1.0^{2,6}]deca- |
| | 2(6),4-diene; |
| 86. | E or Z-1-{5-(2,4-Diflurophenyl)-4,5-diazatricyclo[5.2.1.0^{2,6}.]deca-2(6),3- |
| | dien-3-yl]}-3,3,-dimethyl-1-butanone-O-methyl-oxime; |
| 87. | 5-[4-(tert-Butyl)phenyl]-3-(2,4-difluorophenyl)--3,4- |
| | diazatricyclo[5.2.1.0^{2,6}]deca-2(6)4-diene; |
| 88. | 3-[5-(2,4-Difluorophenyl)-4,5-diazatricyclo[5.2.1.0^{2,6}]deca-2(6),3-dien-3- |
| | yl]benzaldehyde; |
| 89. | 3-[5-(2,4-Difluorophenyl)-4,5-diazatricyclo[5.2.1.0^{2,6}]deca-2(6),3-dien-3- |
| | yl]phenylmethanol; |
| 90. | N1-(tert-Butyl)-3-[4-(2,4-Difluorophenyl)-4,5- |
| | diazatricyclo[5.2.1.0^{2,6}]deca-2(6),3-dien-3-yl]propanamide or N1-(tert- |
| | Butyl)-3-[5-(2,4-Difluorophenyl)-4,5-diazatricyclo[5.2.1.0^{2,6}]deca-2(6),3- |
| | dien-3-yl]propanamide; |
| 91. | N1-(tert-Butyl)-3-[5-(2,4-Difluorophenyl)-4,5- |
| | diazatricyclo[5.2.1.0^{2,6}]deca-2(6),3-dien-3-yl]propanamide or N1-(tert- |
| | Butyl)-3-[4-(2,4-Difluorophenyl)-4,5-diazatricyclo[5.2.1.0^{2,6}]deca-2(6),3- |
| | dien-3-yl]propanamide; |
| 146. | 2-[5-(2,4-diflurophenyl)-4,5-diazatricyclo[5.2.1.0^{2,6}]deca-2(6),3-dien-3- |
| | yl]-4,4-dimethyl-4,5-dihydro-1,3-oxazole; |

and pharmaceutically acceptable salts thereof, pharmaceutically acceptable solvates thereof, regioisomers thereof, stereoisomers thereof, prodrugs thereof, and N-oxides thereof.

3. The compound of claim 1, wherein the compound has formula (Ia) 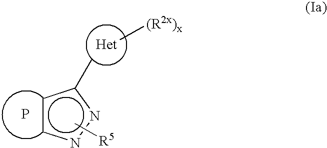
or a pharmaceutically acceptable salt thereof, a pharmaceutically acceptable solvate thereof, a regioisomer thereof, a stereo isomer thereof, a prodrug thereof, or a N-oxide thereof,
wherein
‘Het’ is a 5-membered heteroaryl or heterocyclyl;
each occurrence of R^{2x} is independently an optionally substituted alkyl, cycloalkyl, or aryl, where the optional substituents are selected from hydroxy, halogen, nitro, alkyl, alkoxy, COOR″ (where R″ is hydrogen or alkyl), and CONH₂;
ring P and R⁵ is as defined with respect to formula (I); and
x is an integer selected from 0-3.

4. A compound of formula (Ib) 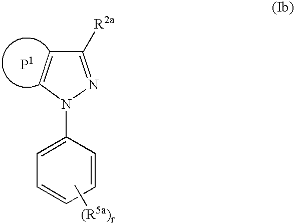
or a pharmaceutically acceptable salt thereof, a pharmaceutically acceptable solvate thereof, a regioisomer thereof, a stereoisomer thereof, a prodrug thereof, or a N-oxide thereof,
wherein
ring P¹ is 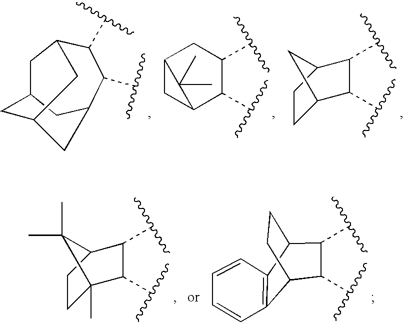
R^{2a} is selected from
i) an optionally substituted group selected form alkyl and aryl, wherein the optional substituents are hydroxy, CH₂OH, halogen, formaldehyde, and alkyl;
ii) COR^{3a1} wherein R^{3a1} is selected from an optionally substituted alkyl or aryl, wherein the optional substituents are selected from hydroxy and halogen;
iii) (CH₂)_{q1}—NR^{3a2}R^{3a3} wherein R^{3a2} and R^{3a3} are independently selected from hydrogen, substituted or unsubstituted alkyl, and substituted or unsubstituted arylalkyl, and q1 is 0 or 1;
iv) (CH₂)_{q2}—OR^{3a4} wherein R^{3a4} is arylalkyl and q2 is 1 or 2;
v) (CH₂)_{q3}—CONHR^{3a5} wherein R^{3a5} is alkyl and q3 is 1 or 2;
vi) NHCOR^{3a6} wherein R^{3a6} is alkyl;
vii) NHSO₂R^{3a7} wherein R^{3a7} is aryl;
viii) (CH═CH)—R^{3a8} wherein R^{3a8} is alkyl;
ix) COOR^{3a9} wherein R^{3a9} is alkyl;
x) C≡C—R^{3a10} wherein R^{3a10} is selected from alkyl and aryl;
xi) an optionally substituted 5-membered heteroaryl or an optionally substituted 5-membered heterocyclyl, wherein the optional substituents are selected from alkyl, aryl, cycloalkyl (e.g., bridged cycloalkyl), each of which is optionally substituted with hydroxy, halogen, COOH, or CONH₂;
xii) C(═NOR^{3a11})R^{3a12} wherein R^{3a11} and R^{3a12} are independently selected from hydrogen and optionally substituted alkyl;
xiii) CF₂R^{4a}; and
xiv) CHFR^{4a};
each occurrence of R^{5a} is selected from nitro, halogen, and alkoxy; and
r is an integer selected from 0-3.

5. A compound of formula (IIa) 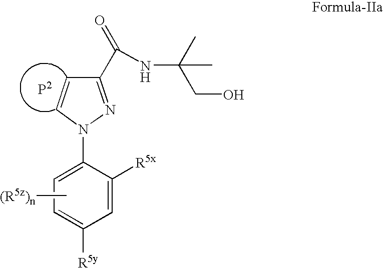
or a pharmaceutically acceptable salt thereof, a pharmaceutically acceptable solvate thereof, a regioisomer thereof, a stereoisomer thereof, a prodrug thereof, or a N-oxide thereof,
wherein 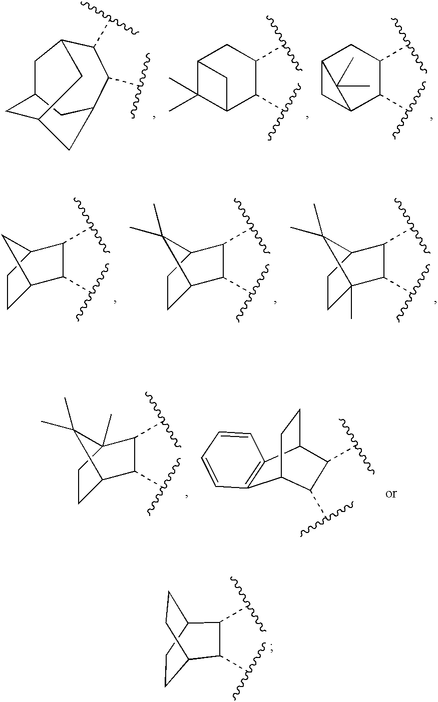
R^{5x} and R^{5y} are independently hydrogen, halogen, nitro, C₁-C₄ alkyl, C₁-C₄ alkoxy, trifluoromethyl, C(O)O(C₁-C₃ alkyl), N(H)C(O)O(C₁-C₃ alkyl) or NHC(O)CH₃;
R^{5z} is halogen; and
n is 0, 1 or 2.

6. The compound of claim 5, wherein the compound is selected from

| Compound | |
|---|---|
| No | Compound Name |
| 93. | N5-(2-hydroxy-1,1-dimethylethyl)-3-(2,4-diflurophenyl)-3,4- |
| | diazatricyclo[5.2.1.0^{2,6}]deca-2(6),4-diene-5-carboxamide; |
| 94. | (1R,7S)-N5-(2-hydroxy-1,1-dimethylethyl)-3-(2,4-difluorophenyl)-3,4- |
| | diazatricyclo[5.2.1.0^{2,6}]deca-2(6),4-diene-5-carboxamide; |
| 95. | (1S,7R)-N5-(2-Hydroxy-1,1-dimethylethyl)-3-(2,4-difluorophenyl)-3,4- |
| | diazatricyclo [5.2.1.0^{2,6}] deca-2(6),4-diene-5-carboxamide; |
| 96. | N5-(2-Hydroxy-1,1-dimethylethyl)-3-(4-chlorophenyl)-3,4- |
| | diazatricyclo[5.2.1.0^{2,6}]deca-2(6),4-diene-5-carboxamide; |
| 97. | (1R,7S)- or (1S,7R)-N5-(2-Hydroxy-1,1-dimethylethyl)-3-(4- |
| | chlorophenyl)-3,4-diazatricyclo[5.2.1.0^{2,6}]deca-2(6),4-diene-5-carboxamide; |
| 98. | (1S,7R)- or (1R,7S)-N5-(2-Hydroxy-1,1-dimethylethyl)-3-(4- |
| | chlorophenyl)-3,4-diazatricyclo[5.2.1.0^{2,6}]deca-2(6),4-diene-5-carboxamide; |
| 99. | N5-(2-Hydroxy-1,1-dimethylethyl)-3-(4-bromophenyl)-3,4- |
| | diazatricyclo[5.2.1.0^{2,6}]deca-2(6),4-diene-5-carboxamide; |
| 100. | N5-(2-Hydroxy-1,1-dimethylethyl)-3-(4-chloro-2-fluorophenyl)-3,4- |
| | diazatricyclo[5.2.1.0^{2,6}]deca-2(6),4-diene-5-carboxamide; |
| 101. | N5-(2-Hydroxy-1,1-dimethylethyl)-3-(2,4,6-trifluorophenyl)- |
| | 3,4diazatricyclo[5.2.1.0^{2,6}]deca-2(6),4-diene-5-carboxamide; |
| 134. | N5-(2-Hydroxy-1,1-dimethylethyl)-3-(2,4-difluorophenyl)-3,4- |
| | diazatricyclo[5.2.2.0^{2,6}]undeca-2(6),4-diene-5-carboxamide; |
| 135. | N5-(2-Hydroxy-1,1-dimethylethyl)-3-(4-fluorophenyl)-3,4-diazatricyclo |
| | [5.2.2.0^{2,6}] undeca-2(6),4-diene-5-carboxamide; |
| 141. | N5-(2-Hydroxy-1,1-dimethyl)-3-(2,4-difluorophenyl)-1,10,10-trimethyl- |
| | 3,4-diazatricyclo[5.2.1.0^{2,6}]deca-2(6),4-diene-5-carboxmide; |

and pharmaceutically acceptable salts thereof, pharmaceutically acceptable solvates thereof, regioisomers thereof, stereoisomers thereof, prodrugs thereof, and N-oxides thereof.

7. A compound of formula (IIb) 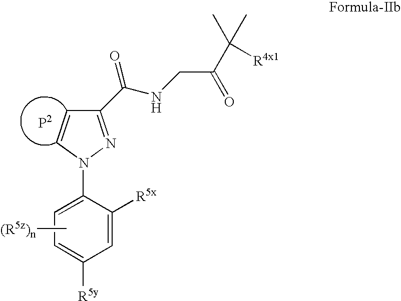
or a pharmaceutically acceptable salt thereof, a pharmaceutically acceptable solvate thereof, a regioisomer thereof, a stereoisomer thereof, a prodrug thereof, or a N-oxide thereof,
wherein ring P² is 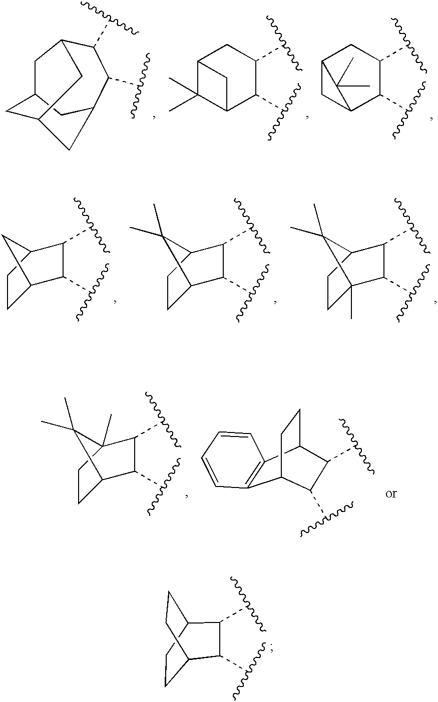
R^{4x1} is independently selected from H and COOR′″ wherein R′″ is H or alkyl;
R^{5x} and R^{5y} are independently hydrogen, halogen, nitro, C₁-C₄ alkyl, C₁-C₄ alkoxy, trifluoromethyl, C(O)O(C₁-C₃ alkyl), N(H)C(O)O(C₁-C₃ alkyl) or NHC(O)CH₃;
R^{5z} is halogen; and
n is 0, 1 or 2.

8. The compound of claim 7, wherein the compound is selected from

| Compound | |
|---|---|
| No | Compound Name |
| 36. | N5-(3,3-Dimethyl-2-oxobutyl)-3-(2,4-difluorophenyl)-3,4-diazatricyclo |
| | [5.2.1.0^{2,6}]deca-2(6),4-dien-5-carboxamide; |
| 37. | N5-(3,3-Dimethyl-2-oxobutyl)-3-(1R,7S)-(2,4-Difluorophenyl)-3,4- |
| | diazatricyclo [5.2.1.0^{2,6}]deca-2(6),4-dien-5-carboxamide; |
| 38. | N5-(3,3-Dimethyl-2-oxobutyl)-3-(1S,7R)-(2,4-Difluorophenyl)-3,4- |
| | diazatricyclo[5.2.1.0^{2,6}]deca-2(6),4-dien-5-carboxamide; |
| 39. | N5-(3,3-Dimethyl-2-oxobutyl)-3-(4-chlorophenyl)-3,4-diazatricyclo |
| | [5.2.1.0^{2,6}]deca-2(6),4-dien-5-carboxamide; |
| 40. | N5-(3,3-Dimethyl-2-oxobutyl)-3-(4-chloro-2-fluorophenyl)-3,4- |
| | diazatricyclo [5.2.1.0^{2,6}]deca-2(6),4-dien-5-carboxamide; |
| 41. | N5-(3,3-Dimethyl-2-oxobutyl)-3-(2,4,6-trifluorophenyl)-3,4-diazatricyclo |
| | [5.2.1.0^{2,6}]deca-2(6),4-dien-5-carboxamide; |
| 42. | Ethyl 4-[5-(2,4-difluorophenyl)-4,5-diazatricyclo[5.2.1.0^{2,6}]deca-2(6),3- |
| | dien-3-yl-carboxamido]-2,2-dimethyl-3-oxobutanoate; |
| 43. | Ethyl 4-[(1S, 7R)-5-(2,4-difluorophenyl)-4,5-diazatricyclo[5.2.1.0^{2,6}]deca- |
| | 2(6),3-dien-3-yl-carboxamido]-2,2-dimethyl-3-oxobutanoate; |
| 44. | Ethyl 4-[(1R,7S) 5-(2,4-difluorophenyl)-4,5-diazatricyclo [5.2.1.0^{2,6}]deca- |
| | 2(6),3-dien-3-yl-carboxamido]-2,2-dimethyl-3-oxobutanoate; |
| 45. | N5-(3,3-Dimethyl-2-oxobutyl)-3-(4-chloro-2-fluorophenyl)-3,4- |
| | diazatricyclo [5.2.1.0^{2,6}]deca-2(6),4-dien-5-carboxamide; |
| 46a | (1S,7R)- or (1R,7S)-N5-(3,3-Dimethyl-2-oxobutyl)-3-(4-chloro-2- |
| | fluorophenyl)-3,4-diazatricyclo[5.2.1.0^{2,6}]deca-2(6),4-dien-5- |
| | carboxamide; |
| 46b | (1R,7S) or (1S,7R)-N5-(3,3-Dimethyl-2-oxobutyl)-3-(4-chloro-2- |
| | fluorophenyl)-3,4-diazatricyclo[5.2.1.0^{2,6}]deca-2(6),4-dien-5- |
| | carboxamide; |
| 47. | N5-(3,3-Dimethyl-2-oxobutyl)-3-(4-methoxyphenyl)-3,4-diazatricyclo |
| | [5.2.1.0^{2,6}]deca-2(6),4-dien-5-carboxamide; |
| 48. | N5-(3,3-Dimethyl-2-oxobutyl)-3-(4-bromophenyl)-3,4-diazatricyclo |
| | [5.2.1.0^{2,6}]deca-2(6),4-dien-5-carboxamide; |
| 49. | N5-(3,3-Dimethyl-2-oxobutyl)-3-(4-nitrophenyl)-3,4-diazatricyclo |
| | [5.2.1.0^{2,6}]deca-2(6),4-dien-5-carboxamide; |
| 50. | N5-(3,3-Dimethyl-2-oxobutyl)-3-(4-fluorophenyl)-3,4-diazatricyclo |
| | [5.2.1.0^{2,6}]deca-2(6),4-dien-5-carboxamide; |
| 51. | N5-(3,3-Dimethyl-2-oxobutyl)-3-(2,4-difluorophenyl)-1,10,10-trimethyl- |
| | 3,4-diazatricyclo[5.2.1.0^{2,6}]deca-2(6),4-dien-5-carboxamide; |
| 52. | N12-(3,3-Dimethyl-2-oxobutyl)-10-(2,4-difluorophenyl)-10,11- |
| | diazatetracyclo[6.5.2.1.0^{2,7}]pentadeca-2,4,6,9(13),11-pentaene-12- |
| | carboxamide; |
| 53. | N7-(3,3-Dimethyl-2-oxobutyl)-5-(4-chlorophenyl)-5,6-diazatetracyclo |
| | [7.3.1.1^{3,11}.0^{4,8}]tetradeca-4(8),6-dien-7-carboxamide; |

and pharmaceutically acceptable salts thereof, pharmaceutically acceptable solvates thereof, regioisomers thereof, stereoisomers thereof, prodrugs thereof, and N-oxides thereof.

9. A compound of formula (IIc) 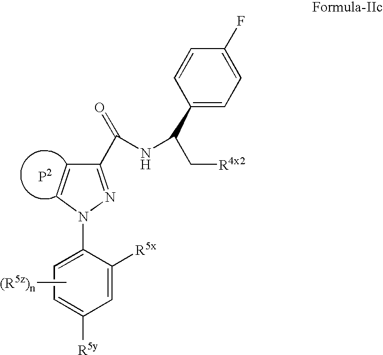
or a pharmaceutically acceptable salt thereof, a pharmaceutically acceptable solvate thereof, a regioisomer thereof, a stereoisomer thereof, a prodrug thereof, or a N-oxide thereof,
wherein P² is 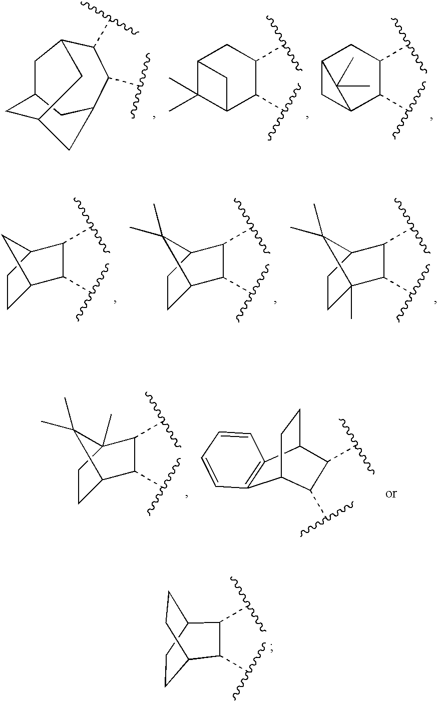
R^{4x2} is hydrogen, OR′″, or COOR′″ wherein R′″ is hydrogen or alkyl;
R^{5x} and R^{5y} are independently hydrogen, halogen, nitro, C₁-C₄ alkyl, C₁-C₄ alkoxy, trifluoromethyl, C(O)O(C₁-C₃ alkyl), N(H)C(O)O(C₁-C₃ alkyl) or NHC(O)CH₃;
R^{5z} is halogen; and
n is 0, 1 or 2.

10. The compound of claim 9, wherein the compound is selected from

| Compound | |
|---|---|
| No | Compound Name |
| 92a. | Methyl (2S)-2-[5-(2,4-difluorophenyl)-4,5-diazatricyclo[5.2.1.0.^{2,6}]deca- |
| | 2(6),3-dien-3-ylcarboxamido]-2-(4-fluorophenyl)ethanoate; |
| 92b. | N5-[(1S)-2-Hydroxy-1-(4-fluorophenyl)ethyl 3-(2,4-difluorophenyl)-3,4- |
| | diazatricyclo[5.2.1.0.^{2,6}]deca-2(6),4-diene-5-carboxamide; |
| 131. | N5-[(1S)-1-(4-fluorophenyl)-2-methoxyethyl]-3-(2,4-difluorophenyl)-3,4- |
| | diazatricyclo[5.2.1.0^{2,6}]deca-2(6),4-diene-5-carboxmide; |

and pharmaceutically acceptable salts thereof, pharmaceutically acceptable solvates thereof, regioisomers thereof, stereoisomers thereof, prodrugs thereof, and N-oxides thereof.

11. A compound of formula (IId) 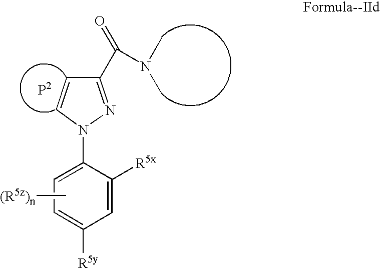
or a pharmaceutically acceptable salt thereof, a pharmaceutically acceptable solvate thereof, a regioisomer thereof, a stereoisomer thereof, a prodrug thereof, or a N-oxide thereof,
wherein, ring P² is 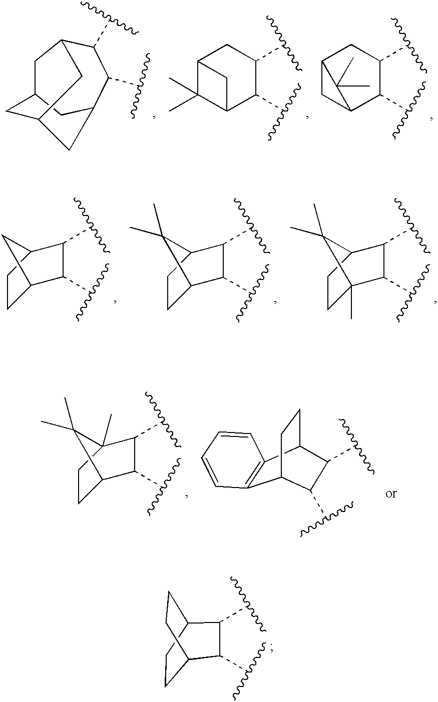 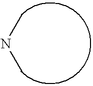
is independently selected from tetrahydroquinoline and tetrahydroisoquinoline;
R^{5x} and R^{5y} are independently hydrogen, halogen, nitro, C₁-C₄ alkyl, C₁-C₄ alkoxy, trifluoromethyl, C(O)O(C₁-C₃ alkyl), N(H)C(O)O(C₁-C₃ alkyl) or NHC(O)CH₃;
R^{5z} is halogen; and
n is 0, 1 or 2.

12. The compound of claim 11, wherein the compound is selected from

| Compound | |
|---|---|
| No | Compound Name |
| 127. | 5-(2,4-Difluorophenyl)-4,5-diazatricyclo[5.2.1.0^{2,6}]deca-2(6),3-dien-3-yl |
| | 1,2,3,4-tetrahydro-2-isoquinolinyl methanone; |
| 128. | 5-(2,4-Difluorophenyl)-4,5-diazatricyclo[5.2.1.0^{2,6}]deca-2(6),3-dien-3-yl |
| | 1,2,3,4-tetrahydro-1-quinolinyl methanone; |

and pharmaceutically acceptable salts thereof, pharmaceutically acceptable solvates thereof, regioisomers thereof, stereoisomers thereof, prodrugs thereof, and N-oxides thereof.

13. A compound of formula (IIe) 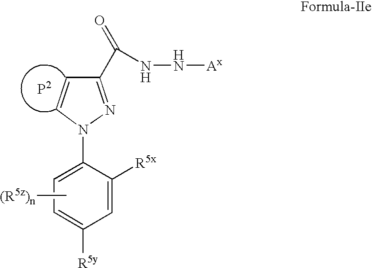
or a pharmaceutically acceptable salt thereof, a pharmaceutically acceptable solvate thereof, a regioisomer thereof, a stereoisomer thereof, a prodrug thereof, or a N-oxide thereof,
wherein ring P² is 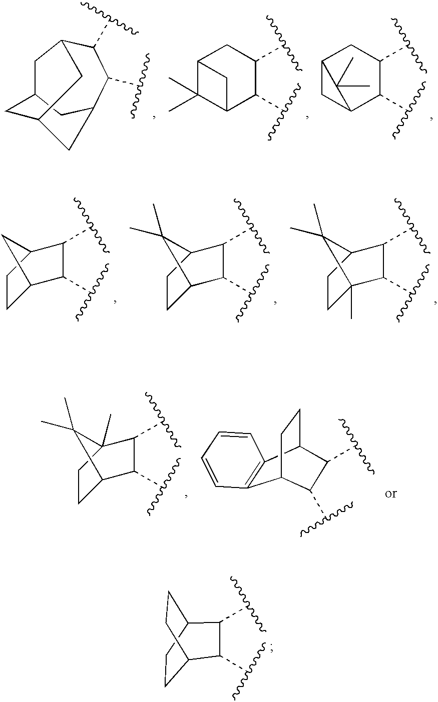
A^{x} is independently selected from alkyl and C(O)A^{y}, where A^{y} is alkyl or cycloalkyl;
R^{5x} and R^{5y} are independently hydrogen, halogen, nitro, C₁-C₄ alkyl, C₁-C₄ alkoxy, trifluoromethyl, C(O)O(C₁-C₃ alkyl), N(H)C(O)O(C₁-C₃ alkyl) or NHC(O)CH₃;
R^{5z} is halogen; and
n is 0, 1 or 2.

14. The compound of claim 13, wherein the compound is selected from

| Compound | |
|---|---|
| No | Compound Name |
| 16. | N′-Pivaloyl-3-(2,4-Diflurophenyl)-3,4-diazatricyclo[5.2.1.0^{2,6}]deca-2(6),4- |
| | diene-5-carbohydrazide; |
| 17. | N′-1-Hexanoyl-3-(2,4-diflurophenyl)-3,4-diazatricyclo[5.2.1.0^{2,6}]deca- |
| | 2(6),4-diene-5-carbohydrazide; |
| 18. | N′-1-(Adamantanecarbonyl)-3-(2,4-diflurophenyl)-3,4- |
| | diazatricyclo[5.2.1.0^{2,6}]deca-2(6),4-diene-5-carbohydrazide; |
| 19. | N′-(Cyclohexanecarbonyl)-3-(2,4-diflurophenyl)-3,4- |
| | diazatricyclo[5.2.1.0^{2,6}]deca-2(6),4-diene-5-carbohydrazide; |
| 130. | 3-(2,4-Difluorophenyl)-3,4-diazatricyclo [5.2.1.0^{2,6}]deca-2(6),4-dien-5- |
| | carboxylic N′-tert-butylhydrazide; |

and pharmaceutically acceptable salts thereof, pharmaceutically acceptable solvates thereof, regioisomers thereof, stereoisomers thereof, prodrugs thereof, and N-oxides thereof.

15. A pharmaceutical composition comprising a compound of any of claims 1, 4, 5, 7, 9, 11, and 13, and optionally together with one or more pharmaceutically acceptable exceipients, carriers, diluents or mixture thereof.

16. A method of treating a cannabinoid receptor mediated disease, disorder or syndrome in a subject in need thereof comprising administering to the subject a therapeutically effective amount of a compound of any of claims 1, 4, 5, 7, 9, 11, and 13.

17. The method of claim 16, wherein the disease, disorder or syndrome is selected from appetite disorders, metabolism disorders, catabolism disorders, diabetes, obesity, ophthalmic diseases, social related disorders, mood disorders, seizures, substance abuse, learning disorders, cognition disorders, memory disorders, organ contraction, muscle spasm, respiratory disorders, disorders and diseases, locomotor activity disorders, movement disorders, immune disorders, inflammation, cell growth, pain and neurodegenerative related syndromes.

18. A method for treating obesity and/or dyslipidemia in a subject in need thereof comprising administering to the subject a therapeutically effective amount of a compound of claim 1.

19. A method for treating pain in a subject in need thereof comprising administering to the subject a therapeutically effective amount of a compound of claim 1.
